SEQUENCE LISTING (168 sequences)

SEQ ID NO: 1 - DNA, 23 bp, Arachis hypogaea
  misc_feature (15)..(15): n = a or g or c or t
gayaargayaaygtnathgayca

SEQ ID NO: 2 - protein, 16 aa, Arachis hypogaea
IFLAGDKDNVIDQIEK

SEQ ID NO: 3 - protein, 16 aa, Arachis hypogaea
KGSEEEGDITNPINLR

SEQ ID NO: 4 - protein, 10 aa, Arachis hypogaea
NNPFYFPSRR

SEQ ID NO: 5 - DNA, 2032 bp, Arachis hypogaea
aataatcatatatattcatcaatcatctatataagtagtagcaggagcaatgagagggagggtttctccactgatgctgttgctagggatccttgtcctggcttcagtttctgcaacgcatgccaagtcatcaccttaccagaagaaaacagagaacccctgcgcccagaggtgcctccagagttgtcaacaggaaccggatgacttgaagcaaaaggcatgcgagtctcgctgcaccaagctcgagtatgatcctcgttgtgtctatgatcctcgaggacacactggcaccaccaaccaacgttcccctccaggggagcggacacgtggccgccaacccggagactacgatgatgaccgccgtcaaccccgaagagaggaaggaggccgatggggaccagctggaccgagggagcgtgaaagagaagaagactggagacaaccaagagaagattggaggcgaccaagtcatcagcagccacggaaaataaggcccgaaggaagagaaggagaacaagagtggggaacaccaggtagccatgtgagggaagaaacatctcggaacaaccctttctacttcccgtcaaggcggtttagcacccgctacgggaaccaaaacggtaggatccgggtcctgcagaggtttgaccaaaggtcaaggcagtttcagaatctccagaatcaccgtattgtgcagatcgaggccaaacctaacactcttgttcttcccaagcacgctgatgctgataacatccttgttatccagcaagggcaagccaccgtgaccgtagcaaatggcaataacagaaagagctttaatcttgacgagggccatgcactcagaatcccatccggtttcatttcctacatcttgaaccgccatgacaaccagaacctcagagtagctaaaatctccatgcccgttaacacacccggccagtttgaggatttcttcccggcgagcagccgagaccaatcatcctacttgcagggcttcagcaggaatacgttggaggccgccttcaatgcggaattcaatgagatacggagggtgctgttagaagagaatgcaggaggtgagcaagaggagagagggcagaggcgatggagtactcggagtagtgagaacaatgaaggagtgatagtcaaagtgtcaaaggagcacgttgaagaacttactaagcacgctaaatccgtctcaaagaaaggctccgaagaagagggagatatcaccaacccaatcaacttgagagaaggcgagcccgatctttctaacaactttgggaagttatttgaggtgaagccagacaagaagaacccccagcttcaggacctggacatgatgctcacctgtgtagagatcaaagaaggagctttgatgctcccacacttcaactcaaaggccatggttatcgtcgtcgtcaacaaaggaactggaaaccttgaactcgtggctgtaagaaaagagcaacaacagaggggacggcgggaagaagaggaggacgaagacgaagaagaggagggaagtaacagagaggtgcgtaggtacacagcgaggttgaaggaaggcgatgtgttcatcatgccagcagctcatccagtagccatcaacgcttcctccgaactccatctgcttggcttcggtatcaacgctgaaaacaaccacagaatcttccttgcaggtgataaggacaatgtgatagaccagatagagaagcaagcgaaggatttagcattccctgggtcgggtgaacaagttgagaagctcatcaaaaaccagaaggaatctcactttgtgagtgctcgtcctcaatctcaatctcaatctccgtcgtctcctgagaaagagtctcctgagaaagaggatcaagaggaggaaaaccaaggagggaagggtccactcctttcaattttgaaggcttttaactgagaatggaggcaacttgttatgtatcgataataagatcacgcttttgtactctactatccaaaaacttatcaataaataaaaacgtttgtgcgttgtttctcc

SEQ ID NO: 6 - DNA, 1949 bp, Arachis hypogaea
caatgagagggagggtttctccactgatgctgttgcttgggatccttgtcctggcttcagtttctgcaacgcaggccaagtcaccttaccggaaaacagagaacccctgcgcccagaggtgcctccagagttgtcaacaggaaccggacgacttgaagcaaaaggcatgcgagtctcgctgcaccaagctcgagtatgatcctcgttgtgtctatgacactggcgccaccaaccaacgtcaccctccaggggagcggacacgtggccgccaacccggagactacgatgatgaccgccgtcaaccccgaagagaggaaggaggccgatggggaccagctgaaccgagggagcgtgaaagagaagaagactggagacaaccaagagaagattggaggcgaccaagtcatcagcagccacggaaaataaggcccgaaggaagagaaggagaacaagagtggggaacaccaggtagcgaggtgagggaagaaacatcacggaacaaccctttctacttcccgtcaaggcggtttagcacccgctacgggaaccaaaacggtaggatccgcgtcctgcagaggtttgaccaaaggtcaaagcagtttcagaatctccagaatcaccgtattgtgcagatcgaggccagacctaacactcttgttcttcccaagcacgctgatgctgataacatccttgttatccagcaaggacaagccaccgtgaccgtagcaaatggcaataacagaaagagctttaatcttgacgagggccatgcactcagaatcccatccggtttcatttcctacatcttgaatcgacatgacaaccagaacctcagagtagctaaaatctccatgcccgttaacacgcccggccagtttgaggatttcttcccggcgagcagccgagaccaatcatcctacttgcagggattcagcaggaatactttggaggccgccttcaatgcggaattcaatgagatacggagggtgctgttagaagagaatgcaggaggagagcaagaggagagagggcagaggcgacggagtactcggagtagtgataatgaaggagtgatagtcaaagtgtcaaaggagcacgttcaagaacttactaagcacgctaaatccgtctcaaagaaaggctccgaagaggaagatatcaccaacccaatcaacttgagagatggcgagcccgatctttctaacaactttgggaggttatttgaggtgaagccagacaagaagaacccccagcttcaggacctggacatgatgctcacctgtgtagagatcaaagaaggagctttgatgctcccacacttcaactcaaaggccatggtcatcgtcgtcgtcaacaaaggaactggaaaccttgaactcgtagctgtaagaaaagagcaacaacagaggggacggcgggaacaagagtgggaagaagaggaggaagatgaagaagaggagggaagtaacagagaggtgcgtaggtacacagcgaggttgaaggaaggcgatgtgttcatcatgccagcagctcatccagtagccatcaacgcttcctccgaactccatctgcttggcttcggtatcaacgctgaaaacaaccacagaatcttccttgcaggtgataaggacaatgtgatagaccagatagagaagcaagcgaaggatttagcattccctggttcgggtgaacaagttgagaagctcatcaaaaaccagagggagtctcactttgtgagtgctcgtcctcaatctcaatctccgtcgtctcctgaaaaagaggatcaagaggaggaaaaccaaggagggaagggtccactcctttcaattttgaaggcttttaactgagaatggaggaaacttgttatgtatccataataagatcacgcttttgtaatctactatccaaaaacttatcaataaataaaaacgtttgtgcgttgtttctcc

SEQ ID NO: 7 - protein, 626 aa, Arachis hypogaea
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGKLFEVKPDKKNPQLQDLDMMLTCVEIKEGALMLPHFNSKAMVIVVVNKGTGNLELVAVRKEQQQRGRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 8 - protein, 614 aa, Arachis hypogaea
MRGRVSPLMLLLGILVLASVSATQAKSPYRKTENPCAQRCLQSCQQEPDDLKQKACESRCTKLEYDPRCVYDTGATNQRHPPGERTRGRQPGDYDDDRRQPRREEGGRWGPAEPREREREEDWRQPREDWRRPSHQQPRKIRPEGREGEQEWGTPGSEVREETSRNNPFYFPSRRFSTRYGNQNGRIRVLQRFDQRSKQFQNLQNHRIVQIEARPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRRSTRSSDNEGVIVKVSKEHVQELTKHAKSVSKKGSEEEDITNPINLRDGEPDLSNNFGRLFEVKPDKKNPQLQDLDMMLTCVEIKEGALMLPHFNSKAMVIVVVNKGTGNLELVAVRKEQQQRGRREQEWEEEEEDEEEEGSNREVRRYTARLKEGDVFIMPAAHPVAINASSELHLLGFGINAENNHRIFLAGDKDNVIDQIEKQAKDLAFPGSGEQVEKLIKNQRESHFVSARPQSQSPSSPEKEDQEEENQGGKGPLLSILKAFN

SEQ ID NO: 9 - protein, 10 aa, Arachis hypogaea
AKSSPYQKKT

SEQ ID NO: 10 - protein, 10 aa, Arachis hypogaea
QEPDDLKQKA

SEQ ID NO: 11 - protein, 10 aa, Arachis hypogaea
LEYDPRLVYD

SEQ ID NO: 12 - protein, 10 aa, Arachis hypogaea
GERTRGRQPG

SEQ ID NO: 13 - protein, 10 aa, Arachis hypogaea
PGDYDDDRRQ

SEQ ID NO: 14 - protein, 10 aa, Arachis hypogaea
PRREEGGRWG

SEQ ID NO: 15 - protein, 10 aa, Arachis hypogaea
REREEDWRQP

SEQ ID NO: 16 - protein, 10 aa, Arachis hypogaea
EDWRRPSHQQ

SEQ ID NO: 17 - protein, 10 aa, Arachis hypogaea
QPRKIRPEGR

SEQ ID NO: 18 - protein, 10 aa, Arachis hypogaea
TPGQFEDFFP

SEQ ID NO: 19 - protein, 10 aa, Arachis hypogaea
SYLQEFSRNT

SEQ ID NO: 20 - protein, 10 aa, Arachis hypogaea
FNAEFNEIRR

SEQ ID NO: 21 - protein, 10 aa, Arachis hypogaea
EQEERGQRRW

SEQ ID NO: 22 - protein, 10 aa, Arachis hypogaea
DITNPINLRE

SEQ ID NO: 23 - protein, 10 aa, Arachis hypogaea
NNFGKLFEVK

SEQ ID NO: 24 - protein, 10 aa, Arachis hypogaea
GTGNLELVAV

SEQ ID NO: 25 - protein, 10 aa, Arachis hypogaea
RRYTARLKEG

SEQ ID NO: 26 - protein, 10 aa, Arachis hypogaea
ELHLLGFGIN

SEQ ID NO: 27 - protein, 10 aa, Arachis hypogaea
HRIFLAGDKD

SEQ ID NO: 28 - protein, 10 aa, Arachis hypogaea
IDQIEKQAKD

SEQ ID NO: 29 - protein, 10 aa, Arachis hypogaea
KDLAFPGSGE

SEQ ID NO: 30 - protein, 10 aa, Arachis hypogaea
KESHFVSARP

SEQ ID NO: 31 - protein, 10 aa, Arachis hypogaea
PEKESPEKED

SEQ ID NO: 32 - protein, 10 aa, Arachis hypogaea
TNQRSPPGER

SEQ ID NO: 33 - protein, 10 aa, Arachis hypogaea
QRSPPGERTR

SEQ ID NO: 34 - protein, 10 aa, Arachis hypogaea
SPPGERTRGR

SEQ ID NO: 35 - protein, 10 aa, Arachis hypogaea
PGERTRGRQP

SEQ ID NO: 36 - protein, 10 aa, Arachis hypogaea
ERTRGRQPGD

SEQ ID NO: 37 - protein, 10 aa, Arachis hypogaea
TRGRQPGDYD

SEQ ID NO: 38 - protein, 10 aa, Arachis hypogaea
GRQPGDYDDD

SEQ ID NO: 39 - protein, 10 aa, Arachis hypogaea
QPGDYDDDRR

SEQ ID NO: 40 - protein, 10 aa, Arachis hypogaea
GDYDDDRRQP

SEQ ID NO: 41 - protein, 10 aa, Arachis hypogaea
YDDDRRQPRR

SEQ ID NO: 42 - protein, 10 aa, Arachis hypogaea
DDRRQPRREE

SEQ ID NO: 43 - protein, 10 aa, Arachis hypogaea
RRQPRREEGG

SEQ ID NO: 44 - protein, 10 aa, Arachis hypogaea
QPRREEGGRW

SEQ ID NO: 45 - protein, 10 aa, Arachis hypogaea
RREEGGRWGP

SEQ ID NO: 46 - protein, 10 aa, Arachis hypogaea
EEGGRWGPAG

SEQ ID NO: 47 - protein, 10 aa, Arachis hypogaea
GGRWGPAGPR

SEQ ID NO: 48 - protein, 10 aa, Arachis hypogaea
RWGPAGPRER

SEQ ID NO: 49 - protein, 10 aa, Arachis hypogaea
GPAGPRERER

SEQ ID NO: 50 - protein, 10 aa, Arachis hypogaea
AGPREREREE

SEQ ID NO: 51 - protein, 10 aa, Arachis hypogaea
PREREREEDW

SEQ ID NO: 52 - protein, 10 aa, Arachis hypogaea
EREREEDWRQ

SEQ ID NO: 53 - protein, 10 aa, Arachis hypogaea
EREEDWRQPR

SEQ ID NO: 54 - protein, 149 aa, Arachis hypogaea
GRREEEEDEDEEEEGSNREVRRYTARLKEGDVFIMPAAHPVAINASSELHLLGFGINAENNHRIFLAGDKDNVIDQIEKQAKDLAFPGSGEQVEKLIKNQKESHFVSARPQSQSQSPSSPEKESPEKEDQEEENQGGKGPLLSILKAFN

SEQ ID NO: 55 - protein, 268 aa, Arachis hypogaea
KIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRKSFNLDEGHALRIPSGFISYILNRHDNQNLRVAKISMPVNTPGQFEDFFPASSRDQSSYLQGFSRNTLEAAFNAEFNEIRRVLLEENAGGEQEERGQRRWSTRSSENNEGVIVKVSKEHVEELTKHAKSVSKKGSEEEGDITNPINLREGEPDLSNNFGK

SEQ ID NO: 56 - protein, 6 aa, Arachis hypogaea
DPYSPS

SEQ ID NO: 57 - protein, 31 aa, Arachis hypogaea
  UNSURE (1)..(1): Xaa = amino acid that was unable to be assigned
XQQXELQXDXXXQSQLDADLRPGEQXLMXKI

SEQ ID NO: 58 - protein, 31 aa, Arachis hypogaea
  UNSURE (1)..(1): Xaa = amino acid that was unable to be assigned
XQQXELQDXEXXQSQERANLRPREQXLMXKI

SEQ ID NO: 59 - DNA, 26 bp, Arachis hypogaea
  misc_feature (21)..(21): n = a or g or c or t
carcartgggarttrcarggngayag

SEQ ID NO: 60 - protein, 17 aa, Arachis hypogaea
QQWELQGDRRRQSQLER

SEQ ID NO: 61 - protein, 13 aa, Arachis hypogaea
ANLRPCEQHLMQK

SEQ ID NO: 62 - DNA, 474 bp, Arachis hypogaea
ctcaccatactagtagccctcgcccttttcctcctcgctgcccacgcatctgcgaggcagcagtgggaactccaaggagacagaagatgccagagccagctcgagagggcgaacctgaggccctgcgagcaacatctcatgcagaagatccaacgtgacgaggattcatatgaacgggacccgtacagccctagtcaggatccgtacagccctagtccatatgatcggagaggcgctggatcctctcagcaccaagagaggtgttgcaatgagctgaacgagtttgagaacaaccaaaggtgcatgtgcgaggcattgcaacagatcatggagaaccagagcgataggttgcaggggaggcaacaggagcaacagttcaagagggagctcaggaacttgcctcaacagtgcggccttagggcaccacagcgttgcgacttggacgtcgaaagtggcggcagagacagatactaa

SEQ ID NO: 63 - protein, 157 aa, Arachis hypogaea
LTILVALALFLLAAHASARQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGGRDRY

SEQ ID NO: 64 - protein, 10 aa, Arachis hypogaea
DSYERDPYSP

SEQ ID NO: 65 - protein, 10 aa, Arachis hypogaea
YERDPYSPSQ

SEQ ID NO: 66 - protein, 10 aa, Arachis hypogaea
RDPYSPSQDP

SEQ ID NO: 67 - protein, 10 aa, Arachis hypogaea
PYSPSQDPYS

SEQ ID NO: 68 - protein, 10 aa, Arachis hypogaea
SPSQDPYSPS

SEQ ID NO: 69 - protein, 10 aa, Arachis hypogaea
SQDPYSPSPY

SEQ ID NO: 70 - protein, 10 aa, Arachis hypogaea
DPYSPSPYDR

SEQ ID NO: 71 - protein, 10 aa, Arachis hypogaea
HASARQQWEL

SEQ ID NO: 72 - protein, 10 aa, Arachis hypogaea
QWELQGDRRC

SEQ ID NO: 73 - protein, 10 aa, Arachis hypogaea
DRRCQSQLER

SEQ ID NO: 74 - protein, 10 aa, Arachis hypogaea
LRPCEQHLMQ

SEQ ID NO: 75 - protein, 10 aa, Arachis hypogaea
KIQRDEDSYE

SEQ ID NO: 76 - protein, 10 aa, Arachis hypogaea
YERDPYSPSQ

SEQ ID NO: 77 - protein, 10 aa, Arachis hypogaea
SQDPYSPSPY

SEQ ID NO: 78 - protein, 10 aa, Arachis hypogaea
DRLQGRQQEQ

SEQ ID NO: 79 - protein, 10 aa, Arachis hypogaea
KRELRNLPQQ

SEQ ID NO: 80 - protein, 10 aa, Arachis hypogaea
QRCDLDVESG

SEQ ID NO: 81 - protein, 83 aa, Arachis hypogaea
ELQGDRRCQSQLERANLRPCEQHLMQKIQRDEDSYERDPYSPSQDPYSPSPYDRRGAGSSQHQERCCNELNEFENNQRCMCEA

SEQ ID NO: 82 - protein, 166 aa, Arachis hypogaea
MASMTGGQMGRDPNSARQQWELQGDRRCQSQLERANLRPCEQHLMQKIQRDEDSYERDPYSPSQDPYSPSPYDRRGAGSSQHQERCCNELNEFENNQRCMCEALQQIMENQSDRLQGRQQEQQFKRELRNLPQQCGLRAPQRCDLDVESGGRDRYAAALEHHHHHH

SEQ ID NO: 83 - protein, 11 aa, Arachis hypogaea
ISFRQQPEENA

SEQ ID NO: 84 - DNA, 19 bp, Arachis hypogaea
gcaccttctggtgactatc

SEQ ID NO: 85 - DNA, 23 bp, Arachis hypogaea
  misc_feature (3)..(3): n = a or g or c or t
cgncagcaaccggaggagaacgc

SEQ ID NO: 86 - DNA, 23 bp, Arachis hypogaea
cgactcactatagggcgaattgg

SEQ ID NO: 87 - DNA, 31 bp, Arachis hypogaea
tatggctagcttccggcagcaaccggaggag

SEQ ID NO: 88 - DNA, 26 bp, Arachis hypogaea
ccgtcgacagccacagccctcggaga

SEQ ID NO: 89 - DNA, 1524 bp, Arachis hypogaea
cggcagcaaccggaggagaacgcgtgccagttccagcgcctcaatgcgcagagacctgacaatcgcattgaatcagagggcggttacattgagacttggaaccccaacaaccaggagttcgaatgcgccggcgtcgccctctctcgcttagtcctccgccgcaacgcccttcgtaggcctttctactccaatgctccccaggagatcttcatccagcaaggaaggggatactttgggttgatattccctggttgtcctagacactatgaagagcctcacacacaaggtcgtcgatctcagtcccaaagaccaccaagacgtctccaaggagaagaccaaagccaacagcaacgagatagtcaccagaaggtgcaccgtttcgatgagggtgatctcattgcagttcccaccggtgttgctttctggctctacaacgaccacgacactgatgttgttgctgtttctcttactgacaccaacaacaacgacaaccagcttgatcagttccccaggagattcaatttggctgggaacacggagcaagagttcttaaggtaccagcaacaaagcagacaaagcagacgaagaagcttaccatatagcccatacagcccgcaaagtcagcctagacaagaagagcgtgaatttagccctcgaggacagcacagccgcagagaacgagcaggacaagaagaagaaaacgaaggtggaaacatcttcagcggcttcacgccggagttcctggaacaagccttccaggttgacgacagacagatagtgcaaaacctaagaggcgagaccgagagtgaagaagagggagccattgtgacagtgaggggaggcctcagaatcttgagcccagatagaaagagacgtgccgacgaagaagaggaatacgatgaagatgaatatgaatacgatgaagaggatagaaggcgtggcaggggaagcagaggcagggggaatggtattgaagagacgatctgcaccgcaagtgctaaaaagaacattggtagaaacagatcccctgacatctacaaccctcaagctggttcactcaaaactgccaacgatctcaaccttctaatacttaggtggcttggacctagtgctgaatatggaaatctctacaggaatgcattgtttgtcgctcactacaacaccaacgcacacagcatcatatatcgattgaggggacgggctcacgtgcaagtcgtggacagcaacggcaacagagtgtacgacgaggagcttcaagagggtcacgtgcttgtggtgccacagaacttcgccgtcgctggaaagtcccagagcgagaacttcgaatacgtggcattcaagacagactcaaggcccagcatagccaacctcgccggtgaaaactccgtcatagataacctgccggaggaggtggttgcaaattcatatggcctccaaagggagcaggcaaggcagcttaagaacaacaaccccttcaagttcttcgttccaccgtctcagcagtctccgagggctgtggcttaa

SEQ ID NO: 90 - protein, 510 aa, Arachis hypogaea
ISFRQQPEENACQFQRLNAQRPDNRIESEGGYIETWNPNNQEFECAGVALSRLVLRRNALRRPFYSNAPQEIFIQQGRGYFGLIFPGCPRHYEEPHTQGRRSQSQRPPRRLQGEDQSQQQRDSHQKVHRFDEGDLIAVPTGVAFWLYNDHDTDVVAVSLTDTNNNDNQLDQFPRRFNLAGNTEQEFLRYQQQSRQSRRRSLPYSPYSPQSQPRQEEREFSPRGQHSRRERAGQEEENEGGNIFSGFTPEFLEQAFQVDDRQIVQNLRGETESEEEGAIVTVRGGLRILSPDRKRRADEEEEYDEDEYEYDEEDRRRGRGSRGRGNGIEETICTASAKKNIGRNRSPDIYNPQAGSLKTANDLNLLILRWLGPSAEYGNLYRNALFVAHYNTNAHSIIYRLRGRAHVQVVDSNGNRVYDEELQEGHVLVVPQNFAVAGKSQSENFEYVAFKTDSRPSIANLAGENSVIDNLPEEVVANSYGLQREQARQLKNNNPFKFFVPPSQQSPRAVA

SEQ ID NO: 91 - protein, 15 aa, Arachis hypogaea
IETWNPNNQEFECAG

SEQ ID NO: 92 - protein, 15 aa, Arachis hypogaea
GNIFSGFTPEFLEQA

SEQ ID NO: 93 - protein, 15 aa, Arachis hypogaea
VTVRGGLRILSPDRK

SEQ ID NO: 94 - protein, 15 aa, Arachis hypogaea
DEDEYEYDEEDRRRG

SEQ ID NO: 95 - protein, 10 aa, Arachis hypogaea
EQEFLRYQQQ

SEQ ID NO: 96 - protein, 10 aa, Arachis hypogaea
FTPEFLEQAF

SEQ ID NO: 97 - protein, 10 aa, Arachis hypogaea
EYEYDEEDRR

SEQ ID NO: 98 - protein, 10 aa, Arachis hypogaea
LYRNALFVAH

SEQ ID NO: 99 - protein, 15 aa, Arachis hypogaea
EEEYDEDEYEYDEED

SEQ ID NO: 100 - protein, 15 aa, Arachis hypogaea
EYDEDEYEYDEEDRR

SEQ ID NO: 101 - protein, 15 aa, Arachis hypogaea
DEDEYEYDEEDRRRG

SEQ ID NO: 102 - protein, 15 aa, Arachis hypogaea
DEYEYDEEDRRRGRG

SEQ ID NO: 103 - protein, 15 aa, Arachis hypogaea
YEYDEEDRRRGRGSR

SEQ ID NO: 104 - protein, 15 aa, Arachis hypogaea
YDEEDRRRGRGSRGR

SEQ ID NO: 105 - protein, 18 aa, Arachis hypogaea
  UNSURE (7)..(7): Xaa = amino acid that was unable to be assigned
SIDETIXTMRLXQNIXQT

SEQ ID NO: 106 - protein, 21 aa, Glycine max
  UNSURE (20)..(20): Xaa = amino acid that was unable to be assigned
GIDETICTMRLRGNIGQNSXP

SEQ ID NO: 107 - protein, 25 aa, Glycine max
  UNSURE (21)..(21): Xaa = amino acid that was unable to be assigned
GIDETICTMRLRQNIGQNSSXDIYN

SEQ ID NO: 108 - protein, 25 aa, Glycine max
GIDETICTMRLRQNIGQNSSPDIYN

SEQ ID NO: 109 - protein, 485 aa, Glycine max
MAKLVLSLCFLLFSGCFALREQAQQNECQIQKLNALKPGNRIESEGGFIETWNPNNKPFQCAGVALSRCTLNRNALRRPSYTNGPQEIYIQQGNGIFGMIFPGCPSTYQEPQESQQRGRSQRPQDRHQKVHRFREGDLIAVPTGVAWWMYNNEDTPVVAVSIIDTNSLENQLDQMPRRFYLAGNQEQEFLKYQQQQQGGSQSQKGKQQEEENEGSNILSGFAPEFLKEAFGVNMQIVRNLQGENEEEDSGAIVTVKGGLRVTAPAMRKPQQEEDDDDEEEQPQCVETDKGCQRQSKRSRNGIDETICTMRLRQNIGQNSSPDIYNPQAGSITTATSLDFPALWLLKLSAQYGSLRKNAMFVPHYTLNANSIIYALNGRALVQVVNCNGERVFDGELQEGGVLIVPQNFAVAAKSQSDNFEYVSFKTNDRPSIGNLAGANSLLNALPEEVIQHTFNLKSQQARQVKNNNPFSFLVPPQESQRRAVA

SEQ ID NO: 110 - protein, 605 aa, Glycine max
MMRARFPLLLLGLVFLASVSVSFGIAYWEKENPKHNKCLQSCNSERDSYRNQACHARCNLLKVEKEECEEGEIPRPRPRPQHPEREPQQPGEKEEDEDEQPRPIPFPRPQPRQEEEHEQREEQEWPRKEEKRGEKGSEEEDEDEDEEQDERQFPFPRPPHQKEERNEEEDEDEEQQRESEESEDSELRRHKNKNPFLFGSNRFETLFKNQYGRIRVLQRFNQRSPQLQNLRDYRILEFNSKPNTLLLPNHADADYLIVILNGTAILSLVNNDDRDSYRLQSGDALRVPSGTTYYVVNPDNNENLRLITLAIPVNKPGRFESFFLSSTEAQQSYLQGFSRNILEASYDTKFEEINKVLFSREEGQQQGEQRLQESVIVEISKEQIRALSKRAKSSSRKTISSEDKPFNLRSRDPIYSNKLGKFFEITPEKNPQLRDLDIFLSIVDMNEGALLLPHFNSKAIVILVINEGDANIELVGLKEQQQEQQQEEQPLEVRKYRAELSEQDIFVIPAGYPVVVNATSNLNFFAIGINAENNQRNFLAGSQDNVISQIPSQVQELAFPGSAQAVEKLLKNQRESYFVDAQPKKKEEGNKGRKGPLSSILRAFY

SEQ ID NO: 111 - protein, 9 aa, Juglans nigra
CIFHTFSLT

SEQ ID NO: 112 - protein, 8 aa, Juglans nigra
VALLFVAN

SEQ ID NO: 113 - protein, 8 aa, Juglans nigra
RRRGEGCQ

SEQ ID NO: 114 - protein, 8 aa, Juglans nigra
NLNHCQYY

SEQ ID NO: 115 - protein, 8 aa, Juglans nigra
QHFRQCCQ

SEQ ID NO: 116 - protein, 8 aa, Juglans nigra
QCEGLRQA

SEQ ID NO: 117 - protein, 8 aa, Juglans nigra
RGEEMEEM

SEQ ID NO: 118 - protein, 9 aa, Juglans nigra
KECGISSQR

SEQ ID NO: 119 - DNA, 27 bp, Artificial Sequence
  Primers used to amplify IgE Fab Fragments
gctactagttttgttgtcgacccagtc

SEQ ID NO: 120 - DNA, 31 bp, Artificial Sequence
  Primers used to amplify IgE Fab Fragments
cgactgtaaactagtcacggtgggcggggtg

SEQ ID NO: 121 - DNA, 53 bp, Artificial Sequence
  Primers used to amplify IgE Fab Fragments
gcgccgtctagaactaacactctcccctgttgaagctctttgtgacgggcaag

SEQ ID NO: 122 - DNA, 58 bp, Artificial Sequence
  Primers used to amplify IgE Fab Fragments
gcgccgtctagaattaacactctcccctgttgaagctctttgtgacgggcgaactcag

SEQ ID NO: 123 - DNA, 30 bp, Artificial Sequence
  Primers used to amplify IgE Fab Fragments
cgccgtctagaattatgaacattctgtagg

SEQ ID NO: 124 - DNA, 29 bp, Artificial Sequence
  Primers used to amplify IgE Fab Fragments
cactcccaggtgcagctgctcgagtctgg

SEQ ID NO: 125 - DNA, 32 bp, Artificial Sequence
  Primers used to amplify IgE Fab Fragments
gtcctgtcccaggtcaacttactcgagtctgg

SEQ ID NO: 126 - DNA, 32 bp, Artificial Sequence
  Primers used to amplify IgE Fab Fragments
gtccaggtggaggtgcagctgctcgagtctgg

SEQ ID NO: 127 - DNA, 32 bp, Artificial Sequence
  Primers used to amplify IgE Fab Fragments
gtgctgtcccaggtgcagctgctcgagtcggg

SEQ ID NO: 128 - DNA, 32 bp, Artificial Sequence
  Primers used to amplify IgE Fab Fragments
gtctgtgcccaggtgcagctgctcgagtctgg

SEQ ID NO: 129 - DNA, 32 bp, Artificial Sequence
  Primers used to amplify IgE Fab Fragments
gtcctgtcacaggtacagctgctcgagtcagg

SEQ ID NO: 130 - DNA, 22 bp, Artificial Sequence
  Primers used to amplify IgE Fab Fragments
aggtgcagctgctcgactctgg

SEQ ID NO: 131 - DNA, 23 bp, Artificial Sequence
  Primers used to amplify IgE Fab Fragments
caggtgcagctgctcgagtcggg

SEQ ID NO: 132 - DNA, 38 bp, Artificial Sequence
  Primers used to amplify IgE Fab Fragments
gagccgcacgagcccgagctccagatgacccagtctcc

SEQ ID NO: 133 - DNA, 24 bp, Artificial Sequence
  Primers used to amplify IgE Fab Fragments
gacatcgagctcacccagtctcca

SEQ ID NO: 134 - DNA, 38 bp, Artificial Sequence
  Primers used to amplify IgE Fab Fragments
gagccgcacgagcccgagctcgtgatgacycagtctcc

SEQ ID NO: 135 - DNA, 24 bp, Artificial Sequence
  Primers used to amplify IgE Fab Fragments
gaaattgagctcacgcagtctcca

SEQ ID NO: 136 - DNA, 38 bp, Artificial Sequence
  Primers used to amplify IgE Fab Fragments
gagccgcacgagcccgagctcgtgwtgacrcagtctcc

SEQ ID NO: 137 - DNA, 24 bp, Artificial Sequence
  Primers used to amplify IgE Fab Fragments
aattttgagctcactcagccccac

SEQ ID NO: 138 - DNA, 27 bp, Artificial Sequence
  Primers used to amplify IgE Fab Fragments
tctgtggagctccagccgccctcagtg

SEQ ID NO: 139 - protein, 371 aa, Phaseolus vulgaris
DNPFYFNSDNSWNTLFKNQYGHIRVLQRFDQQSKRLQNLEDYRLVEFRSKPETLLLPQQADAELLLVVRSGSAILVLVKPDDRREYFFLTSDNPIFSDHQKIPAGTIFYLVNPDPKEDLRIIQLAMPVNNPQIHEFFLSSTEAQQSYLQEFSKHILEASFNSKFEEINRVLFEEEGQQEGVIVNIDSEQIKELSKHAKSSSRKSLSKQDNTIGNEFGNLTERTDNSLNVLISSIEMEEGALFVPHYYSKAIVILVVNEGEAHVELVGPKGNKETLEYESYRAELSKDDVFVIPAAYPVAIKATSNVNFTGFGINANNNNRNLLAGKTDNVISSIGRALDGKDVLGLTFSGSGDEVMKLINKQSGSYFVDAH

SEQ ID NO: 140 - protein, 87 aa, Glycine max
SEGGLIETWNPNNKPFQCAGVALSRCTLNRNALRRPSYTNGPQEIYIQQGKGIFGMIYPGCPSTFEEPQQPQQRGQSSRPQDRHQKI

SEQ ID NO: 141 - protein, 87 aa, Glycine max
SEGGFIETWNPNNKPFQCAGVALSRCTLNRNALRRPSYTNGPQEIYIQQGNGIFGMIFPGCPSTYQEPQESQQRGRSQRPQDRHQKV

SEQ ID NO: 142 - protein, 86 aa, Pisum sativum
SEGGLIETWNPNNKQFRCAGVALSRATLQHNALRRPYYSNAPQEIFIQQGNGYFGMVFPGCPETFEEPQESEQGEGRRYRDRHQKV

SEQ ID NO: 143 - protein, 141 aa, Glycine max
EEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVA

SEQ ID NO: 144 - protein, 137 aa, Glycine max
DDDEEEQPQCVETDKGCQRQSKRSRNGIDETICTMRLRQNIGQNSSPDIYNPQAGSITTATSLDFPALWLLKLSAQYGSLRKNAMFVPHYTLNANSIIYALNGRALVQVVNCNGERVFDGELQEGGVLIVPQNFAVA

SEQ ID NO: 145 - protein, 150 aa, Pisum sativum
DEDEERQPRHQRRRGEEEEEDKKERRGSQKGKSRRQGDNGLEETVCTAKLRLNIGPSSSPDIYNPEAGRIKTVTSLDLPVLRWLKLSAEHGSLHKNAMFVPHYNLNANSIIYALKGRARLQVVNCNGNTVFDGELEAGRALTVPQNYAVA

SEQ ID NO: 146 - protein, 7 aa, Glycine max
GIAYWEK

SEQ ID NO: 147 - protein, 10 aa, Glycine max
SERDSYRNQA

SEQ ID NO: 148 - protein, 23 aa, Glycine max
LKVEKEECEEGEIPRPRPRPQHP

SEQ ID NO: 149 - protein, 10 aa, Glycine max
FPRPQPRQEE

SEQ ID NO: 150 - protein, 10 aa, Glycine max
EEHEQREEQE

SEQ ID NO: 151 - protein, 10 aa, Glycine max
EWPRKEEKRG

SEQ ID NO: 152 - protein, 10 aa, Glycine max
DEDEDEEQDE

SEQ ID NO: 153 - protein, 12 aa, Glycine max
RQFPFPRPPHQK

SEQ ID NO: 154 - protein, 10 aa, Glycine max
KEERNEEEDE

SEQ ID NO: 155 - protein, 10 aa, Glycine max
KPGRFESFFL

SEQ ID NO: 156 - protein, 10 aa, Glycine max
SYLQGFSRNI

SEQ ID NO: 157 - protein, 10 aa, Glycine max
YDTKFEEINK

SEQ ID NO: 158 - protein, 5 aa, Glycine max
QQGEQ

SEQ ID NO: 159 - protein, 7 aa, Glycine max
KPFNLRS

SEQ ID NO: 160 - protein, 10 aa, Glycine max
NKLGKFFEIT

SEQ ID NO: 161 - protein, 10 aa, Glycine max
GDANIELVGL

SEQ ID NO: 162 - protein, 10 aa, Glycine max
RKYRAELSEQ

SEQ ID NO: 163 - protein, 10 aa, Glycine max
NLNFFAIGIN

SEQ ID NO: 164 - protein, 10 aa, Glycine max
QRNFLAGSQD

SEQ ID NO: 165 - protein, 10 aa, Glycine max
ISQIPSQVQE

SEQ ID NO: 166 - protein, 10 aa, Glycine max
QELAFPGSAQ

SEQ ID NO: 167 - protein, 10 aa, Glycine max
RESYFVDAQP

SEQ ID NO: 168 - protein, 6 aa, Glycine max
KKEEGN

(19) United States
(12) Patent Application Publication
(10) Pub. No.: US 2012/0283421 A1
(43) Pub. Date: Nov. 8, 2012

(54) METHODS AND REAGENTS FOR DECREASING CLINICAL REACTION TO ALLERGY

(76) Inventors: Michael J. Caplan, Woodbridge, CT (US); Howard B. Sosin, Fairfield, CT (US); Hugh A. Sampson, Greenwich, CT (US); Gary A. Bannon, Wentzville, MO (US); A. Wesley Burks, JR., Chapel Hill, NC (US); Gael Cockrell, Cabot, AR (US); Cesar M. Compadre, Little Rock, AR (US); Cathie Connaughton, Conway, AR (US); Ricki M. Helm, Little Rock, AR (US); Nina E. King, Mason, OH (US); Randall A. Kopper, Conway, AR (US); Soheila J. Maleki, New Orleans, LA (US); Patrick A. Rabjohn, Little Rock, AR (US); David S. Shin, San Diego, CA (US); J. Steven Stanley, North Little Rock, AR (US)

(21) Appl. No.: 13/183,892

(22) Filed: Jul. 15, 2011

Related U.S. Application Data
(63) Continuation of application No. 10/100,303, filed on Mar. 18, 2002, now abandoned.
Application No. 10/100,303 is a continuation-in-part of application No. 09/494,096, filed on Jan. 28, 2000, now abandoned.
Application No. 09/494,096 is a continuation-in-part of application No. 09/267,719, filed on Mar. 11, 1999, now abandoned.
Application No. 09/267,719 is a continuation-in-part of application No. 09/248,673, filed on Feb. 11, 1999, now abandoned.
Application No. 09/248,673 is a continuation-in-part of application No. 09/248,674, filed on Feb. 11, 1999, now abandoned.
Application No. 09/248,674 is a continuation-in-part of application No. 09/240,557, filed on Jan. 29, 1999, now abandoned.
Application No. 09/240,557 is a continuation-in-part of application No. 09/241,101, filed on Jan. 29, 1999, now abandoned.
Application No. 09/241,101 is a continuation-in-part of application No. 09/191,593, filed on Nov. 13, 1998, now Pat. No. 6,835,824.
Application No. 09/191,593 is a continuation-in-part of application No. 09/141,220, filed on Aug. 27, 1998, now abandoned.
Application No. 09/141,220 is a continuation-in-part of application No. 09/106,872, filed on Jun. 29, 1998, now Pat. No. 6,486,311.
Application No. 09/106,872 is a continuation of application No. 08/717,933, filed on Sep. 23, 1996, now abandoned.
(60) Provisional application No. 60/276,822, filed on Mar. 16, 2001.
(60) Provisional application No. 60/122,450, filed on Mar. 2, 1999.
(60) Provisional application No. 60/122,560, filed on Mar. 2, 1999.
(60) Provisional application No. 60/122,565, filed on Mar. 2, 1999.
(60) Provisional application No. 60/122,566, filed on Mar. 2, 1999.
(60) Provisional application No. 60/122,452, filed on Mar. 2, 1999.
(60) Provisional application No. 60/077,763, filed on Mar. 12, 1998.
(60) Provisional application No. 60/074,590, filed on Feb. 13, 1998.
(60) Provisional application No. 60/074,633, filed on Feb. 13, 1998.
(60) Provisional application No. 60/074,624, filed on Feb. 13, 1998.
(60) Provisional application No. 60/073,283, filed on Jan. 31, 1998.
(60) Provisional application No. 60/009,455, filed on Dec. 29, 1995.

(51) Int. Cl. C07K 14/00

(52) U.S. Cl. 530/403; 530/350

(57) ABSTRACT

It has been determined that allergens, which are characterized by both humoral (IgE) and cellular (T-cell) binding sites, can be modified to be less allergenic by modifying the IgE binding sites. The IgE binding sites can be converted to non-IgE binding sites by altering as little as a single amino acid within the protein, preferably a hydrophobic residue towards the center of the IgE epitope, to eliminate IgE binding. Additionally or alternatively a modified allergen with reduced IgE binding may be prepared by disrupting one or more of the disulfide bonds that are present in the natural allergen. The disulfide bonds may be disrupted chemically, e.g., by reduction and alkylation or by mutating one or more cysteine residues present in the primary amino acid sequence of the natural allergen. In certain embodiments, modified allergens are prepared by both altering one or more linear IgE epitopes and disrupting one or more disulfide bonds of the natural allergen. In certain embodiments, the methods of the present invention allow allergens to be modified while retaining the ability of the protein to activate T-cells, and, in some embodiments by not significantly altering or decreasing IgG binding capacity. The Examples provided herein use peanut allergens to illustrate applications of the invention.

### RELATED APPLICATIONS

The present application is a continuation-in-part of U.S. Ser. No. 09/494,096 filed Jan. 28, 2000 which is in turn a continuation-in-part of U.S. Ser. No. 09/267,719 filed Mar. 11, 1999; U.S. Ser. No. 09/248,674 filed Feb. 11, 1999; U.S. Ser. No. 09/248,673 filed Feb. 11, 1999; U.S. Ser. No. 09/241,101 filed Jan. 29, 1999; U.S. Ser. No. 09/240,557 filed Jan. 29, 1999; U.S. Ser. No. 09/141,220 filed Aug. 27, 1998; U.S. Ser. No. 09/106,872 filed Jun. 29, 1998; and U.S. Ser. No. 09/191,593 filed Nov. 13, 1998 which is in turn a continuation of U.S. Ser. No. 08/717,933 filed Sep. 26, 1996. These applications claim priority to provisional applications U.S. Ser. No. 60/122,450 filed Mar. 2, 1999; U.S. Ser. No. 60/122,452 filed Mar. 2, 1999; U.S. Ser. No. 60/122,560 filed Mar. 2, 1999; U.S. Ser. No. 60/122,565 filed Mar. 2, 1999; U.S. Ser. No. 60/122,566 filed Mar. 2, 1999; U.S. Ser. No. 60/074,633 filed Feb. 13, 1998; U.S. Ser. No. 60/074,624 filed Feb. 13, 1998; U.S. Ser. No. 60/074,590 filed Feb. 13, 1998; U.S. Ser. No. 60/073,283 filed Jan. 31, 1998; and U.S. Ser. No. 60/009,455 filed Dec. 29, 1995. This application also claims priority to co-pending provisional application, U.S. Ser. No. 60/276,822, filed Mar. 16, 2001. These and every other U.S. patent application cited herein are incorporated in their entirety by reference.

### GOVERNMENT FUNDING

The United States government may have rights in this invention by virtue of grants AI-33596, AI-26629, AI-24439, and CA-40406 from the National Institute of Health.

### BACKGROUND OF THE INVENTION

Allergic disease is a common health problem affecting humans and companion animals (mainly dogs and cats) alike. Allergies exist to pollens, mites, animal danders or excretions, fungi, insects, foods, latex, drugs, and other substances present in the environment. It is estimated that up to 8% of young children and 2% of adults have allergic reactions just to foods alone. Some allergic reactions (especially those to insects, foods, latex, and drugs) can be so severe as to be life threatening.

Allergic reactions result when an individual's immune system overreacts, or reacts inappropriately, to an encountered allergen. Typically, there is no allergic reaction the first time an individual is exposed to a particular allergen. However, it is the initial response to an allergen that primes the system for subsequent allergic reactions. In particular, the allergen is taken up by antigen presenting cells (APCs; e.g., macrophages and dendritic cells) that degrade the allergen and then display allergen fragments to T-cells. T-cells, in particular CD4+ “helper” T-cells, respond by secreting a collection of cytokines that have effects on other immune system cells. The profile of cytokines secreted by responding CD4+ T-cells determines whether subsequent exposures to the allergen will induce allergic reactions. Two classes of CD4+ T-cells (Th1 and Th2; T-lymphocyte helper type) influence the type of immune response that is mounted against an allergen.

The Th1-type immune response involves the stimulation of cellular immunity to allergens and infectious agents and is characterized by the secretion of IL-2, IL-6, IL-12, IFNγ, and TNFβ by CD4+ T helper cells and the production of IgG antibodies. Exposure of CD4+ T-cells to allergens can also activate the cells to develop into Th2 cells, which secrete IL-4, IL-5, IL-10, and IL-13. One effect of IL-4 production is to stimulate the maturation of B cells that produce IgE antibodies specific for the allergen. These allergen-specific IgE antibodies attach to receptors on the surface of mast cells and basophils, where they act as a trigger to initiate a rapid immune response to the next exposure to allergen. When the individual encounters the allergen a second time, the allergen is quickly bound by these surface-associated IgE molecules. Each allergen typically has more than one IgE binding site, so that the surface-bound IgE molecules quickly become crosslinked to one another through their simultaneous (direct or indirect) associations with allergen. Such cross-linking induces mast cell and basophil degranulation, resulting in the release of histamines and other substances that trigger allergic reactions. Individuals with high levels of IgE antibodies are known to be particularly prone to allergies.

The Th1- and Th2-type responses are antagonistic. In other words, one response inhibits secretions characterized by the other immune response. Thus, therapies to control the Th1- and Th2-mediated immune responses are highly desirable to control immune responses to allergens.

Other than avoidance, and drugs (e.g., antihistamines, decongestants, and steroids) that only treat symptoms, can have unfortunate side effects, and often only provide temporary relief, the only currently medically accepted treatment for allergies is immunotherapy. Immunotherapy involves the repeated injection of allergen extracts, over a period of years, to desensitize a patient to the allergen. Unfortunately, traditional immunotherapy is time consuming, usually involving years of treatment, and often fails to achieve its goal of desensitizing the patient to the allergen. Furthermore, it is not the recommended treatment for food allergies, such as peanut allergies, due to the risk of anaphylaxis, a systemic and potentially lethal type of allergic reaction.

Noon first introduced allergen injection immunotherapy in 1911, a practice based primarily on empiricism with non-standardized extracts of variable quality (Noon, Lancet 1:1572, 1911). More recently the introduction of standardized extracts has made it possible to increase the efficacy of immunotherapy, and double-blind placebo-controlled trials have demonstrated the efficacy of this form of therapy in allergic rhinitis, asthma and bee-sting hypersensitivity (BSAC Working Party, Clin. Exp. Allergy 23:1, 1993). However, increased risk of anaphylactic reactions has accompanied this increased efficacy. For example, initial trials of immunotherapy to food allergens has demonstrated an unacceptable safety to efficacy ratio (Oppenheimer et al., J. Allergy Clin. Immun. 90:256, 1992; Sampson, J. Allergy Clin. Immun. 90:151, 1992; and Nelson et al., J. Allergy Clin. Immun. 99:744, 1996). Results like these have prompted investigators to seek alternative forms of immunotherapy as well as to seek other forms of treatment.

Initial trials with allergen-non-specific anti-IgE antibodies to deplete the patient of allergen-specific IgE antibodies have shown early promise (Boulet et al., American J. Respir. Crit. Care Med. 155:1835, 1997; Fahy et al., American J. Respir. Crit. Care Med. 155:1828, 1997; and Demoly and Bousquet American J. Resp. Grit. Care Med. 155:1825, 1997). On the other hand, trials utilizing immunogenic peptides that represent T-cell epitopes have been disappointing (Norman et al., J. Aller. Clin. Immunol. 99:S127, 1997). Another form of allergen-specific immunotherapy which utilizes injection of plasmid DNA (Raz et al., Proc. Nat. Acad. Sci. USA 91:9519, 1994 and Hsu et al., Int. Immunol. 8:1405, 1996) remains unproven.

There remains a need for a safe and efficacious therapy for allergies, especially anaphylactic allergies where traditional immunotherapy is ill advised due to risk to the patient or lack of efficacy.

### SUMMARY OF THE INVENTION

It has been determined that allergens, which are characterized by both humoral (IgE) and cellular (T-cell) binding sites, can be modified to be less allergenic by modifying the IgE binding sites. Binding sites are identified using known techniques, such as by binding with antibodies in pooled sera obtained from individuals known to be immunoreactive with the allergen to be modified. The IgE binding sites can be converted to non-IgE binding sites by altering as little as a single amino acid within the protein, preferably a hydrophobic residue towards the center of the IgE epitope, to eliminate IgE binding. Additionally or alternatively a modified allergen with reduced IgE binding may be prepared by disrupting one or more of the disulfide bonds that are present in the natural allergen. The disulfide bonds may be disrupted chemically, e.g., by reduction and alkylation or by mutating one or more cysteine residues present in the primary amino acid sequence of the natural allergen. In certain embodiments, modified allergens are prepared by both altering one or more linear IgE epitopes and disrupting one or more disulfide bonds of the natural allergen.

In certain embodiments, the methods of the present invention allow allergens to be modified while retaining the ability of the protein to activate T-cells, and, in some embodiments by not significantly altering or decreasing IgG binding capacity. Proteins that are modified to alter IgE binding may for be screened for binding with IgG and/or activation of T-cells. Additionally, modified allergens may be screened using standard techniques such as a skin test for wheal and flare formation and/or a basophil histamine release assay can be used to assess decreased allergenicity of modified proteins, created as described in the Examples. In certain embodiments, the modified allergens are screened for their ability to alleviate allerguic symptoms in an animal model, e.g., as described in Example 27.

Peanut allergens (Ara h 1, Ara h 2, and Ara h 3) have been used in the Examples to demonstrate alteration of IgE binding sites while retaining binding to IgG and activation of T-cells. The critical amino acids within each of the IgE epitopes of the peanut protein that are important to immunoglobulin binding were determined. Substitution of even a single amino acid within each of the epitopes led to loss of IgE binding. Although the epitopes shared no common amino acid sequence motif, the hydrophobic residues located in the center of the epitope appeared to be most critical to IgE binding.

The immunotherapeutics can be delivered by standard techniques in free form or as a pharmaceutical composition, using injection, by aerosol, sublingually, topically (including to a mucosal surface), etc. and by gene therapy (for example, by injection of the gene encoding the immunotherapeutic into muscle or skin where it is transiently expressed for a time sufficient to induce tolerance).

This method and the criteria for identifying and altering allergens can be used to design useful modified allergens (including nucleotide molecules encoding these allergens) for use in immunotherapy, to make a vaccine and to genetically engineer organisms such as plants and animals which then produce proteins with less likelihood of eliciting an IgE response. Techniques for engineering plants and animals are well known. Based on the information obtained using the method described in the examples, one can engineer plants or animals to cause either site specific mutations in the gene encoding the protein(s) of interest, or to knock out the gene and then insert the gene encoding the modified protein.

### BRIEF DESCRIPTION OF THE DRAWINGS

FIG. 1 shows anion exchange chromatography results of a defatted crude peanut extract fractionated over an FLPC Mono Q 10/10 column. The elution pattern of proteins (A₂₈₀) is illustrated by the solid line. A stepwise salt gradient of 0 to 1.5 mol/L of NaCl is illustrated by the dotted line. Fractions were pooled as numbered (fraction 2 is divided into 2a and 2b).

FIG. 2 shows an SDS-PAGE analysis of the same defatted crude peanut extract of FIG. 1 stained with Coomassie blue (lane 1) and immunoblotted for anti-peanut specific IgE (lane 2) with the pooled serum from the patients with atopic dermatitis and positive DPCFCs to peanut; MW=molecular weight markers.

FIG. 3 shows an SDS-PAGE gel of fraction 3 from the FPLC of FIG. 1. The gel stained with Coomassie blue (lane 1) and the IgE-specific immunoblot (lane 2) with the pooled serum from the patients with atopic dermatitis and positive DPCFCs to peanut; MW=molecular weight markers.

FIG. 4 shows anti-peanut IgE-specific ELISA (ng/ml) results against a defatted crude peanut extract and fractions 1-7 from the FPLC of FIG. 1.

FIG. 5 shows IgE ELISA inhibition results of crude peanut extract and fraction 3 (63.5 kd fraction) from the FPLC of FIG. 1 in ELISA for crude peanut.

FIG. 6 shows a Coomassie blue stained thin layer electrofocused gel (pH 3.5 to 6.85) of fraction 3 from the FPCL of FIG. 1 (lane 1); pI, standards.

FIG. 7a illustrates four distinct IgG epitopes on Ara h 1 (A-D) identified from the site specificity of the seven Ara h 1 mAbs listed in Table 4.

FIG. 7b illustrates three distinct IgE epitopes on Ara h 1 (X-Z) identified from the site specificity of the seven Ara h 1 mAbs as shown in FIG. 8.

FIG. 8 shows the site specificity of seven Ara h 1 mAbs inhibiting ant-peanut specific IgE binding to Ara h 1. Values are expressed as a percent of the anti-peanut specific IgE binding to Ara h 1 in the absence of each inhibiting mAb.

FIG. 9 shows SDS-PAGE gels of Ara h 1 allergen that has been eluted from an immuno-affinity column (lane 1) and IgE immunoblot of the same allergen with challenge-positive peanut serum pool (lane 2). MW, molecular weight markers (left lane, top-to-bottom—-106 kd, 80 kd, 49.5 kd, 32.5 kd, 27.5 kd, 18.5 kd).

FIG. 10 shows the nucleotide sequence of cDNA clone P41b of Ara h 1 (SEQ ID NO. 5).

FIG. 11 shows the nucleotide sequence of cDNA clone P17 of Ara h 1 (SEQ ID NO. 6).

FIG. 12 shows an alignment of the nucleotide sequences of cDNA clones P41b and P17 of Ara h 1.

FIG. 13 shows the predicted amino acid sequence of the protein encoded by cDNA clone P41b of Ara h 1 (SEQ ID NO. 7). The positions of peptides I, II, and III from Table 6 are boxed.

FIG. 14 shows the predicted amino acid sequence of the protein encoded by cDNA clone P17 of Ara h 1 (SEQ ID NO. 8). The positions of peptides I, II, and III from Table 6 are boxed.

FIG. 15 compares SDS-PAGE gels of whole peanut extract (lane A), purified Ara h 1 (lane B), recombinant Ara h 1 produced from cDNA clone P17 (lanes C and D), and E. coli extract (lane E). Note that the full length cDNA clone P17 produces small quantities of a truncated recombinant protein (lane C) that disappear when the first 93 bases of this clone are removed (lane D). The recombinant Ara h 1 (lane D, 68 kd) is larger than the purified Ara h 1 (lane B, 65 kd) because the recombinant Ara h 1 includes 37 amino acids of beta galactosidase. Note that the serum IgE pool does not recognize any proteins in the E. coli extract (lane E) and therefore the other bands in lane D are truncated versions of Ara h 1.

FIG. 16 shows immunoblots of recombinant Ara h 1 (upper panel) and purified Ara h 1 (lower panel) when they are contacted with serum IgE from individual patients (A-R) with peanut hypersensitivity.

FIG. 17 shows the predicted and determined IgE-binding regions on Ara h 1. Predicted regions (P1-P11) are boxed and determined regions (D1-D12) are shaded.

FIG. 18 shows IgE binding levels to Ara h 1 fragments produced from shortened clones of Ara h 1. The pluses (+) on the right hand side indicate the extent of IgE binding to the protein product of each construct. All constructs bound IgE until they were reduced to the extreme carboxyl (5′ Exo III) or amino (3′ Exo III) terminal end of the molecule.

FIG. 19 illustrates the mapping of IgE epitopes on Ara h 1 using a set of overlapping 8 mers offset by 2 amino acids that span the entire Ara h 1 amino acid sequence. Epitopes 4, 5, 6, and 7 are shaded.

FIG. 20 shows the relative IgE binding to each of the peptides (1-23) of Table 8 when each peptide was probed with serum IgE from 10 individual patients with peanut hypersensitivity. The relative intensity of IgE binding to each peptide is expressed as a percentage of the patient's total IgE binding to all of the Ara h 1 peptides.

FIG. 21 is an immunoblot showing binding of pooled IgE to 11 mutants of peptide 1 (from Table 8) each with a different single alanine substitution. The letters across the top of the panel indicate the one-letter amino acid code for the residue normally at that position and the amino acid that was substituted for this residue. The numbers indicate the position of each residue in the Ara h 1 protein (SEQ ID NO. 7). WT, indicates the wild-type peptide with no amino acid substitutions.

FIG. 22 illustrates the relative intensity of IgE binding to single amino acid mutants of the immunodominant peptides of Ara h 1 (peptides 1, 3, 4, and 17). The relative intensity of IgE binding to each peptide is expressed as a ratio of IgE binding to the non-mutated peptide (WT). The letters across the top of the panel indicate the one-letter amino acid code for the residue normally at that position and the amino acid that was substituted for this residue. The numbers indicate the position of each residue in the Ara h 1 protein (SEQ ID NO. 7). WT, indicates the wild-type peptide with no amino acid substitutions.

FIG. 23 is an immunoblot showing binding of pooled IgE to 11 mutants of peptide 9 (from Table 8) each with a different single alanine (Panel A) or methionine (Panel B) substitution. The letters across the top of each panel indicate the one-letter amino acid code for the residue normally at that position and the amino acid that was substituted for this residue. The numbers indicate the position of each residue in the Ara h 1 protein (SEQ ID NO. 7). WT, indicates the wild-type peptide with no amino acid substitutions.

FIG. 24 is a graph showing the number of hydrophobic (G, P, F, L, I, A, W, V, and M), polar (Q, S, N, Y, T, and C), and charged (R, E, D, K, and H) amino acids that were found within the IgE epitopes of Ara h 1. The shaded boxes represent the total number of times a given type of amino acid residue was found within the IgE epitopes of Ara h 1. The open boxes represent the number of times that mutation of a given type of amino acid residue resulted in the loss of IgE binding. The data suggests that hydrophobic residues are more important for IgE binding than polar or charged residues.

FIG. 25 shows an alignment of the amino acid sequences of Ara h 1 (SEQ ID NO. 7) and the amino acid sequence of phaseolin A chain (GenBank 2PHLA). Structurally conserved residues are highlighted with a star (*).

FIG. 26 shows the α-carbon alignment of a three dimensional model of Ara h 1 versus the phaseolin A chain.

FIG. 27 is a Ramachandran plot of the Phi/Psi torsion angles of the amino acids in the predicted three dimensional model of Ara h 1 shown in FIG. 26. Major outliers are indicated by their three letter amino acid code and position.

FIG. 28 is a ribbon diagram of the predicted Ara h 1 tertiary structure. The numbered areas are IgE binding peptides 1-23 of Table 8. Peptide 13, and portions of peptides 14 and 15 lie in an area of structural uncertainty.

FIG. 29 is a space filling model of the predicted Ara h 1 tertiary structure. The darkened areas represent the IgE binding peptides.

FIG. 30 illustrates fluorescence polarization measurements (mP) made over a low range of Ara h 1 concentrations (1 nM-1 μM). Each point represent the average of three different experiments. The inset shows SDS-PAGE gels of the 200 nM sample after being subjected to cross-linking conditions for varying lengths of time. Protein bands were visualized with Coomassie staining. The lower arrow indicates the Ara h 1 monomer (−60 kd), and the upper arrow indicates the Ara h 1 trimer (−180 kd).

FIG. 31 compares fluorescence anisotropy measurements (mA) made over a low (1 nM-1 upper panel) and a high (1 μM-200 mM, lower panel) range of Ara h 1 concentrations. Each line represents data from samples placed in buffers with various concentrations of NaCl (100, 400, 600, 800, 1100, 1300, and 1800 mM).

FIG. 32 shows SDS-PAGE gels of Ara h 1 after different length of enzymatic digestion under native (left panel) and denaturing (right panel) conditions.

FIG. 33 shows SDS-PAGE gels of Ara h 1 digestion resistant fragments stained with Coomassie blue (left panel) and immunoblotted with pooled IgE serum from peanut-sensitive patients (right panel).

FIG. 34 compares the position of the 20 kd and 22 kd digestion resistant fragments of Ara h 1 within SEQ ID NO. 7 with the position of peptides 1-23 of Table 8.

FIG. 35 shows anion exchange chromatography results of a defatted crude peanut extract fractionated over an FLPC PL-SAX column. The elution pattern of proteins (A₂₈₀) is illustrated by the solid line. A stepwise salt gradient of 0 to 1.5 mol/L of NaCl is illustrated by the dotted line. Fractions were pooled as numbered.

FIG. 36 shows SDS-PAGE gels of a defatted crude peanut extract stained with Coomassie blue (lane 1) and immunoblotted for anti-peanut specific IgE (lane 2) with pooled serum from patients with atopic dermatitis and positive DBPCFCs to peanut. MW, molecular weight markers 1, 50 kd; 2, 39 kd; 3, 27.5 kd; and 4, 14.5 kd.

FIG. 37 shows anti-peanut IgE-specific ELISA (ng/ml) results against a defatted crude peanut extract and fractions 1-7 from the FPLC of FIG. 35.

FIG. 38 shows IgE ELISA inhibition results of crude peanut extract and fraction 4 from the FPLC of FIG. 35 in the ELISA for crude peanut.

FIG. 39 shows a Coomassie blue stain of a two-dimensional gel with fraction 4 from the FPLC of FIG. 35. MW, molecular weight markers: 1, 112 kd; 2, 75 kd; 3, 50 kd; 4, 39 kd; 5, 27.5 kd; and 6, 17 kd.

FIG. 40 shows the nucleotide sequence of the open reading frame of a cDNA clone of Ara h 1 (SEQ ID NO. 62).

FIG. 41 shows the predicted amino acid sequence (SEQ ID NO. 63) of the protein encoded by the cDNA clone of Ara h 2 shown in FIG. 40. The positions of peptides I and II from Table 22 are shown boxed.

FIG. 42 shows peanut-specific IgE immunoblots of a series of overlapping 15 mers (1-19) offset by 8 amino acids that span the Ara h 2 amino acid sequence (upper panel). The positions of peptides 1-19 in the Ara h 2 amino acid sequence (SEQ ID NO. 63) are shown in the lower panel. The shaded areas correspond to the determined IgE-binding regions.

FIG. 43 illustrates the mapping of IgE epitopes on Ara h 2 using a set of overlapping 8 mers offset by 2 amino acids that span the entire Ara h 2 amino acid sequence. Epitopes 6 and 7 are shaded.

FIG. 44 shows the relative IgE binding to each of the peptides (1-10) of Table 24 when each peptide was probed with serum IgE from 10 individual patients with peanut hypersensitivity (Panel B). The relative intensity of IgE binding to each peptide is expressed as a percentage of the patient's total IgE binding to all of the Ara h 2 peptides. Panel A shows a representative immunoblot containing peptides 1-10 of Table 24 and probed with serum IgE from a single patient.

FIG. 45 includes an immunoblot showing binding of pooled IgE to 10 mutants of peptide 7 (from Table 24) each with a different single alanine substitution (Panel A). The letters across the top the immunoblot indicate the one-letter amino acid code for the residue normally at that position and the amino acid that was substituted for this residue. The numbers indicate the position of each residue in the Ara h 2 protein (SEQ ID NO. 63). WT, indicates the wild-type peptide with no amino acid substitutions. Panel B summarizes the mutation results for each of the 10 IgE binding peptides of Ara h 2.

FIG. 46 shows the mean T-cell proliferation (stimulation index, SI) and standard error for T-cell lines established from 17 peanut allergic individuals (upper panel) and 5 non-allergic individuals (lower panel) when they are contacted with each of the 29 overlapping peptides that span the Ara h 2 protein (peptides 904-932).

FIG. 47 shows the percentage of T-cell lines found to include CD4+ or CD8+ surface marker that were established from various non-allergic (Panel A) and allergic individuals (Panel B).

FIG. 48 is a graph of the mean IL-4 concentration (pg/ml) collected from T-cells that were stimulated with various immunodominant peptides spanning one of the determined T-cell epitopes. T-cell lines established from allergic and non-allergic patients are compared.

FIG. 49 shows the amino acid sequence of Ara h 2 (SEQ ID NO. 63). The 10 IgE epitopes of Ara h 2 are underlined and labeled 1-10. The 5 T-cell epitopes of Ara h 2 are overlined and labeled I-V. The amino acid sequence of a 10 kd protease resistant fragment (amino acids 23 to 105 of SEQ ID NO. 63) is highlighted in gray. The 10 kd fragment includes the immunodominant IgE epitopes 3, 6, and 7.

FIG. 50 illustrates the expression construct that was used to prepare recombinant Ara h 2.

FIG. 51 shows the amino acid sequence of the expressed T7 tag/His tag construct that was used for expression of recombinant proteins of Ara h 2 (SEQ ID NO. 81).

FIG. 52 shows SDS-PAGE gels of fractions obtained during purification of recombinant Ara h 2 proteins on a Ni²⁺-column: lane 1 (cell lysate); lane 2 (unbound fraction); lane 3 (20 mM imidazole was fraction); lane 4-6 (100 mM imidazole elution fractions).

FIG. 53 compares Western blots of wild-type Ara h 2 (WT, with 10 wild-type epitopes), MUT4 (with 6 wild-type epitopes), and MUT10 (with 0 wild-type epitopes) incubated with T7 tage antibody (left panel) or patient IgE serum (right panel).

FIG. 54 compares the IgE binding levels of wild-type (WT), MUT4, and MUT10 recombinant Ara h 2 proteins that were obtained by Western blot analysis using different individual sera. Each line represents IgE binding for the individual patient.

FIG. 55 compares the inhibition of IgE binding to purified Ara h 2 by recombinant wild-type, MUT4, MUT10 Ara h 2, native Ara h 2, rice protein, and recombinant wild-type Ara h 1.

FIG. 56 compares the stimulation index (SI) that was obtained when recombinant wild-type, MUT4, and MUT10 Ara h 2 was contacted with T-cell lines established from four different allergic patients.

FIG. 57 is a graph showing the % of IgE antibodies that were found to bind recombinant MUT5 Ara h 2 allergen (relative to the wild-type Ara h 2 allergen) when IgE serum taken from 10 peanut sensitive individuals (denoted A-J) was contacted with the Ara h 2 allergens.

FIG. 58 is a graph comparing the results of T-cell proliferation assays using crude peanut, purified wild-type Ara h 2 allergen, recombinant MUT5 Ara h 2 allergen, and recombinant wild-type Ara h 2 allergen.

FIG. 59 shows SDS-PAGE gels of Ara h 2 in the presence and absence of the reducing agent dithiothreitol (DTT).

FIG. 60 shows SDS-PAGE gels of Ara h 2 after various digestion times under native or reducing conditions.

FIG. 61 shows SDS-PAGE gels of Ara h 2 after digestion in different oxidation states.

FIG. 62 shows a Western blot of Ara h 2 after various digestion times using IgE sera from peanut sensitive patients.

FIG. 63 shows IgE binding levels to soy (solid line) and peanut (dashed line) determined by ELISA using non-adsorbed sera and after successive passes over a soy-affinity chromatography column. Panel A is from patient BP. Panel B is from patient BM. Panel C is from patient DH. Panel D is from patient AT. Panel E is from patient DT.

FIG. 64 illustrates IgE binding to whole peanut extract, purified Ara h 1, and purified Ara h 2 by ELISA before and after soy-specific antibody adsorption over a soy-affinity chromatography column. Each square represents optical density (O. D.) readings for a specific patient.

FIG. 65 shows tricine-SDS polyacrylaminde gels of soy and peanut extracts stained with 0.1% amido black. MWM, molecular weight markers.

FIG. 66 shows IgE binding to peanut fractions isolated on a tricine-SDS polyacrylamide gel for two patients allergic to peanut and soy (BP and BM), and three patients allergic to peanut only (DH, AT, and DT). The first lane for each patient was reacted with non-adsorbed serum, and the second lane was reacted with soy-adsorbed serum.

FIG. 67 shows glycine-SDS-PAGE gels of soy and peanut extracts. The first three lanes represent molecular weight markers (MWM), soy and peanut stained with 0.1% amido black, respectively. The next three sets of lanes show IgE antibody binding to soy and peanut protein fractions with non-adsorbed serum from patient BP, serum passed twice over a soy-affinity chromatography column, and serum passed five times over the column.

FIG. 68A shows the nucleotide sequence of the open reading frame (ORF) of a cDNA clone of Ara h 2 (SEQ ID NO. 89).

FIG. 68B shows the predicted amino acid sequence of the Ara h 3 protein (SEQ ID NO. 90) encoded by the ORF of FIG. 68A. The sequenced amino terminus is shown boxed.

FIG. 69A shows an alignment of a conserved region near the amino terminus region of the acidic region of the amino acid sequences of Ara h 3 (SEQ ID NO. 90), G1 Soy (GenBank P04776), G2 Soy (GenBank A91341), and A2 Pea (GenBank X17193). Amino acids from the glycinin signature sequence are shaded.

FIG. 69B shows an alignment of a conserved region near the amino terminus region of the basic region of the amino acid sequences of Ara h 3 (SEQ ID NO. 90), G1 Soy (GenBank P04776), G2 Soy (GenBank A91341), and A2 Pea (GenBank X17193). Amino acids from the glycinin signature sequence are shaded.

FIG. 70 shows bacterial and immunoblot analysis of recombinant Ara h 3. Panel A shows SDS-PAGE gels of bacterial extract samples stained with Coomassie blue: 4 hours induction of vector containing no insert (lane A); uninduced Ara h 3 (vector with insert) (lane B); after 1 hour induction (lane C); after 2 hours induction (lane D); after 3 hours induction (lane E); and after 4 hours induction (lane F). Panel B shows immunoblots of the gels in Panel A with a pool of patient serum. Panel C compares immunoblots of recombinant Ara h 3 with serum IgE from individual patients (lanes A-R were patients with documented peanut hypersensitivity), a pool of serum IgE from peanut-hypersensitive patients (lane S), and serum IgE from a patient with elevated serum IgE which served as a negative control (lane T).

FIG. 71 illustrates the position of IgE binding regions in the amino acid sequence of Ara h 3 (R1-R4, shaded in SEQ ID NO. 90).

FIG. 72 illustrates the mapping of the IgE epitopes of Ara h 3 using overlapping 15 mers offset by 2 amino acids (Panel B). The data shown represents peptides spanning amino acids 299-323 of SEQ ID NO. 90. Panel A shows IgE SPOT immunoblots for the six peptides shown in Panel A.

FIG. 73 includes an immunoblot showing binding of pooled IgE to 15 mutants of peptide 4 (from Table 29A) each with a different single alanine substitution. The letters along the side of the immunoblot indicate the one-letter amino acid code for the residue normally at that position and the amino acid that was substituted for this residue. The numbers indicate the position of each residue in the Ara h 3 protein (SEQ ID NO. 90). WT, indicates the wild-type peptide with no amino acid substitutions.

FIG. 74 bacterial and immunoblot analysis of recombinant mutant Ara h 3. Panel A shows SDS-PAGE gels of bacterial extract samples stained with Coomassie blue. Panel B shows immunoblots of the gels in Panel A with a pool of patient serum.

FIG. 75 compares relative quantities of IgE binding to whole soy (solid line) and peanut (dotted line) protein by ELISA after successive passes over a peanut-affinity column. Panel A is from patient BP. Panel B is from patient DT.

FIG. 76 compares IgE binding to tricine-SDS polyacrylamide peanut and soy immunoblots for one patient allergic to peanut and soy (BP) and one patient allergic to peanut only (DT). The first lane for each patient was reacted with non-adsorbed serum. The second lane was reacted with peanut-adsorbed serum.

FIG. 77 compares IgE binding to tricine-SDS polyacrylamide soy immunoblots for two patients allergic to peanut and soy (BP and BM), and three patients only allergic to peanut (DH, AT, and DT). The first lane for each patient was reacted with non-adsorbed serum. The second lane was blotted with soy-adsorbed serum.

FIG. 78 shows the amino acid sequencing results of the N-terminus of a 22 kd soybean allergen.

FIG. 79 shows the amino acid sequence of soybean allergen glycinin subunit A2B1a (SEQ ID NO. 109). The soybean IgE positive regions (R1-R6) are shaded. The location of the sequenced N-terminus of the 22 kd fragment identified in FIG. 78 is shown boxed.

FIG. 80 shows an alignment of the amino acid sequences of Ara h 3 (SEQ ID NO. 90) and glycinine subunit A2B1a (SEQ ID NO. 109). Conserved residues are indicated with a star (*).

FIG. 81 shows an alignment of the amino acid sequences of Ara h 1 (SEQ ID NO. 7) and β-conglycinin (GenBank AAB01374, SEQ ID NO. 110). The positions of the Ara h 1 IgE epitopes are underlined and labeled 1-23. The positions of the soybean and peanut IgE positive binding regions are also indicated on the β-conglycinin sequence.

FIG. 82 compares the sequence homology of the Ara h 1 IgE epitopes (1-23) with homologous regions of β-conglycinin. Conserved residues are indicated with a star (*).

FIG. 83 lists the primers that were used to amplify IgE Fab fragments in the construction of a cDNA library of Fabs to peanut allergens.

FIG. 84 shows electrophoresed agarose gels of expressed Fabs that have been probed for the presence of primer specific amplification products.

FIG. 85 is a schematic illustrating the steps involved in the construction of a recombinant IgE Fab library.

FIG. 86 shows electrophoresed agarose gels of nineteen clones that were randomly picked from the recombinant IgE Fab library and analyzed by restriction enzyme digestion. Heavy chain inserts were released by digestion with SpeI and XhoI and light chain inserts were released by digestion with Sad and XboI. Fifteen out of the nineteen clones (i.e., 79%) contained both heavy and light chain inserts.

FIG. 87 shows SDS-PAGE gels of peanut allergens Ara h 1 and Ara h 2 that have been purified from defatted peanut powder and Ara h 3 that was expressed recombinantly and purified using affinity chromatography.

FIG. 88 compares binding of IgE Fab fragments to Ara h 2 detected using an ELISA assay. The IgE Fab fragments were produced by clones that were selected using Ara h 2 (clones 1, 2, 3, 8, 10, 16, 25, and 26). IgE bound to Ara h 2 from the serum of a peanut sensitive patient is included for comparison. Results are shown expressed as a fold increase over binding when no primary antibody is used.

FIG. 89 describes the ten groups of mice (G1-010) that were used for the in vivo desensitization experiments. The 5 week old female C3H/HeJ mice (approx. 10 per group) were first sensitized with crude peanut extract and cholera toxin over a period of 8 weeks (W0-W8). The mice were then treated according to ten different desensitization protocols at weeks 10, 11, and 12 (W10-W12). Finally the mice were challenged with crude peanut extract at week 13 (W13). G1 mice were sham desensitized at weeks 10-12, i.e., treated with a placebo. G2, G3, and G4 mice were desensitized via the subcutaneous (sc) route with Heat Killed E. coli (HKEc) expressing modified Ara h 1, 2, and 3 (30, 15, and 5 μg of each, respectively). G5 mice were desensitized via the intragastric (ig) route with Heat Killed E. coli (HKEc) expressing modified Ara h 1, 2, and 3 (50 μg of each). G6 mice were desensitized via the rectal (pr) route with Heat Killed E. coli (HKEc) expressing modified Ara h 1, 2, and 3 (30 μg of each). G7 mice were desensitized via the rectal (pr) route with modified Ara h 1, 2, and 3 (30 μg of each) alone. G8 mice were naïve, i.e., were not sensitized with crude peanut extract and cholera toxin during weeks 0-8. G9 mice were desensitized via the subcutaneous (sc) route with Heat Killed Listeria (HKL) alone. G10 mice were desensitized via the subcutaneous (sc) route with Heat Killed Listeria (HKL) expressing modified Ara h 1, 2, and 3 (30 μg of each).

FIG. 90 is a graph comparing the average IgE levels at weeks 3, 8, 12, and 14 for the ten groups of mice (G1-G10) described in FIG. 89.

FIG. 91 is a graph comparing the individual (symbols) and average (solid line) symptom scores (0-5) at week 14 for eight (G1-G8) of the ten groups of mice described in FIG. 89.

FIG. 92 is a graph comparing the individual (symbols) and average (solid line) symptom scores (0-5) at week 14 for four (G1, and G8-G10) of the ten groups of mice described in FIG. 89.

FIG. 93 is a graph comparing the individual (symbols) and average (solid line) body temperatures (° C.) at week 14 for eight (G1-G8) of the ten groups of mice described in FIG. 89.

FIG. 94 is a graph comparing the individual (symbols) and average (solid line) body temperatures (° C.) at week 14 for four (G1, and G8-G10) of the ten groups of mice described in FIG. 89.

FIG. 95 is a graph comparing the individual (symbols) and average (solid line) airway responses (peak respiratory flow in ml/min) at week 14 for eight (G1-G8) of the ten groups of mice described in FIG. 89.

FIG. 96 is a graph comparing the individual (symbols) and average (solid line) airway responses (peak respiratory flow in ml/min) at week 14 for four (G1, and G8-G10) of the ten groups of mice described in FIG. 89.

FIG. 97 is a graph comparing the plasma histamine concentrations (nM) at week 14 for the ten groups of mice (G1-G10) described in FIG. 89.

FIG. 98 is a graph comparing the plasma IL-4 concentrations (pg/ml) at week 14 for the ten groups of mice (G1-G10) described in FIG. 89.

FIG. 99 is a graph comparing the plasma IL-5 concentrations (pg/ml) at week 14 for the ten groups of mice (G1-G10) described in FIG. 89.

FIG. 100 is a graph comparing the plasma IFNγ concentrations (pg/ml) at week 14 for the ten groups of mice (G1-G10) described in FIG. 89.

### DEFINITIONS

“Animal”: The term “animal”, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). An animal may be a transgenic animal.

“Antigen”: The term “antigen”, as used herein, refers to a molecule that elicits production of an antibody (i.e., a humoral response) and/or an antigen-specific reaction with T-cells (i.e., a cellular response) in an animal.

“Allergen”: The term “allergen”, as used herein, refers to a subset of antigens which elicit the production of IgE in addition to other isotypes of antibodies. The terms “allergen”, “natural allergen”, and “wild-type allergen” may be used interchangeably. Preferred allergens for the purpose of the present invention are protein allergens.

“Allergic reaction”: The phrase “allergic reaction”, as used herein, relates to an immune response that is IgE mediated with clinical symptoms primarily involving the cutaneous (e.g., uticana, angiodema, pruritus), respiratory (e.g., wheezing, coughing, laryngeal edema, rhinorrhea, watery/itching eyes), gastrointestinal (e.g., vomiting, abdominal pain, diarrhea), and cardiovascular (i.e., if a systemic reaction occurs) systems. For the purposes of the present invention, an asthmatic reaction is considered to be a form of allergic reaction.

“Anaphylactic allergen”: The phrase “anaphylactic allergen”, as used herein, refers to a subset of allergens that are recognized to present a risk of anaphylactic reaction in allergic individuals when encountered in its natural state, under natural conditions. For example, for the purposes of the present invention, pollen allergens, mite allergens, allergens in animal danders or excretions (e.g., saliva, urine), and fungi allergens are not considered to be anaphylactic allergens. On the other hand, food allergens, insect allergens, and rubber allergens (e.g., from latex) are generally considered to be anaphylactic allergens. Food allergens are particularly preferred anaphylactic allergens for use in the practice of the present invention. In particular, nut allergens (e.g., from peanut, walnut, almond, pecan, cashew, hazelnut, pistachio, pine nut, brazil nut), dairy allergens (e.g., from egg, milk), seed allergens (e.g., from sesame, poppy, mustard), soybean, wheat, and fish allergens (e.g., from shrimp, crab, lobster, clams, mussels, oysters, scallops, crayfish) are anaphylactic food allergens according to the present invention. Particularly interesting anaphylactic allergens are those to which reactions are commonly so severe as to create a risk of death.

“Anaphylaxis” or “anaphylactic reaction”: The phrase “anaphylaxis” or “anaphylactic reaction”, as used herein, refers to a subset of allergic reactions characterized by mast cell degranulation secondary to cross-linking of the high-affinity IgE receptor on mast cells and basophils induced by an anaphylactic allergen with subsequent mediator release and the production of severe systemic pathological responses in target organs, e.g., airway, skin digestive tract, and cardiovascular system. As is known in the art, the severity of an anaphylactic reaction may be monitored, for example, by assaying cutaneous reactions, puffiness around the eyes and mouth, vomiting, and/or diarrhea, followed by respiratory reactions such as wheezing and labored respiration. The most severe anaphylactic reactions can result in loss of consciousness and/or death.

“Antigen presenting cell”: The phrase “antigen presenting cell” or “APC”, as used herein, refers to cells which process and present antigens to T-cells to elicit an antigen-specific response, e.g., macrophages and dendritic cells.

“Associated with”: When two entities are “associated with” one another as described herein, they are linked by a direct or indirect covalent or non-covalent interaction. Preferably, the association is covalent. Desirable non-covalent interactions include, for example, hydrogen bonding, van der Walls interactions, hydrophobic interactions, magnetic interactions, etc.

“Decreased anaphylactic reaction”: The phrase “decreased anaphylactic reaction”, as used herein, relates to a decrease in clinical symptoms following treatment of symptoms associated with exposure to an anaphylactic allergen, which can involve exposure via cutaneous, respiratory, gastrointestinal, and mucosal (e.g., ocular, nasal, and aural) surfaces or a subcutaneous injection (e.g., via a bee sting).

“Epitope”: The term “epitope”, as used herein, refers to a binding site including an amino acid motif of between approximately six and fifteen amino acids which can be bound by an immunoglobulin (e.g., IgE, IgG, etc.) or recognized by a T-cell receptor when presented by an APC in conjunction with the major histocompatibility complex (MHC). A linear epitope is one where the amino acids are recognized in the context of a simple linear sequence. A conformational epitope is one where the amino acids are recognized in the context of a particular three dimensional structure.

“Fragment”: An allergen “fragment” according to the present invention is any part or portion of the allergen that is smaller than the intact natural allergen. In preferred embodiments of the invention, the allergen is a protein and the fragment is a peptide.

“Immunodominant epitope”: The phrase “immunodominant epitope”, as used herein, refers to an epitope which is bound by antibody in a large percentage of the sensitized population or where the titer of the antibody is high, relative to the percentage or titer of antibody reaction to other epitopes present in the same antigen. Preferably, an immunodominant epitope is bound by antibody in more than 50% of the sensitive population, more preferably more than 60%, 70%, 80%, 90%, 95%, or 99%.

“Immunostimulatory sequences”: The phrase “immunostimulatory sequences” or “ISS”, as used herein, relates to oligodeoxynucleotides of bacterial, viral, or invertebrate origin that are taken-up by APCs and activate them to express certain membrane receptors (e.g., B7-1 and B7-2) and secrete various cytokines (e.g., IL-1, IL-6, IL-12, TNF). These oligodeoxynucleotides contain unmethylated CpG motifs and when injected into animals in conjunction with an antigen, appear to skew the immune response towards a Th1-type response. See, for example, Yamamoto et al., Microbiol. Immunol. 36:983, 1992; Krieg et al., Nature 374:546, 1995; Pisetsky, Immunity 5:303, 1996; and Zimmerman et al., J. Immunol. 160:3627, 1998.

### DETAILED DESCRIPTION OF THE INVENTION

The present application mentions various patents, scientific articles, and other publications. The contents of each such item are hereby incorporated by reference. In addition, the contents (as of the filing date of the application) of all websites referred to herein are incorporated by reference.

### A. Natural Allergens

### Introduction

Many allergens are known that elicit allergic responses, which may range in severity from mildly irritating to life-threatening. Exemplary lists of protein allergens are presented as Appendices 1-9. This list was adapted on Jul. 22, 1999, from the world wide web at ftp://biobase.dk/pub/who-iuis/allergen.list, which provides lists of known allergens. Of particular interest are anaphylactic allergens, e.g., food allergens, insect allergens, and rubber allergens (e.g., from latex).

Food allergies are mediated through the interaction of IgE to specific proteins contained within the food. Examples of common food allergens include proteins from nuts (e.g., from peanut, walnut, almond, pecan, cashew, hazelnut, pistachio, pine nut, brazil nut), dairy products (e.g., from egg, milk), seeds (e.g., from sesame, poppy, mustard), soybean, wheat, and fish (e.g., shrimp, crab, lobster, clams, mussels, oysters, scallops, crayfish). The IgE epitopes from the major allergens of cow milk (Ball et al., Clin. Exp. Allergy 24:758, 1994), egg (Cooke and Sampson, J. Immunol. 159:2026, 1997), codfish (Aas and Elsayed, Dev. Biol. Stand. 29:90, 1975), hazel nut (Elsayed et al., Int. Arch. Allergy Appl. Immunol. 89:410, 1989), peanut (Burks et al., Eur. J. Biochemistry 245:334, 1997 and Stanley et al., Arch. Biochem. Biophys. 342:244, 1997), soybean (Herein et al., Int. Arch. Allergy Appl. Immunol. 92:193, 1990), and shrimp (Shanty et al., J. Immunol. 151:5354, 1993) have all been elucidated, as have others. Insect allergens include proteins from insects such as fleas, ticks, ants, cockroaches, and bees.

The majority of allergens discussed above elicit a reaction when ingested, inhaled, or injected. Allergens can also elicit a reaction based solely on contact with the skin. Latex is a well known example. Latex products are manufactured from a milky fluid derived from the rubber tree (Hevea brasiliensis) and other processing chemicals. A number of the proteins in latex can cause a range of allergic reactions. Many products contain latex, such as medical supplies and personal protective equipment. Two types of reactions can occur in persons sensitive to latex: local allergic dermatitis and immediate systemic hypersensitivity (or anaphylaxis).

Local allergic dermatitis develops within a short time after exposure to latex and generally includes symptoms of urticaria or hives. The reaction is allergic and triggered by direct contact, not inhalation (Sussman et al., JAMA 265:2844, 1991). The symptoms of immediate systemic hypersensitivity vary from skin and respiratory problems (e.g., urticaria, hives, rhinoconjunctivitis, swelling of lips, eyelids, and throat, wheezing, and coughing) to anaphylaxis which may progress to hypotension and shock. The reaction may be triggered by inhalation or skin exposure to the allergen.

Proteins found in latex that interact with IgE antibodies have been characterized by two-dimensional electrophoresis. Protein fractions of 56, 45, 30, 20, 14, and less than 6.5 kd were detected (Posch et al., J. Allergy Clin. Immunol. 99:385, 1997). Acidic proteins in the 8-14 kd and 22-24 kd range that reacted with IgE antibodies were also identified (Posch et al., 1997, supra). The proteins prohevein and hevein, from Hevea brasiliensis, are known to be major latex allergens and to interact with IgE (Alenius et al., Clin. Exp. Allergy 25:659, 1995 and Chen et al., J. Allergy Clin. Immunol. 99:402, 1997). Most of the IgE binding domains have been shown to be in the hevein domain rather than the domain specific for prohevein (Chen et al., 1997, supra). The main IgE epitope of prohevein is thought to be in the N-terminal, 43 amino acid fragment (Alenius et al., J. Immunol. 156:1618, 1996). The hevein lectin family of proteins has been shown to have homology with potato lectin and snake venom disintegrins (platelet aggregation inhibitors) (Kielisqewski et al., Plant J. 5:849, 1994).

### Cloning and Sequencing of Natural Allergens

It will be appreciated that a variety of methods for cloning and sequencing protein allergens are known in the art. The present invention is not limited in any way to a specific cloning or sequencing method and may use any method known now or later discovered including, but not limited to, those methods described in reviews, e.g., Crameri, Allergy 56:S30, 2001; Appenzeller et al., Arch. Immunol. Ther. Exp. 49:19, 2001; Deviller, Allerg. Immunol. (Paris) 27:316, 1995; and Scheiner, Int. Arch. Allergy Immunol. 98:93, 1992; in reference collections, e.g., Current Protocols in Molecular Biology Ed. by Ausubel et al., John Wiley & Sons, New York, N. Y., 1989 and Molecular Cloning: A Laboratory Manual Ed. by Sambrook et al., Cold Spring Harbor Press, Plainview, N. Y., 1989; and in the references cited in Appendix 10. In certain embodiments cDNA cloning and amino acid sequencing of purified allergens (e.g., fragmentation followed by Edman degradation and/or mass spectrometry) are combined. In particular, amino acid sequences predicted from cDNA clones are preferably compared with N-terminal and/or C-terminal sequences determined by amino acid sequencing. As is well known in the art, such comparisons allow post-translational modifications (e.g., N-terminal proteolytic cleavage) to be identified and hence mature allergens to be fully characterized.

### Characterization of Allergen Fragments

The amino acid sequence and structure of an allergen encountered by an APC in vivo (i.e., within an exposed animal) may, in certain cases, differ from that of the natural allergen. For example, instead of encountering the natural allergen, APCs may encounter fragments of the allergen. This is particularly the case for food allergens that must negotiate the acidic environment of the stomach and a variety of proteolytic enzymes on their journey from ingestion to absorption. Accordingly, in certain embodiments, it may prove advantageous to identify and characterize the amino acid sequence and structure of an allergen or its fragments subsequent to processing within an animal. In certain embodiments, allergen fragments may be isolated from in vivo samples using standard purification techniques (e.g., samples taken from the blood, the gastrointestinal tract, the lungs, etc. of an animal that has been exposed to the natural allergen). As described in greater detail in Examples 7 and 14, the fragments can also be studied in vitro, e.g., by identifying and sequencing the products of in vitro proteolytic digestion of a natural allergen (i.e., by gastric, pancreatic, and intestinal proteases such as pepsin, parapepsin I and II, trypsin, chymotrypsin, elastase, carboxypeptidases, enterokinase, aminopeptidases, and dipeptidases).

### Characterization and Isolation of Immunoglobulins

In certain embodiments it may be of value to distinguish and/or isolate immunoglobulins (e.g., IgE or IgG) which interact with conformational and linear epitopes of a given allergen. It may, for example, prove advantageous to use an assay with immunoglobulins that interact with conformational epitopes instead of linear epitopes when attempting to identify the precise amino acids that are involved in conformational epitopes. Due to the complexity and heterogeneity of patient serum, it may be difficult to employ a standard immobilized allergen affinity-based approach to directly isolate immunoglobulins in quantities sufficient to permit their characterization. These problems can be avoided by isolating some or all of the immunoglobulins which interact with conformational epitopes from a combinatorial immunoglobulin phage display library.

Steinberger et al. prepared a combinatorial IgE phage display library from mRNA isolated from the peripheral blood mononuclear cells of a patient allergic to the major Timothy Grass pollen antigen (Steinberger et al., J. Biol. Chem. 271:10967, 1996). Allergen-specific IgEs were selected by panning filamentous phage expressing IgE Fabs on their surfaces against allergen immobilized on the wells of 96 well microtiter plates. cDNAs were then isolated from allergen-binding phage and transformed into E coli for the production of large quantities of monoclonal, recombinant, allergen-specific IgE Fabs.

If native allergen or full length recombinant allergen is used in the panning step to isolate phage, then Fabs corresponding to IgEs specific for conformational epitopes should be included among the allergen-specific clones identified. By screening the individual recombinant IgE Fabs against denatured antigen or against the relevant linear epitopes identified for a given antigen, the subset of conformation-specific clones which do not bind to linear epitopes can be defined.

To determine whether the library screening has yielded a complete inventory of the allergen-specific IgEs present in patient serum, an immunocompetition assay can be performed. Pooled recombinant Fabs would be preincubated with immobilized allergen. After washing to remove unbound Fab, the immobilized allergen would then be incubated with patient serum. After washing to remove unbound serum proteins, an incubation with a reporter-coupled secondary antibody specific for IgE Fc domain would be performed. Detection of bound reporter would allow quantitation of the extent to which serum IgE was prevented from binding to allergen by recombinant Fab. Maximal, uncompleted serum IgE binding would be determined using allergen which had not been preincubated with Fab or had been incubated with nonsense Fab. If IgE binding persists in the face of competition from the complete set of allergen-specific IgE Fab clones, this experiment can be repeated using denatured antigen to determine whether the epitopes not represented among the cloned Fabs are linear or conformational. The preparation of a library of Fabs to peanut allergens is described in Example 26.

### Identification of Epitopes

The majority of natural allergens include linear and/or conformational epitopes for immunoglobulins (e.g., IgE and IgG) and T-cells. A variety of methods are known in the art that can be used to identify the amino acids involved in these epitopes (see, for example, Benjamin et al., Ann. Rev. Immunol. 2:67, 1984; Atassi, Eur. J. Biochem. 145:1, 1984; Getzoff et al., Adv. Immunol. 43:1, 1988; Jemmerson and Paterson, Biotechniques 4:18, 1986; Geysen et al., J. Immunol. Methods 102:259, 1987; see also, Current Protocols in Immunology Ed. by Coligan et al., John Wiley & Sons, New York, N. Y., 1991).

Linear epitopes can be determined using a technique commonly referred to as “scanning” (see Geysen et al., 1987, supra). As described in greater detail in Examples 4, 11, 17, 20, 21, 23, and 25, the approach uses collections of overlapping peptides that span the entire length of the allergen. The peptides may be chosen such that they span the length of the amino acid sequence predicted from a cDNA clone; the length of the mature protein (i.e., including any post-translational modifications); or the length of an allergen fragment (e.g., a digestion resistant fragment). The approximate location of linear epitopes within a given amino acid sequence can, for example, be determined using peptides that are 8-15 amino acids in length and offset by 1-5 residues. It is to be understood that peptides having any length and offset may be used according to the present invention; however, the use of longer peptides decreases the resolution of individual epitopes and the use of shorter peptides increases the risk of missing an epitope. For long amino acid sequences, where cost of peptide synthesis is a major consideration, longer peptides and offsets are preferred. Peptides that include a linear IgE epitope are identified using a standard immunoassay with IgE serum taken from an individual or a pool of individuals that are known to be allergic to the allergen. It will be recognized that different individuals may generate IgE that recognize different epitopes on the same allergen. Thus, it is typically desirable to expose the peptides to a representative pool of serum samples, e.g., taken from at least 5-10, preferably at least 15, individuals with demonstrated allergy to the allergen. Once peptides that include a linear IgE epitope have been identified, the specific amino acids that are involved in each of the linear IgE epitopes can be determined by repeating the process using different sets of shorter overlapping peptides that span the length of these peptides. In preferred embodiments, once the specific amino acids that are involved in each of the linear IgE epitopes have been identified, sets of peptides that cover each linear IgE epitope are prepared that each include a single mutation (e.g., but not limited to substitution with alanine or methionine, deletion, etc.). As described in Examples 4, 11, and 17 these mutants can be used to identify those amino acids that are most important for IgE binding and hence which when modified cause the largest reduction in IgE binding. It will be appreciated that identification of these amino acid positions will facilitate the preparation of modified allergens with reduced IgE binding.

It is to be understood that a similar approach can be used to detect IgG epitopes. As described in greater detail in Example 12T-cell epitopes can also be detected in this manner using, for example, a T-cell proliferation assay. In certain embodiments, the methods of the present invention include a step of comparing the locations of the IgE, IgG, and T-cell epitopes within the sequence of a natural allergen of interest.

Conformational epitopes can be determined using phage display libraries (see, for example, Eichler and Houghten, Molecular Medicine Today 1:174, 1995 and Jensen-Jarolim et al., J. Appl. Clin. Immunol. 101:5153a, 1997) and by cross-linking antibodies to whole protein or protein fragments, typically antibodies obtained from a pooled patient population known to be allergic to the natural allergen.

### Identification of Native Disulfide Bonds

For natural allergens that include cysteine residues, it may prove advantageous to further predict and/or identify the disulfide bonds that are present within the native natural allergen. Preferably the natural allergen has been cloned and/or sequenced. Fariselli et al. have described a theoretical model for predicting the disulfide bonding states of cysteine residues in a protein based on a known amino acid sequence (see Fariselli et al., Proteins 36:340, 1999; see also the world wide web at http://prion.biocomp.unibo.it/cyspred.html).

Additionally or alternatively, the disulfide bonds present within a natural allergen may be determined experimentally using any of the techniques known in the art. Disulfide bonds have traditionally been located by cleaving a protein between the half-cystinyl residues with highly specific cleavage reagents, e.g., trypsin or cyanogen bromide, with subsequent isolation and identification of disulfide containing peptides by their amino acid sequence or composition (see Creighton, Methods Enzymol. 107:305, 1984 and Gray et al., Biochem. 23:2796, 1984). Zhou and Smith introduced an approach that instead uses partial acid hydrolysis to cleave proteins between half-cystinyl residues (see Zhou and Smith, J. Prot. Chem. 9:523, 1990). Gray et al. further pioneered a technique that involves partially reducing proteins at pH 3 with tris-(2-carboxyethyl)-phosphine (TCEP) to generate a series of intermediates containing both disulfides and thiol. Separation of these intermediates at pH 2 by reversed-phase HPLC is then followed by alkylation of free thiols and amino acid sequencer analysis to determine the location of labeled thiols. Performing each step in an acidic medium limits disulfide exchange reactions and hence allows partially reduced proteins to be prepared and subsequently separated (see Gray et al., Protein Sci. 2:1732, 1993). Wu et al. have developed a technique that also relies on low pH to prevent scrambling of disulfide bonds but uses mass spectrometry to characterize intermediates (see Wu and Watson, Protein Sci. 6:391, 1997 and Wu et al., Anal. Biochem. 235:161, 1996). The procedure also involves subjecting a protein to limited chemical reduction using TCEP at pH 3 to produce a mixture of singly reduced protein isomers. The nascent sulfhydryls are then cyanylated by 2-nitro-5-thiocyanobenzoic acid (NTCB) under alkaline conditions or more preferably by 1-cyano-4-dimethylamino-pyridinium tetrafluoroborate (CDAP) under acidic conditions and the resulting isomers are separated by reversed-phase HPLC. Under alkaline conditions, the cleavage of the peptide bond occurs on the N-terminal side of cyanylated cysteines to form truncated peptides which after reduction of the remaining disulfide bonds can be mass mapped by desorption ionization mass spectrometry (MALDI-MS). The masses of the fragments can be related to the location of the paired cysteines that have undergone reduction, cyanylation, and cleavage. It will be appreciated, that in order to minimize structural diversity of disulfide bonds, proteins under study are preferably denatured (e.g., by dissolution in a chaotropic agent such as guanidine hydrochloride, urea, etc.) so that differences in the accessibility of reducing and cyanylating agents to each disulfide bond are minimized.

### B. Modified Allergens

### Introduction

It is desirable to modify natural allergens to diminish binding to IgE. In some embodiments, this is achieved while retaining the ability of the allergens to activate T-cells and/or by not significantly altering or decreasing IgG binding capacity. This requires modification of one or more IgE epitopes in the natural allergen. It will be appreciated, that for natural allergens that include one or more native disulfide bonds, this may be achieved by disrupting one or more disulfide bonds of the natural allergen. Indeed, the tertiary structure of proteins is determined in part by disulfide bonds.

A preferred modified allergen is one that can be used with a majority of patients having a particular allergy. Use of pooled sera from allergic patients allows determination of one or more immunodominant epitopes in the allergen. Once some or all of the IgE binding sites are known, it is possible to modify the gene encoding the allergen, using site directed mutagenesis by any of a number of techniques, to produce a modified allergen as described below, and thereby express modified allergens. Alternatively, when the modified allergen is only being modified chemically (e.g., by reduction and alkylation) one may prepare modified allergens directly from natural allergens that have been purified from natural extracts.

### Recombinantly Modified Allergens

A mutated allergen may be made using recombinant techniques, e.g. using oligonucleotide-directed mutagenisis as described in Examples 5, 13, and 18. Expression in a prokaryotic or eukaryotic host including bacteria, yeast, and baculovirus-insect T-cell systems may be used to produce large (mg) quantities of the mutated allergen. Methods for preparing recombinant proteins in these hosts are well known in the art and are described in great detail in Current Protocols in Molecular Biology Ed. by Ausubel et al., John Wiley & Sons, New York, N. Y., 1989 and Molecular Cloning: A Laboratory Manual Ed. by Sambrook et al., Cold Spring Harbor Press, Plainview, N. Y., 1989.

Transgenic plants or animals expressing the modified allergens can also be used as a source of mutated allergen for use in immunotherapy. Methods for engineering of plants and animals are well known and have been for a decade. For example, for plants see Day, Crit. Rev. Food Sci. & Nut. 36:S549, 1996. See also Fuchs and Astwood, Food Tech. 83-88, 1996. Methods for making recombinant animals are also well established. See, for example, Colman, Biochem. Soc. Symp. 63:141, 1998; Espanion and Niemann, DTW Dtxch. Tierarztl. Wochenschr. 103:320, 1996; and Colman, Am. J. Clin. Nutr. 63:639 S, 1996. One can also induce site specific changes using homologous recombination and/or triplex forming oligomers. See, for example, Rooney and Moore, Proc. Nad. Acad. Sci. USA 92:2141, 1995 and Agrawal et al., Bio World Today, vol. 9, no. 41, p. 1.

It will be appreciated that it is also possible to make the mutated allergen synthetically, if the allergen is not too large, for example, less than about 25-40 amino acids in length. Such peptides may utilize only naturally-occurring amino acids, or may include one or more non-natural amino acid analog or other chemical compound capable of being incorporated into a peptide chain. Non-natural amino acids are amino acids that do not occur in nature but that can be incorporated into a polypeptide chain (e.g., the amino acids shown on the world wide web at http://www.cco.caltech.edu/˜dadgrp/Unnatstmet.gif, which displays structures of non-natural amino acids that have been successfully incorporated into functional ion channels).

In preferred embodiments the modified allergen includes one or more mutations that disrupt one or more of the linear IgE epitopes. It is to be understood that the mutations may involve substitutions for any other amino acid and that the methods are in no way limited to substitutions with alanine or methionine residue as described in the Examples (see Examples 5, 13, and 18). Additionally or alternatively, the mutations may involve one or more deletions within one or more linear IgE epitopes. Typically linear IgE epitopes are about 6 to about 10 amino acids in length. As shown in Examples 4, 11, and 17, single mutations within these linear epitopes can dramatically reduce IgE binding. Accordingly, in certain embodiments of the present invention one need only modify between 1 in 6 (i.e., about 17%) and 1 in 10 (i.e., about 10%) of the amino acids in a linear IgE epitope to reduce IgE binding. In other embodiments, one may modify 2 (i.e., between about 20-34%), 3 (i.e., between about 30-50%), 4 (i.e., between about 40-67%), (i.e., between about 50-83%), or more amino acids within a linear IgE epitope.

Mutations involving cysteine residues may be used to disrupt one or more disulfide bonds. Preferred substituents for cysteine include but are not limited to serine, threonine, alanine, valine, glycine, leucine, isoleucine, histidine, tyrosine, phenylalanine, tryptophan, and methionine. Alternatively, one or more cysteine residues may be substituted with a synthetic amino acid which has a side chain with the formula —[CH₂]ₙ—R wherein n is an integer between 1 and 5 and R is a 1-3 carbon moiety selected from the group consisting of alkyl groups (e.g., methyl, ethyl, n-propyl, etc.); carboxy alkyl groups (e.g., carboxymethyl, carboxyethyl, etc.); cyano alkyl (e.g., cyanomethyl, cyanoethyl, etc.); alkoxycarbonyl alkyl groups (e.g., ethoxycarbonylmethyl, ethoxycarbonylethyl, etc.); carbomoylalkyl groups (e.g., carbamoylmethyl, etc.); and alkylamine groups (e.g., methylamine, ethylamine, etc.).

### Reduced and Alkylated Allergens

In certain embodiments, the modified allergens of the present invention may be reduced and alkylated in order to disrupt one or more disulfide bonds that are present in the natural allergen. Methods for reducing and alkylating proteins have been described in the art, e.g., for a review see Herbert et al., Electrophoresis 22:2046, 2001. Examples of reducing agents that may be used include but are not limited to 2-mercaptoethanol, dithiothreitol, dithioerythritol, iodoacetamide, and tributylphosphosphine. Alkylation can then be performed by blocking the SH radicals resulting from the cleavage of the disulfide bonds in a conventional manner, e.g., using iodoacetamide, iodoacetic acid, or derivatives thereof. More generally, at least one disulfide bond can be reduced and alkylated to produce cysteine residues with side chains having the chemical formula —CH₂—S—[CH₂]ₙ—R′ wherein n is an integer between 1 and 5 and R′ is selected from the 1-5 carbon groups consisting of alkyl groups (e.g., methyl, ethyl, n-propyl, etc.); carboxy alkyl groups (e.g., carboxymethyl, carboxyethyl, etc.); cyano alkyl groups (e.g., cyanomethyl, cyanoethyl, etc.); alkoxycarbonyl alkyl groups (e.g., ethoxycarbonylmethyl, ethoxycarbonylethyl, etc.); carbomoylalkyl groups (e.g., carbamoylmethyl, etc.); and alkylamine groups (e.g., methylamine, ethylamine, etc.).

### Additional or Alternative Modifications

It is to be understood that one or more of the amino acids in an inventive peptide may be further modified, for example, by the addition of a chemical entity such as a carbohydrate group, a phosphate group, a farnesyl group, an isofarnesyl group, a fatty acid group, a linker for conjugation, functionalization, or other modification, etc. Alternatively or additionally, inventive modified allergens may be produced as a fusion with another polypeptide chain. In some embodiments, it may be desirable to include a cleavage site within such a fusion peptide, that can be activated by an enzyme, a chemical, or by experimental conditions (e.g., pH).

Alternatively or additionally, the disulfide bonds of modified allergens may be oxidatively denatured as described in U.S. Pat. No. 5,061,790 to Elting et al. According to the methods provided therein, oxidizing agents that have an oxidation potential which is sufficient to cleave disulfide bonds (e.g., but not limited to, periodate, peroxodisulfate, hypochlorite, chromate, and perchlorate) may be used to disrupt disulfide bonds. The cysteine residues are thereby chemically oxidized to amino acids that include a side chain with the chemical formula —CH₂—X where X is SO₃⁻ or S—SO₃⁻.

### C. Assays for Screening Modified Allergens

Assays to assess an immunologic change after the administration of the modified allergen are known to those skilled in the art. Conventional assays include RAST (Sampson and Albergo, 1984), ELISAs (Burks et al., 1986), immunoblotting (Burks et al., 1988), in vivo skin tests (Sampson and Albergo 1984), and basophil histamine release assays (Nielsen, Dan. Med. Bull. 42:455, 1995 and du Buske, Allergy Proc. 14:243, 1993). Objective clinical symptoms can be monitored before and after the administration of the modified allergen to determine any change in the clinical symptoms.

Certain preferred modified allergens of the present invention are characterized by their ability to suppress a Th2-type response and/or to stimulate a Th1-type response preferentially as compared with their ability to stimulate a Th2-type response. Th1 and Th2-type responses are well-established alternative immune system responses that are characterized by the production of different collections of cytokines and/or cofactors that can be assayed for. For example, Th1-type responses are generally associated with production of cytokines such as IL-1β, IL-2, IL-12, IL-18, IFNα, IFNγ, TNFβ, etc; Th2-type responses are generally associated with the production of cytokines such as IL-4, IL-5, IL-10, etc. The extent of T-cell subset suppression or stimulation may be determined by any available means including, for example, intra-cytoplasmic cytokine determination. In preferred embodiments of the invention, Th2 suppression is assayed, for example, by quantitation of IL-4, IL-5, and/or IL-13 in stimulated T-cell culture supernatant or assessment of T-cell intra-cytoplasmic (e.g., by protein staining or analysis of mRNA) IL-4, IL-5, and/or IL-13; Th1 stimulation is assayed, for example, by quantitation of IFNα, IFNγ, IL-2, IL-12, and/or IL-18 in activated T-cell culture supernatant or assessment of intra-cytoplasmic levels of these cytokines.

### D. Pharmaceutical Compositions

### Introduction

As discussed above, the present invention provides modified allergens which have biological properties which make them of interest for the treatment of allergies and in particular anaphylactic reactions. Accordingly, in another aspect of the present invention, pharmaceutical compositions are provided, wherein these compositions comprise a modified allergen, and optionally comprise a pharmaceutically acceptable carrier and/or an adjuvant. It will be appreciated that certain of the modified allergens of present invention can exist in free form for treatment or may be provided as crude preparations, such as a chemical or proteolytic digestion of a food extract (see, for example, Hong et al., J. Allergy Clin. Immunol. 104:473, 1999). Those of ordinary skill in the art will also appreciate that inventive modified allergens may be provided by combination or association with one or more other agents such as targeting agents or may be encapsulated (e.g., within a liposome, nanoparticle, or a live, preferably attenuated, infectious organism such as a bacterium or a virus), as discussed in more detail below.

### Carriers

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995, discloses various carriers used in formulating pharmaceutical compositions and known techniques for the preparation thereof. Except insofar as any conventional carrier medium is incompatible with the modified protein allergen of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Adjuvants

In certain preferred embodiments of the invention, the modified allergens are provided with one or more immune system adjuvants. A large number of adjuvant compounds are known; a useful compendium of many such compounds is prepared by the NIH and can be found on the world wide web at http://www.niaid.nih.gov/daids/vaccine/pdf/compendium.pdf (see also Allison, Dev. Biol. Stand 92:3, 1998; Unkeless et al., Annu. Rev. Immunol. 6:251, 1998; and Phillips et al., Vaccine 10:151, 1992). Preferred adjuvants are characterized by an ability to stimulate a Th1-type response preferentially over Th2-type response and/or to down regulate a Th2-type response. In fact, in certain preferred embodiments of the invention, adjuvants that are known to stimulate Th2-type responses are avoided. Particularly preferred adjuvants include, for example, preparations (including heat-killed samples, extracts, partially purified isolates, or any other preparation of a microorganism or macroorganism component sufficient to display adjuvant activity) of microorganisms such as Listeria monocytogenes, Escherichia coli or others (e.g., bacille Calmette-Guerin (BCG), Corynebacterium species, Mycobacterium species, Rhodococcus species, Eubacteria species, Bortadella species, and Nocardia species), and preparations of nucleic acids that include unmethylated CpG motifs (see, for example, U.S. Pat. No. 5,830,877; and published PCT applications WO96/02555, WO98/18810, WO98/16247, and WO98/40100). Other preferred adjuvants reported to induce Th1-type responses and not Th2-type responses include, for example, AVRIDINE™ (N,N-dioctadecyl-N′N′-bis(2-hydroxyethyl)propanediamine) available from M6 Pharmaceuticals of New York, N. Y.; niosomes (non-ionic surfactant vesicles) available from Proteus Molecular Design of Macclesfield, UK; and CRL 1005 (a synthetic ABA non-ionic block copolymer) available from Vaxcel Corporation of Norcross, Ga.

In some embodiments of the invention, the adjuvant is associated (covalently or non-covalently, directly or indirectly) with the modified allergen so that adjuvant and modified allergen can be delivered substantially simultaneously to the individual, optionally in the context of a single composition. In other embodiments, the adjuvant is provided separately. Separate adjuvant may be administered prior to, simultaneously with, or subsequent to modified allergen administration. In certain preferred embodiments of the invention, a separate adjuvant composition is provided that can be utilized with multiple different modified allergen compositions.

Where adjuvant and modified allergen are provided together, any association sufficient to achieve the desired immunomodulatory effects may be employed. Those of ordinary skill in the art will appreciate that covalent associations will sometimes be preferred. For example, where adjuvant and modified allergen are both polypeptides, a fusion polypeptide may be employed. To give another example, CpG-containing nucleotides may readily be covalently linked with modified allergens. Those of ordinary skill in the art will be aware of other potential desirable covalent linkages.

### Targeting Agents

Inventive modified allergens may desirably be associated with a targeting agent that will ensure delivery to a particular desired location. In preferred embodiments of the invention, the modified allergen is targeted for uptake by APCs. For example, a modified allergen could be targeted to dendritic cells or macrophages via association with a ligand that interacts with an uptake receptor such as the mannose receptor or an Fc receptor. A modified allergen could be targeted to other APCs via association with a ligand that interacts with the complement receptor. A modified allergen could be specifically directed to dendritic cells through association with a ligand for DEC205, a mannose-like receptor that is specific for these cells.

Alternatively or additionally, a modified allergen could be targeted to particular vesicles within APCs. Those of ordinary skill in the art will appreciate that any targeting strategy should allow for proper uptake and processing of the modified allergen by the APCs.

A modified allergen of the present invention can be targeted by association of the modified allergen containing composition with an Ig molecule, or portion thereof. Ig molecules are comprised of four polypeptide chains, two identical “heavy” chains and two identical “light” chains. Each chain contains an amino-terminal variable region, and a carboxy-terminal constant region. The four variable regions together comprise the “variable domain” of the antibody; the constant regions comprise the “constant domain”. The chains associate with one another in a Y-structure in which each short Y arm is formed by interaction of an entire light chain with the variable region and part of the constant region of one heavy chain, and the Y stem is formed by interaction of the two heavy chain constant regions with one another. The heavy chain constant regions determine the class of the antibody molecule, and mediate the molecule's interactions with class-specific receptors on certain target cells; the variable regions determine the molecule's specificity and affinity for a particular antigen.

Class-specific antibody receptors, with which the heavy chain constant regions interact, are found on a variety of different cell types and are particularly concentrated on professional antigen presenting cells (pAPCs), including dendritic cells. According to the present invention, inventive compositions, and particularly modified allergen-containing compositions, may be targeted for delivery to pAPCs through association with an Ig constant domain. In one embodiment, an Ig molecule is isolated whose variable domain displays specific affinity for the modified allergen to be delivered, and the allergen is delivered in association with the Ig molecule. The Ig may be of any class for which there is an Ig receptor, but in certain preferred embodiments, is an IgG. Also, it is not required that the entire Ig be utilized; any piece including a sufficient portion of the Ig heavy chain constant domain is sufficient. Thus, Fc fragments and single-chain antibodies may be employed in the practice of the present invention.

In one embodiment of the invention, a modified allergen is prepared as a fusion molecule with at least an Ig heavy chain constant region (e.g., with an Fc fragment), so that a single polypeptide chain, containing both modified allergen and Ig heavy chain constant region components, is delivered. This embodiment allows increased flexibility of allergen selection because the length and character of the modified allergen is not constrained by the binding requirements of the Ig variable domain cleft. In particularly preferred versions of this embodiment, the modified allergen portion and the Fc portion of the fusion molecule are separated from one another by a severable linker that becomes cleaved when the fusion molecule is taken up into the pAPC. A wide variety of such linkers are known in the art. Fc fragments may be prepared by any available technique including, for example, recombinant expression (which may include expression of a fusion protein) proteolytic or chemical cleavage of Ig molecules (e.g., with papain), chemical synthesis, etc.

### Encapsulation

In one particularly preferred embodiment of the invention, the inventive modified allergen is provided in association with an encapsulation device (see, for example, U.S. Patent Application Ser. No. 60/169,330 entitled “Encapsulation of Antigens”, filed on Dec. 6, 1999, and incorporated herein by reference herewith). Preferred encapsulation devices are biocompatible and stable inside the body so that the modified allergen is not released until after the encapsulation device is taken up into an APC. For example, preferred systems of encapsulation are stable at physiological pH and degrade at acidic pH levels comparable to those found in the endosomes of APCs. Preferably, the encapsulation device is taken up into APC via endocytosis in clathrin-coated pits. Particularly preferred encapsulation compositions included but are not limited to ones containing liposomes, polylactide-co-glycolide (PLGA), chitosan, synthetic biodegradable polymers, environmentally responsive hydrogels, and gelatin PLGA nanoparticles. Inventive modified allergens may be encapsulated in combination with one or more adjuvants, targeting entities, or other agents including, for example, pharmaceutical carriers, diluents, excipients, oils, etc. Alternatively or additionally the encapsulation device itself may be associated with a targeting agent and/or an adjuvant.

In one particularly preferred embodiment of the invention, the encapsulation device comprises a live, preferably attenuated, infectious organism (i.e., a bacterium or a virus). The modified allergen may be introduced into the organism by any available means. In preferred embodiments of the invention, the organism is genetically engineered so that it synthesizes the modified allergen itself. For example, genetic material encoding a modified allergen may be introduced into the organism according to standard techniques (e.g., transfection, transformation, electroporation, injection, etc.) so that it is expressed by the organism and the modified allergen is produced. In particularly preferred embodiments of the invention, the modified allergen is engineered to be secreted from the organism (see, for example, published PCT application WO98/23763). Those of ordinary skill in the art will appreciate that analogous systems can be engineered using any of a variety of other bacterial or viral systems. Any such system may be employed in the practice of the present invention.

The advantages of utilizing a bacterium or virus as an encapsulation system include (i) integrity of the system prior to endocytosis, (ii) known mechanisms of endocytosis (often including targeting to particular cell types), (iii) ease of production of the delivered modified allergen (typically made by the organism), (iv) experimental accessibility of the organisms, including ease of genetic manipulation, (v) ability to guarantee release (e.g., by secretion) of the antigen fragment after endocytosis, and (vi) the possibility that the encapsulating organism will also act as an adjuvant (e.g., Listeria monocytogenes, Escherichia coli, etc.).

### E. Uses of Pharmaceutical Compositions Introduction

In yet another aspect, according to the methods of treatment of the present invention, an individual who suffers from or is susceptible to an allergy may be treated with a pharmaceutical composition, as described herein. It will be appreciated that an individual can be considered susceptible to allergy without having suffered an anaphylactic reaction to the particular allergen in question. For example, if the individual has suffered an allergic or anaphylactic reaction to a related allergen (e.g., one from the same source or one for which shared allergies are common), that individual will be considered susceptible to anaphylactic reaction to the relevant allergen. Similarly, if members of an individual's family react to a particular allergen, the individual may be considered to be susceptible to anaphylactic reaction to that allergen.

In general, it is believed that the inventive modified allergens will be clinically useful in treating or preventing allergic reactions associated with any natural allergen, in particular anaphylactic allergens including but not limited to food allergens, insect allergens, and rubber allergens (e.g., latex).

It will be appreciated that therapy or desensitization with the modified allergens can be used in combination with other therapies, such as allergen-non-specific anti-IgE antibodies to deplete the patient of allergen-specific IgE antibodies (see, Boulet et al., Am. J. Respir. Crit. Care Med. 155:1835, 1997; Fahy et al., Am. J. Respir. Crit. Care Med. 155:1828, 1997; and Demoly and Bousquet, Am J. Resp. Grit. Care Med. 155:1825, 1997), or by the pan specific anti-allergy therapy described in U.S. Ser. No. 08/090,375 filed Jun. 4, 1998.

It will further be appreciated that the therapeutic and prophylactic methods encompassed by the present invention are not limited to treating allergic reactions in humans, but may be used to treat wounds in any animal including but not limited to mammals, e.g., bovine, canine, feline, caprine, ovine, porcine, murine, and equine species.

### Therapeutically Effective Dose

Thus, the invention provides methods for the treatment of allergies comprising administering a therapeutically effective amount of a pharmaceutical composition comprising active agents that include a modified allergen to an individual in need thereof, in such amounts and for such time as is necessary to achieve the desired result. It will be appreciated that this encompasses administering an inventive pharmaceutical as a therapeutic measure to treat an individual who suffers from an allergy or as a prophylactic measure to desensitize an individual that is susceptible to an allergy. In certain embodiments of the present invention a “therapeutically effective amount” of the pharmaceutical composition is that amount effective for preventing an allergic reaction in an individual who suffers from an allergy or an individual who is susceptible to an allergy. The pharmaceutical compositions, according to the method of the present invention, may be administered using any amount and any route of administration effective for preventing an allergic reaction. Thus, the expression “amount effective for preventing an allergic reaction”, as used herein, refers to a sufficient amount of pharmaceutical composition to prevent an allergic reaction. The exact dosage is chosen by the individual physician in view of the patient to be treated. Dosage and administration are adjusted to provide sufficient levels of the active agent(s) or to maintain the desired effect. Additional factors which may be taken into account include the severity of the allergic reaction; age, weight and gender of the individual; diet, time and frequency of administration, therapeutic combinations, reaction sensitivities, and tolerance/response to therapy. Long acting pharmaceutical compositions might be administered every 3 to 4 days, every week, or once every two weeks depending on half-life and clearance rate of the particular formulation. In general, effective amounts will be in the picogram to milligram range, more typically microgram to milligram. Treatment will typically be between twice/weekly and once a month, continuing for up to three to five years, although this is highly dependent on the individual patient response. In certain embodiments, the active agents of the invention may be administered rectally at dosage levels of about 0.01 mg/kg to about 50 mg/kg and preferably from about 1 mg/kg to about 25 mg/kg, of subject body weight per day, one or more times a day, to obtain the desired therapeutic effect.

The active agents of the invention are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions of the present invention will be decided by the attending physician within the scope of sound medical judgment. For any active agent, the therapeutically effective dose can be estimated initially either in cell culture assays or in non-human animal models, usually mice, rabbits, dogs, or pigs. The non-human animal model is also used to achieve a desirable concentration range and route of administration. Such information can then be used to determine useful doses and routes for administration in humans. A therapeutically effective dose refers to that amount of active agent which ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and non-human animal studies is used in formulating a range of dosage for human use.

### Administration of Pharmaceutical Compositions

After formulation with an appropriate pharmaceutically acceptable carrier in a desired dosage, the pharmaceutical compositions of this invention can be administered to humans and other mammals topically (as by powders, ointments, or drops), orally, rectally, parenterally, intracisternally, intravaginally, intraperitoneally, subcutaneously, intramuscularly, intragastrically, bucally, ocularly, or nasally, depending on the severity and location of the allergic reaction being treated or prevented.

Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, ocular or cutaneous infections may be treated with aqueous drops, a mist, an emulsion, or a cream.

The ointments, pastes, creams, and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the agents of this invention, excipients such as lactose, talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

### EXAMPLES

Peanut allergy is one of the most common and serious of the immediate hypersensitivity reactions to foods in terms of persistence and severity of reaction. Unlike the clinical symptoms of many other food allergies, the reactions to peanuts are rarely outgrown, therefore, most diagnosed children will have the disease for a lifetime (Sampson and Burks, Annu. Rev. Nutr. 16:161, 1996 and Bock, J. Pediatr. 107:676, 1985). The majority of cases of fatal food-induced anaphylaxis involve ingestion of peanuts (Sampson et al., NEJM 327:380, 1992 and Kaminogawa, Biosci. Biotech. Biochem. 60:1749, 1996). The only effective therapeutic option currently available for the prevention of a peanut hypersensitivity reaction is food avoidance. Unfortunately, for a ubiquitous food such as a peanut, the possibility of an inadvertent ingestion is great.

Peanut allergens were therefore chosen along with other food allergens (e.g., soybean, wheat, and walnut allergens) to illustrate the various aspects of the present invention. Examples 1-18 provided below describe how the methods of the present invention have been used to prepare modified versions of peanut allergens Ara h 1, Ara h 2 and Ara h 3 with reduced IgE binding. Examples 1-7 describe the isolation, purification, characterization, and modification of the major peanut allergen Ara h 1, a member of the vicilin family of seed storage proteins. Examples 8-14 describe the isolation, purification, characterization, and modification of the major peanut allergen Ara h 2, a member of the conglutin family of seed storage proteins. Examples 15-18 describe the isolation, purification, characterization, and modification of the major peanut allergen Ara h 3, a member of the glycinin family of seed storage proteins. Examples 19-23 describe the isolation, purification, and characterization of various soybean allergens. Examples 24 and 25 describe the isolation, purification, and characterization of wheat and walnut allergens, respectively. Example 26 describes the preparation of an IgE Fab cDNA library to peanut allergens. Finally, Example 27 describes the evaluation of heat killed E. coli expressing modified Ara h 1, 2, and 3 for the desensitization of peanut-allergic mice.

### Example 1

### Purification and Isolation of Ara h 1 Using Pooled IgE Sera

### 1.1 Introduction

Purification and isolation of a major peanut allergen was accomplished using anion-exchange column chromatography, sodium dodecyl sulfate-polyacrylamide gel electrophoresis, ELISA, thin-layer isoelectric focusing, and IgE-specific immunoblotting. Anion-exchange chromatography revealed several fractions that bound IgE from the serum of a challenge-positive patient pool. By measuring anti-peanut-specific IgE in the ELISA and in IgE-specific immunoblotting, we identified an allergenic component with two Coomassie brilliant blue staining bands by sodium dodecyl sulfate-polyacrylamide gel electrophoresis with a mean molecular weight of 63.5 kd. By examining this fraction using the IgE anti-peanut ELISA with individual serum and the ELISA-inhibition assay with pooled serum, we identified this fraction as a major allergen. Thin-layer isoelectric focusing and immunoblotting of this 63.5 kd fraction revealed it to have an isoelectric point of 4.55. Based on allergen nomenclature of the IUIS Subcommittee for Allergen Nomenclature, this allergen is designated, Ara h 1 (Arachis hypogaea).

### 1.2 Methods

### Peanut-Sensitive Patients

Approval for this study was obtained from the Human Research Advisory Committee at the University of Arkansas for Medical Sciences. Nine patients (mean age, 4.2 years) with AD and a positive immediate prick skin test to peanut had either a positive DBPCFC or a convincing history of peanut anaphylaxis (the allergic reaction was potentially life threatening, that is, laryngeal edema, severe wheezing and/or hypotension). Details of the challenge procedure and interpretation have been discussed previously (Burks et al., J. Pediatr. 113:447-451, 1988a). Five milliliters of venous blood was obtained from each patient and allowed to clot, and then the serum was collected. An equal volume of serum from each donor was mixed to prepare a nine-person, peanut-specific, IgE Ab pool.

### Crude Peanut Extract

Three commercial lots of southeastern runners (Arachis hypogaea) (Florunner), medium grade from 1979 crop (North Carolina State University), were used in this study. The peanuts were stored in the freezer at −18° C. until they were roasted. The three lots were combined in equal proportions and blended before defatting. The defatting process (defatted with hexane after roasting for 13 to 16 minutes at 163° to 177° C.) was done in the laboratory of Dr. Clyde Young (North Carolina State University). The powdered crude peanut was extracted according to the recommendations of Yunginger and Jones (“A review of peanut chemistry: implications for the standardization of peanut extracts” in Proceedings of the 4^{th} International Paul Ehrlich Seminar on the Regulatory Control and Standardization of Allergenic Extracts. Bethesda, Md., Oct. 16-17, 1985. Published by Gustav Fischer Verlag, Stuttgart, 1987:251-264) in 1 mol/L of NaCl to 20 mmol/L of sodium phosphate (pH 7.0), with the addition of 8 mol/L of urea for 4 hours at 4° C. The extract was isolated by centrifugation at 20,000 g for 60 minutes at 4° C.

### Chromatography

Analytic and preparative anion-exchange chromatography was performed with the FPLC system (Pharmacia, Piscataway, N. J.). Anion-exchange chromatography was used with the Mono Q 5/5 and 10/10 columns (Pharmacia). The crude peanut extract was dialyzed against 20 mmol/L of Tris-bis-propane (pH 7.2) and 8 mol/L of urea, and 40 mg was loaded onto the Mono Q 10/10 column. A stepwise salt gradient of 0 to 1.5 mol/L of NaCl was applied. All fractions of each resolved peak were pooled, dialyzed, and lyophilized.

Dot blotting was done to determine which fractions from the anion-exchange column chromatogram contained IgE-binding material. The collected fractions (200 μl) were blotted with the Mini Blot apparatus (Schleicher & Schuell Inc., Keene, N. H.) onto 0.45 micron nitrocellulose membranes (Bio-Rad Laboratories, Richmond, Calif.). After membranes were dried, the remaining active sites were blocked with 20 ml of blocking solution (0.5% gelatin with 0.001% thimerosal in 500 ml of PBS) for 1 hour. The procedure is then identical to the immunoblotting of IgE.

### Electrophoresis and Immunoblotting

The electrophoresis procedure (Laemmli, Nature 227:680-685, 1970) was a modification of the method of Sutton et al (J. Immunol. Methods 52:183-186, 1982). SDS-PAGE was performed with a 12.5% polyacrylamide separating gel and a stacking gel of 3%. Twenty microliters of a 1 mg/ml solution of each protein was applied to each well. Replicate samples were applied for independent analysis. Electrophoresis was performed for 4 hours at 0.030 A per gel (E-C Apparatus Corp., St. Petersburg, Fla.) for the 14 cm by 12 cm gels, and for 1 hour at 175 V per gel for the 8 cm by 7.5 cm gels (Mini-Protean II system, Bio-Rad Laboratories). To assure proper protein separation and visualization, Coomassie brilliant blue (Sigma Chemical Co., St. Louis, Mich.) stains were done on gels. For detection of carbohydrate staining material, gels were stained with the modified PAS stain according to the method of Kapitany and Zebrowski (Anal. Biochem. 56:361-369, 1973).

Proteins were electrophorectically transferred from the separating gel to a nitrocellulose membrane in a transfer buffer (Tris-glycine) with 10% SDS and 40% methanol (Towbin et al., Proc. Natl. Acad. Sci. USA 76:4350-4354, 1979). The procedure was done in a transblot apparatus (Bio-Rad Laboratories) for 2 hours (0.150 A) (regular size transfer apparatus for crude peanut and minitransfer apparatus for fraction 3). An amido black stain (Bio-Rad Laboratories) was done to assure transfer of the protein.

After removal from the transblot apparatus, the nitrocellulose was placed in blocking solution overnight at 4° C. The nitrocellulose blot was then washed three times with PBS (PBS with 0.05% Tween 20) and incubated with the pooled serum (1:20 vol/vol dilution) for 2 hours at 4° C. with rocking. After the nitrocellulose blot was again washed with PBS three times, alkaline phosphatase-conjugated goat antihuman IgE (1:1000 vol/vol of PBS, Bio-Rad Laboratories) was added and incubated at room temperature with rocking for 2 hours. After an additional wash with PBS three times, the blot was developed with 250 μl of 30 mg of nitro blue tetrazolium in 70% dimethylformamide and 250 μl of 15 mg of 5-bromo-4-chloro-3-indolyl-phosphate in 70% dimethylformamide (Bio-Rad Laboratories) solutions in 25 ml of carbonate buffer (0.2 mol/L, pH 9.8) at room temperature for 15 minutes. The reaction was then stopped by decanting the 30 mg of nitro blue tetrazolium in 70% dimethylforamide/15 mg of 5-bromo-4-chloro-3-indolyl-phosphate in 70% dimethylforamide solution and incubating the nitrocellulose for 10 minutes with distilled water. The blot was then air-dried.

### ELISA for IgE

A biotin-avidin ELISA was developed to quantify IgE antipeanut protein Abs with modifications from an assay previously published (Burks et al., N Engl. J. Med. 314:560-564, 1986). The upper two rows of a 96-well microtiter plate (Gibco, Santa Clara, Calif.) were coated with 100 μl each of equal amounts (1 μg/ml) of antihuman IgE MAbs, 7.12 and 4.15 (kindly provided by Dr. A. Saxon) in coating buffer (0.1 mol/L of sodium carbonate-bicarbonate buffer, pH 9.5). The remainder of the plate was coated with one of the peanut extracts at a concentration of 1 μg/ml in coating buffer. The plate was incubated at 37° C. for 1 hour and then was washed five times with rinse buffer (PBS, pH 7.4, containing 0.05% Tween 20; Sigma Chemical Co) immediately and between subsequent incubations. In the upper two rows we used a secondary standard IgE reference to generate a curve for IgE ranging from 0.05 to 25 ng/ml.

The serum pool and individual patient serum samples were diluted (1:20 vol/vol) and dispensed in duplicate in the lower portion of the plate. After incubation for 1 hour at 37° C. and a subsequent washing, biotinylated, affinity-purified, goat antihuman IgE (KPL, Gaithersburg, Md.) (1:1000 vol/vol of PBS) was added to all wells. Plates were incubated again for 1 hour at 37° C. and washed, and 100 μl of horseradish peroxidase-avidin conjugate (Vector Laboratories, Burlingame, Calif.) was added for 30 minutes. After plates were washed, they were developed by the addition of a buffer containing o-phenylenediamine (Sigma Chemical Co.). The reaction was stopped by the addition of 100 μl of 2-N-hydrochloric acid to each well, and absorbance was read at 492 nm (Titertek Multiscan, Flow Laboratories, McLean, Va.). The standard curve was plotted on a log-logit scale by means of simple linear regression, and values for the pool and individual patient samples were read from the curve as “nanogram-equivalent units” per milliliter (nanogram per milliliter) (Burks et al., J. Allergy Clin. Immunol. 81:1135-1142, 1988b and Burks et al., J. Allergy Clin. Immunol. 85:921-927, 1990).

### ELISA Inhibition

A competitive ELISA-inhibition analysis was done with the FPLC fractions. One hundred microliters of pooled serum (1:20 vol/vol) from the positive-challenge patients was incubated with various concentrations of the FPLC protein fractions (0.00005 to 50 ng/ml) for 18 hours at 4° C. The inhibited pooled serum was then used in the ELISA described above. The percent inhabitation was calculated by taking the food-specific IgE value minus the incubated food-specific IgE value divided by the food-specific IgE value. This number is multiplied by 100 to get the percentage of inhibition.

### Isoelectric Focusing

The samples were focused with the LKB Multiphor system with LKB PAG plates, pH gradient, 3.5 to 6.85 (LKB, Bromma, Sweden). Five microliters of sample (100 μg of protein) was applied and an electric current of 200 V was applied for 30 minutes and then increased to 900 to 1200 V for 30 minutes. The gel was fixed and stained with Coomassie brilliant blue following the standard protocol (LKB). For IgE immunoblotting, the protein was transferred to nitrocellulose by capillary transfer (Reinhart et al., Anal. Biochem. 123:229-235, 1982) and stained as described in the immunoblotting section above.

### 1.3 Results

### Chromatography

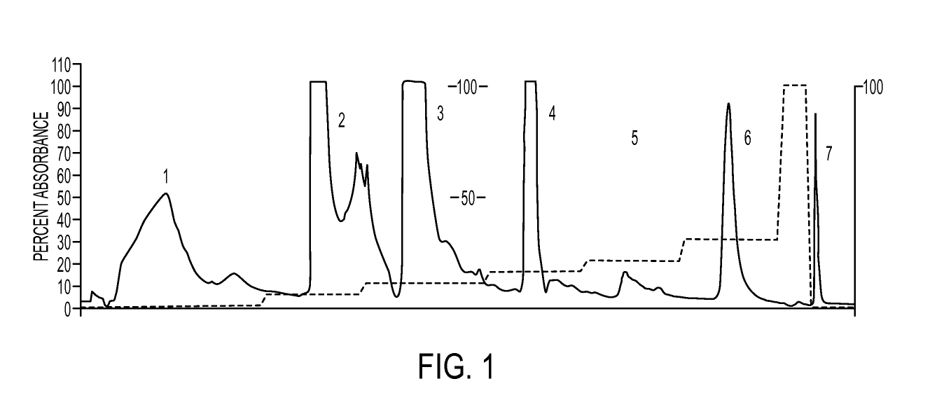

Pilot experiments were conducted with the analytical Mono Q5/5 anion-exchange column to determine the optimal buffer system and salt gradient. Scale up and optimization was completed with the Mono Q 10/10, with a stepwise salt gradient (0 to 1.5 mol/L of NaC1). This procedure separated the crude peanut extract into seven peaks (FIG. 1). Preliminary dot blotting from this separation identified IgE-binding material in each peak (data not presented). Multiple runs of this fractionation procedure were performed, and each isolated peak was pooled, dialyzed against 100 mmol/L of NH₄HCO₃, and lyophilized. Preliminary separation of crude peanut extract with gel filtration (Superose) did not significantly enrich the purification process.

### Electrophoresis and Immunoblotting

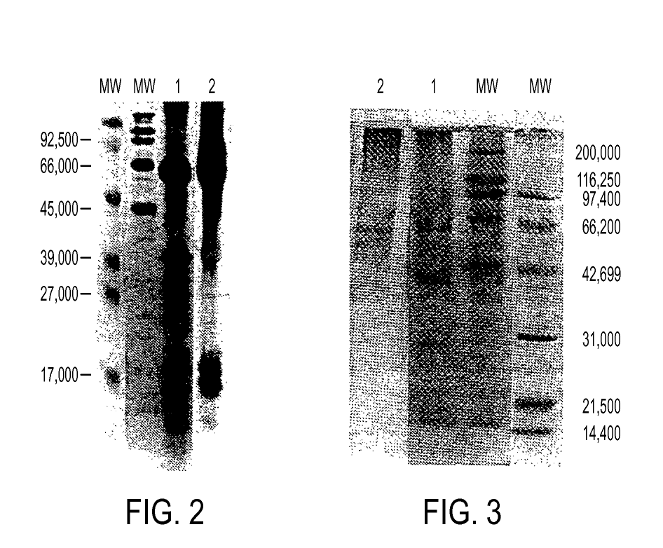

Initial SDS-PAGE and immunoblotting of the crude peanut extract revealed multiple protein fractions with several IgE-staining bands (FIG. 2). Aliquots of the seven lyophilized fractions from the anion-exchange column were analyzed by SDS-PAGE (data not presented). Immunoblotting for specific IgE with the pooled serum revealed two closely migrating bands that bound significant IgE in FIG. 3. Preliminary blots with normal control serum and with serum from patients with elevated serum IgE values revealed no non-specific binding to this fraction. The two bands in fraction 3 stained positive for PAS (data not presented). In addition, this fraction did not bind to Con A (after staining with biotinylated Con A and alkaline phosphatase-conjugated antibiotin) (data not presented).

### ELISA and ELISA Inhibition

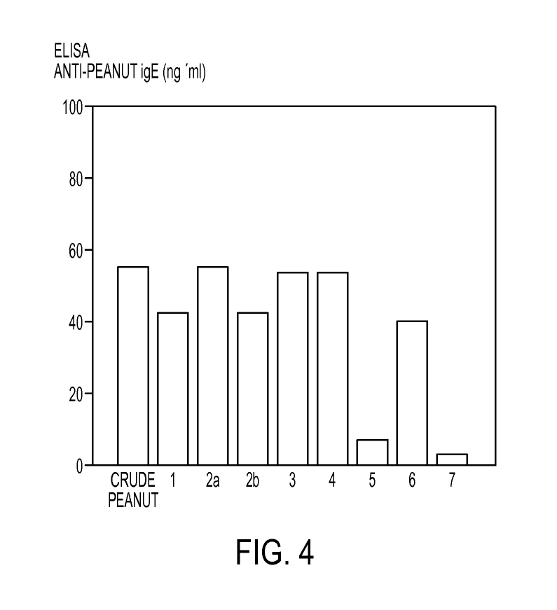

ELISA results comparing the crude peanut extract to each isolated fraction are illustrated in FIG. 4. Mono Q 10/10 fractions 2a, 3, and 4 had significant amounts (>50 ng/ml) of IgE binding compared to the crude peanut extract. We additionally tested the serum of six patients (members of the pooled serum) to determine the relative IgE binding to both the crude and the enriched allergen fraction containing the 63.5 kd component (fraction 3). The results are presented in Table 1.

TABLE 1
Individual IgE Ab to peanut allergens (ng/ml)

| Patient | Crude Peanut | 63.5 kd |
|---|---|---|
| 1 | 4.2 | 4.6 |
| 2 | 7.0 | 13.0 |
| 3 | 285.2 | 380.0 |
| 4 | 1.0 | 3.2 |
| 5 | 11.4 | 17.0 |
| 6 | 5.8 | 9.8 |
| 7 | ND | ND |
| 8 | ND | ND |

ND, Not detectable.
IgE-specific ELISAs to the crude peanut extract and the anion exchange fraction containing the 63.5 kd fraction.
Patients 1 to 6 are from the patients with AD and positive DBPCFCs to peanut.
Patient 7 is a patient with AD who had positive DBPCFC to milk and elevated serum
IgE values but was not skin test positive or challenge positive to peanut (n = 2).
Patient 8 is a healthy control patient from the serum bank in the ACH Special
Immunology Laboratory (n = 2).

Each patient's serum had measurable amounts of peanut-specific IgE to both the crude extract and the 63.5 kd fraction. Serum from patients with AD, elevated serum IgE values, and positive DBPCFCs to milk (patient No. 7) and from healthy normal controls (patient No. 8) did not have detectable peanut-specific IgE to this allergen.

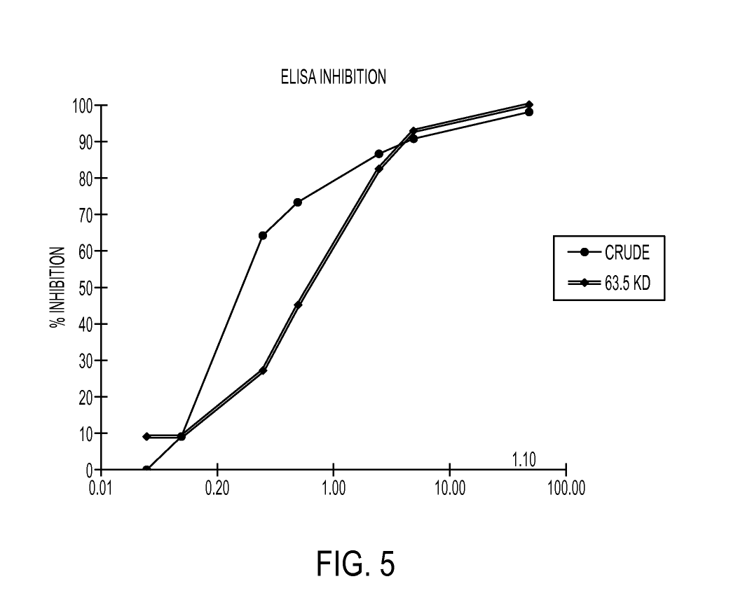

The ELISA-inhibition results are illustrated in FIG. 5. The concentration of the 63.5 kd fraction required to produce 50% inhibition was 5.5 ng/ml compared to 1.4 ng/ml of the crude peanut extract (Jusko, J. Clin. Pharmacol. 30:303-310, 1990). Control experiments with other food proteins did not demonstrate significant inhibition, demonstrating the specificity of the inhibition assay (data not presented).

### Isoelectric Focusing

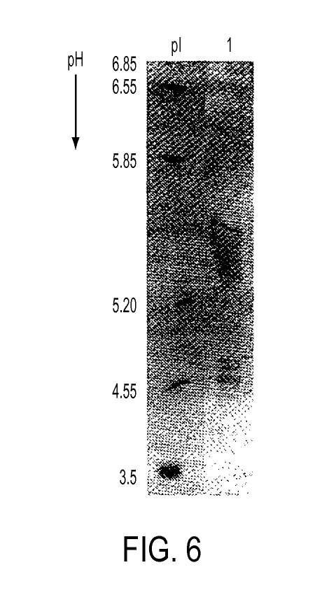

Because immunoblotting and ELISAs of the various anion-exchange fractions demonstrated that fraction 3 contained a major allergen, IEF and immunoblotting were done on this fraction. In FIG. 6, the two bands can be observed in this allergen that migrated closely together at 63.5 kd on SDS-PAGE, stained with Coomassie brilliant blue, to have a mean pI of 4.55 (FIG. 6). This protein fraction readily binds IgE form the pooled serum (data not presented).

### 1.4 Conclusion

In this study preliminary IgE blotting identified several IgE binding fractions in crude peanut extract. IgE-specific ELISA and immunoblotting of SDS-PAGE revealed two major allergenic bands migrating with an apparent mean molecular weight of 63.5 kd. We have designated this fraction Ara h 1. When used in an ELISA inhibition assay, Ara h 1 was found to significantly inhibit IgE binding to the crude peanut extract. Immunoblotting after IEF suggests that Ara h 1 has an approximate pI of 4.55. PAS staining suggests that Ara h 1 is a glycoprotein.

### Example 2

### Purification and Isolation of Ara h 1 Using Murine Mabs

### 2.1 Introduction

The antigenic and allergenic structure of the peanut allergen Ara h 1 (identified in Example 1) was investigated with the use of seven monoclonal antibodies obtained from BALC/c mice immunized with purified and isolated Ara h 1. When used as a solid phase in an ELISA, these monoclonal antibodies captured peanut allergen, which bound human IgE from patients with positive results to challenges to peanuts. The Ara h 1 monoclonal antibodies were found to be specific for peanut allergens when binding for other legumes was examined. In ELISA inhibition studies with the monoclonal antibodies we identified four different antigenic sites on Ara h 1. In related studies with pooled human IgE serum from patients with positive results to challenges to peanuts, we identified three similar IgE-binding epitopes. As a means of purifying the Ara h 1 allergen, we prepared an immunoaffinity column with monoclonal antibody 8D9. We eluted from this column the allergen Ara h 1, which had a mean molecular weight of 63.5 kd and which bound human IgE form individual and pooled serum of patients with peanut sensitivity.

### 2.2 Methods

Patients with Positive Results to Peanut Challenge

Approval for this study was obtained from the Human Use Committee at the University of Arkansas for Medical Sciences. Nine patients (mean age, 4.2 years) with atopic dermatitis and a positive immediate prick skin test result to peanut had either a positive double-blind, placebo-controlled food challenge (DBPCFC) to peanut or a convincing history of peanut anaphylaxis (the allergic reaction was potentially life-threatening, i.e. laryngeal edema, severe wheezing, and/or hypotension). Details of the challenge procedure and interpretation have been previously discussed (see Example 1). Five milliliters of venous blood was drawn from each patient and allowed to clot, and the serum was collected. An equal volume of serum from each donor was mixed to prepare a nine-person peanut-specific IgE antibody pool.

### Monoclonal Antibodies

Mouse hybridoma cell lines were prepared by standard hypoxanthine, aminopterin, and thymidine selection after polyethylene glycol-mediated cell fusion as described by de St. Groth and Scheidegger (J. Immunol. Methods 35:1-21, 1980). P3×63-Ag8.653 mouse/myeloma cells were fused with immune splenocytes from female BALB/c mice hyperimmunized with Ara h 1 (see Example 1). Hybridoma cell supernatants were screened by ELISA, and cell lines were cloned by limiting dilution (Kohler et al., Nature 256:495-497, 1975). The antibodies secreted by the monoclonal hybridoma cell lines were isotyped according to the directions provided (ScreenType, Boehringer Mannheim, Indianapolis, Ind.). Ascites fluid produced in pristane-primed BALB/c mice was purified with Protein C Superose, as outlined by the manufacturer (Pharmacia). Purified monoclonal antibodies from selected cell lines were used in an ELISA, and ELISA inhibition, and an immunoblot analysis for affinity purification of Ara h 1.

### ELISA

A biotin-avidin ELISA was developed to quantify anti-peanut (IgE) protein antibodies with modifications from an assay described previously (Burks et al., 1986, supra). The upper two rows of a 96-well microtiter plate (Gibco) were coated with 100 μl each of equal amounts (1 μg/ml) of anti-human IgE monoclonal antibodies 7.12 and 4.15 (kindly provided by Dr. A Saxon) in coating buffer (0.1 mol/L sodium carbonate-bicarbonate buffer, pH 9.5). The remainder of the plate was coated with 100 μl of Ara h 1 at a concentration of 1 μg/ml in coating buffer. The plate was incubated at 37° C. for 1 hour and was washed five times with rinse buffer (phosphate-buffered saline (PBS), pH 7.4, containing, 0.05% Tween 20) immediately and between subsequent incubations. In the upper two rows, a secondary IgE reference standard, ranging from 0.05 to 25 ng/ml, was used to generate a curve for IgE.

The peanut challenge-positive serum pool and patients' serum samples were diluted (1:20 vol/vol bovine serum albumin) and dispensed in duplicate in the lower portion of the plate. After incubation for 1 hour at 37° C. and washing, biotinylated, affinity-purified goat anti-human IgE (KPL, Gaitherburg, Md.) (1:2500 vol/vol bovine serum albumin) was added to all wells. Plates were incubated for 1 hour at 37° C. and washed; 100 μl of horseradish peroxidase-avidin conjugate (1:2500 vol/vol PBS) (Vector Laboratories, Burlingame, Calif.) was added for 5 minutes. After washing, the plates were developed by the addition of a buffer containing o-phenylenediamine (Sigma Chemical Co.). The reaction was stopped by the addition of 100 μl 2N-hydrochloric acid to each well, and absorbance was read at 492 nm with a Microplate Reader (model 450; Bio-Rad Laboratories). The standard curve was plotted on log-logit graph by means of simple linear regression analysis, and the antigen-specific values for the pool and for individual patients were read from the curve as a percent of the peanut-positive antibody pool.

An indirect ELISA was used to determined the ability of the various monoclonal antibody preparations to capture peanut antigen that could bind human IgE directed toward Ara h 1. In a 96-well microtiter plate (Gibco) 100 μl of the monoclonal antibody (at varying concentrations) was incubated in coating buffer (carbonate buffer, pH 9.6) for 1 hour at 37° C. After washing, crude peanut extract was added in diluent buffer (2% or bovine serum albumin and 0.05% Tween 20) for 1 hour at 37° C. Next, human serum containing IgE antibodies to Ara h 1 was added (either the pooled peanut-positive serum or serum from individual patients) for 1 hour at 37° C. After washing, biotinylated, affinity-purified goat anti-human IgE was added for 1 hour at 37° C. The plate was then developed as in the ELISA described previously.

### ELISA Inhibition

An ELISA inhibition assay was developed to examine the site specificity of the monoclonal antibodies generated to Ara h 1. One hundred microliters of Ara h 1 protein (1 mg/ml) was added to each well of a 96-well microtiter plate (Gibco) in coating buffer (carbonate buffer, pH 9.6) for 1 hour at 37° C. Next 100 μl of differing concentrations (up to 1000 times excess) of the seven monoclonal antibodies was added to each well for 1 hour at 37° C. After washing, a standard concentration of the biotinylated monoclonal antibody preparation was added for 1 hour at 37° C. The assay was developed by the addition of the avidin substrate as in the ELISA described previously.

A similar ELISA inhibition was performed with the peanut-positive serum IgE pool instead of the biotinylated monoclonal antibody to determine the ability of each monoclonal antibody to block specific IgE binding.

### Preparation of Anti-Peanut-Specific, IgE₁ Immunoaffinity Columns

Purified monoclonal antibody preparations from four cell lines were used to prepare immunoaffinity columns. Ten grams of freeze-dried cyanogen bromide-activated Sepharose (Sigma Chemical Co.) was swollen and washed in 2 L of 1 mmol/L HCl for 2 hours at room temperature. Swollen beads were collected in a scintered glass funnel and washed two times with an additional 2 L of 1 mol/L HCl to form a moist cake. The activated beads were then added to a monoclonal antibody solution (5 to 10 mg protein per milliliter of gel) dissolved in coupling buffer (0.1 mol/L NaHCO₃, 0.5 mol/L NaCl, pH 8.3) at room temperature for 2 hours. The unbound supernatant fraction was collected by centrifugation (1500 rpm for 10 minutes) and saved for residual antibody concentration analysis. The antibody-coupled gel was mixed with 100 ml 0.2 mol/L glycine at room temperature for 2 hours. The unbound supernatant fraction was again separated from the gel by centrifugation and saved for analysis. The immunoaffinity gel was then equilibrated with digestion buffer (1 mol/L NaCl, 20 mmol/L NaH₂PO₄). All supernatant fractions used to prepare the immunoaffinity column were analyzed for antibody (280 nm absorption), and the binding efficiency was determined by subtracting the unbound concentration from the total applied antibody concentration divided by total antibody times 100%.

### Affinity Purification of Ara h 1

One hundred milliliters of crude peanut extract (10 to 20 mg/ml) in digestion buffer was added (2 ml/min) to a peanut-specific immunoaffinity gel column (160×30 mm) incorporated into the fast protein liquid chromatography system (Pharmacia). Digestion buffer was passed through the column until the protein absorption (280 nm) reached baseline. Ara h 1 was eluted with 100 mmol/L triethylamine (pH 11.5) at a flow rate of 1 ml/min into test tubes containing 100 μl of 1 mol/L NaH₂PO₄ buffer to neutralize the eluate. Ara h 1 eluted in this manner from multiple runs was pooled, dialyzed against 100 mmol/L ammonium bicarbonate buffer, and lyophilized for storage before analysis.

### Electrophoresis and Immunoblotting

The electrophoresis procedure (Laemmli, 1970, supra) was a modification of that of Sutton et al. (Sutton et al., 1982, supra) Sodium dodecylsulfate-polyacrylamide gel electrophoresis (SDS-PAGE) was carried out with a 12.5% polyacrylamide separating gel and a stacking gel of 3%. Twenty microliters of a 1 mg/ml solution of each protein was applied to each well. Electrophoresis was performed for 4 hours at 0.030 A per gel (E-C Apparatus Corp., St. Petersburg, Fla.) for the 14×12 cm gels. To assure proper protein separation and visualization, Coomassie Brilliant Blue (Imperil Chemical Industries, Ltd., Macclesfield, Cheshire, England) stains were done on gels.

Proteins were transferred (Towbin et al., 1979, supra) from the separating gel to a nitrocellulose membrane in a transfer buffer (trisglycine) with 10% sodium dodecylsulfate and 40% methanol. The procedure was done in a transblot apparatus (Hoefer Scientific Instruments, San Francisco, Calif.) for 30 to 60 minutes (0.15 A). An amido black stain (Sigma Chemical Co.) was done to assure transfer of the protein.

After removal from the transblot apparatus, the nitrocellulose was placed in blocking solution overnight (0.5% gelatin, 0.05% Tween 20, thimerosal). The nitrocellulose blot was then washed three times with rinse buffer (PBS with 0.05% Tween 20) and incubated with the pooled serum (1:20 vol/vol) overnight at 4° C. with shaking. After washing again with PBS three times, alkaline phosphatase-conjugated goat anti-human IgE (1:1000 vol/vol PBS, 0.5% gelatin, thimerosal; KPL) was added and incubated at room temperature with shaking for 2 hours. After washing with PBS three times, the blot was developed by the addition of 250 μl nitroblue tetrazolium (Bio-Rad Laboratories) and 250 μl 5-bromo-4-chloro-3-indolyl phosphate (Bio-Rad Laboratories) solutions in 25 ml carbonate buffer (Bio-Rad Laboratories) at room temperature for 15 minutes. The reaction was then stopped by decanting the nitroblue tetrazolium/5-bromo-4-chloro-3-indolyl phosphate solution and incubating the nitrocellulose for 10 minutes was distilled water. The blot was then air dried.

Similar immunoblots were used for the screening of the monoclonal antibodies for Ara h 1. After the nitrocellulose was removed from the transblot apparatus and placed in blocking solution overnight, the supernatant from the hybridoma-secreting cell line was incubated overnight at 4° C. with shaking. The rest of the development procedure was identical to that for the IgE immunoblot.

### 2.3 Results

### Hybridomas Specific for Ara h 1

Cell fusions between spleen cells obtained from female BALB/c mice immunized with Ara h 1 and the mouse myeloma cells resulted in a series of hybridomas specific for Ara h 1. Thirteen monoclonal antibody-producing lines, which originated in five separate microtiter wells during the initial plating, were chosen for further study. In preliminary studies all 13 hybridoma-secreting cell lines had antibodies that bound Ara h 1, as determined by ELISA and immunoblot analysis. On the basis of different binding studies, seven of these hybridoma cell lines were chosen for further studies. As determined by isotype immunoglobulin-specific ELISA, all seven hybridoma-secreting cell lines typed as IgG₁.

ELISA with Monoclonal Antibody as Solid Phase

Seven monoclonal antibody preparations, 8D9, 8F10, 2E9, 7B3, 1B6, 6B5, and 6F9, were used as capture antibodies in an ELISA with Ara h 1 as the antigen. Serum from individual patients who had positive challenge results to peanut, was used to determine the amount of IgE binding to each peanut fraction captured by each specific monoclonal antibody (Table 2). A reference peanut-positive pool was used as the control serum for 100% binding. Six patients were chosen who had positive DBPCFCs to peanut (Patients No. 1-6). All six patients had elevated levels of anti-peanut-specific IgE to the peanut antigen presented by each of the seven monoclonal antibodies compared with the control sera (Patients No. 7 and 8). Titration curves were performed to show that limited amounts of antigen binding were not responsible for similar antibody binding. The amount of anti-peanut-specific IgE antibody to each peanut antigen presented individually by each monoclonal antibody did not differ significantly. However, there were individual patient differences in response to each set of peanut antigens presented by the monoclonal antibodies.

TABLE 2
Peanut-specific IgE to antigen presented
by seven monoclonal antibodies

| | Capture antibody (%) | | | | | | |
|---|---|---|---|---|---|---|---|
| Patient No. | 8D9 | 8F10 | 2E9 | 7B3 | 1B6 | 6B5 | 6F9 |
| 1 | 38 | 28 | 35 | 32 | 188 | 214 | 121 |
| 2 | 228 | 156 | 282 | 164 | 75 | 148 | 240 |
| 3 | 61 | 82 | 38 | 27 | 36 | 68 | 84 |
| 4 | 18 | 14 | 13 | 13 | 7 | 6 | 17 |
| 5 | 21 | 24 | 38 | 23 | 86 | 155 | 62 |
| 6 | 57 | 71 | 56 | 64 | 45 | 87 | 124 |
| 7 | 7 | 7 | 0 | 0 | 0 | 0 | 10 |
| 8 | 7 | 1 | 4 | 3 | 0 | 1 | 9 |

Ara h 1 monoclonal antibodies used as capture antibodies in ELISA with Ara h 1 as the antigen. Values are expressed as a percent of binding compared with challenge-positive peanut pool.
Patients 1 to 6 had positive DBPCFC results to peanut; patient 7 is the patient without peanut sensitivity with elevated serum IgE; and patient 8 is the patient without peanut sensitivity with normal serum IgE.

### Food Antigen Specificity of Monoclonal Antibodies to Ara h 1

To determine the specificity of the monoclonal antibodies prepared against Ara h 1 to detect peanut antigen, an ELISA was developed with the pooled peanut-specific IgE (from patients who had positive DBPCFC results to peanut). All seven monoclonal antibodies that were fully characterized bound only peanut antigen (Table 3 shows four of these monoclonal antibodies to Ara h 1). There was minimal binding in the ELISA to peas and chick peas but none to soy, green beans, lima beans, or ovalbumin. When the normal serum pool was used in this ELISA, no peanut-specific IgE could be detected to either Ara h 1 or crude peanut extract. In the United States three varieties of peanuts are commonly consumed—Virginia, Spanish, and Runner. In a similar ELISA, we attempted to determine whether there were differences in monoclonal antibody binding to the three varieties of peanuts. There was only a minor variation in the ability of the peanut-specific IgE to bind to the captured peanut antigen (data not show).

TABLE 3
IgE-specific binding to legumes captured
by Ara h 1 monoclonal antibodies

Capture antibody

| Protein | 8D9 | 8F10 | 2E9 | 7B3 |
|---|---|---|---|---|
| Pooled serum | | | | |
| Crude peanut | 0.854 | 0.868 | 0.875 | 0.883 |
| 63.5 kd (Ara h 1) | 0.834 | 0.846 | 0.903 | 0.884 |
| Soy | 0.125 | 0.132 | 0.133 | 0.122 |
| Peas | 0.254 | 0.231 | 0.256 | 0.233 |
| Chick peas | 0.238 | 0.196 | 0.198 | 0.244 |
| Green beans | 0.096 | 0.145 | 0.138 | 0.122 |
| Lima beans | 0.121 | 0.098 | 0.093 | 0.126 |
| Ovalbumin | 0.092 | 0.139 | 0.127 | 0.131 |
| Normal serum | | | | |
| Crude peanut | 0.122 | 0.094 | 0.125 | 0.099 |
| 63.5 kd (Ara h 1) | 0.131 | 0.096 | 0.092 | 0.126 |

Pooled serum is from patients with positive responses to peanut challenge. Ara h 1 monoclonal antibodies used as capture antibodies in ELISA with various legumes as the antigen. Values are expressed as optical density units.

### Site Specificity of Seven Hybridoma Antibodies

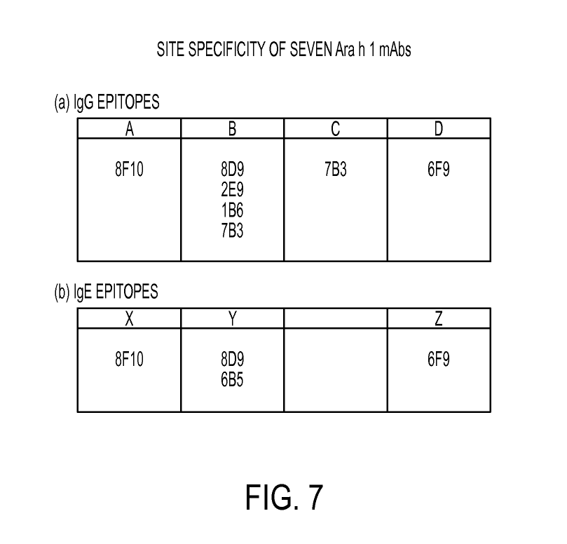

An ELISA inhibition assay was used to determine the site specificity of the seven monoclonal antibodies to Ara h 1. As determined by ELISA inhibition analysis there are at least four different epitopes, which could be recognized by the various monoclonal antibodies (Table 4). FIG. 7 depicts a schematic that incorporates the ELISA inhibition data to suggest at least four different epitopes on this allergen.

TABLE 4
ELISA inhibition for seven monoclonal antibodies to Ara h 1

| Biotinylated | Inhibiting antibody (%) | | | | | | | |
|---|---|---|---|---|---|---|---|---|
| mAb | 8F10 | 8D9 | 2E9 | 7B3 | 1B6 | 6B5 | 6F9 | Alt 1 |
| 8F10 | 71 | 10 | 11 | 11 | 2 | 5 | 5 | 0 |
| 8D9 | 31 | 82 | 34 | 0 | 28 | 26 | 5 | 0 |
| 2E9 | 26 | 35 | 53 | 15 | 29 | 27 | 10 | 0 |
| 7B3 | 22 | 4 | 0 | 50 | 16 | 13 | 10 | 0 |
| 1B6 | 0 | 43 | 39 | 0 | 55 | 34 | 6 | 0 |
| 6B5 | 22 | 52 | 35 | 18 | 52 | 75 | 8 | 0 |
| 6F9 | 20 | 20 | 12 | 12 | 35 | 27 | 54 | 0 |

Site specificity of seven Ara h 1 monoclonal antibodies as determined by ELISA inhibition analysis. Values are expressed as percent inhibition.

### Site Specificity of Human IgE

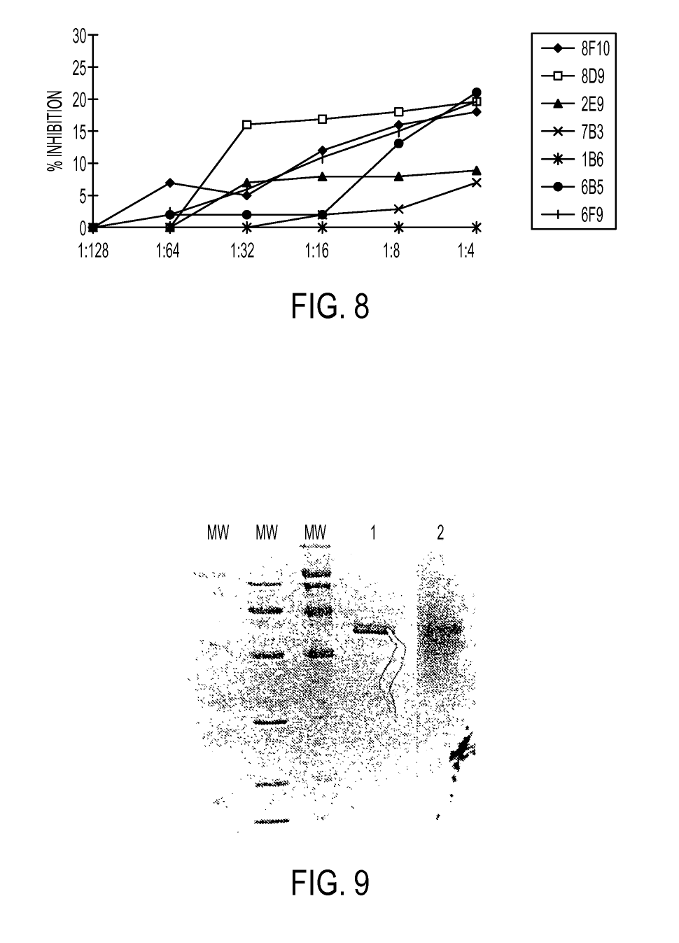

Results of inhibition assays with monoclonal antibodies to inhibit IgE binding to Ara h 1 are shown in FIG. 8. Monoclonal antibodies 8F10, 8D9, 6B, and 6F9 showed significant inhibition to IgE binding. These three inhibition sites correspond with three of the four different IgG epitopes recognized by the monoclonal antibodies in the inhibition experiments (FIG. 7).

### Immunoaffinity Purification of Ara h 1

A crude Florunner peanut extract was passed through an affinity column with monoclonal antibody 8D9 coupled as the immunoabsorbent. After extensive washing, the bound allergen was eluted with 100 mmol/L triethylamine, pH 11.5. This single-step purification resulted in an allergen of more than 90% purity on SDS-PAGE (FIG. 9). The eluted fraction had one major band on SDS-PAGE at a molecular weight of 63.5 kd. This eluted allergen was comparable in molecular weight to the original fast protein liquid chromatography-purified Ara h 1 allergen. An IgE-specific immunoblot was done to ensure that this eluted allergen bound IgE from the pooled peanut-specific IgE serum (FIG. 9). The eluted allergen bound IgE from the peanut-positive pool in an IgE immunoblot. IgE from patients with elevated serum IgE values who were not sensitive to peanuts and from patients with normal serum IgE values did not bind to this allergen (picture not shown).

Anti-Peanut-Specific IgE ELISA with Eluted Allergen

An IgE-specific ELISA with the individual peanut-specific IgE serum was developed to examine the eluted allergen from the monoclonal anti-body column. The six patients who had positive DBPCFC results to peanut had measurable amounts of anti-peanut-specific IgE in their serum, whereas two patients, who had an elevated serum IgE level but were not sensitive to peanut, had no detectable levels of anti-peanut IgE (Table 5).

TABLE 5
Individual anti-peanut-specific IgE binding to eluted Ara h 1

| Patient No. | 1 | 2 | 3 | 4 | 5 | 6 | 7 | 8 |
|---|---|---|---|---|---|---|---|---|
| Percentage of | 250 | 537 | 375 | 859 | 188 | 176 | ND | ND |
| peanut | | | | | | | | |
| sensitive pool | | | | | | | | |

ND, Not determined.
Individual anti-peanut specific IgE values to eluted Ara h 1 allergen expressed as a percent of binding compared with the challenge-positive peanut serum pool.
Patients 1 to 6 had positive DBPCFC results to peanut; patient 7 is the patient without peanut sensitivity with elevated serum IgE; and patient 8 is the patient without peanut sensitivity with normal serum IgE.

### 2.4 Conclusion

In this study seven monoclonal antibodies to Ara h 1 were extensively characterized. All seven monoclonal antibodies produced to Ara h 1, when used as capture antibodies in an ELISA, presented antigens that bound IgE from patients with positive challenge results to peanut. There were no significant differences in the binding of IgE from and one patient to the allergen presented by any monoclonal antibody. When used in separate ELISA experiments, these monoclonal antibodies did not bind to other legume allergens, except for minimal binding to peas and chick peas (e.g., soy, green beans). Also, the monoclonal antibodies did not bind to one variety of peanuts preferentially over another.

To determine the site specificity of these monoclonal antibodies ELISA inhibition assays were done. At least four different and distinct IgG epitopes could be identified in the experiments with the Allergen Ara h 1. Similarly, in the IgE inhibition experiments there were three recognizable and distinct IgE epitopes. Monoclonal antibody 8F10 appeared to inhibit IgE binding significantly more than the other monoclonal antibodies, but there was still considerable inhibition at the other two IgE epitope sites. To future define the allergen Ara h 1 and to determine methods that would allow faster purification, an immunoaffinity column was prepared with monoclonal antibody 8D9. This immunoaffinity column eluted the 63.5 kd peanut allergen, which then bound IgE from the pooled peanut positive serum.

### Example 3

### Cloning and Sequencing of Ara h 1

### 3.1 Introduction

Serum IgE from patients with documented peanut hypersensitivity reactions and a peanut cDNA expression library were used to identify clones that encode peanut allergens. One of the major peanut allergens, Ara h 1, was selected from these clones using Ara h 1 specific oligonucleotides and polymerase chain reaction technology. The Ara h 1 clone identified a 2.3-kb mRNA species on a Northern blot containing peanut poly (A)⁺ RNA. DNA sequence analysis of the cloned inserts revealed that the Ara h 1 allergen has significant homology with the vicilin seed storage protein family found in most higher plants. The isolation of the Ara h 1 clones allowed the synthesis of this protein in E. coli cells and subsequent recognition of this recombinant protein in immunoblot analysis using serum IgE from patients with peanut hypersensitivity.

### 3.2 Methods

### Patients

Serum from eighteen patients with documented peanut hypersensitivity (mean age, 25 years) was used to identify peanut allergens. Each of these individuals had a positive immediate prick skin test to peanut and either a positive double blind, placebo controlled, food challenge (DBPCFC) or a convincing history of peanut anaphylaxis (laryngeal edema, severe wheezing, and/or hypotension). One individual with elevated serum IgE levels (who did not have peanut specific IgE or peanut hypersensitivity) was used as a control in these studies. Details of the challenge procedure and interpretation have been discussed previously (see Example 1). At least five milliliters of venous blood were drawn from each patient and allowed to clot, and the serum was collected. All studies were approved by the Human Use Advisory Committee at the University of Arkansas for Medical Sciences.

### Isolation and Amino Acid Sequence Analysis of Peanut Allergen Ara h 1

Ara h 1 was purified to near homogeneity from whole peanut extracts according to the methods described in Example 1. Purified Ara h 1 was electrophoresed on 12.5% acrylamide mini-gels (Bio-Rad Laboratories) in Tris glycine buffer. The gels were stained with 0.1% Coomassie blue in 10% acetic acid, 50% methanol, and 40% water for 3 hours with continuous shaking. Gel slices containing Ara h 1 were sent to the W. M. Keck Foundation (Biotechnology Resource Laboratory, Yale University, New Haven, Conn.) for amino acid sequencing. Initial sequencing indicated that the amino terminal end of Ara h 1 was blocked. In order to obtain protein sequencing data Ara h 1 was treated with trypsin and peptides were selected for further analysis. Amino acid sequencing of tryptic peptides was performed on an Applied Biosystems sequencer with an on-line HPLC column that was eluted with increasing concentrations of acetonitrile.

### Peanut RNA Isolation and Northern (RNA) Gels

Three commercial lots from the 1979 crop of medium grade peanut species, Arachis hypogaea (Florunner) were obtained from North Carolina State University for this study. Total RNA was isolated from one gram of this material according to procedures described by Larsen (Larsen et al., Mol. Immunol. 29:703-711, 1992). Poly (A)⁺ RNA was isolated using a purification kit supplied by Collaborative Research (Bedford, Mass.) according to manufacturer's instructions. Poly (A)⁺ RNA was subjected to electrophoresis in 1.2% formaldehyde agarose gels, transferred to nitrocellulose, and hybridized with ³²P-labeled probes according to the methods of Bannon et al. (Bannon et al., Nucleic Acids Res. 11:3903-3917, 1983).

cDNA Expression Library Construction And Screening

Peanut poly(A)+ RNA was used to synthesize double-stranded cDNA according to the methods of Watson and Jackson (pp. 79-88 of Vol. 1 of “DNA Cloning” Ed. by D. M. Glover, IRL Press, 1985) and Huynh et al. (pp. 49-78 of Vol. 1 of “DNA Cloning” Ed. by D. M. Glover, IRL Press, 1985). The cDNA was treated with EcoRI methylase and then ligated with EcoRI and XhoI linkers. The DNA was then ligated with EcoRI-XhoI cut, phosphatase treated Lambda ZAP XR phage arms (Stratagene, LaJolla, Calif.) and in vitro packaged. The library was 95% recombinants carrying an average insert size of >400 bp as determined by sizing of randomly selected clones. The library was screened using an IgE antibody pool consisting of an equal volume of serum from each patient with peanut hypersensitivity. Detection of the primary antibody was either with alkaline phosphatase labeled anti-IgE or ¹²⁵I-labeled anti-IgE antibody performed according to manufacturer's instructions. Positive plaques were subjected to subsequent screens using the same pooled serum until all nonreacting plaques were removed. The remaining positive plaques were then rescreened with serum from a patient with elevated total serum IgE who did not have peanut specific IgE to ensure that we were not isolating non-specific, IgE binding clones.

PCR Amplification of the Ara h 1 mRNA Sequence

Using the oligonucleotide GA (TC) AA (AG) GA (TC) AA (TC) GTNAT (TCA) GA (TC) CA (SEQ ID NO. 1) derived from amino acid sequence analysis of the Ara h 1 (63.5 kd) peanut allergen as one primer and a 27 nucleotide long oligo dT stretch as the second primer a portion of the nucleotide sequence that encodes this protein was amplified from peanut cDNA. Reactions were carried out in a buffer containing 3 mM MgCl₂, 500 mM KCl, 100 mM Tris-HCl, pH 9.0. Each cycle of the polymerase chain reaction consisted of 1 minute at 94° C., followed by 2 minutes at 42° C., and 3 minutes at 72° C. Thirty cycles were performed with both primers present in all cycles. From this reaction a 400 bp fragment was amplified and subsequently cloned into a TA vector by standard protocols (Promega, Madison, Wis.).

### DNA Sequencing and Analysis

Sequencing was done according to the methods of Sanger et al. (Proc. Natl. Acad. Sci. USA 74:5463-5467, 1977) using a series of clones constructed by ExoIII digestion of the original DNA isolate or oligonucleotide primers directed to different regions of the clone. Sequence analysis was done on the University of Arkansas for Medical Science's Vax computer using the Wisconsin DNA analysis software package.

### Production of Recombinant Ara h 1 Protein.

The Ara h 1 cDNA was ligated into the EcoRI site of a pBluescript vector (Stratagene). This vector contains 111 nucleotides of the Beta galactosidase gene before the EcoRI site. When E. coli JM109 cells carrying this construct are induced with IPTG they produce a fusion protein consisting of 37 amino acids derived from Beta galactosidase followed by the Ara h 1 protein. Exponentially growing cells are induced with 1 mM IPTG for 4 h at 37° C. Cells are then pelleted and resuspended in SDS-sample buffer, placed in a boiling water bath for 5 minutes and then either used immediately for immunoblot analysis or stored at −20° C. until needed.

### IgE Immunoblot Analysis

SDS-PAGE was performed by the method of Laemmli (Laemmli, 1970, supra). All gels were composed of a 10% acrylamide resolving gel and 4% acrylamide stacking gel. Electrophoretic transfer and immunoblotting on nitrocellulose paper were performed by the procedures of Towbin et al. (Towbin et al., 1979, supra). The blots were incubated with antibodies diluted in a solution containing TBS and 1% bovine serum albumin for at least 12 hours at 4° C. or for 2 hours at room temperature. Detection of the primary antibody was with ¹²⁵I-labeled anti-IgE antibody.

### 3.3 Results

Isolation and Partial Amino Acid Sequencing of Peptides Derived from Ara h 1

Purified and isolated Ara h 1 protein was treated with tryspin and the peptide products separated from one another by HPLC. Three peptide fractions, selected on the basis of their separation from each other and other fractions in the mix, were used for amino acid sequence determination. During the course of sequencing it was noted that fraction I and III consisted of a single peptide species (peptide I, SEQ ID NO. 2 and peptide III, SEQ ID NO. 4, respectively). Fraction II consisted of one major peptide (peptide II, SEQ ID NO. 3) with numerous minor peptide contaminants which complicated sequence determination. However, it was possible to determine the first 16 residues of the major peptide in fraction I and II and the first 10 residues of the major peptide in fraction III. The amino acid sequence determined for each peptide is noted in Table 6.

TABLE 6
Amino acid sequence of Ara h 1 peptides

| Peptide | SEQ ID NO. | Amino acid sequence |
|---|---|---|
| I | 2 | IFLAGDKDNVIDQIEK |
| II | 3 | KGSEEEGDITNPINLR |
| III | 4 | NNPFYFPSRR |

The amino acid sequence of three tryptic peptides derived from purified and isolated Ara h 1 protein was determined. The sequence is shown as the one letter amino acid code.

Isolation of Clones that Produce Antigens Recognized by Peanut-Specific IgE

RNA isolated from the peanut species, Arachis hypogaea (Florunner) was used to construct an expression library for screening with serum IgE from patients with peanut hypersensitivity. Numerous IgE-binding clones were isolated from this library after screening 10 clones with serum IgE from a pool of patients with reactivity to most peanut allergens by western blot analysis. Since the number of plaques reacting with serum IgE was too large to study all in detail we randomly selected a small portion of the positive plaques for further purification. Phage positive for IgE binding were plaque purified to homogeneity and then tested for their ability to react with serum IgE collected from a patient without peanut hypersensitivity. All of the selected clones were intensely positive when incubated with serum IgE from patients with peanut hypersensitivity. In contrast, these same clones did not react with control serum IgE. These results show that we have isolated numerous clones capable of producing IgE recognizable antigens specific to patients who have peanut hypersensitivity.

Identification and Characterization of Clones that Encode Peanut Allergen Ara h 1

To help identify which of the many IgE positive clones encoded the Ara h 1 allergen, a hybridization probe was constructed using an oligonucleotide developed from Ara h 1 amino acid sequence and PCR technology. The oligonucleotide sequence GA (TC) AA (AG) GA (TC) AA (TC) GTNAT (TCA) GA (TC) CA (SEQ ID NO. 1) was derived from amino acid residues located within peptide I (SEQ ID NO. 2, Table 6) of the Ara h 1 peanut allergen. Utilizing this oligonucleotide as one primer and a 27-nucleotide oligo dT stretch as the second primer a portion of the mRNA sequence that encodes this protein was amplified from peanut cDNA. This 400 bp DNA fragment was subsequently cloned and sequenced by the Sanger dideoxy (Towbin et al., 1979, supra) method. DNA sequence analysis revealed that the 400 bp DNA fragment contained a poly A stretch on one end and the Ara h 1 specific nucleotide sequence on the other end. In addition, this clone contained nucleotide sequence correctly encoding the remaining carboxy terminal portion of peptide I (SEQ ID NO. 2). Thus, an Ara h 1 specific clone has been isolated and it can be used as a hybridization probe to identify which of the many IgE positive clones selected encodes the Ara h 1 allergen.

We hybridized a Southern blot containing four of the IgE selected cloned DNAs with a ³²P-labeled, Ara h 1 PCR amplification product to determine which of the isolated clones encoded the Ara h 1 peanut allergen. All of the clones were positive for hybridization with this probe. In addition we screened 200,000 clones from the peanut cDNA library using ³²P-labeled Ara h 1 clone as a probe. From this screen, over 100 Ara h 1 positive clones were identified (data not shown). These results indicate that the mRNA encoding the Ara h 1 allergen is an abundant message within this library.

To determine what size mRNA these clones identify, a ³²P-labeled insert from one of the largest cDNA clones (clone P41b) was used as a hybridization probe of a Northern blot containing peanut poly(A)+ RNA (data not shown). This insert hybridized to an ˜2.3 kb mRNA, indicating that this insert probably represented the entire mRNA.

### Peanut Allergen Ara h 1 is a Vicilin-Like Seed Storage Protein

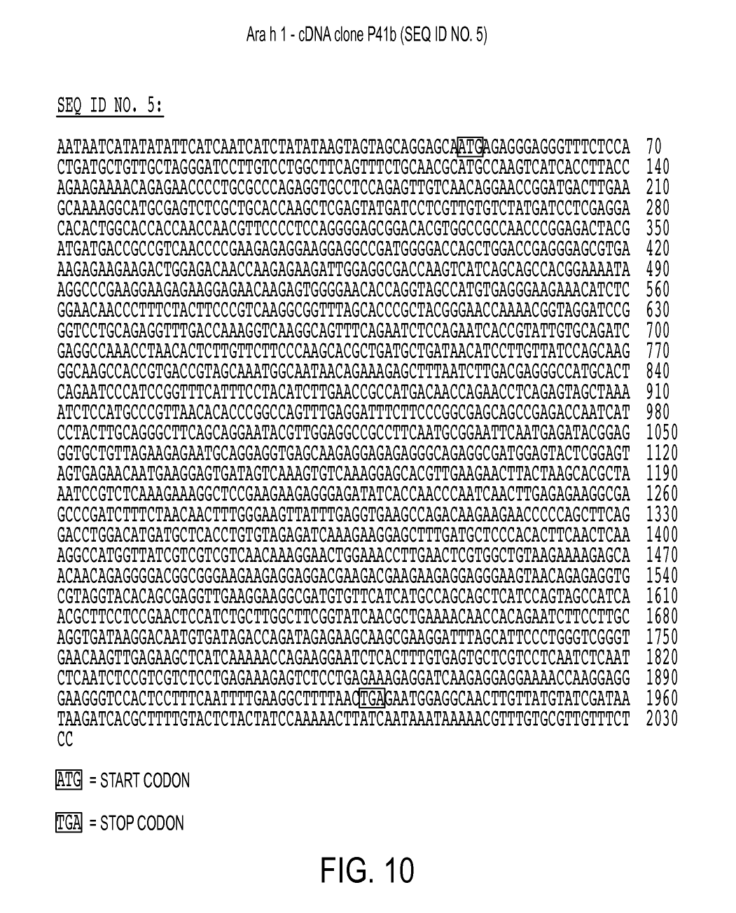

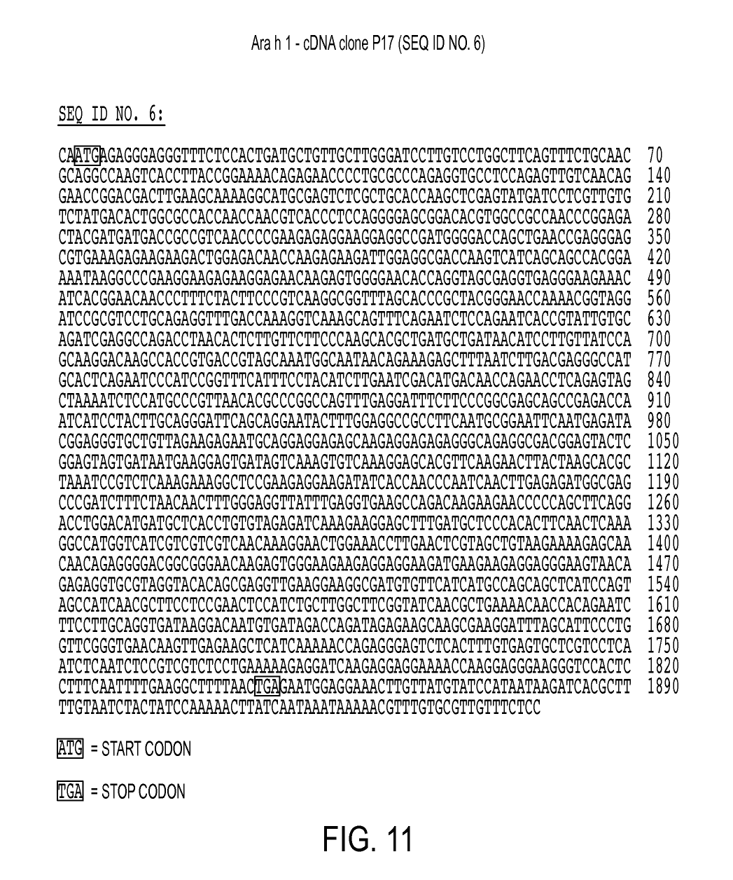

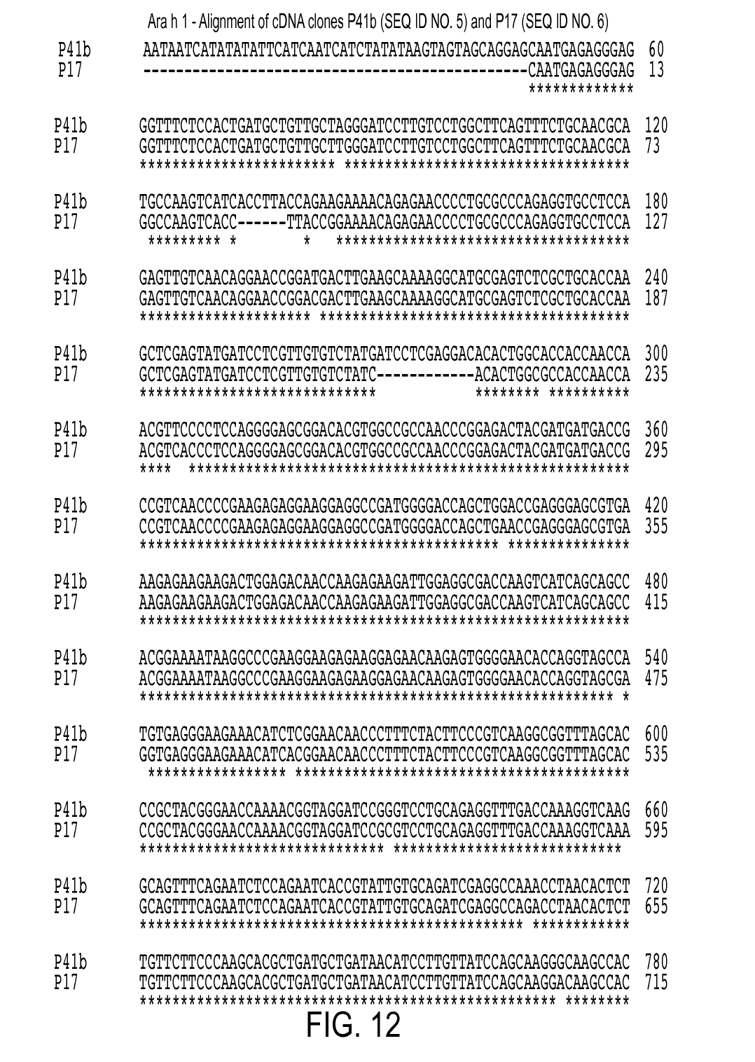

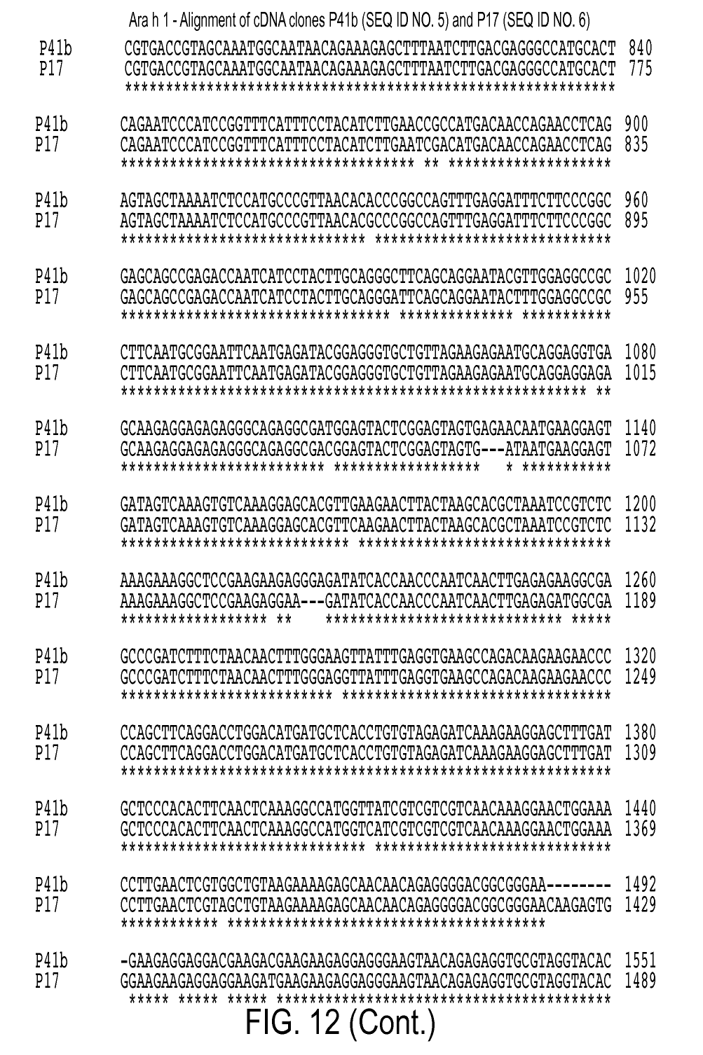

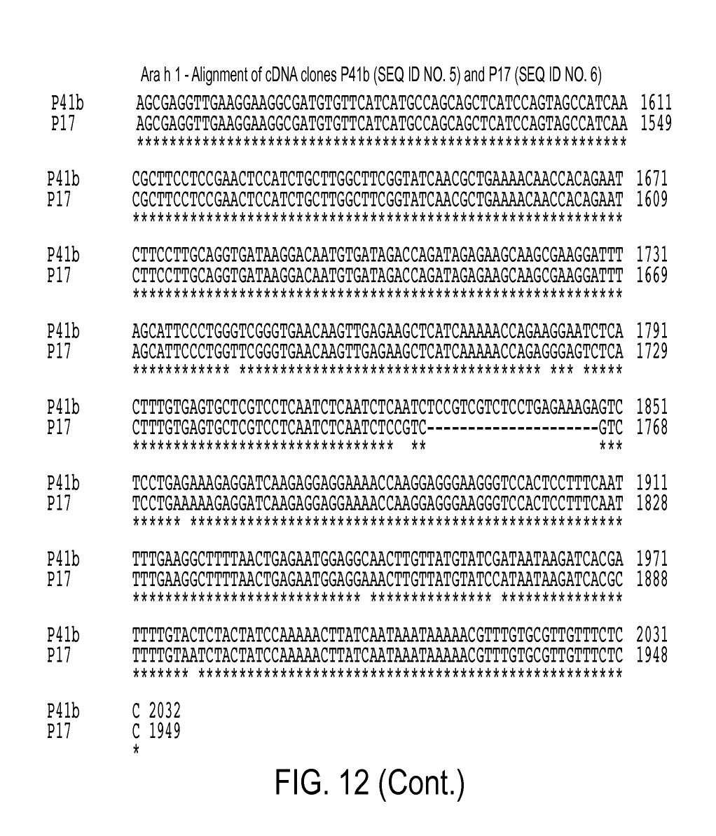

The primary DNA sequences of two of the largest cDNA clones selected (clone P41b, SEQ ID NO. 5 shown in FIG. 10 and clone P17, SEQ ID NO. 6 shown in FIG. 11) were determined by Sanger dideoxy sequencing using oligonucleotide primers directed to different regions on the insert or a series of subclones constructed by ExoIII digestion of the inserts. Clone P41b carried a 2,032-base insert (SEQ ID NO. 5) while clone P17 carried a 1,949 base insert (SEQ ID NO. 6). The ATG protein synthesis start codons are located between nucleotide positions 50-52 of SEQ ID NO. 5 and between nucleotide positions 3-5 of SEQ ID NO. 6. The sequence around these codons agrees with the translation initiation sequence found in most eukaryotic mRNAs (Kozak, Nucleic Acids Res. 12:857-872, 1984). Each of the inserts contained a large open reading frame starting with the ATG start codon and ending with a TGA stop codon (nucleotides 50-1930 of SEQ ID NO. 5 and nucleotides 3-1847 of SEQ ID NO. 6). As shown in the alignment of FIG. 12, there was more than 97% sequence homology between the two inserts.

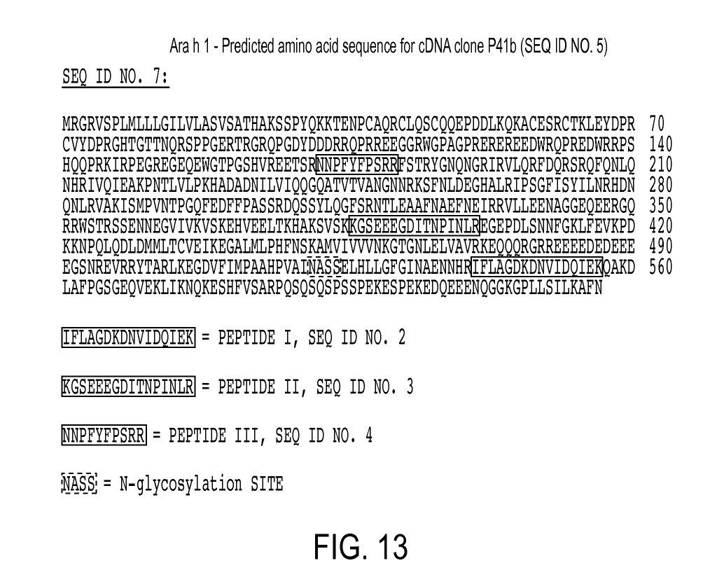

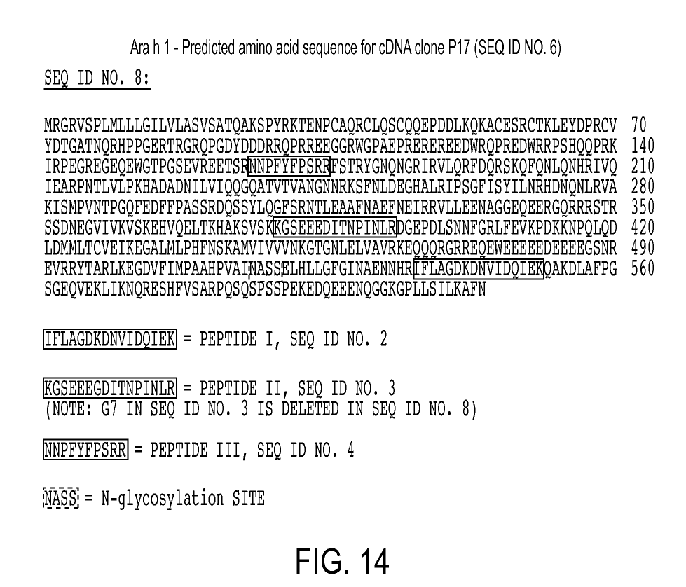

Both clones encode a protein of 68 kd (clone P41b encodes a protein of 626 amino acids, SEQ ID NO. 7 shown in FIG. 13 while clone P17 encodes a protein of 614 amino acids, SEQ ID NO. 8 shown in FIG. 14). The amino acid sequences of peptides I, II, and III (SEQ ID NOs. 2-4) are found in the predicted amino acid sequences of both clones (FIGS. 13 and 14, note that the predicted amino acid sequence of clone P17 lacks the glycine residue at position 7 of peptide II, SEQ ID NO. 3). In addition, both proteins have a signal peptide at the amino terminus (Coleman et al., Cell 43:351-360, 1985) and a single N-glycosylation site (consensus sequence: N-{P}-[ST]-{P} between amino acids 521-524 of SEQ ID NO. 7 and 516-519 of SEQ ID NO. 8). These data confirm and extend our conclusion that these clones encode the Ara h 1 allergen.

A search of the GenBank database revealed significant sequence homology between the Ara h 1 cDNA clones and a class of seed storage proteins called vicilins. There was 60-65% homology over >750 bases when the Ara h 1 DNA sequences were compared with the broad bean and pea vicilins (Table 7). These results indicate that the Ara h 1 allergen belongs to a vicilin-like multi-gene family encoding similar but not identical proteins.

TABLE 7
Homology of the Ara h 1 gene to plant vicilin genes

| | cDNA clone P41b - | | cDNA clone P17 - | |
|---|---|---|---|---|
| | SEQ ID NO. 5 | | SEQ ID NO. 6 | |
| | bp overlap | % homology | bp overlap | % homology |
| Broad bean | 1,081 | 64.3 | 985 | 62.3 |
| Pea | 1,078 | 64.2 | 961 | 62.5 |
| Soybean | 323 | 65.9 | 815 | 61.5 |

The Wisconsin DNA analysis software package was used to search for homology between the Ara h 1 nucleotide sequence and any DNA sequence contained in the data base. Significant homology was observed between Ara h 1 and the plant vicilins.

### Recognition of Recombinant Ara h 1 by Patient Sera in an IgE Immunoblot Assay

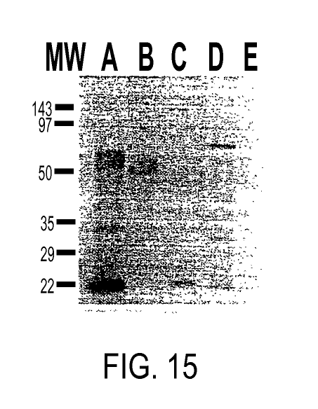

IgE immunoblot analysis was initially performed using serum IgE from a pool of patients with peanut hypersensitivity to determine the molecular weight of the recombinant protein and the specificity of the IgE recognition reaction. FIG. 15 (lanes A and B) shows that the IgE pool recognized whole peanut extract and purified native Ara h 1 protein as expected, but did not react with any proteins from an E. coli lysate that was prepared from cells carrying vector alone (FIG. 15, lane E). However, instead of the IgE pool recognizing a 68 kd protein produced from clone P17, an unexpectedly small protein was identified (FIG. 15, lane C). On further analysis, we noted that by eliminating the first 93 bases (31 amino acids, 5% of Ara h 1) of this clone we could produce full length Ara h 1 protein (68 kd) with numerous truncated products that migrated as smaller IgE reactive peptides (FIG. 15, lane D). The presence of truncated Ara h 1 products could be the result of inefficient translation of the amino terminal portion of this protein (Schatzman and Rosenberg, Methods Enzymol. 152:661, 1987 and Wood et al., Nucleic Acids Res. 12:3937, 1984) caused by rare codons, numerous cysteine residues, or secondary structure of the mRNA.

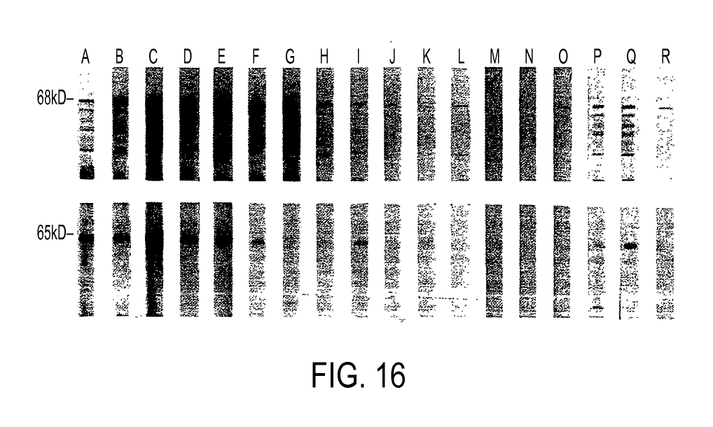

FIG. 16 shows eighteen immunoblot strips of recombinant Ara h 1 (upper panel) or native Ara h 1 (lower panel) that have been incubated with different patient sera. 94% (17/18) of the patients that showed IgE binding to the native allergen also showed some level of binding to the recombinant Ara h 1 protein. Of the 18 patient sera tested in this manner there were varying intensities of IgE binding to the recombinant and native allergen. In general, there was good agreement between the level of IgE binding of recombinant and native Ara h 1 for any individual patient. For example, patients who had high levels of IgE which bound native protein (FIG. 16, lower panel, lanes A-F) also showed high immunoreactivity with recombinant Ara h 1 protein (FIG. 16, upper panel, lanes A-F). Patients who had low levels of IgE which bound native allergen (FIG. 16, lower panel, lanes L-R) showed low reactivity with the recombinant protein (FIG. 16, upper panel, lanes L-R). One peanut sensitive individual (lane K) who had serum specific IgE to native Ara h 1 had no detectable IgE which recognized the recombinant protein (FIG. 16, upper panel, lane K). The differences we have noted between peanut hypersensitive patients could be due to the amount of peanut-specific IgE in individual patients, differences in affinity of patient-specific IgE for peanut, or that some patients recognize only certain peanut proteins.

### 3.4 Conclusion

The Ara h 1 nucleotide sequences identified in this report have significant sequence homology with the vicilin family of seed storage proteins of other legumes (soybean, pea, common bean, etc.). The major seed storage proteins of legumes are globulins that are represented in most legumes by two different types of polypeptides, the nonglycosylated legumins and glycosylated vicilins. The genes for the glycosylated seed storage proteins of higher plants code for proteins that are classified by their size into small (50 kd) and large (70 kd) vicilins (Chee and Slightom, Sub-Cellular Biochemistry 17:31-52, 1991). A comparison of the vicilin amino acid sequences reveals considerable amino acid homology between the small and large vicilins in the carboxy terminal portion of these molecules. The major difference between the large and small vicilin preproteins is the existence of an additional tract of amino acids at the amino terminal end of the large vicilins (Dure, New Bio. 2:487-493, 1990). The information generated in our laboratory demonstrating that the major peanut allergens are vicilin-like proteins may explain why patients with peanut hypersensitivity and peanut-specific IgE tend to have serum IgE to multiple other legume proteins. Since the vicilins of most major plants share significant sequence homology in their carboxy terminal portion, it is not surprising that serum specific IgE would tend to bind to several vicilin proteins from different sources. However, despite patients with legume hypersensitivity having IgE to multiple legume proteins (peanuts, soybeans, peas, etc.) they generally have clinical food hypersensitivity to only one food in the legume family. Because the amino terminal domains of the large glycosylated (vicilin) proteins share little or no homology, the immune response to this portion of the protein may be responsible for the severe and chronic hypersensitivity response characteristic of peanuts.

We have demonstrated that the cloned Ara h 1 gene is capable of producing a protein product in prokaryotic cells that is recognized by serum IgE from a large proportion of individuals with documented peanut hypersensitivity. These results are significant in that they indicate that some of the allergenic epitopes responsible for this reaction are linear amino acid sequences that do not include a carbohydrate component.

### Example 4

### Mapping and Mutational Analysis of the Linear IgE Epitopes of Ara h 1

### 4.1 Introduction

Serum IgE from patients with documented peanut hypersensitivity reactions and overlapping peptides were used to identify the IgE-binding epitopes on the major peanut allergen, Ara h 1. At least twenty-three different linear IgE-binding epitopes, located throughout the length of the Ara h 1 protein, were identified. All of the epitopes were 6-10 amino acids in length, but there was no obvious sequence motif shared by all peptides. Four of the peptides appeared to be immunodominant IgE-binding epitopes in that they were recognized by serum from more than 80% of the patients tested and bound more IgE than any of the other Ara h 1 epitopes. Mutational analysis of the immunodominant epitopes revealed that single amino acid changes within these peptides had dramatic effects on IgE-binding characteristics.

### 4.2 Methods

### Serum IgE

Serum from 15 patients with documented peanut hypersensitivity reactions (mean age, 25 years) was used to identify the Ara h 1 IgE-binding epitopes. Each of these individuals had a positive immediate prick skin test to peanut and either a positive double-blind placebo-controlled food challenge or a convincing history of peanut anaphylaxis (laryngeal edema, severe wheezing, and/or hypotension). Representative individuals with elevated serum IgE levels (who did not have peanut-specific IgE or peanut hypersensitivity) were used as controls in these studies. In some instances, a serum pool was made by mixing equal aliquots of serum IgE from each of the 15 patients with peanut hypersensitivity. This pool was then used in immunoblot analysis experiments to determine the IgE-binding characteristics of the population. At least 5 ml venous blood was drawn from each patient and allowed to clot, and the serum collected. All studies were approved by the Human Use Advisory Committee at the University of Arkansas for Medical Sciences.

### Computer Analysis of Ara h 1 Sequence

Analysis of the Ara h 1 amino acid sequence (see Example 3, clone P41b, SEQ ID NO. 7) and peptide sequences was performed on the University of Arkansas for Medical Sciences' Vax computer using the Wisconsin DNA analysis software package. The predicted antigenic regions on the Ara h 1 protein are based on algorithms developed by Jameson and Wolf (Comput. Appl. Biosci. 4:181-0.186, 1988) that relate antigenicity to hydrophilicity, secondary structure, flexibility, and surface probability.

### Peptide Synthesis

Individual peptides were synthesized on a cellulose membrane containing free hydroxyl groups using Fmoc-amino acids according to the manufacturer's instructions (Genosys Biotechnologies). Synthesis of each peptide was started by esterification of an Fmoc-amino acid to the cellulose membrane. After washing, all residual amino functions on the sheet were blocked by acetylation to render them unreactive during the subsequent steps. Fmoc protective groups were then removed by addition of piperidine to render nascent peptides reactive. Each additional Fmoc-amino acid is esterified to the previous one by this same process. After addition of the last amino acid in the peptide, the amino acid side chains were de-protected using a mixture of 1:1:0.05 (by volume) dichloromethane/trifluoroacetic acid/triisobutylsilane, followed by washing with dichloromethane and methanol. Membranes containing synthesized peptides were either probed immediately with serum IgE or stored at −20° C. until needed.

### IgE-Binding Assay

Cellulose membranes containing synthesized peptides were incubated with the serum pool or individual serum from patients with peanut hypersensitivity diluted (1:5) in a solution containing Tris/NaCl (10 mM Tris/HCl, 500 mM NaCl, pH 7.5) and 1% bovine serum albumin for at least 12 h at 4° C. or 2 hours at room temperature. The primary antibody was detected with ¹²⁵I-labeled anti-IgE antibody (Sanofi Pasteur Diagnostics).

### 4.3 Results

Identification of Multiple IgE-Binding Regions within Ara h 1

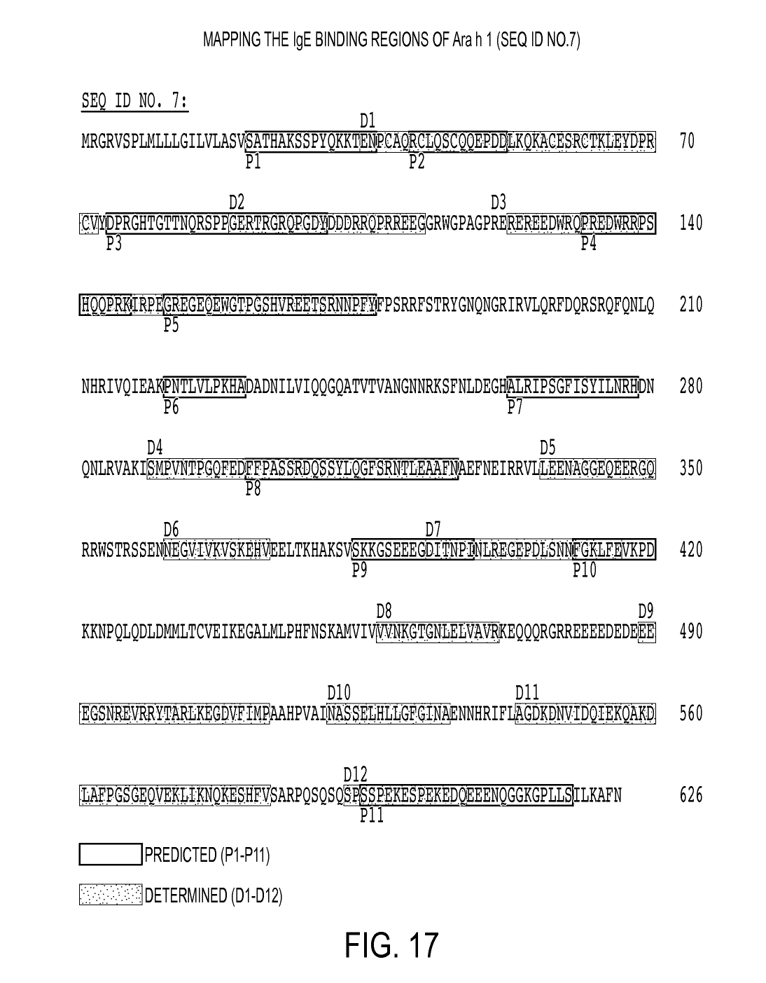

The Ara h 1 amino acid sequence (SEQ ID NO. 7) was first analyzed for potential antigenic epitopes using computer-based algorithms. There were 11 possible antigenic regions, each containing multiple antigenic sites, predicted by this analysis along the entire length of the molecule (FIG. 17, boxed areas P1-P11).

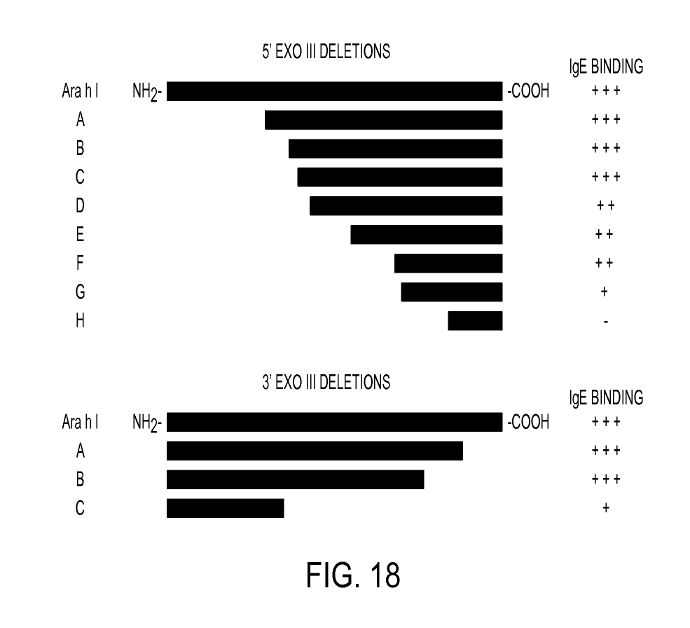

Preliminary experiments were then performed to map the major IgE binding regions of Ara h 1. Exo III digestion from the 5′ or 3′ end of a full length Ara h 1 cDNA clone was used to produce shortened clones whose protein products could then be tested for IgE binding by immunoblot analysis (FIG. 18). The pluses (+) on the right side of FIG. 18 indicate the extent of IgE binding to the protein product of each construct. All constructs bound IgE until they were reduced to the extreme carboxyl terminal (5′ Exo III) or amino terminal (3′ Exo III) end of the molecule. These results indicate that there are multiple IgE epitopes on the Ara h 1 allergen.

77 overlapping peptides representing the entire length of the Ara h 1 protein were then synthesized to characterize the IgE binding regions in greater detail. Each peptide was 15 amino acids long and offset from the previous peptide by eight amino acids. In this manner, the entire length of the Ara h 1 protein could be studied in large overlapping fragments. These peptides were then probed with a pool of serum IgE from 15 patients with documented peanut hypersensitivity or with serum IgE from a representative control patient with no food allergy. Serum IgE from the control patients did not recognize any of the synthesized peptides. In contrast, there are 12 IgE-binding regions (D1-D12) along the entire length of the Ara h 1 protein recognized by IgE from this population of patients with peanut hypersensitivity (FIG. 17, shaded areas D1-D12). These IgE-binding regions represent amino acid residues 35-72, 89-112, 121-176, 289-326, 337-350, 361-374, 393-416, 457-471, 489-513, 521-535, 544-583, and 593-607 of SEQ ID NO. 7. In general, the predicted antigenic regions (FIG. 17, boxed areas P1-P11) contained or were part of those that were determined by actual IgE binding (FIG. 17, shaded areas D1-D12). However, there were two predicted antigenic regions (between amino acids 221-230 and 263-278 of SEQ ID NO. 7, FIG. 17) that were not recognized by serum IgE from peanut hypersensitive individuals. In addition, there were numerous IgE-binding regions found in the Ara h 1 protein between amino acids 450-600 of SEQ ID NO. 7 (FIG. 17).

To determine the amino acid sequence of the IgE-binding epitopes, small overlapping peptides spanning each of the larger IgE-binding regions identified in FIG. 17 were synthesized. By synthesizing smaller peptides (10 amino acids long) that were offset from each other by only two amino acids, it was possible to identify individual IgE-binding epitopes within the larger IgE-binding regions of the Ara h 1 molecule (Table 8).

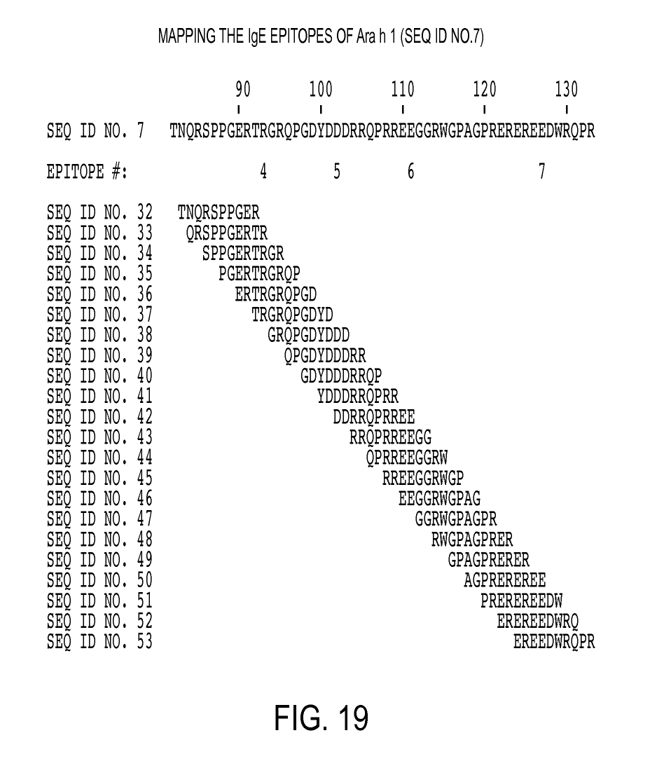

FIG. 19 illustrates the approach for the binding region D2-D3 (amino acids 82-133 of SEQ ID NO. 7). Four epitopes (FIG. 19, epitopes 4-7) were identified in this region. Similar blots were completed for the remaining IgE-binding regions to identify the core amino acid sequences for each IgE epitope. Table 8 summarizes the 23 IgE-binding epitopes (SEQ ID NO. 9-31) and their respective positions in the Ara h 1 protein (SEQ ID NO. 7).

The most common amino acids found were acidic (D, E) and basic (K, R) residues comprising 40% of all amino acids found in the IgE epitopes. There were no obvious amino acid sequence motifs shared by all the IgE epitopes.

TABLE 8
Ara h 1 IgE-binding epitopes

| SEQ | | Amino | |
|---|---|---|---|
| ID | | acid | Ara h 1 |
| NO. | Peptide | sequence¹ | positions² |
| 9 | 1 | AKSSPYQKKT | 25-34 |
| 10 | 2 | QEPDDLKQKA | 48-57 |
| 11 | 3 | LEYDPRLVYD | 65-74 |
| 12 | 4 | GERTRGRQPG | 89-98 |
| 13 | 5 | PGDYDDDRRQ | 97-106 |
| 14 | 6 | PRREEGGRWG | 107-116 |
| 15 | 7 | REREEDWRQP | 123-132 |
| 16 | 8 | EDWRRPSHQQ | 134-143 |
| 17 | 9 | QPRKIRPEGR | 143-152 |
| 18 | 10 | TPGQFEDFFP | 294-303 |
| 19 | 11 | SYLQEFSRNT | 311-320 |
| 20 | 12 | FNAEFNEIRR | 325-334 |
| 21 | 13 | EQEERGORRW | 344-353 |
| 22 | 14 | DITNPINLRE | 393-402 |
| 23 | 15 | NNFGKLFEVK | 409-418 |
| 24 | 16 | GTGNLELVAV | 461-470 |
| 25 | 17 | RRYTARLKEG | 498-507 |
| 26 | 18 | ELHLLGFGIN | 525-534 |
| 27 | 19 | HRIFLAGDKD | 539-548 |
| 28 | 20 | IDQIEKQAKD | 551-560 |
| 29 | 21 | KDLAFPGSGE | 559-568 |
| 30 | 22 | KESHFVSARP | 578-587 |
| 31 | 23 | PEKESPEKED | 597-606 |

¹The underlined portions of each peptide are the smallest IgE-binding sequences as determined by the analysis described in FIG. 19.
²The Ara h 1 amino acid positions are taken from SEQ ID NO. 7.

### Identification of Immunodominant Ara h 1 Epitopes

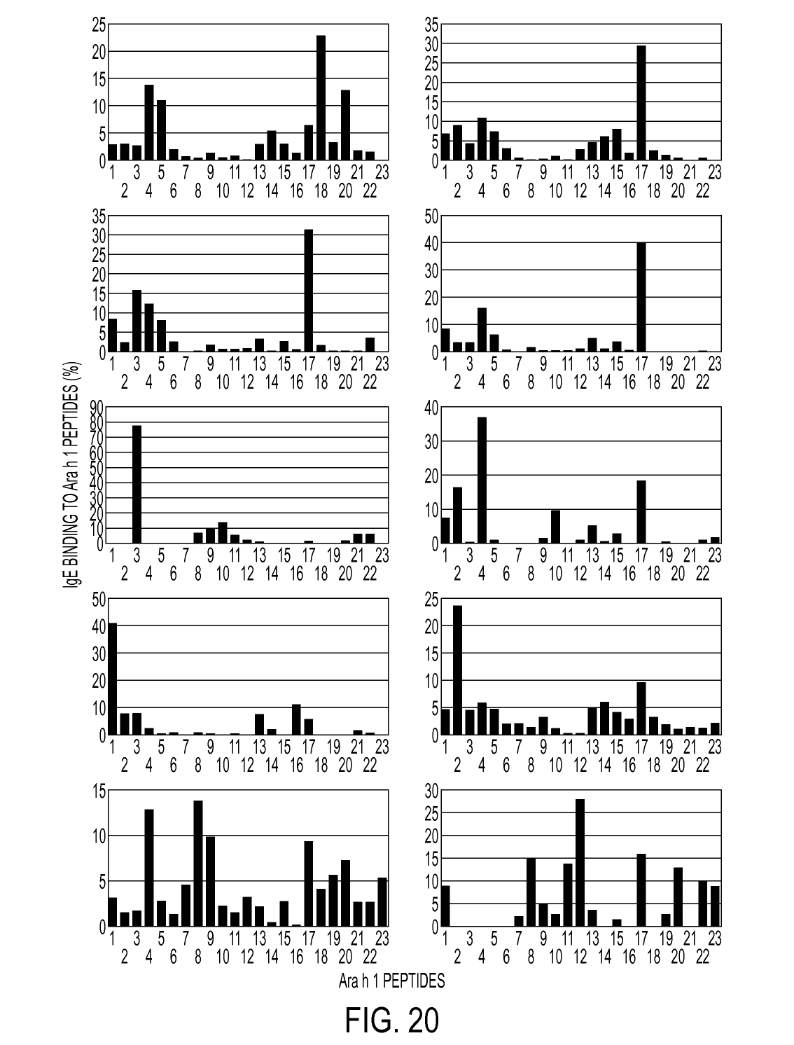

In an effort to determine which, if any, of the 23 epitopes was immunodominant, each set of 23 peptides was probed individually with serum IgE from ten different patients. An epitope can be considered immunodominant if it is recognized by serum IgE from the majority of patients with peanut hypersensitivity or if the serum IgE that recognizes a peptide represents the majority of Ara h 1-specific IgE found in a patient. Serum from five individuals randomly selected from the 15 patient serum pool and an additional five sera from peanut-hypersensitive patients not represented in the serum pool were used to identify the commonly recognized epitopes. Immunoblot strips containing peptides 1-23 (see Table 8) were incubated with each individual patient's serum. The intensity of IgE binding to each spot was determined as a function of that patient's total IgE binding to these 23 epitopes (FIG. 20). All of the patient sera tested (10/10) recognized multiple peptides. The most commonly recognized peptides were those that contained epitopes 1, 3, 4, 13, 17 and 22. These epitopes were recognized by IgE from at least 80% of the patient sera tested (8/10). In addition, epitopes 1-4,8,12, and 17, when recognized, bound more serum IgE from individual patients than any of the other epitopes. These results indicate that peptides 1, 3, 4, and 17 contain the immunodominant epitopes of the Ara h 1 protein.

### Amino Acids Essential to IgE Binding

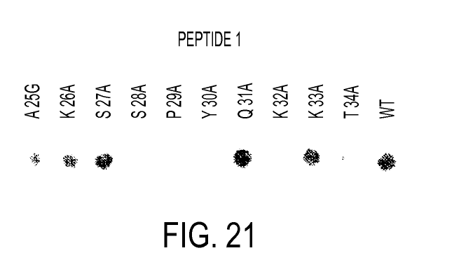

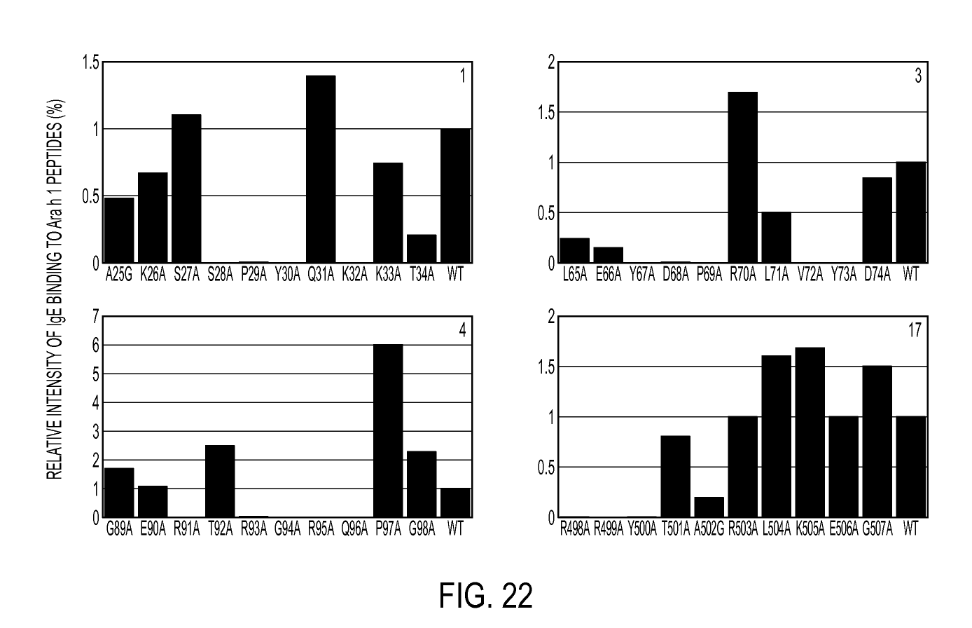

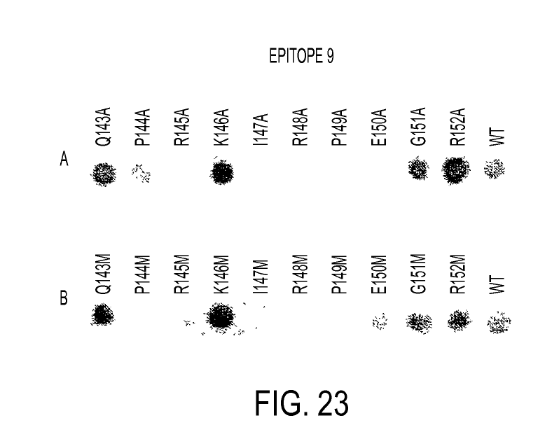

The amino acids essential to IgE binding in the Ara h 1 epitopes were determined by synthesizing duplicate peptides with single amino acid changes at each position. These peptides were then probed with pooled serum IgE from 15 patients with peanut hypersensitivity to determine if the changes affected peanut-specific IgE binding. An immunoblot strip containing the wild-type and mutated peptides of immunodominant epitope 1 is shown in FIG. 21. The pooled serum IgE did not recognize this peptide, or binding was drastically reduced, when alanine was substituted for each of the amino acids at positions 28-30, or 32 of SEQ ID NO. 7. In contrast, the substitution of an alanine for glutamine residue at position 31 of SEQ ID NO. 7 resulted in increased IgE binding. Results for the remaining immunodominant Ara h 1 epitopes 3, 4, and 17 are shown in FIG. 22. Immunoblot strips containing the wild-type and mutated peptides of non-immunodominant epitope 9 are shown in FIG. 23. Binding of pooled serum IgE to these individual peptides was dramatically reduced when either alanine or methionine was substituted for each of the amino acids at positions 144, 145, and 147-150 of SEQ ID NO. 7. Changes at positions 144, 145, 147 and 148 of SEQ ID NO. 7 had the most dramatic effect when methionine was substituted for the wild-type amino acid, resulting in less than 1% of peanut-specific IgE binding to these peptides. In contrast, the substitution of an alanine for arginine at position 152 of SEQ ID NO. 7 resulted in increased IgE binding.

In general, each epitope could be mutated to a non-IgE-binding peptide by the substitution of an alanine or methionine for a single amino acid residue. There was no obvious position within each peptide that, when mutated, would result in loss of IgE binding. Furthermore, there was no consensus in the type of amino acid that, when changed to alanine or methionine, would lead to loss of IgE binding. Table 9 summarizes these results.

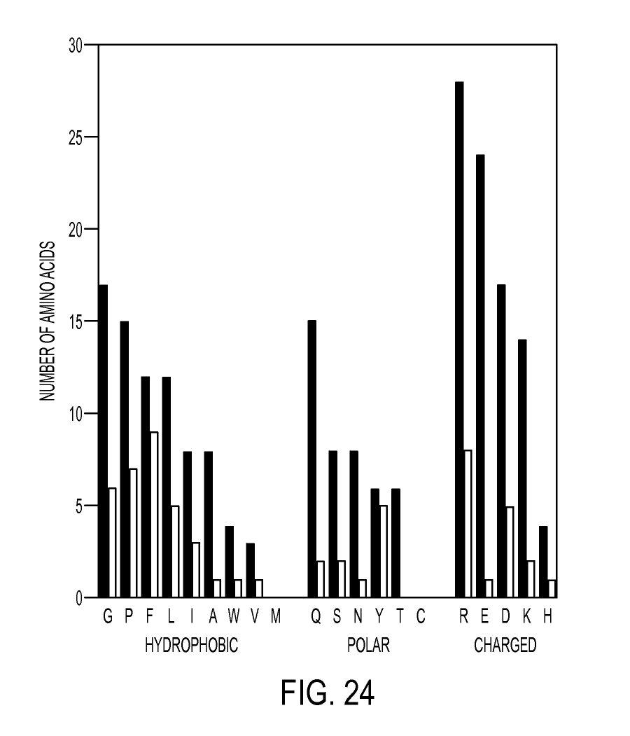

The amino acids within each epitope were classified according to whether they were hydrophobic, polar, or charged residues (FIG. 24). There were a total of 196 amino acids present in the 21 epitopes of Ara h 1 that were studied (epitopes 16 and 23 were not included in this study because they were recognized by a single patient who was no longer available to the study). Charged residues occurred most frequently (89/196), with hydrophobic residues (71/196) being the next frequent type of amino acid in the epitopes, and polar residues representing the least frequent amino acid group (36/196). Thirty-five percent of the mutated hydrophobic residues resulted in loss of IgE binding (<1% IgE binding), whereas only 25 and 17% of mutated polar and charged residues, respectively, had a similar effect. These results indicated that the hydrophobic amino acid residues within these IgE binding epitopes were the most sensitive to changes. In addition results form this analysis indicated that the amino acids located near the center of the epitope were more critical for IgE binding.

TABLE 9
Amino acids critical to IgE binding
in Ara h 1

| SEQ | | Amino | |
|---|---|---|---|
| ID | | acid | Ara h 1 |
| NO. | Peptide | sequence¹ | positions² |
| 9 | 1 | AKSSPYQKKT | 25-34 |
| 10 | 2 | QEPDDLKQKA | 48-57 |
| 11 | 3 | LEYDPRLVYD | 65-74 |
| 12 | 4 | GERTRGRQPG | 89-98 |
| 13 | 5 | PGDYDDDRRQ | 97-106 |
| 14 | 6 | PRREEGGRWG | 107-116 |
| 15 | 7 | REREEDWRQP | 123-132 |
| 16 | 8 | EDWRRPSHQQ | 134-143 |
| 17 | 9 | QPRKIRPEGR | 143-152 |
| 18 | 10 | TPGQFEDFFP | 294-303 |
| 19 | 11 | SYLQEFSRNT | 311-320 |
| 20 | 12 | FNAEFNEIRR | 325-334 |
| 21 | 13 | EQEERGQRRW | 344-353 |
| 22 | 14 | DITNPINLRE | 393-402 |
| 23 | 15 | NNFGKLFEVK | 409-418 |
| 25 | 17 | RRYTARLKEG | 498-507 |
| 26 | 18 | ELHLLGFGIN | 525-534 |
| 27 | 19 | HRIFLAGDKD | 539-548 |
| 28 | 20 | IDQIEKQAKD | 551-560 |
| 29 | 21 | KDLAFPGSGE | 559-568 |
| 30 | 22 | KESHFVSARP | 578-587 |

The Ara h 1 IgE binding epitopes are indicated as the single letter amino acid code. The position of each peptide with respect to the Ara h 1 protein coding sequence is indicated in the right hand column.
¹The amino acids that, when altered, lead to loss of IgE binding are shown as the bold, underlined residues. Epitopes 16 and 23 were not included in this study because they were recognized by a single patient who was no longer available to the study.
²The Ara h 1 amino acid positions are taken from SEQ ID NO. 7.

### 4.4 Conclusion

In the present study, we have determined that multiple antigenic sites are predicted for the Ara h 1 allergen. There are at least 23 different IgE recognition sites on the major peanut allergen Ara h 1. These sites are distributed throughout the protein.

Four of the Ara h 1 epitopes appear to be immunodominant IgE-binding epitopes in that they are recognized by more than 80% of patient sera tested. Interestingly, epitope 17, which is located in the C-terminal end of the protein (amino acids 498-507 of SEQ ID NO. 7), is in a region that shares significant sequence similarity with vicilins from other legumes (Gibbs et al., Mol. Biol. Eva 6:614-623, 1989). The amino acids important for IgE binding also appear to be conserved in this region and may explain the possible cross-reacting antibodies to other legumes that can be found in sera of patients with a positive double-blind placebo-controlled food challenge to peanuts. Epitopes 1, 3, and 4 located in the N-terminal portion of the protein (amino acids 25-34, 65-74, and 89-98 of SEQ ID NO. 7), appear to be unique to this peanut vicilin and do not share any significant sequence similarity with vicilins from other legumes (Gibbs et al., 1989, supra). The amino acids important to IgE binding in this region are not conserved. We have also determined that, once an IgE binding site has been identified, it is the hydrophobic amino acid residues that appear to play a critical role in immunoglobulin binding.

Our data show that it may be possible to mutate the Ara h 1 allergen to a protein that no longer binds IgE. This raises the possibility than an altered Ara h 1 gene could be used to replace its allergenic homologue in the peanut genome.

### Example 5

### Ara h 1 Mutant Protein with Reduced IgE Binding

### 5.1 Introduction

We constructed a mutant recombinant Ara h 1 protein with single alanine point mutations in epitopes 1, 2, 3, 4, 5, 6, and 17. Epitopes 1-6 were chosen because they lie within the variable N-terminal domain and are not conserved between vicilins and therefore may be responsible for the peanut's extreme allergenicity. Assays utilizing serum from patient with peanut hypersensitivity and the wild-type and mutant recombinant proteins revealed a significant decrease in IgE binding to the mutant protein in 50% of the patients tested.

### 5.2 Methods

Recombinant wild-type Ara h 1 was prepared as described in Example 3 (i.e., by inserting cDNA clone P41b into the pBluescript expression vector from Stratagene). The mutant Ara h 1 was constructed by inserting a PCR product of Ara h 1 (with mutations shown in Table 10) into the pET24b expression vector from Novagen, Madison, Wis.

TABLE 10
Mutated Ara h 1 protein (SEQ ID NO.7)

| Epitope | Mutation |
|---|---|
| 1 | K32A |
| 2 | D52A |
| 3 | V72A |
| 4 | R91A |
| 5 | D103A |
| 6 | R109A |
| 17 | R499A |

A western blot control was performed on the wild-type and mutant Ara h 1 recombinant proteins to ensure that an equal amount of each protein was used in these studies. Equal amounts of wild-type and mutant Ara h 1 were detected and both proteins migrated at their expected molecular weights (65 kd).

### 5.3 Results

Western blots of wild-type and mutant recombinant proteins probed with individual peanut-sensitive patient sera were performed. The results are summarized in Table 11. Data for each patient is numbered 1-10 in the first column. The second column lists the epitopes that each patient recognized in the wild-type protein that were changed in the mutant protein. The third column lists the epitopes that each patient recognized in the wild-type protein that were not changed in the mutant protein. The fourth column shows the relative IgE binding affinity of the mutant protein vs. the wild-type protein. In 50% of cases IgE binding to the mutant protein was significantly reduced.

TABLE 11
Relative affinity of IgE to wild-type and mutant Ara h 1

| Patient | Mutated epitopes | Wild-type epitopes | Relative binding |
|---|---|---|---|
| 1 | 1, 4, 5, 17 | 8, 13 | Decreased |
| 2 | 2, 3, 4, 17 | 14, 18 | Equal |
| 3 | 4, 5, 17 | 11, 14, 18-20, 22 | Increased |
| 4 | 2, 4, 5, 17 | 9, 23 | Decreased |
| 5 | 1, 4, 17 | 9, 10, 12-15, 18, 21, 22 | Equal |
| 6 | 4, 17 | 8, 9, 20, 23 | Decreased |
| 7 | 1, 2, 4, 17 | 13 | Equal |
| 8 | 1, 3, 4, 17 | 13, 22 | Equal |
| 9 | 1, 2, 4, 17 | 10 | Decreased |
| 10 | 3, 17 | 8, 9, 10, 11 | Decreased |

### 5.4 Conclusion

These results indicate that it is possible to produce a recombinant Ara h 1 protein that will bind substantially lower amounts of serum IgE from peanut sensitive patients. This may present a safe alternative therapeutic reagent that could be used to desensitize peanut allergic patients.

### Example 6

### Biochemical and Structural Characterization of Ara h 1

### 6.1 Introduction

The position of each of the IgE binding epitopes on a homology-based molecular model of Ara h 1 shows that they are clustered into two main regions, despite their more even distribution in the primary sequence. Using a fluorescence assay we also show that Ara h 1 aggregates to form trimers and hexamers at high concentrations.

### 6.2 Methods

### Homology-Based Model of Ara h 1

Molecular modeling and computations were performed on Silicon Graphics workstations running IRIX 6.2. The Wisconsin Genetic Computer Group (GCG) software package (Devereux et al., Nucleic Acids Res. 12:387-395, 1984) was also used on a digital ALPHA workstation using OpenVMS Version 6.1.

The X-ray crystal structure of the phaseolin A chain (Protein Data Bank Accession Code 2PHL A) from Phaseolus vulgaris was used as the template for homology-based modeling (Lawrence et al., J. Mol. Biol. 238:748-776, 1994; Abola et al., pp. 107-132 in “Protein Data Bank in Crystallographic Databases-Information Content, Software Systems, Scientific Applications” Ed. by F. H. Allen et al., Data Commissioner of the International Union of Crystallography, Bonn, 1987; and Bernstein et al., J. Mol. Biol. 112:535-542, 1977). Ara h 1 was modeled as a monomer using the COMPOSER module of SYBYL Version 6.3 from Tripos Inc. (St. Louis, Mo.). Phaseolin is a smaller protein than Ara h 1, and it only allowed for the modeling of the region between amino acid residues 127-586 of SEQ ID NO. 7. Residues Ser²¹¹-Asp²¹⁹ Asn281-Lys²⁸² on the structure of phaseolin have not been solved because of low electron density (Lawrence et al., 1994, supra). Before attempting to use the structure for modeling, the regions were constructed using the protein loop search option in SYBYL and minimized using local annealing and the Powell algorithm.

Alignment between Ara h 1 and phaseolin A chain (GenBank 2PHLA) was determined using COMPOSER and was optimized with information from alignment of Ara h 1 to other vicilin homologs using the GCG pileup program. Following alignment, structurally conserved regions were constructed. Loops were then added using orientations to fragments from X-ray crystal structures in the SYBYL data based following homology searches and fitting screens. The model was minimized with the CHARMM force field using the Adopted Basis Newton-Raphson method using QUANTA Version 96 from Molecular Simulations Inc./BIOSYM (Burlington, Mass.). The protein backbone was given a harmonic force constraint constant of 500 to hold it rigid during the first 400 iterations of minimization, followed by relaxation with 100 steps each at constraints of 400, 300, 200, and 100 and a final 400 steps with a constraint of 10 (Brooks et al., J. Comput. Chem. 4:187-217, 1983 and Carlson et al., Hypertension 7:13-26, 1985).

### Fluorescence Polarization of Ara h 1 Higher Order Structure

Ara h 1 was purified to >95% homogeneity from crude peanut extract and labeled with fluorescein. A constant amount of the labeled protein, 10 nM, in binding buffer (50 mM Tris, 1 mM EDTA, 100 mM NaCl, 2 mM dithiothreitol, 5% glycerol, pH 7.5) was mixed with serial dilutions (by 0.5 or 0.8 increments) of unlabeled Ara h 1 to analyze oligomer formation. Fluorescence measurements were made using a Beacon fluorescence polarization spectrometer (Pan Vera, Madison, Wis.) with fixed excitation (490 nm) and emission (530 nm) wavelengths at room temperature (24° C.) in a final volume of 1.1 ml (Royer and Beechem, Methods Enzymol. 210:481-505, 1992 and Lundbald et al., Mol. Endocrinol. 10:607-612, 1996). Each data point is an average of three independent measurements. The intensity of fluorescence remained constant throughout the polarization measurements.

### Cross-Linking Experiments

Cross-linking experiments were done exactly as described in Maleki et al. (Biochemistry 36:6762-6767, 1997). Briefly, proteins were desalted into phosphate-buffered saline, pH 8.0, using disposable PD-10 gel filtration columns. The protein cross-linking reagent utilized was dithio-bis(succinimidyl propionate) (DSP). Limited cross-linking was performed so the monomer disappearance could be observed and to minimize the formation of nonspecific complexes.

### 6.3 Results

Location of the IgE binding epitopes on the three-dimensional structure of Ara h 1

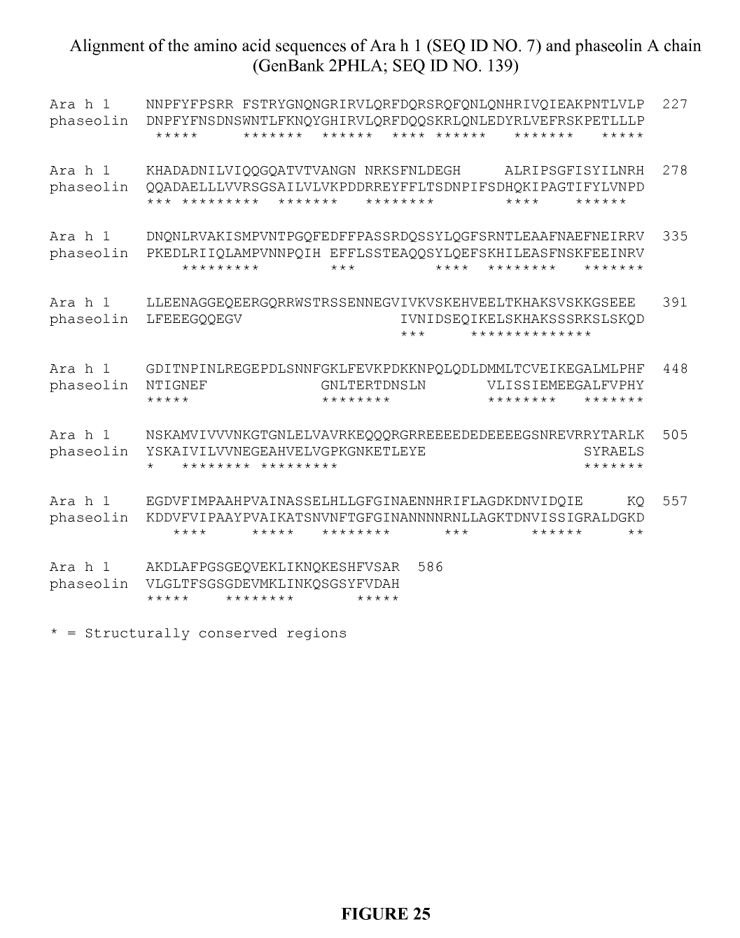

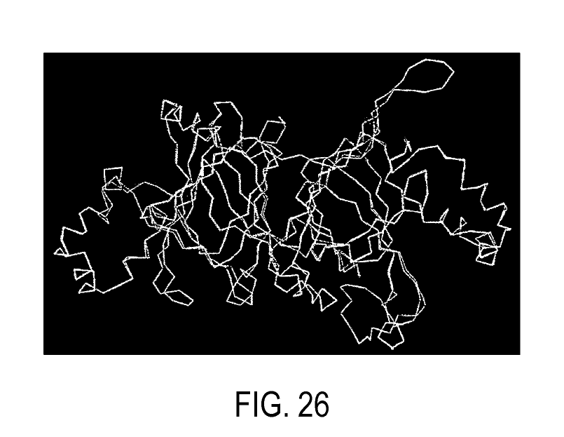

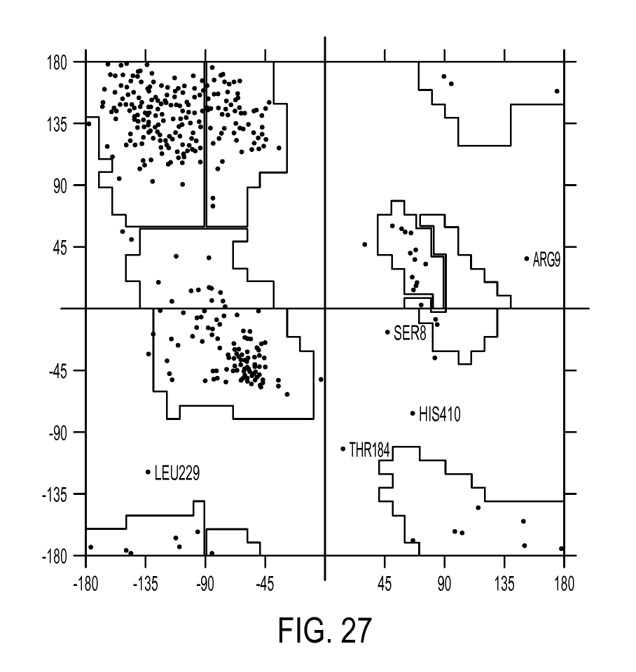

A homology-based model of Ara h 1 tertiary structure was generated to determine the location of the epitopes on this relatively large allergenic molecule. To construct this model, the primary amino acid sequence of Ara h 1 was aligned to the highly homologous protein phaseloin (GenBank 2PHLA, FIG. 25), for which x-ray crystal structure data was available (Protein Data Bank 2PHLA, FIG. 26). The quality of the Ara h 1 model was assessed using the protein health module of QUANTA and PROCHECK Version 2.1.4 (Laskowski et al., J. Appl. Crystallogr. 26:283-291, 1993) from Oxford Molecular Inc. (Palo Alto, Calif.) and compared with the quality of the structures of phaseolin and canavalin (Protein Data Bank 1CAU) (Abola et al., 1987, supra; Bernstein et al., 1977, supra; and Ko et al., Plant Physiol. 101:729-744, 1993). Most of the backbone torsion angles for non-glycine residues lie within the allowed regions of the Ramachandran plot (FIG. 27). Only 1.4% of the amino acids in the Ara h 1 model have torsion angles that are disallowed as compared with 0.3 and 0.6% of amino acids in phaseolin and canavalin, respectively (Table 12). In addition, the number of buried polar atoms, buried hydrophilic residues, and exposed hydrophobic residues in the Ara h 1 model are comparable with those found in the structures of phaseolin and canavalin (Table 12).

TABLE 12
Comparison of structures of Ara h 1, phaseolin, and canavalin.

| | Ara h 1 | Phaseolin | Canvalin |
|---|---|---|---|
| Buried polar atoms | 52 | 42 | 67 |
| Buried hydrophilic | 16 | 7 | 10 |
| Exposed hydrophobic | 2 | 2 | 3 |
| Ramachandran highly favored | 309 | 280 | 250 |
| Ramachandran allowed | 56 | 40 | 71 |
| Ramachandran disallowed | 5 | 1 | 2 |

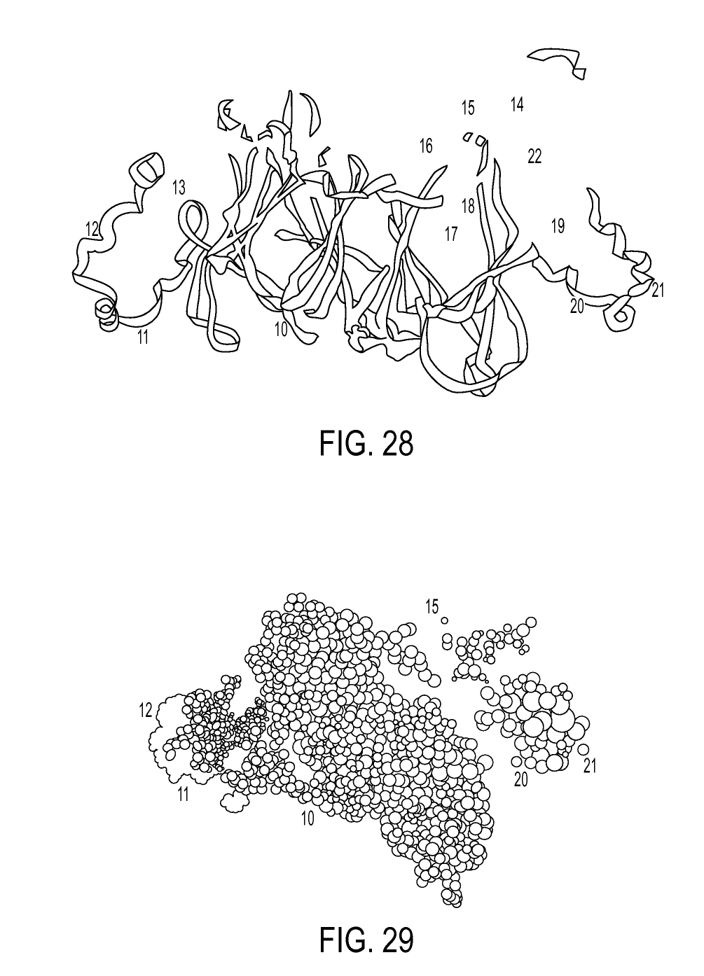

Taken together, these data indicate that the homology-based model of Ara h 1 tertiary structure is reasonable and similar to the structures of other homologous proteins that have been solved. The global fold of the Ara h 1 molecule and the position of epitopes 10-22 are shown in FIG. 28. The tertiary structure of the molecule consists of two sets of opposing anti-parallel β-sheets in Swiss roll topology joined by an interdomain linker. The terminal regions of the molecule consist of α-helical bundles containing three helices each. Epitope 12 resides on an N-terminal α-helix while epitopes 20 and 21 are located on C-terminal α-helices. Epitopes 14, 15 and 18 are primarily β-strands on the inner faces of the domain, and epitopes 16, 17, 19, and 22 are β-strands on the outer surface of the domain. The remainder of the epitopes are without a predominant type of higher secondary structure. A space-filled model depicting the surface accessibility of the epitopes and critical amino acids is shown in FIG. 29. Of the 35 residues that affected IgE binding, 10 were buried beneath the surface of the molecule, and 25 were exposed on the surface.

### Ara h 1 Aggregates to Form Stable Trimeric and Hexameric Structures

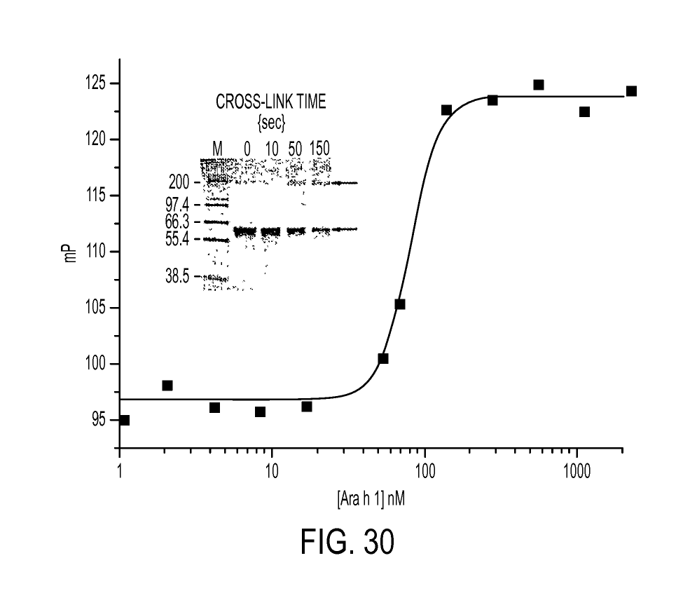

A rapid, reproducible fluorescence assay was developed in order to determine if Ara h 1 formed higher order structures similar to those observed for soybean vicilins. Purified, fluorescein-labeled Ara h 1, 10 nM, was mixed with various concentrations of unlabeled Ara h 1. The fluorescence polarization observed at each concentration was then determined and plotted as milli-polarization units (mP in arbitrary units) vs. the concentration of Ara h 1 (FIG. 30). Measurement of fluorescence reveals the average angular displacement of the fluorphor, which is dependent on the rate and extent of rotational diffusion. An increase in the size of the macromolecule through complex formation results in decreased rotational diffusion of the labeled species, which in turn results in an increase in polarization. The plateaus observed at protein concentrations between 0 and 20 nM and between 200 nM and 2 μM indicate the presence of a homogeneous species at these concentrations. The sharp increase in polarization observed at concentrations of Ara h 1 above 50 nM indicates that a highly cooperative interaction between Ara h 1 monomers had occurred that results in the formation of a stable homo-oligomeric structure. In order to determine the stoichiometry of this interaction, cross-linking experiments were performed followed by SDS-polyacrylamide gel electrophoresis analysis of the cross-linked products (FIG. 30, inset). Ara h 1 oligomers representing samples taken at the 200 nM concentration were subjected to limited chemical cross-linking with DSP. Cross-linked and non cross-linked samples were resolved by SDS-polyacrylamide gel electrophoresis and visualized by Coomassie staining of the gel. We found that limited cross-linking at 1 μM DSP results in the formation of an electrophoretically stable complex with an apparent molecular mass of ˜180 kd, appropriate for an Ara h 1 trimer.

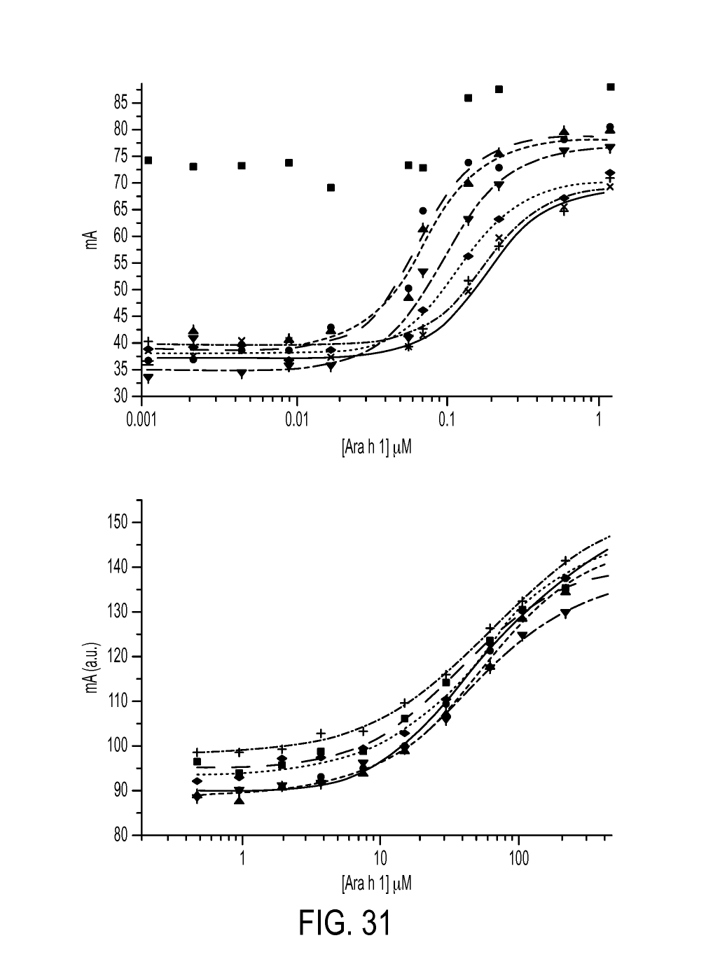

As shown in FIG. 31, fluorescence anisotropy measurements were also performed over a low and a high range of Ara h 1 concentrations (1-1000 nM and 1-200 μM, respectively) using a variety of NaCl concentrations (0-1800 mM). Each data point is an average of three independent measurements. The midpoint of the monomer to trimer transition remains at about 100-150 nM (FIG. 31, upper panel). At higher concentrations (above 40 mM), Ara h 1 aggregates further to form a stable hexameric structure of ˜360 kd (FIG. 31, lower panel). Table 13 summarizes the affinity constants (Kₐₚₚ) and ρ-values for the monomer to trimer and trimer to hexamer transitions that were obtained using a standard curve fitting procedure.

TABLE 13
Summary of Kₐₚₚ and ρ-values for Ara h 1 oligomer formation

| Ara h 1 oligomer | Salt conc. (mM) | Kₐₚₚ (μM) | ρ-value (coop.) |
|---|---|---|---|
| monomer to trimer | 0 | * | * |
| monomer to trimer | 100 | 0.065 | 2.40 |
| monomer to trimer | 300 | 0.070 | 2.25 |
| monomer to trimer | 500 | 0.095 | 2.10 |
| monomer to trimer | 900 | 0.120 | 2.10 |
| monomer to trimer | 1400 | 0.170 | 2.20 |
| monomer to trimer | 1800 | 0.170 | 2.10 |
| trimer to hexamer | 100 | 32.60 | 1.10 |
| trimer to hexamer | 400 | 36.00 | 1.03 |
| trimer to hexamer | 600 | 41.00 | 1.13 |
| trimer to hexamer | 800 | 45.00 | 1.00 |
| trimer to hexamer | 1100 | 48.00 | 0.90 |
| trimer to hexamer | 1300 | 54.00 | 0.90 |
| trimer to hexamer | 1800 | 65.00 | 0.80 |

* These values cannot be determined using a fitting program.

### 6.4 Conclusion

The characteristics that have been attributed to allergenic proteins include their abundance in the food source, their resistance to food processing, and their stability to digestion by the gastrointestinal tract (Astwood et al., Nature Biotechnology 14:1269-1273, 1996 and Veiths et al., pp. 130-149 in “Food Allergies and Intolerances”, Ed. by G. Eisenband, VCH Verlagsgesellschaft mbH, Weinheim, Germany, 1996). The major peanut allergen, Ara h 1, has been shown to be an abundant protein (see Example 1) that survives intact in most food processing methods (Lehrer et al., Crit. Rev. Food Sci. Nutr. 36:553-564, 1996) and is stable to digestion in in vitro systems designed to mimic the gastrointestinal tract (Becker, Monogr. Allergy 32:92-98, 1996). However, the physical characteristics that allow this protein to exhibit these properties have not previously been examined. Our observations on the tertiary structure of the Ara h 1 monomer and the determination that this protein readily forms a trimeric complex may help to determine why this protein is allergenic. For example, we have described the tertiary structure of the Ara h 1 protein as consisting of two sets of opposing antiparallel β-sheets in Swiss roll topology with the terminal regions of the molecule consisting of a α-helical bundles containing three helices apiece. While there are numerous protease digestion sites throughout the length of this protein, the structure may be so compact that potential cleavage sites are inaccessible until the protein is denatured. In addition, the formation of trimeric complexes and further higher order aggregation may also afford the molecule some protection from protease digestion and denaturation and allow passage of Ara h 1 across the small intestine. It has been shown that some atopic individuals transfer more antigen across the small intestine in both the intact and partially degraded state (Majamaa and Isolauri, J. Allergy Clin. Immunol. 97:985-990, 1996). These physical attributes of the Ara h 1 molecule may help to explain the extreme allergenicity exhibited by this protein.

### Example 7

### Effects of Enzymatic Digestion of Ara h 1

### 7.1 Introduction

As was shown in Example 6, Ara h 1 forms stable trimers at concentrations above about 0.1 μM. In this trimeric form, Ara h 1 was found to be extremely resistant to the proteolytic enzymes found throughout the digestive tract. Upon treatment with trypsin, chymotrypsin, and pepsin, a number of large fragments are produced which are strongly resistant to further enzymatic digestion. These resistant Ara h 1 peptide fragments contain intact IgE binding epitopes and several potential enzyme cut sites which are protected from the enzyme by the compact trimeric structure of the protein. Amino acid sequence analysis of the resistant protein fragments indicate that they contain most of the immunodominant IgE binding epitopes. The enzyme treated allergen remains essentially intact despite the action of the proteases until the fragments are dissociated with a detergent.

### 7.2 Methods and Results

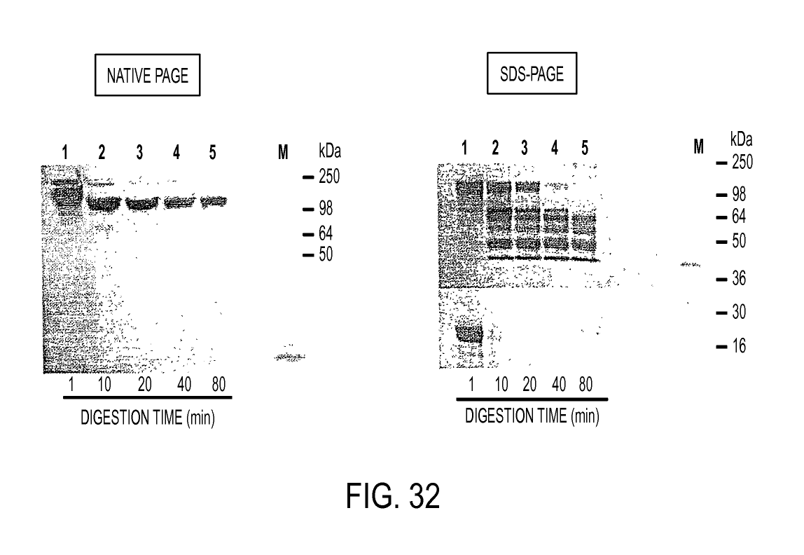

Ara h 1 protein was digested with trypsin and loaded on a native gel (Native PAGE, FIG. 32A). The same digestion samples were loaded onto a denaturing gel (SDS-PAGE, FIG. 32B) to see the digested subunits. The Ara h 1 trimer remained associated even after 80 minutes of digestion.

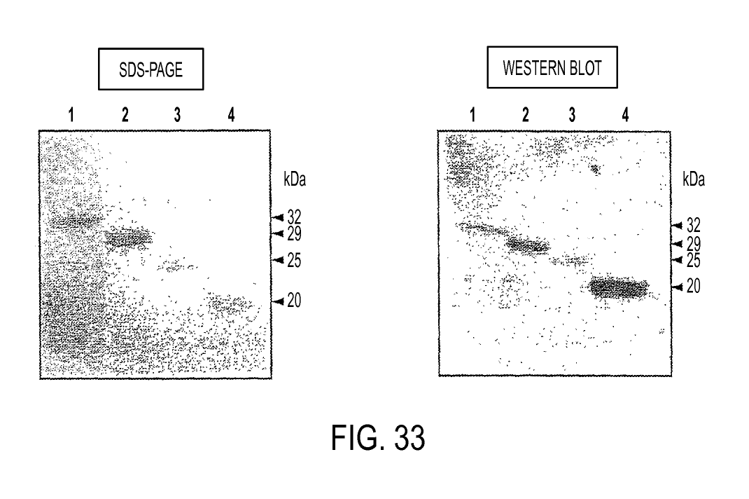

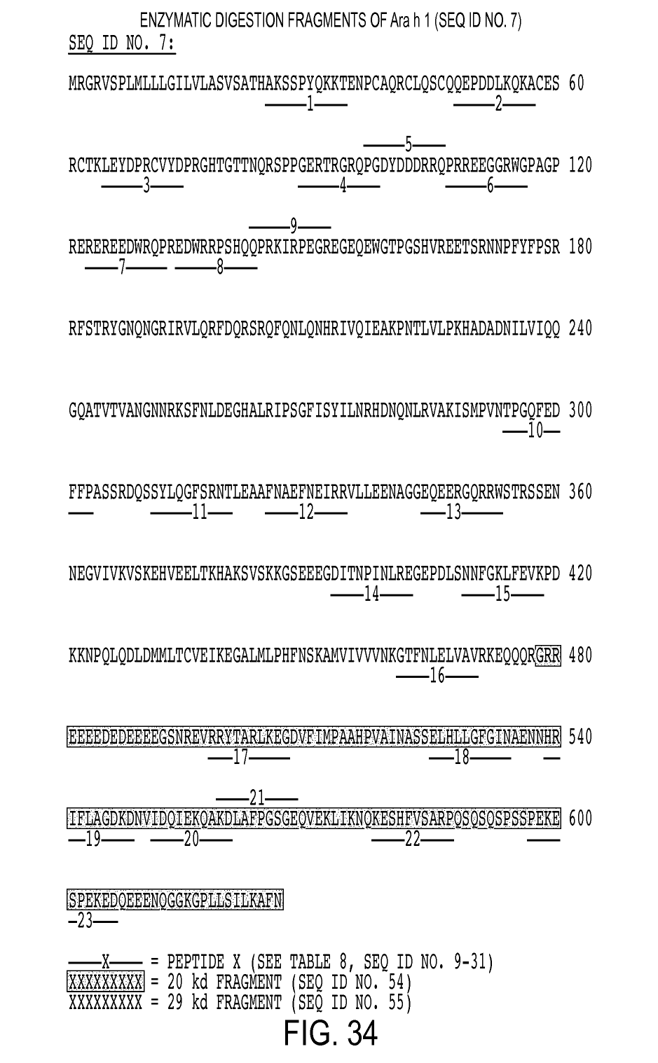

Protease resistant fragments of Ara h 1 were further purified by SDS-PAGE (FIG. 33A) and the same samples were transferred to PVDF membrane to be analyzed by western blot using pooled IgE sera from peanut sensitive allergenic individuals. The bound IgE were detected using ¹²⁵I-labeled anti-IgE by autoradiography (FIG. 33B). The 20 kd fragment was observed as the most IgE reactive followed by the 29 kd fragment. These two fragments were N-terminally sequenced to locate their respective positions in Ara h 1 (FIG. 34).

The IgE binding epitopes that were determined in Example 4 are underlined in FIG. 34. The amino acid sequence of the most reactive protease resistant fragments are highlighted. The 20 kd fragment (SEQ ID NO. 54 covering amino acids 478-626 of SEQ ID NO.7) contains the highest number of epitopes and corresponds to the C-terminal end of the protein. The 20 kd fragment also contains immunodominant epitope 17. This fragment is involved in monomer-monomer interactions to form a trimer along with the second epitope rich 29 kd fragment (SEQ ID NO. 55 which covers amino acids 146-413 of SEQ ID NO.7).

### 7.3 Conclusion

The trimeric structure of Ara h 1 plays a significant role in its stability to protease digestion. Immunodominant IgE binding epitopes of Ara h 1 may be determined by this structure.

### Example 8

### Purification and Isolation of Ara h 2 Using Pooled IgE Sera

### 8.1 Introduction

Serum from nine patients with atopic dermatitis and a positive double-bind, placebo-controlled, food challenge to peanut were used in the process of identification and purification of the peanut allergens. Identification of a second major peanut allergen was accomplished with use of various biochemical and molecular techniques. Anion exchange chromatography of the crude peanut extract produced several fractions that bound IgE from the serum of the patient pool with positive challenges. By measuring anti-peanut specific IgE and by IgE-specific immunoblotting we have identified an allergic component that has two closely migrating bands with a mean molecular weight of 17 kd. Two-dimensional gel electrophoresis of this fraction revealed it to have a mean isoelectric point of 5.2. According to allergen nomenclature of this IUIS Subcommittee for Allergen Nomenclature this allergen is designated, Ara h 2 (Arachis hypogaea).

### 8.2 Methods

### Patients Sensitive to Peanuts

Approval for this study was obtained from the Human Use Committee at the University of Arkansas for Medical Sciences. Nine patients (mean age, 4.2 years) with AD and a positive immediate prick skin test to peanut had either a positive DBPCFC or a convincing history of peanut anaphylaxis (the allergic reaction was potentially life threatening, that is with laryngeal edema, severe wheezing, and/or hypotension) (7 patients had positive DBPCFCs). Details of the challenge procedure and interpretation have been previously discussed (Burks et al., 1988a, supra). Five milliliters of venous blood were drawn from each patient, allowed to clot, and the serum was collected. An equal volume of serum from each donor was mixed to prepare a nine-person peanut-specific IgE antibody pool.

### Crude Peanut Extract

Three commercial lots of southeastern runners (Arachis hypogaea), medium grade, from the 1979 crop (North Carolina State University) were used in this study. The peanuts were stored in the freezer at −18° C. until roasted. The three lots were combined in equal proportions and blended before defatting. The defatting process (defatted with hexane after roasting for 13 to 16 minutes at 163° to 177° C.) was done in the laboratory of Dr. Clyde Young (North Carolina State University). The powdered crude peanut was extracted per the recommendations of Yunginger and Jones (1987, supra) in 1 mol/L NaCl to 20 mmol/L sodium phosphate (pH 7.0) with the addition of 8 mol/L urea for 4 hours at 4° C. The extract was isolated by centrifugation at 20,000 g for 60 minutes at 4° C. The total protein determination was done by the (BCA) method (Bio-Rad Laboratories, Richmond, Calif.).

### Chromatography

Analytic and preparative anion-exchange chromatography was performed with the FPLC system (Pharmacia, Piscataway, N. J.). Anion-exchange chromatography used the PL-SAX column (anion exchange column, Polymer Laboratories, Amherst, Mass.). The crude peanut extract was dialyzed against 20 mmol/L of Tris-bis-propane (pH 7.2) without urea and 40 mg loaded on the PL-SAX column. A stepwise salt gradient of 0 to 1.5 mol/L NaCl was applied. All fractions of each resolved peak were pooled, dialyzed, and lyophilized.

Dot blotting was done to determine which fractions from the anion exchange column chromatogram contained IgE-binding material. Two hundred microliters of each fraction were blotted with the Mini Blot apparatus (Schleicher and Schuell, Keene, N. H.) onto 0.45 um nitrocellulose membranes (Bio-Rad Laboratories). After the membranes were dried, the remaining active sites were blocked with 20 ml of blocking solution (0.5% gelatin with 0.001% thimerosal in 500 ml of PBS) for 1 hour. The procedure is then identical to the immunoblotting of IgE.

### Electrophoresis and Immunoblotting

The electrophoresis procedure is a modification of Sutton et al. (Laemmli, 1970, supra and Sutton et al., 1982, supra). SDS-PAGE was carried out with a 12.5% polyacrylamide separating gel and a stacking gel of 3%. Twenty microliters of a 1 mg/ml solution of each fraction was applied to each well. Replicate samples were applied for independent analysis. Electrophoresis was performed for 4 hours at 0.030 A per gel (E-C Apparatus Corp., St. Petersburg, Fla.) for the 14 cm by 12 cm gels, and for 1 hour at 175 V per gel for the 8 cm by 7.5 cm gels (Mini-Protean II system, Bio-Rad Laboratories). To assure proper protein separation and visualization, Coomassie brilliant blue (Sigma Chemical Co., St. Louis, Mo.) stains were done on gels. For detection of carbohydrate staining material, gels were stained with the modified PAS stain according to the method of Kapitany and Zebrowski (1973, supra)

Proteins were transferred from the separating gel to a nitrocellulose membrane in a transfer buffer (tris-glycine) with 10% SDS and 40% methanol. (Towbin et al., 1979, supra) The procedure was done in a transblot apparatus (Bio-Rad Laboratories) for 2 hours (0.150 A). An amido black stain (Bio-Rad Laboratories) was done to assure transfer of the protein.

After removal from the transblot apparatus, the nitrocellulose was placed in blocking solution overnight. The nitrocellulose blot was then washed three times with PBS (PBS with 0.05% Tween 20) and incubated with the pooled peanut-sensitive IgE serum (1:20 dilution) for 2 hours at 4° C. with rocking. After washing again with PBS three times, alkaline phosphatase-conjugated goat antihuman IgE (1:1000 vol/vol of PBS, Bio-Rad Laboratories) was added and incubated at room temperature with rocking for 2 hours. After again washing with PBS three times, the blot was developed with 250 μl of 30 mg nitro blue tetrazolium in 70% dimethylformamide (NBT) (Bio-Rad Laboratories) and 250 μl of 15 mg of 5-bromo-4-chloro-3-indolyl-phosphate in 70% dimethylformamide (BCIP) (Bio-Rad Laboratories) solutions in 25 ml carbonate buffer (0.2 mol/L, pH 9.8) at room temperature for 15 minutes. The reaction was then stopped by decanting the NBT/BCIP solution and incubating the nitrocellulose for 10 minutes with distilled water. The blot was air-dried for visual analysis.

### ELISA for IgE

A biotin-avidin ELISA was developed to quantify IgE antipeanut protein antibodies with modifications from an assay previously published (Burks et al., 1986, supra). The upper two rows of a 96-well microtiter plate (Gibco, Santa Clara, Calif.) were coated with 100 μl each of equal amounts (1 μg/ml) of antihuman IgE monoclonal antibodies, 7.12 and 4.15 (kindly provided by Dr. A. Saxon). The remainder of the plate was coated with one of the peanut products at a concentration of 1 μg/ml in coating buffer (0.1 mol/L sodium carbonate-bicarbonate buffer, pH 9.5). The plate was incubated at 37° C. for 1 hour and then was washed five times with rinse buffer (PBS, pH 7.4, containing 0.05% Tween 20; Sigma Chemical Co.) immediately and between subsequent incubations. The upper two rows used a secondary standard reference to generate a curve for IgE, ranging from 0.05 to 25 ng/ml.

The serum pool and patient serum samples were diluted (1:20 vol/vol) and dispensed in duplicate in the lower portion of the plate. After incubation for 1 hour at 37° C. and washing, biotinylated, affinity-purified goat antihuman IgE (KPL, Gaithersburg, Md.) (1:1000 vol/vol PBS) was added to all wells. Plates were incubated for 1 hour at 37° C., washed, and 100 μl horseradish peroxidase-avidin conjugate (Vector Laboratories, Burlingame, Calif.) added for 30 minutes. After washing, the plates were developed by the addition of a buffer containing O-phenylenediamine (Sigma Chemical Co.). The reaction was stopped by the addition of 100 μl 2-N-hydrochloric acid to each well, and absorbance was read at 492 nm (Titertek Multiscan, Flow Laboratories, McLean, Va.). The standard curve was plotted on log-logit scale by means of simple linear regression, and values for the pool and individual patient samples were read from the curve as “nanogram-equivalent units” per milliliter (nanogram per milliliter). (Burks et al., 1988b, supra and Burks et al., 1990, supra)

### ELISA Inhibition

A competitive ELISA inhibition was done with the FPLC fractions. One hundred microliters of pooled serum (1:20) from the patients with positive challenges was incubated with various concentrations of the FPLC protein fractions (0.00005 to 50 ng/ml) for 18 hours. The inhibited pooled serum was then used in the ELISA described above. The percent inhibition was calculated by taking the food-specific IgE value minus the incubated food-specific IgE value divided by the food-specific IgE value. This number is multiplied by 100 to get the percentage of inhibition.

### Isoelectric Focusing

The samples were focused with the LKB Multiphor system using LKB PAG plates, pH gradient 3.5 to 9.5 (LKB, Bromma, Sweden). Five microliters of sample (1 mg/ml) was applied, and an electric current of 200 V was applied for 30 minutes and then increased to 900 to 1200 V for 30 minutes. The gel was fixed and stained with Coomassie brilliant blue following the standard protocol (LKB).

### Two-Dimensional Gel Electrophoresis

The samples were run according to the method of O'Farrell et al. (J. Biol. Chem. 250:4007-4021, 1975). The first dimension is an isoelectric focusing gel in glass tubing. After making the gel mixture the samples are loaded with overlay solution and 0.02 mol/L NaOH. The samples are run at 400 V for 12 hours and at 800 V for 1 hour. After removing the gel from the tube, the isoelectric focusing gel is equilibrated for 2 hours in SDS sample buffer. The second dimension is 14 cm by 12 cm, 12.5% polyacrylamide gel described in the electrophoresis section. The gels were stained with the pooled peanut-positive serum for IgE-specific bands as above.

### Amino Acid Analysis, Amino Acid Sequencing, and Carbohydrate Analysis

The 17 kd fraction was run on a 10% mini-gel (Bio-Rad Laboratories) in triglycine buffer and stained with Rapid Reversible Stain (Diversified Biotech, Newton Centre, Mass.). The two bands were cut separately from the gel and electroluted in tris-glycine SDS buffer. After lyophilization the bands were sequenced individually. Automated gas-phase sequencing was performed on an Applied Biosystems model 475A sequencing system (Dr. Bill Lewis, University of Wyoming, Laramie, Wyo.). Amino acid analysis was done with a Hitachi (Hitachi Instruments, Inc., Danbury, Conn.) HPLC L5000 LC controller with a C18 reverse-phase column.

The electroluted 17 kd fraction was analyzed for carbohydrate analysis (Dr. Russell Carlson, Complex Carbohydrate Research Center, University of Georgia, Athens, Ga.). Glycosyl composition analysis on these samples was performed by the preparation and analysis of trimethylsiyl methylglycosides.

### 8.3 Results

### Chromatography

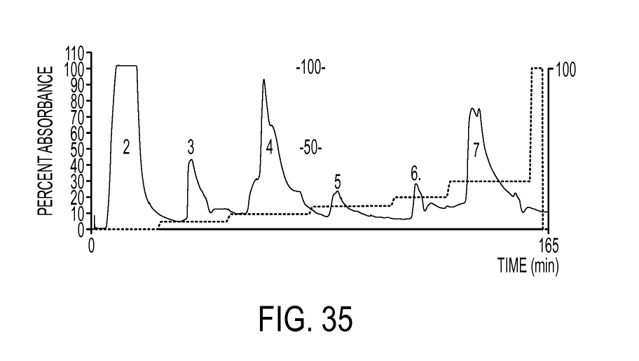

Pilot experiments were conducted with the analytical Mono Q 5/5 (Pharmacia) anion exchange column to determine the optimal buffer system and salt gradient. Screening for IgE-specific peanut binding components was done by dot blotting of these fractions. Scale up and optimization was completed with the PL-SAX column (anion exchange), with a stepwise salt gradient (0 to 1.5 mol/L NaCl). This procedure separated the crude peanut extract into seven major peaks (FIG. 35). Preliminary dot blotting from this separation identified IgE-binding material in each peak (picture not shown). Multiple runs of this fractionation procedure were performed, and each isolated peak was pooled, dialyzed against 100 mmol/L NH₄HCO₃, and lyophilized.

### Electrophoresis and Immunoblotting

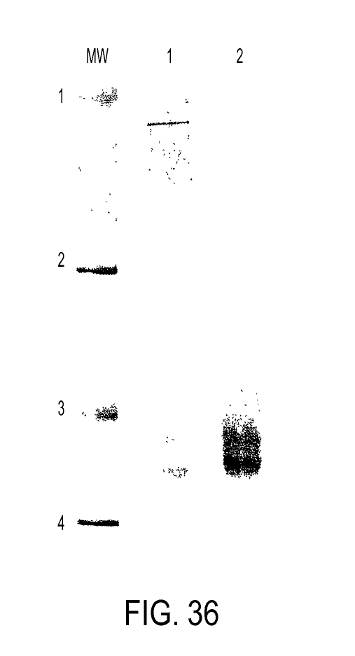

Initial SDS-PAGE and immunoblotting of the crude peanut extract revealed multiple fractions with several IgE-staining bands (see Example 1). Aliquots of the seven lyophilized fractions from the anion exchange column were analyzed by SDS-PAGE (data not shown). Each fraction showed 2 to 5 Coomassie brilliant blue staining protein bands. Immunoblotting for specific IgE with the pooled serum revealed IgE-staining bands in each fraction. Fraction 4 showed two large, closely migrating, IgE-specific bands with a mean molecular weight of 17 kd (FIG. 36) (6% by weight of crude peanut extract).

### ELISA and ELISA Inhibition

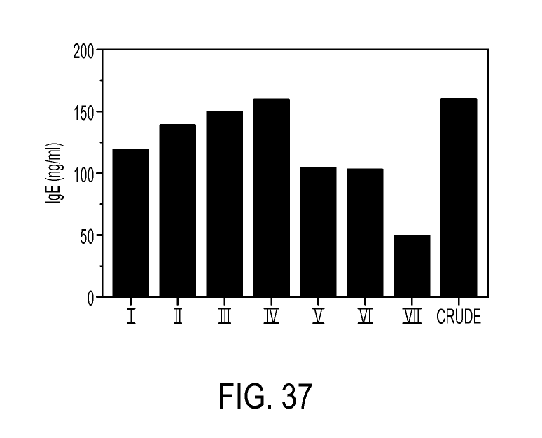

ELISA results comparing the crude peanut extract with each isolated fraction are shown in FIG. 37. Fractions 1 through 7 all had IgE-binding from the peanut-positive serum pool. We tested individually the serum of six patients (members of pooled serum) to determine the relative IgE-binding material to both the crude peanut, fraction 4 (which contained the 17 kd component), and Ara h 1 (major component, 63.5 kd fraction). Each patient's serum had measurable amounts of peanut-specific IgE to each. Three of the patients had more peanut-specific IgE (ng/ml) to the 17 kd fraction than to the 63.5 kd fraction (Table 14).

TABLE 14
Concentrations (ng/ml) of peanut-specific lgE binding

| | To crude peanut | To Ara h 1 | To fraction 4 |
|---|---|---|---|
| Patient | (ng/ml) | (ng/ml) | (ng/ml) |
| 1 | 4.2 | 21.0 | 14.6 |
| 2 | 7.0 | 11.4 | 13.0 |
| 3 | 285.2 | 285.8 | 380.0 |
| 4 | 1.0 | 2.0 | 3.2 |
| 5 | 11.4 | 19.4 | 17.0 |
| 6 | 5.8 | 12.0 | 9.8 |
| 7 | <0.05 | <0.05 | <0.05 |
| 8 | <0.05 | <0.05 | <0.05 |
| Normals | <0.05 | <0.05 | <0.05 |

Patients 1 to 6 are patients with AD and positive DBPCFCs to peanut.
Patient 7 is a patient with AD who had positive DBPCFC to milk and elevated serum IgE values but did not have positive skin test results or positive challenge to peanut (n = 2).
Patient 8 is a healthy control patient from the serum bank in the ACH Special Immunology Laboratory (n = 2).

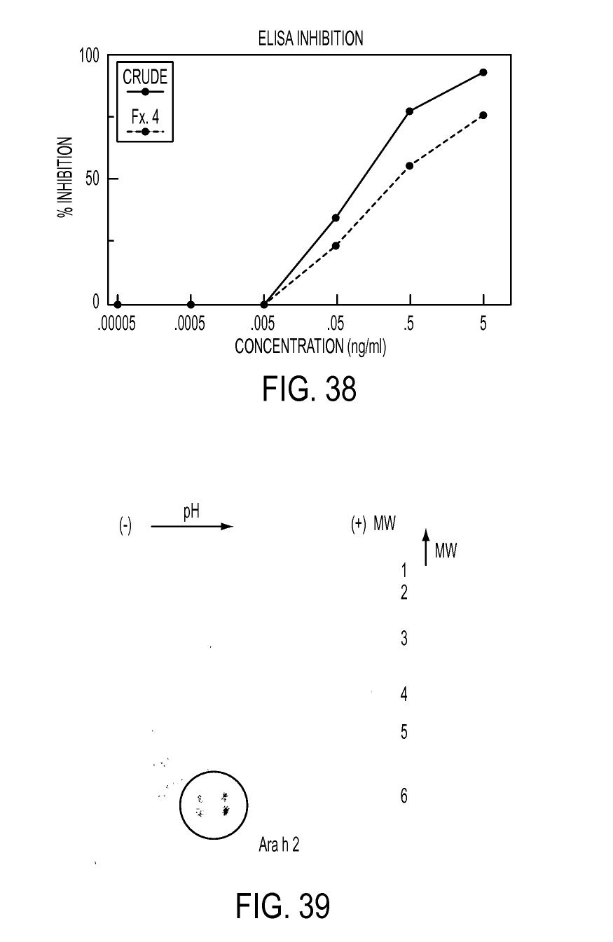

The ELISA inhibition results are shown in FIG. 38. The concentration of the 17 kd fraction required to produce 50% inhibition was 0.4 ng/ml compared with 0.1 ng/ml of the crude peanut extract (Jusko, 1990, supra).

### Two-Dimensional Gel Electrophoresis

Because immunoblotting and ELISAs of the various anion exchange fractions suggested that fraction 4 appeared to contain a major allergen, isoelectric focusing was done on this fraction. The two bands in this allergen, which migrated closely together at a mean molecular weight of 17 kd on SDS-PAGE stained with Coomassie brilliant blue, had a pI of 5.2 (gel not shown). FIG. 39 shows the Coomassie-stained gel of the 17 kd fraction. One can see the protein divided into four distinct areas at a mean molecular weight of 17 kd and a mean pl of 5.2.

### Amino Acid Analysis, Amino Acid Sequencing, and Carbohydrate Analysis

Table 15 shows the complete amino acid analysis of the purified peanut fraction. The fraction was particularly rich in glutamic acid, aspartic acid, glycine, and arginine.

TABLE 15
Amino acid analysis of Ara h 2

| Amino acid | Residues/molecule |
|---|---|
| Asp | 12.2 |
| Glu | 24.8 |
| Ser | 9.8 |
| His | 1.3 |
| Gly | 11.3 |
| Thr | 2.2 |
| Arg | 10.8 |
| Ala | 5.4 |
| Tyr | 3.9 |
| Met | 2.7 |
| Val | 2.4 |
| Phe | 2.4 |
| Ile | 2.9 |
| Leu | 7.9 |

The amino acid sequences for both 17 kd bands are shown in Table 16. Molecular weight discrepancies may be a result of carbohydrate composition in the two isoallergens. There were no known similar N-terminal sequences found in PIR, GenBank, or SwissProt.

TABLE 16
Sequencing of the upper and lower bands
of electroluted 17 kd peanut allergen

| | SEQ | |
|---|---|---|
| | ID | |
| Band | NO. | Amino acid sequence |
| Upper | 57 | XQQXELQXDXXXQSQLDADLRPGEQXLMXKI |
| | | ++ +++ ++ + +++ ++ ++ ++ |
| Lower | 58 | XQQXELQDXEXXQSQERANLRPREQXLMXKI |

X = Unable to identify amino acid.

The 17 kd fraction was found to be 20% carbohydrate with significant levels of galacuronic acid, arabinose, and xylose (Table 17).

TABLE 17
Glycosyl composition analysis of 17 kd allergen

| Glycosyl residue | Ara h 2 (μg/total) |
|---|---|
| Arabinose | 14.0 |
| Rhamose | 2.8 |
| Fucose | 0.6 |
| Xylose | 9.3 |
| Mannose | 2.5 |
| Galactose | 4.4 |
| Glucose | 5.0 |
| Galacuronic acid | 41.0 |
| Galactosamine | ND |

ND, Not determined

### 8.4 Conclusion

The allergen described in this report has two major bands, with an apparent mean molecular weight of 17 kd on SDS-PAGE and a mean pI of 5.2. This fraction bound specific anti-peanut IgE from the peanut-positive pool in the ELISA and in the immunoblotting experiments. When used in the ELISA-inhibition studies, the 17 kd fraction significantly inhibited the IgE binding from the peanut-positive pool. In preliminary studies we have used the 17 kd allergen to inhibit binding from the pooled peanut-positive IgE serum to Ara h 1. There does not appear to be a moderate amount of inhibition of IgE binding to Ara h 1 produced by the 17 kd allergen.

According to recent recommendations by a recent international committee (IUIS) for proper identification of allergens we have designated this fraction Ara h 2 (Chapman, Curr. Opin. Immunol. 1:647-653, 1989). This fraction has been purified from a crude peanut extract from Florunner peanuts (Arachis hypogaea) by anion exchange chromatography. The fraction was identified as a major allergen by SDS-PAGE, ELISA, ELISA inhibition, TLIEF, amino acid analysis, and sequencing, carbohydrate analysis, and two-dimensional gels.

### Example 9

### Purification and Isolation of Ara h 2 Using Murine Monoclonal Antibodies

### 9.1 Introduction

The antigenic and allergenic structure of Ara h 2, a major allergen of peanuts, was investigated with the use of four monoclonal antibodies obtained from BALB/c mice immunized with purified Ara h 2. When used as a solid phase in an ELISA, these monoclonal antibodies captured peanut antigen, which bound human IgE from patients with positive peanut challenge responses. The Ara h 2 monoclonal antibodies were found to be specific for peanut antigens when binding for other legumes was examined. In ELISA inhibition studies with the monoclonal antibodies, we identified two different antigenic sites on Ara h 2. In similar studies with pooled human IgE serum from patients with positive challenge responses to peanuts, we identified two closely related IgE-binding epitopes.

### 9.2 Methods

### Patients With Positive Peanut Challenge Responses

Approval for this study was obtained from the Human Use Advisory Committee at the University of Arkansas for Medical Sciences. Twelve patients with atopic dermatitis and a positive immediate prick skin test response to peanut had either a positive response to double-blind placebo-controlled food challenge (DBPCFC) or a convincing history of peanut anaphylaxis (the allergic reaction was potentially life-threatening, that is with laryngeal edema, severe wheezing, and/or hypotension). Details of the challenge procedure and interpretation have been previously discussed (see Example 1). Five milliliters of venous blood was drawn from each patient and allowed to clot, and the serum was collected. An equal volume of serum from each donor was mixed to prepare a peanut-specific IgE antibody pool.

### Crude Peanut Extract

Three commercial lots of Southeastern Runners peanuts (Arachis hypogaea), medium grade, from the 1979 crop (North Carolina State University) were used in this study. The peanuts were stored in the freezer at −18° C. until they were roasted. The three lots were combined in equal proportions and blended before defatting. The defatting process (defatted with hexane after roasting for 13 to 16 minutes at 163° C. to 177° C.) was done in the laboratory of Dr. Clyde Young (North Carolina State University). The powdered crude peanut was extracted in 1 mol/L NaCl, 20 mmol/L sodium phosphate (pH 7.0), and 8 mol/L urea for 4 hours at 4° C. The extract was clarified by centrifugation at 20,000 g for 60 minutes at 4° C. The total protein determination was done by the bicinchoninic acid method (Pierce Laboratories. Rockville, Ill.).

### Monoclonal Antibodies

Mouse hybridoma cell lines were prepared by standard selection after polyethylene glycol-mediated cell fusion was carried out as previously described (Rouse et al., Infect. Immun. 58:1445-1499, 1990). Sp²/0-Ag14 mouse/myeloma cells were fused with immune splenocytes from female BALB/c mice hyperimmunized with Ara h 2. Hybridoma cell supernatants were screened by ELISA and Western blotting, and cell lines were cloned by limiting dilution. The antibodies secreted by the monoclonal hybridoma cell lines were isotyped according to the directions provided (Screen Type; Boehringer Mannheim, Indianapolis, Ind.). Ascites fluid produced in BALB/c mice was purified with Protein G Superose, as outlined by the manufacturer (Pharmacia, Uppsala, Sweden). Purified monoclonal antibodies were used in ELISA and ELISA inhibition assays.

### ELISA for IgE

A biotin-avidin ELISA was developed to quantify IgE anti-peanut protein antibodies with modifications from an assay previously described. The upper 2 rows of a 96-well microtiter plate (Gibco, Santa Clara, Calif.) were coated with 100 μl each of equal amounts (1 μl/ml) of anti-human IgE monoclonal antibodies, 7.12 and 4.15 (kindly provided by Dr. Andrew Saxon). The remainder of the plate was coated with the peanut protein at a concentration of 1 μl/ml in coating buffer (0.1 mol/L sodium carbonate-bicarbonate buffer, pH 9.6). The plate was incubated at 37° C. for 1 hour and then washed five times with rinse buffer (phosphate-buffered saline, pH 7.4, containing 0.05% Tween 20; Sigma Chemical Co., St. Louis, Mo.) immediately and between subsequent incubations. A secondary IgE reference standard was added to the upper 2 rows to generate a curve for IgE, ranging from 0.05 to 25 ng/ml.

The serum pool and individual patient serum samples were diluted (1:20 vol/vol) and dispensed into individual wells in the lower portion of the plate. After incubation for 1 hour at 37° C. and washing, biotinylated, affinity-purified goat anti-human IgE (KPL, Gaithersburg, Md.) (1:1000 vol/vol of bovine serum albumin) was added to all wells. Plates were incubated for 1 hour at 37° C. and washed, and 100 μl horseradish peroxidase-avidin conjugate (Vector Laboratories, Burlingame, Calif.) was added for 5 minutes. After washing, the plates were developed by the addition of a citrate buffer containing o-phenylenediamine (Sigma Chemical Co.). The reaction was stopped by the addition of 100 μl of 2N hydrochloric acid to each well, and absorbance was read at 490 nm (Bio-Rad Microplate reader model 450: Bio-Rad Laboratories Diagnostic Group, Hercules, Calif.). The standard curve was plotted on a log-logit scale by means of simple linear regression analysis, and values for the pooled serum and individual samples were read from the curve.

### ELISA Inhibition

An inhibition ELISA was developed to examine the site specificity of the monoclonal antibodies generated to Ara h 2. One hundred microliters of Ara h 2 protein (1 mg/ml) was added to each well of a 96-well microtiter plate (Gibco) in coating buffer (carbonate buffer, pH 9.6) for 1 hour at 37° C. Next, 100 μl of differing concentrations (up to 1000-fold excess) of each of the monoclonal antibodies was added to each well for 1 hour at 37° C. After washing, a standard concentration of the biotinylated monoclonal antibody preparation was added for 1 hour at 37° C. The assay was developed by the addition of the avidin substrate as in the ELISA above.

A similar ELISA inhibition was performed with the peanut-positive serum IgE pool instead of the biotinylated monoclonal antibody to determine the ability of each monoclonal antibody to block specific IgE binding.

### 9.3 Results

### Hybridomas Specific for Ara h 2

Cell fusions between spleen cells obtained from female BALB/c mice immunized with Ara h 2 and the mouse myeloma cells resulted in a series of hybridomas specific for Ara h 2. Seven monoclonal antibody-producing lines were chosen for further study. In preliminary studies all seven hybridoma-secreting cell lines had antibodies that bound Ara h 2, as determined by ELISA and immunoblot analysis (Sutton et al., 1982, supra and Towbin et al., 1979, supra). On the basis of different binding studies, four of the hybridomas were used for further analysis. As determined by isotype immunoglobulin-specific ELISA, all four hybridoma-secreting cell lines typed as IgG₁.

ELISA with Monoclonal Antibody as Solid Phase

Four monoclonal antibody preparations (4996D6, 4996C3, 5048B3, and 4996D5) were used as capture antibodies in an ELISA with Ara h 2 as the antigen. Serum from individual patients, who had positive challenge responses to peanut, was used to determine the amount of IgE binding to each peanut fraction captured by the Ara h 2-specific monoclonal antibody (Table 18). A reference peanut-positive serum pool was used as the control serum for 100% binding. Seven patients who had positive DBPCFC ism responses to peanut were chosen.

TABLE 18
Peanut-specific IgE to antigen presented
by four monoclonal antibodies

Capture antibody (%)

| Patient No. | 4996D6 | 4996C3 | 5048B3 | 4996D5 |
|---|---|---|---|---|
| 1 | 95 | 80 | 80 | 91 |
| 2 | 94 | 66 | 72 | 90 |
| 3 | 96 | 114 | 87 | 96 |
| 4 | 98 | 116 | 76 | 96 |
| 5 | 97 | 74 | 130 | 107 |
| 6 | 94 | 63 | 76 | 86 |
| 7 | 109 | 123 | 104 | 116 |
| 8 | 0 | 0 | 0 | 0 |
| 9 | 0 | 0 | 0 | 0 |

Ara h 2 monoclonal antibodies used as capture antibodies in ELISA with Ara h 2 as the antigen. Values are expressed as a percent of binding compared with challenge-positive peanut pool.
Patients 1 to 7 has positive DBPCFC responses to peanut; patient 8 is the patient without peanut sensitivity with elevated serum IgE; and patient 9 is the patient without peanut sensitivity with normal serum IgE.

All seven patients had significant amounts of anti-peanut-specific IgE to the peanut antigen presented by each of the four monoclonal antibodies compared with the control sera (patient 8 without peanut sensitivity who had elevated serum IgE values, patient 9 without peanut sensitivity who had normal serum IgE values). Titration curves were performed to show that limited amounts of antigen binding were not responsible for similar antibody binding. There were no significant differences in the levels of anti-peanut-specific IgE antibody to the peanut antigens presented by each monoclonal antibody. Most patients had their highest value for IgE binding to the peanut antigen presented by either 4996D6 or 4996C3, whereas no patient had his or her highest percentage of IgE binding to the peanut antigen presented by monoclonal antibody 4996D5.

### Food Antigen Specificity of Monoclonal Antibodies to Ara h 2

To determine whether the monoclonal antibodies to Ara h 2 would bind to only peanut antigen, an ELISA was developed with the pooled peanut-specific IgE from patients who had positive DBPCFC responses to peanut. All four monoclonal antibodies that were fully characterized bound only peanut antigen (Table 19). In the ELISA no binding to soy, lima beans, or ovalbumin occurred. When the normal serum pool was used in the ELISA, no peanut-specific IgE to either Ara h 2 or crude peanut could be detected.

In the United States, three varieties of peanuts are commonly consumed: Virginia, Spanish, and Runner. In an ELISA, we attempted to determine whether there were differences in monoclonal antibody binding to the three varieties of peanuts. There was only a minor variation with the ability of the peanut-specific IgE to bind to the captured peanut antigen (data not shown).

TABLE 19
IgE-specific binding to legumes captured
by Ara h 2 monoclonal antibodies

Capture antibody (%)

| Protein | 4996D6 | 4996C3 | 5048B3 | 4996D5 |
|---|---|---|---|---|
| Pooled serum | | | | |
| Crude peanut | 0.137 | 0.409 | 0.161 | 0.170 |
| 17 kd (Ara h 2) | 0.451 | 0.565 | 0.235 | 0.381 |
| Soy | 0.053 | 0.055 | 0.055 | 0.015 |
| Lima beans | 0.033 | 0.026 | 0.029 | 0.025 |
| Ovalbumin | 0.028 | 0.029 | 0.029 | 0.035 |
| Normal serum | | | | |
| Crude peanut | 0.017 | 0.027 | 0.028 | 0.024 |
| 17 kd (Ara h 2) | 0.024 | 0.031 | 0.038 | 0.033 |

Pooled serum is from patients with positive responses to peanut challenge.
Values are expressed as optical density units.

### Site Specificity of Four Monoclonal Antibodies

An inhibition of ELISA was used to determine the site specificity of the four monoclonal antibodies to Ara h 2 (Table 20). As determined by ELISA inhibition analysis, there are at least two different epitopes on Ara h 2, which could be recognized by the various monoclonal antibodies (e.g., epitope 1 recognized by mAb 4996C3 and epitope 2 recognized by mAbs 4996D6, 5048B3, and 4996D5).

Seven different monoclonal antibodies generated to Ara h 1, a 63.5 kd peanut allergen (see Example 2), were used to inhibit the binding of the four Ara h 2 monoclonal antibodies to the Ara h 2 protein. None of the Ara h 1 monoclonal antibodies inhibited any binding of the Ara h 2 monoclonal antibodies.

TABLE 20
ELISA inhibition for four monoclonal antibodies to Ara h 2

| | Inhibiting antibody (%) | | | | |
|---|---|---|---|---|---|
| Biotinylated mAb | 4996C3 | 4996D6 | 5048B3 | 4996D5 | Alt 1 |
| 4996C3 | 99 | 8 | 6 | 3 | 1 |
| 4996D6 | 0 | 53 | 31 | 18 | 9 |
| 5048B3 | 30 | 83 | 100 | 100 | 3 |
| 4996D5 | 1 | 44 | 56 | 64 | 8 |

Site specificity of four Ara h 2 monoclonal antibodies as determined by ELISA inhibition analysis. Values are expressed as percent inhibition.

### Site Specificity of Peanut-Specific Human IgE

Results of inhibition assays with monoclonal antibodies to inhibit IgE binding from the IgE pool (from patients with peanut hypersensitivity) to Ara h 2 are shown on Table 21.

TABLE 21
Individual anti-peanut-specific IgE binding to Ara h 2

Serum dilution

| | 1:320 | 1:100 | 1:80 | 1:40 | 1:20 | 1:5 |
|---|---|---|---|---|---|---|
| 4996D6 | 0 | 0 | 0 | 0 | 3 | 5 |
| 4996C3 | 14 | 10 | 10 | 12 | 10 | 24 |
| 5048B3 | 0 | 5 | 5 | 5 | 7 | 11 |
| 4996D5 | 0 | 10 | 10 | 22 | 23 | 25 |

Site specificity of four Ara h 2 monoclonal antibodies inhibiting anti-peanut- specific IgE (serum pool from patients with peanut hypersensitivity) binding to Ara h 2. Values are expressed as percent of anti-peanut-specific IgE binding to Ara h 2 without inhibiting monoclonal antibody.

Monoclonal antibodies 4996C3 and 4996D5 inhibited the peanut-specific IgE up to approximately 25%. Monoclonal antibodies 4996D6 and 5048B3 did not inhibit peanut-specific IgE binding. These two inhibition sites correspond to the two different IgG epitopes recognized by the monoclonal antibodies in the inhibition experiments.

### 9.4 Conclusion

In this study four monoclonal antibodies to Ara h 2 were extensively characterized. All four monoclonal antibodies produced to Ara h 2. when used as capture antibodies in an ELISA presented antigens that bound IgE from patients with positive challenge responses to peanut. No significant differences were detected in the binding of IgE from any one patient to the allergen presented by the individual monoclonal antibodies. In separate ELISA experiments, the four monoclonal antibodies generated to Ara h 2 did not bind to other legume allergens and did not bind to one variety of peanuts preferentially.

To determine the epitope site specificity of these monoclonal antibodies, inhibition ELISAs were done. At least two different and distinct IgG epitopes could be identified in experiments with the allergen, Ara h 2. In related experiments done with pooled serum from patients with positive DBPCFC responses to peanut, two similar IgE epitopes were identified.

### Example 10

### Cloning and Sequencing of Ara h 2

### 10.1 Introduction

Using N-terminal amino acid sequence data from purified Ara h 2, oligonucleotide primers were synthesized and used to identify a clone from a peanut cDNA library. This clone was capable of encoding a 17.5 kd protein with homology to the conglutin family of seed storage proteins.

### 10.2 Methods

### Serum IgE

Serum from 15 patients with documented peanut hypersensitivity (mean age, 25 years) was used to identify peanut allergens. Each of these individuals had a positive immediate skin prick test to peanut and either a positive double-blind, placebo-controlled, food challenge or a convincing history of peanut anaphylaxis (laryngeal edema, severe wheezing, and/or hypotension). Details of the challenge procedure and interpretation have been discussed previously (see Example 1). Representative individuals with elevated serum IgE levels (who did not have peanut specific IgE or peanut hypersensitivity) were used as controls in these studies. At least 5 ml of venous blood was drawn from each patient and allowed to clot, and the serum was collected. All studies were approved by the Human Use Advisory Committee at the University of Arkansas for Medical Sciences.

### Isolation and Amino Acid Sequence Analysis of Peanut Allergen Ara h 2

Ara h 2 was purified to near homogeneity from whole peanut extracts according to the methods described in Example 8. Purified Ara h 2 was electrophoresed on 12.5% acrylamide mini-gels (Bio-Rad Laboratories, Hercules, Calif.) in Tris/SDS/glycine buffer. The gels were stained with 0.1% Coomassie blue in 10% acetic acid and 50% methanol and destained in 40% methanol for 3 h with continuous shaking. Gel slices containing Ara h 2 were sent to the W. M. Keck Foundation (Biotechnology Resource Laboratory, Yale University, New Haven, Conn.) for amino acid sequencing. Amino acid sequencing of intact Ara h 2 and tryptic peptides of this protein was performed on an Applied Biosystems sequencer with an on-line HPLC column that was eluted with increasing concentrations of acetonitrile.

### Peanut RNA Isolation and Northern (RNA) Gels

Three commercial lots from the 1979 crop of medium grade peanut species, Arachis hypogaea (Florunner), were obtained from North Carolina State University for this study. Total RNA was isolated from 1 g of this material according to procedures described by Larsen (Larsen et al., 1992, supra). Poly(A)⁺ RNA was isolated using a purification kit (Collaborative Research, Bedford, Mass.) according to manufacturer's instructions. Poly(A)⁺ RNA was subjected to electrophoresis in 1.2% formaldehyde agarose gels, transferred to nitrocellulose, and hybridized with ³²P-labeled probes according to the methods of Bannon et al. (Bannon et al., 1983, supra).

### Computer Analysis of Ara h 2 Sequence

Sequence analysis of the Ara h 2 gene was done on the University of Arkansas for Medical Science's Vax computer using the Wisconsin DNA analysis software package. The algorithm of Needleman and Wunsch was used to align the complete amino acid sequence of Ara h 2 with homologous proteins before determining the percent identity.

cDNA Expression Library Construction and Screening

Peanut poly(A)⁺ RNA was used to synthesize double-stranded cDNA according to the methods of Watson and Jackson (Watson and Jackson, 1985, supra) and Huynh et al. (Huynh et al., 1985, supra). The cDNA was treated with EcoRI methylase and then ligated with EcoRI and XhoI linkers. The DNA was then ligated with EcoRI-XhoI cut, phosphatase treated X-ZAP XR phage arms (Stratagene, LaJolla, Calif.), and in vitro packaged. The library was 95% recombinants carrying insert sizes >400 bp. The library was screened using an IgE antibody pool consisting of an equal volume of serum from each patient with peanut hypersensitivity. Detection of primary antibody was with 125I-labeled anti-IgE antibody performed according to the manufacturer's instructions (Sanofi, Chaska, Minn.).

PCR Amplification of the Ara h 2 mRNA Sequence

Using the oligonucleotide CA (AG) CA (AG) TGGGA (AG) TT (AG) CA (AG) GG (N) GA (TC) AG (SEQ ID NO. 59) derived from amino acid sequence analysis of the Ara h 2 peanut allergen as one primer and a 23-nt primer which hybridizes to the pBluescript vector, the cDNA that encodes Ara h 2 was amplified from the IgE-positive clones. Reactions were carried out in a buffer containing 3 mM MgCl₂, 500 mM KCl, and 100 mM Tris-HCl, pH 9.0. Each cycle of the polymerase chain reaction consisted of 30 s at 95° C. followed by 1 minute at 56° C., and 2 minute at 72° C. Thirty cycles were performed with both primers present in all cycles. From this reaction, a clone carrying an approximately 700 bp insert was identified.

### DNA Sequencing and Analysis

DNA sequencing was done according to the methods of Sanger et al. (Sanger et al., 1977, supra) using either ³²P-end labeled oligonucleotide primers or an automated ABI model 377 DNA sequencer using fluorescent tagged nucleotides. Most areas of the clone were sequenced at least twice and in some cases in both directions to ensure an accurate nucleotide sequence for the Ara h 2 gene.

### 10.3 Results

### Isolation and Partial Amino Acid Sequence Determination of the Ara h 2 Protein

The amino terminus of the purified Ara h 2 protein, or peptides resulting from trypsin digestion of this protein, were used for amino acid sequence determination. The amino acid sequences representing the amino terminus of the Ara h 2 protein (peptide I, SEQ ID NO. 60) and a tryptic peptide fragment (peptide II, SEQ ID NO. 61) are noted in Table 22.

TABLE 22
Amino acid sequence of
Ara h 2 peptides

| | SEQ | |
|---|---|---|
| | ID | Amino acid |
| Peptide | NO. | Sequence |
| I | 60 | QQWELQGDRRRQSQLER |
| II | 61 | ANLRPCEQHLMQK |

The amino acid sequence of the amino terminus (peptide I) and a tryptic peptide (peptide II) derived from Ara h 2 protein was determined The sequence is shown as the one-letter amino acid code.

It was possible to determine the first 17 residues from peptide I (SEQ ID NO. 60) and the first 13 residues from peptide II (SEQ ID NO. 61) of the major peptide in each fraction. These results confirm and extend the previous amino acid sequence analysis of the Ara h 2 protein made in Example 8 (see Table 16).

Identification and Characterization of Clones that Encode Peanut Allergen Ara h 2

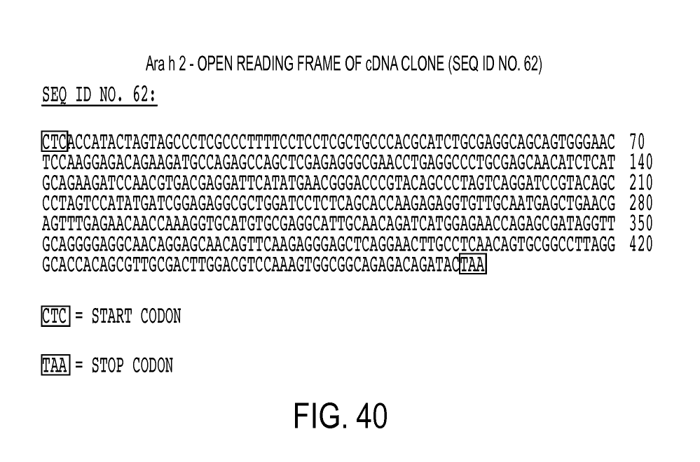

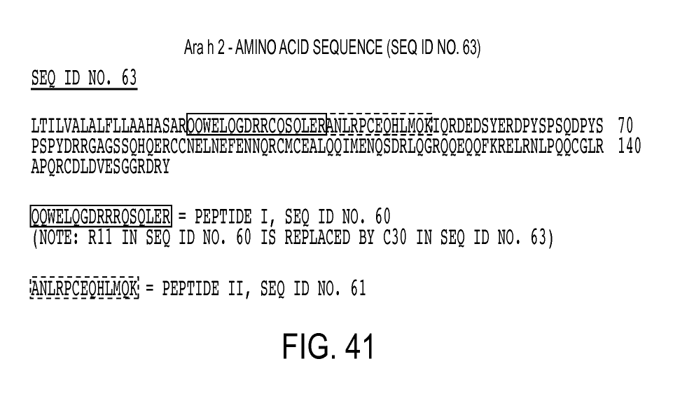

RNA isolated from the Florunner variety of peanuts (Arachis hypogaea) was used to construct an expression library for screening with serum IgE from patients with peanut hypersensitivity. Numerous IgE binding clones were isolated from this library after screening 10⁶ clones with serum IgE from a pool of patients with reactivity to most peanut allergens by Western blot analysis. Since the number of plaques reacting with serum IgE was too large to study all in detail, we randomly selected 63 positive clones for further analysis. The inserts from each of these clones were then amplified using vector-specific primers and PCR, separated by agarose gel electrophoresis, and blotted onto nitrocellulose. To identify which of the clones encoded the Ara h 2 allergen, a hybridization probe was constructed using a radioactive oligonucleotide CA (AG) CA (AG) TGGGA (AG) TT (AG) CA (AG) GG (N) GA (TC) AG (SEQ ID NO. 59) developed from amino acid sequence determined for peptide I (SEQ ID NO. 60) and used to probe the amplified inserts. Utilizing this approach, two plaques with ˜700 bp inserts were identified. DNA sequence revealed that the selected clones carried identical 741 base inserts which included a 21 base poly(A) tail and a 240 base 3′ non-coding region. This insert contained a large open reading frame starting with an CTC codon and ending with a TAA stop codon at nucleotide position 474 (FIG. 40, SEQ ID NO. 62). The open reading frame codes for a 157 amino acid protein (FIG. 41, SEQ ID NO. 63) with a molecular weight of ˜17.5 kd, which is in good agreement with the molecular weight of Ara h 2 that has been determined experimentally (see Example 8). With the exception of a single cysteine residue at position 30 of SEQ ID NO. 63, the amino acid sequences that were determined from the purified Ara h 2 protein (i.e., peptides I and II, Table 22) were found in this clone (FIG. 41). The additional coding region on the amino terminal end (encoding amino acid residues 1-19 of SEQ ID NO. 63) of this clone probably represents a signal peptide which is cleaved from the mature Ara h 2 allergen.

To determine what size mRNA this clone identified, a ³²P-labeled insert was used a hybridization probe of a Northern blot containing peanut poly(A)⁺ RNA (data not shown). This insert hybridized to an ˜0.7 kb mRNA. The size of the cloned insert and the size of the mRNA were in good agreement. In addition, there was good agreement between the calculated and determined size of the Ara h 2 protein. Furthermore, the identity of the determined amino acid sequence from the Ara h 2 peptides agreed with that which was determined from the clone. From these data we concluded that an Ara h 2 specific clone has been isolated.

### Peanut Allergen Ara h 2 is a Conglutin-Like Seed Storage Protein

A search of the GenBank, Swiss-Prot, and EMBL databases revealed significant amino acid sequence homology between the Ara h 2 protein and seed storage proteins from a variety of different plants (Table 23).

TABLE 23
Ara h 2 sequence similarities

| Protein | Source | Similarity (%) |
|---|---|---|
| Conglutin-δ | Lupin | 39 |
| Mabinlin I (chain B) | Caper | 32-35 |
| 2S albumin | Sunflower | 34 |
| 2S albumin | Castor bean | 30 |
| α-Amylase inhibitor | Wheat | 29 |
| CM3 protein | Wheat | 27 |

The Ara h 2 nucleotide sequence (SEQ ID NO. 62) and derived amino acid sequence (SEQ ID NO. 63) were used to search the GenBank, Swiss-Prot, and EMBL databases for any homologous proteins. The table lists the proteins that had the highest similarity to the Ara h 2 sequence, the plant source of those proteins, and the percentage similarity between that protein and Ara h 2.

The highest percent identity (40%) was observed between the Ara h 2 protein and conglutin-δ, a sulfur-rich protein from the lupin seed (Gayler et al., Plant Mol. Biol. 15:879-893, 1990). 2S albumins and mabinlins also had a high degree of homology (30-35%) with the Ara h 2 protein sequence (Nirasawa et al., Eur. J. Biochem. 223:989-995, 1994). Interestingly, the Ara h 2 protein had some similarity (26-29%) with α-amylase inhibitors from wheat (Garcia-Maroto et al., Plant Mol. Biol. 14:845-853, 1990 and Joudrier et al., DNA Seq. 5:153-162, 1995), which are the major allergens in baker's asthma (Sanchez-Monge et al., Biochem. J. 281:401-405, 1992 and Armentia et al., Clin. Exp. Allergy 23:410-415, 1993) and are important allergens in patients experiencing hypersensitivity reactions following the ingestion of wheat protein (James et al., J. Allergy Clin. Immunol. 99:239-244, 1996).

### 10.4 Conclusion

The Ara h 2 nucleotide sequence identified in this report has sequence homology with another class of seed storage proteins called conglutins (Gayler et al., 1990, supra). It is interesting to note that two of the major peanut allergens thus far identified are seed storage proteins that have sequence homology with proteins in other plants. This may explain the cross-reacting antibodies to other legumes that are found in the sera of patients that manifest clinical symptoms to only one member of the legume family (Bernhisel-Broadbent et al., J. Allergy Clin. Immunol. 84:701-709, 1989).

### Example 11

### Mapping and Mutational Analysis of the Linear IgE Epitopes of Ara h 2

### 11.1 Introduction

The major linear IgE-binding epitopes of this allergen were mapped using overlapping peptides synthesized on an activated cellulose membrane and pooled serum IgE from 15 peanut-sensitive patients. Ten IgE-binding epitopes were identified, distributed throughout the length of the Ara h 2 protein. Sixty-three percent of the amino acids represented in the epitopes were either polar uncharged or apolar residues. In an effort to determine which, if any, of the 10 epitopes were recognized by the majority of patients with peanut hypersensitivity, each set of 10 peptides was probed individually with serum IgE from 10 different patients. All of the patient sera tested recognized multiple epitopes. Three epitopes (amino acids 27-36, amino acids 57-66, and amino acids 65-74 of SEQ ID NO. 63) were recognized by all patients tested. In addition, these three peptides bound more IgE than all the other epitopes combined, indicating that they are the immunodominant epitopes of the Ara h 2 protein. Mutational analysis of the Ara h 2 epitopes indicate that single amino acid changes result in loss of IgE binding. Two epitopes in region amino acids 57-74 of SEQ ID NO. 63 contained the amino acid sequence DPYSPS (SEQ ID NO. 56) that appears to be necessary for IgE binding.

### 11.2 Methods

### Peptide Synthesis

Individual peptides were synthesized on a derivatised cellulose membrane using Fmoc amino acid active esters according to the manufacturer's instructions (Genosys Biotechnologies, Woodlands, Tex.). Fmoc-amino acid derivatives were dissolved in 1-methyl-2-pyrrolidone and loaded on marked spots on the membrane. Coupling reactions were followed by acetylation with a solution of 4% (v/v) acetic anhydride in N,N-dimethylformamide (DMF). After acetylation, Fmoc groups were removed by incubation of the membrane in 20% (v/v) piperidine in DMF. The membrane was then stained with bromophenol blue to identify the location of the free amino groups. Cycles of coupling, blocking, and deprotection were repeated until the peptides of the desired length were synthesized. After addition of the last amino acid in the peptide, the amino acid side chains were deprotected using a solution of dichloromethane/trifluoroacetic acid/triisobutlysilane (1/1/0.05). Membranes were either probed immediately or stored at −20° C. until needed.

### IgE Binding Assay

Cellulose membranes containing synthesized peptides were washed with Tris-buffered saline (TBS) and then incubated with blocking solution overnight at room temperature. After blocking, the membranes were incubated with serum from patients with peanut hypersensitivity diluted (1:5) in a solution containing TBS and 1% bovine serum albumin for at least 12 h at 4° C. or 2 h at room temperature. Primary antibody was detected with ¹²⁵I-labeled anti-IgE antibody (Sanofi).

### 11.3 Results

### Multiple IgE Binding Epitopes on the Ara h 2 Protein

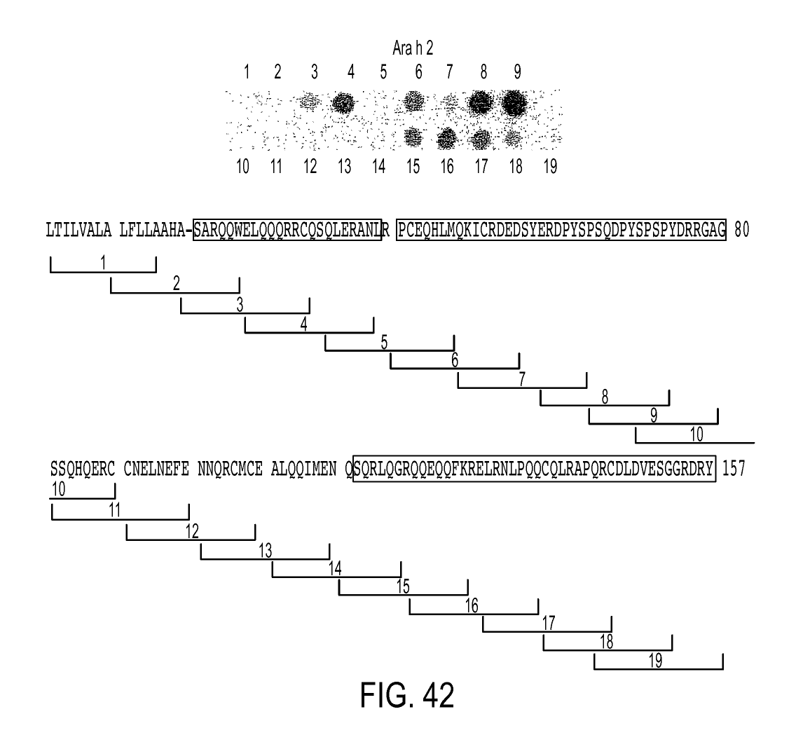

Nineteen overlapping peptides representing the derived amino acid sequence of the Ara h 2 protein were synthesized to determine which regions were recognized by serum IgE. Each peptide was 15 amino acids long and was offset from the previous peptide by 8 amino acids. In this manner, the entire length of the Ara h 2 protein could be studied in large overlapping fragments. These peptides were then probed with a pool of serum from 15 patients with documented peanut hypersensitivity or serum from a representative control patient with no peanut hypersensitivity. Serum IgE from the control patient did not recognize any of the synthesized peptides (data not shown). In contrast, FIG. 42 shows that there are three IgE binding regions along the entire length of the Ara h 2 protein that are recognized by this population of patients with peanut hypersensitivity. These IgE-binding regions represent amino acid residues 17-39, 41-80, and 114-157 of SEQ ID NO. 63.

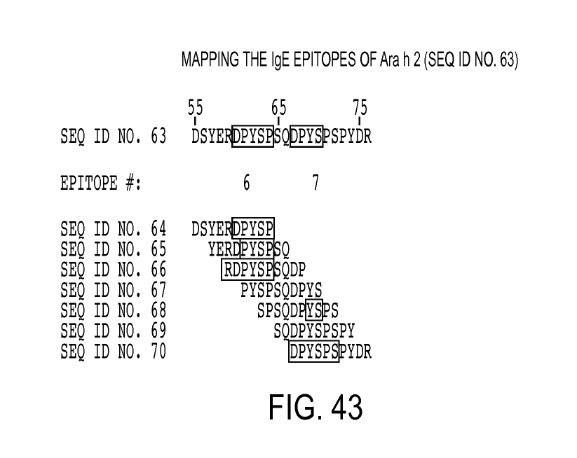

In order to determine the exact amino acid sequence of the IgE binding regions, small peptides (10 amino acids long offset by two amino acids) representing the larger IgE-binding regions were synthesized. In this manner it was possible to identify individual IgE-binding epitopes within the larger IgE-binding regions of the Ara h 2 molecule (FIG. 43). The 10 IgE-binding epitopes that were identified in this manner are shown in Table 24. The size of the epitopes ranged from 6 to 10 amino acids in length.

TABLE 24
Ara h 2 IgE binding epitopes

| SEQ | | | |
|---|---|---|---|
| ID | | Amino acid | Ara h 2 |
| NO. | Peptide | sequence¹ | positions² |
| 71 | 1 | HASARQQWEL | 15-24 |
| 72 | 2 | QWELQGDRRC | 21-30 |
| 73 | 3 | DRRCQSQLER | 27-36 |
| 74 | 4 | LRPCEQHLMQ | 39-48 |
| 75 | 5 | KIQRDEDSYE | 49-58 |
| 76 | 6 | YERDPYSPSQ | 57-66 |
| 77 | 7 | SQDPYSPSPY | 65-74 |
| 78 | 8 | DRLQGRQQEQ | 115-124 |
| 79 | 9 | KRELRNLPQQ | 127-136 |
| 80 | 10 | QRCDLDVESG | 143-152 |

¹The underlined portions of each peptide are the smallest IgE-binding sequences as determined by the analysis described in FIG. 43.
²The Ara h 2 amino acid positions are taken from SEQ ID NO. 63.

Three epitopes (amino acids 15-24, amino acids 21-30, and amino acids 27-36 of SEQ ID NO. 63), which partially overlapped with each other, were found in the region of amino acid residues 17-39 of SEQ ID NO. 63. Four epitopes (amino acids 39-48, amino acids 49-58, amino acids 57-66, and amino acids 65-74 of SEQ ID NO. 63) were found in the region represented by amino acid residues 41-80 of SEQ ID NO. 63. Finally, three epitopes (amino acids 115-124, amino acids 127-136, and amino acids 143-152 of SEQ ID NO. 63) were found in the region represented by amino acid residues 114-157 of SEQ ID NO. 63. Sixty-three percent of the amino acids represented in the epitopes were either polar uncharged or apolar residues. There was no obvious amino acid sequence motif that was shared by all the epitopes, with the exception of epitopes 6 and 7, which contained the sequence DPYSPS (SEQ ID NO. 56).

### Identification of the Immunodominant Ara h 2 Epitopes

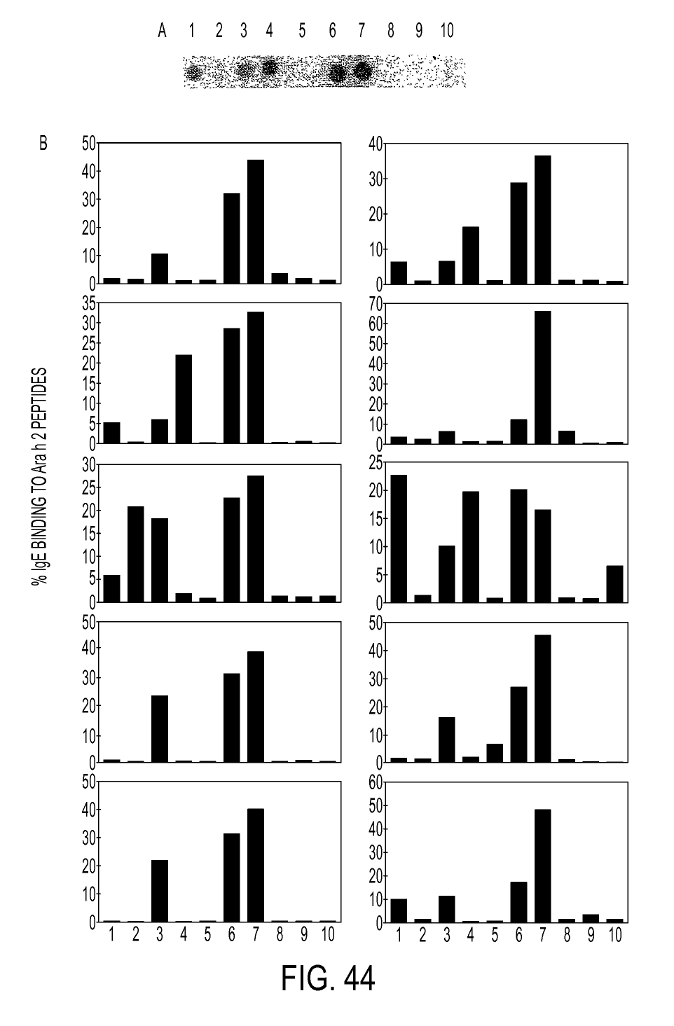

In an effort to determine which, if any, of the 10 epitopes was immunodominant, each set of 10 peptides was probed individually with serum IgE from 10 different patients. Five patients were randomly selected from the pool of 15 patients used to identify the common epitopes, and 5 patients were selected from outside this pool. FIG. 44A shows an immunoblot strip containing these peptides that has been incubated with an individual patient's serum. This patient's serum IgE recognized peptides 1, 3, 4, 6, and 7 of Table 24. The remaining patients serum IgE were tested in the same manner and the intensity of IgE binding to each spot was determined as a function of that patient's total IgE binding to these 10 epitopes (FIG. 44B) All of the patient sera tested (10/10) recognized multiple peptides. Peptides 3, 6 and 7 were recognized by serum IgE of all patients tested (10/10). In addition, serum IgE that recognizes these peptides represent the majority of Ara h 2 specific IgE found in these patients. These results indicate that peptides 3, 6, and 7 contain the immunodominant epitopes of the Ara h 2 protein.

### Mutational Analysis of Ara h 2 IgE Epitopes

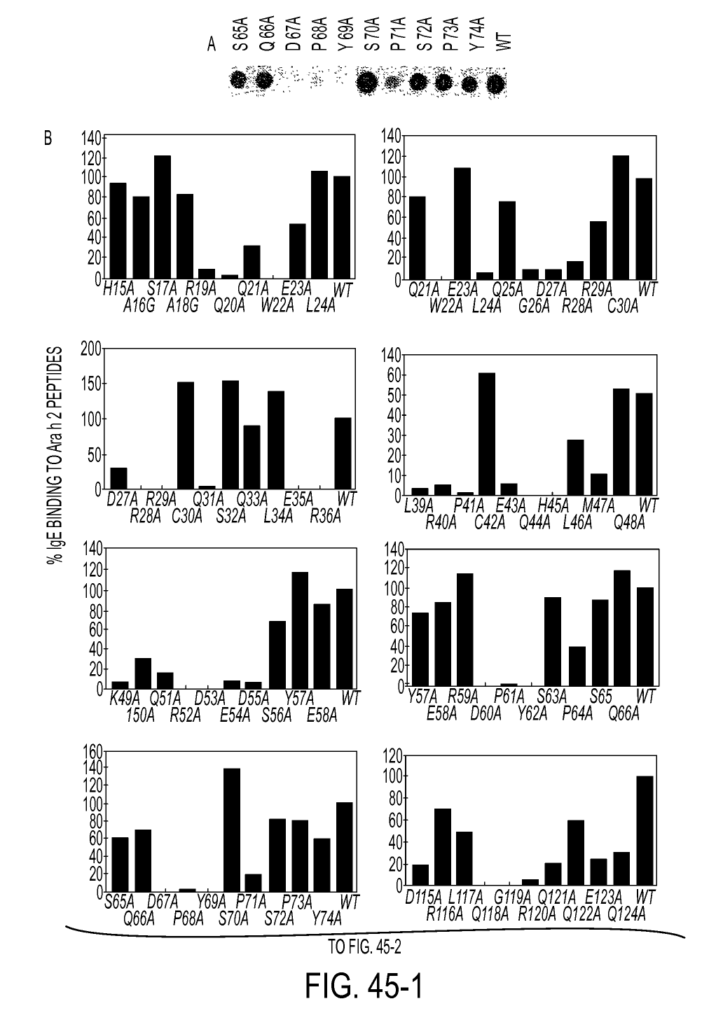

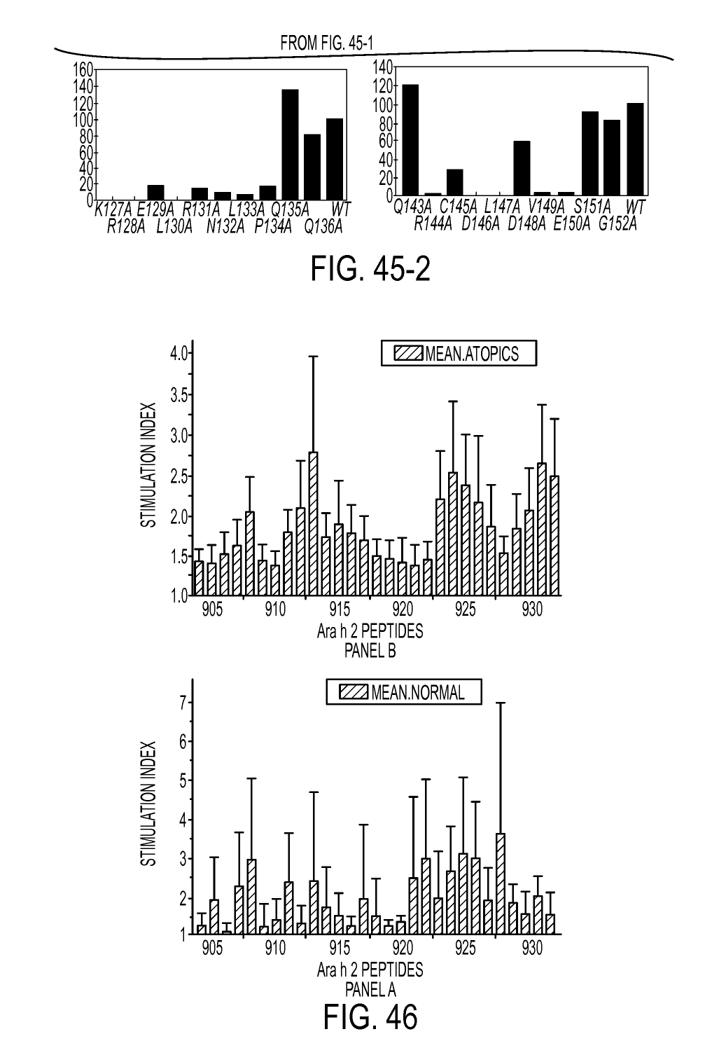

To assess the importance of individual amino acids in each of the Ara h 2 epitopes they were synthesized as 10 amino acid residue peptides with alanine residues being substituted one at a time for each of the amino acids in the epitope. These peptides were then probed with pooled serum IgE from 15 patients with documented peanut hypersensitivity. FIG. 45A shows an immunoblot strip containing the wild-type and mutated peptides of epitope 7. The pooled serum IgE did not recognize this peptide or binding was drastically reduced when alanine was substituted for amino acids at position 67, 68, or 69 of SEQ ID NO. 63. In contrast, the substitution of an alanine for serine residue at position 70 resulted in increased IgE binding. The remaining Ara h 2 epitopes were tested in the same manner and the intensity of IgE binding to each spot was determined as a percentage of IgE binding to the wild-type peptide (FIG. 45B). Table 25 summarizes these results.

In general, each epitope could be mutated to a non-IgE-binding peptide by the substitution of an alanine for a single amino acid residue. There was no obvious position within each peptide that, when mutated, would result in loss of IgE binding. Furthermore, there was no consensus in the type of amino acid that, when changed to alanine, would lead to loss of IgE binding.

TABLE 25
Amino acids critical to IgE
binding in Ara h 2

| SEQ | | Amino | |
|---|---|---|---|
| ID | | acid | Ara h 2 |
| NO. | Peptide | sequence¹ | positions² |
| 71 | 1 | HASARQQWEL | 15-24 |
| 72 | 2 | QWELQGDRRC | 21-30 |
| 73 | 3 | DRRCQSQLER | 27-36 |
| 74 | 4 | LRPCEQHLMQ | 39-48 |
| 75 | 5 | KIQRDEDSYE | 49-58 |
| 76 | 6 | YERDPYSPSQ | 57-66 |
| 77 | 7 | SQDPYSPSPY | 65-74 |
| 78 | 8 | DRLQGRQQEQ | 115-124 |
| 79 | 9 | KRELRNLPQQ | 127-136 |
| 80 | 10 | QRCDLDVESG | 143-152 |

¹The amino acids that, when altered, lead to loss of IgE binding are shown as the bold, underlined residues.
²The Ara h 2 amino acid positions are taken from SEQ ID NO. 63.

### 11.4 Conclusion

There are at least 10 IgE recognition sites distributed throughout the major peanut allergen Ara h 2. In the present study, two epitopes in Ara h 2 share a hexameric peptide (DPYSPS, SEQ ID NO. 56). It is significant to note that these peptides are recognized by serum IgE from all the peanut hypersensitive patients tested in this study. In addition, serum IgE that recognize these peptides represent the majority of Ara h 2-specific IgE found in these patients.

### Example 12

### Mapping of the Linear T-Cell Epitopes of Ara h 2

### 12.1 Introduction

We have used overlapping synthetic peptides spanning the entire protein to determine the T-cell epitopes of Ara h 2. Peanut specific T-cell lines were established from the peripheral blood of 12 atopic patients and 4 non-atopic controls. All of the cell lines were shown to consist of predominantly CD4+ T-cells. The proliferation of the T-cells in response to the 29 individual peptides was measured. Four immunodominant T-cell epitopes were identified for Ara h 2, epitope 1 (residues 18-28 of SEQ ID NO. 63), epitope 2 (residues 45-55 of SEQ ID NO. 63), epitope 3 (residues 95-108 of SEQ ID NO. 63), and epitope 4 (residues 134-144 of SEQ ID NO. 63). While T-cell epitopes 1, 2, and 4 have overlapping sequences with the linear IgE epitopes determined in Example 11, epitope 3 does not therefore providing a possibility for the development of a non-anaphylactic, T-cell directed, immunotherapeutic peptide.

12.2 Methods and results

### Identification of Ara h 2 T-Cell Epitopes

In order to determine the T-cell epitopes of peanut allergen Ara h 2, 29 different peptides representing the entire protein were synthesized. Each peptide was 20 amino acids long and was offset from the previous peptide by 5 amino acids. In this manner we were able to cover the entire protein sequence by overlapping peptides. The individual peptides were numbered 904-932 from amino terminus to carboxy terminus.

T-cells were isolated from 17 peanut allergic individuals and 5 non-peanut allergic individuals and placed into 96 well plates at 4×10⁴ cells/well and treated in triplicates with media or Ara h 2 peptides (10 μg/ml). The cells were allowed to proliferate for 6 days and then incubated with ³H-thymidine (1 μCi/well) at 37° C. for 6-8 hours and then harvested onto glass fiber filters. T-cell proliferation was estimated by quantitating the amount of ³H-thymidine incorporation into the DNA of proliferating cells. ³H-thymidine incorporation is reported as stimulation index (SI) above media treated control cells. FIG. 46 shows the mean proliferation (SI) and standard error of 17 peanut allergic individuals (upper panel) and 5 non-allergic individuals (lower panel) plotted for each of the 29 overlapping peptides that span the Ara h 2 protein.

Four immunodominant T-cell epitopes have been identified for Ara h 2 using T-cells isolated from 17 atopic individuals, namely epitope 1 shared by peptides 907-908 (residues 18-28 of SEQ ID NO. 63), epitope 2 shared by peptides 911-914 (residues 45-55 of SEQ ID NO. 63), epitope 3 shared by peptides 923-926 (residues 95-108 of SEQ ID NO. 63), and epitope 4 shared by peptides 930-932 (residues 134-144 of SEQ ID NO. 63). Similar T-cell epitopes were identified for Ara h 2 using T-cells isolated from 5 non-atopic individuals

### The CD4+ and CD8+ Profiles of the T-Cell Lines of Peanut Allergic Individuals

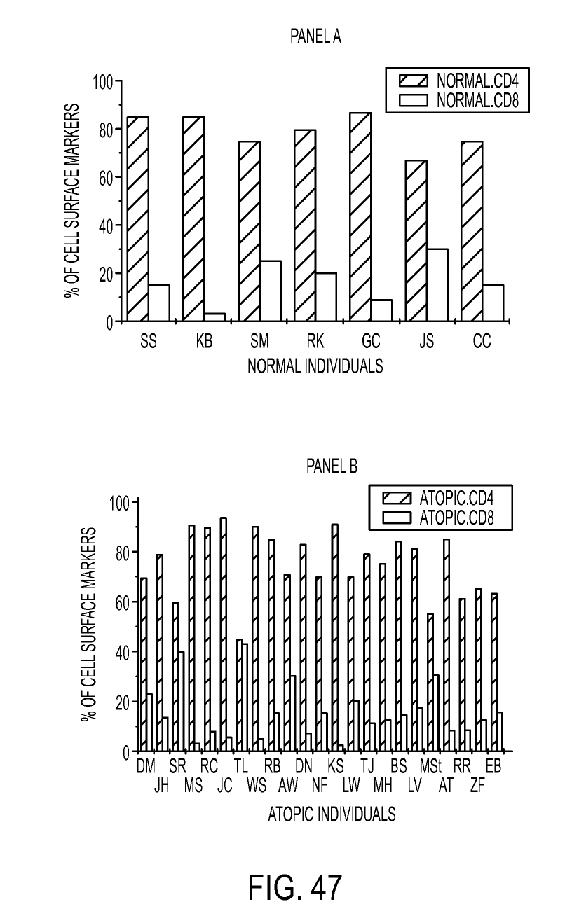

T-cells were stained with FITC-labeled anti-CD4 and FITC-labeled anti-CD8 antibodies in order to determine the phenotype of the peanut specific T-cell lines established. FACS analysis was used to determine the percent of CD4+ and CD8+ cells in the peanut specific T-cell lines utilized in Ara h 2 epitope mapping and plotted versus the initials of the individual patients used to establish these cell lines. Panel A of FIG. 47 represents the CD4+/CD8+ profiles of T-cell lines established from allergic individuals while panel B represents the CD4+/CD8+ profiles of T-cell lines established from non-allergic individuals. T-cell lines established from both atopic and non-atopic individuals were primarily CD4⁺.

### The IL-4 Secretion Profiles of T-Cells Lines of Peanut Allergic Individuals

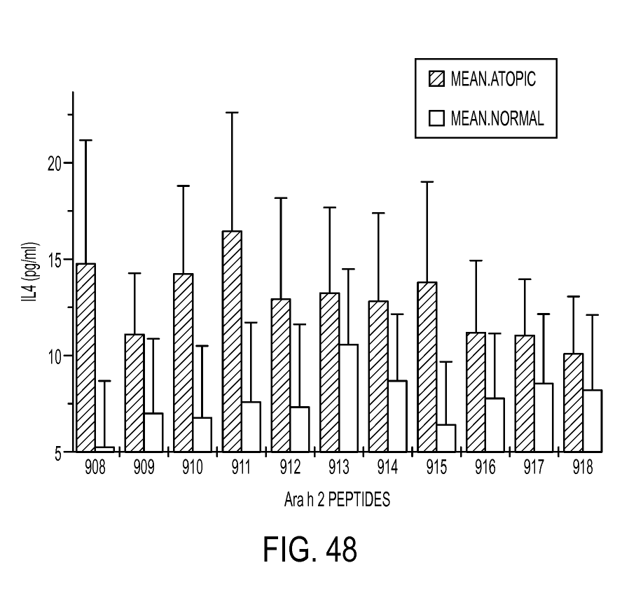

The supernatant was also collected from T-cells stimulated with immunodominant peptides and an ELISA assay was utilized to measure IL-4 concentrations in the media. In FIG. 48, IL-4 concentration is plotted versus some immunodominant peptides. T-cells from both atopic and non-atopic individuals seemed to secrete IL-4 in response to treatment with immunodominant peptides. However, T-cells of non-atopic individuals seemed to secrete more IL-4 in response to T-cell epitope 2 than T-cell epitope 1. On average T-cells of the non-atopic individuals secreted lower levels of IL-4 than the T-cells of atopic individuals.

### Comparison of the T-Cell and IgE Epitopes of Ara h 2

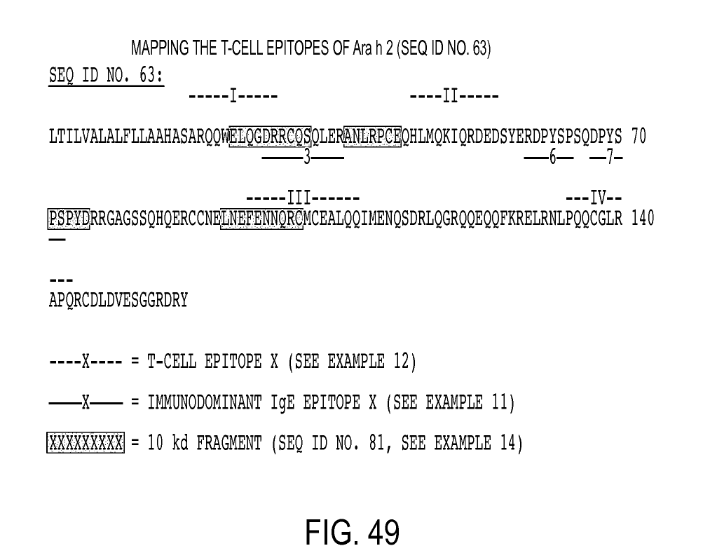

In FIG. 49, the primary amino acid sequence of the Ara h 2 protein is represented as the one letter amino acid code. The T-cell epitopes of Ara h 2 that have been identified in this study and the immunodominant IgE epitopes determined in Example 11 (Table 24) are depicted. In general, the immunodominant IgE binding epitopes do not overlap with the T-cell epitopes. This is very important in the development of peptide mediated immunotherapies towards modulating Th-2 cell development to favor Th-1 type cytokine responses.

### Example 13

### Ara h 2 Mutant Protein with Reduced IgE Binding

### 13.1 Introduction

To modulate IgE reactivity of the allergen, we constructed a variety of recombinant Ara h 2 proteins with mutations in the immunodominant IgE binding epitopes. The abilities of wild-type and mutant recombinant Ara h 2 proteins to react with IgE were tested in Western blot analysis with sera from peanut sensitive individuals. As compared to wild-type Ara h 2, the mutant Ara h 2 proteins bound less IgE, similar amounts of IgG, and exhibited a comparable ability to stimulate T-cell proliferation.

### 13.2 Results

### Expression and Purification of Recombinant Ara h 2 Proteins

Amino acids important for IgE binding in Ara h 2 were mutated to alanine by single-stranded mutagenesis (epitopes 3, 4, and 6) or by PCR (epitopes 1, 2, 5, 7, 8, 9, and 10). Mutations were confirmed by sequence analyses of recombinant Ara h 2 cDNA clones. Three different mutants MUT4, MUT5, and MUT10 were initially prepared that included mutations in a total of 4, 5, and 10 epitopes, respectively. The mutations and their locations within the Ara h 2 sequence (SEQ ID NO. 63) are listed in Table 26.

TABLE 26
Mutant Ara h 2 proteins

| Mutated epitope² | Mutation¹ | MUT4 | MUT5 | MUT10 |
|---|---|---|---|---|
| 1, 2 | Q20A | | X² | |
| 1, 2 | W22A | | | X² |
| 3 | Q31A | | X | |
| 3 | E35A | X | | X |
| 4 | P41A | X | | X |
| 5 | D53A | | | X |
| 6 | D60A | X | X | X |
| 7 | D67A | X | X | X |
| 8 | R120A | | | X |
| 9 | L130A | | | X |
| 10 | L147A | | | X |

¹The Ara h 2 amino acid positions are taken from SEQ ID NO. 63.
²Epitopes 1 and 2 overlap and are both affected by the Q20A and W22A mutations.

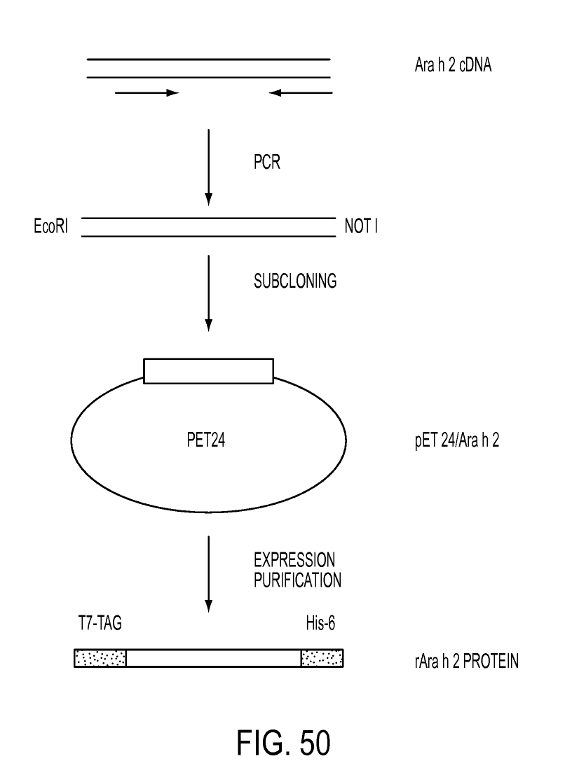

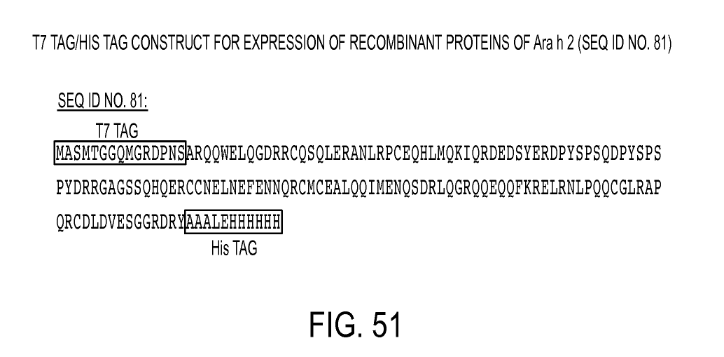

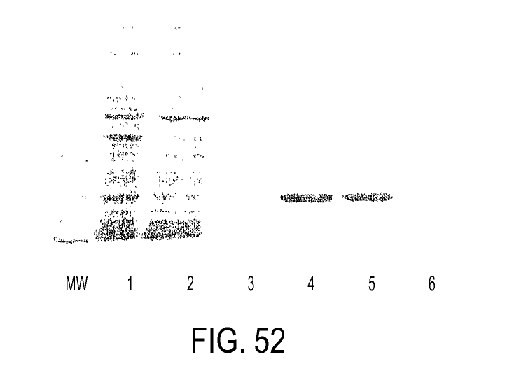

The portion of Ara h 2 sequence excluding the first 54 nucleotides, which encodes the signal peptide, was amplified by PCR. The PCR product was ligated to the EcoRI-NotI sites of pET24(a). This vector encodes a T7-tag at the N-terminus and a His-tag at the C-terminus of expressed fusion proteins (FIGS. 50 and 51). E. coli BL21(DE3) cells were transformed with the Ara h 2 constructs and exponentially growing cells were induced with 1 mM IPTG. Cells were pelleted and the recombinant Ara h 2 proteins were purified by affinity chromatography on a Ni²⁺-resin column. FIG. 52 shows SDS-PAGE of fractions, obtained during purification of recombinant Ara h 2 proteins on the Ni²⁺-column: lane 1 is the cell lysate, lane 2 is the unbound fraction, lane 3 is the 20 mM imidazole wash fraction, lanes 4-6 are the 100 mM imidazole elution fractions.

IgE Binding to MUT4 and MUT10 Vs. Wild-Type Ara h 2 Using Pooled Sera

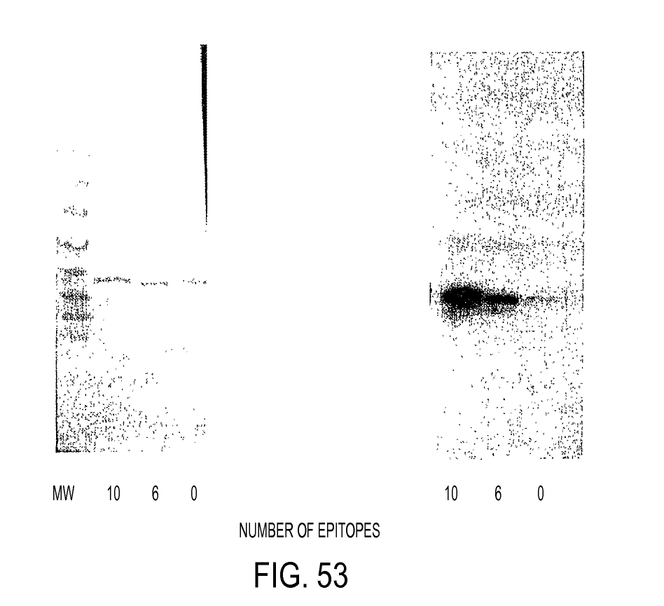

Equal amounts of purified wild-type and mutant Ara h 2 proteins (MUT4 and MUT10) were separated by gradient (4-20%) PAGE and electrophoretically transferred onto nitrocellulose paper. The blots were incubated with antibody directed against N-terminal T7-tag or pooled serum from peanut sensitive patients (FIG. 53). While binding to the T7 tag remains relatively constant, IgE binding is dramatically decreased in the mutants.

IgE Binding to MUT4 and MUT10 Vs. Wild-Type Ara h 2 Using Individual Sera

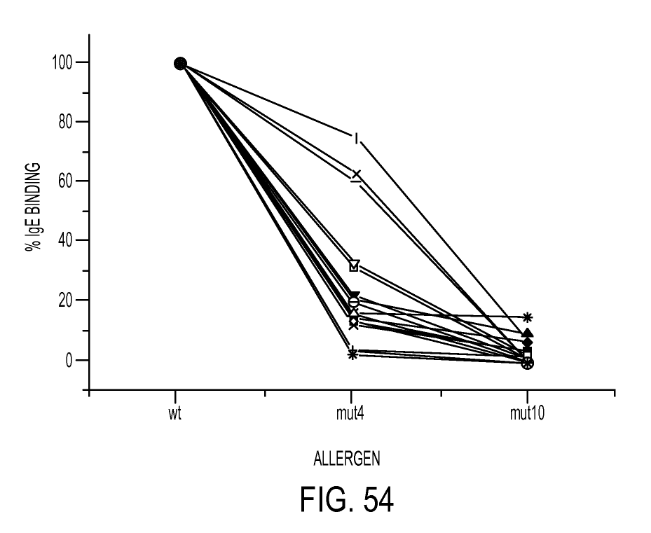

IgE binding to mutated recombinant Ara h 2 proteins as compared to the wild-type was then examined in Western blot analysis using individual patient sera (FIG. 54). Laser densitometry was used to quantitate relative IgE binding. Each line represents IgE binding for an individual patient in the group. While IgE binding to MUT10 is dramatically reduced for each individual, some differences are observed between the different individuals in the group with MUT4.

### Inhibition of IgE Binding to Native Ara h 2

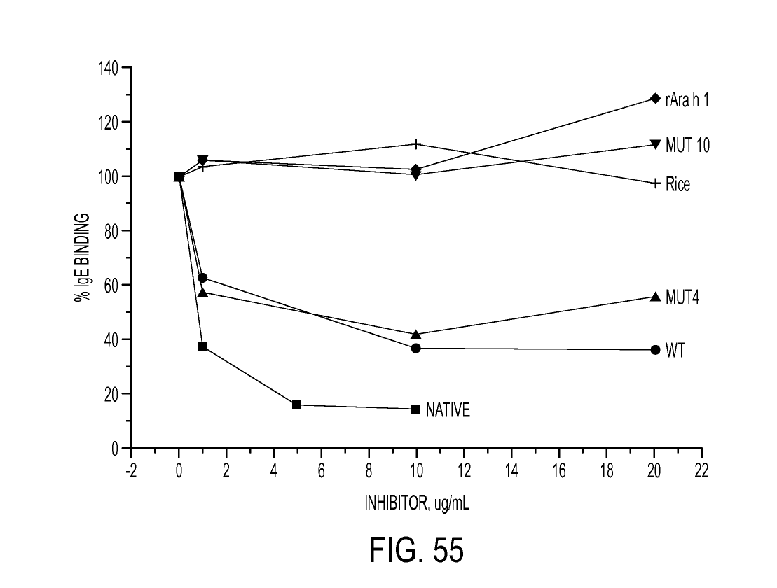

To further characterize binding of IgE to MUT4 and MUT10, an inhibition binding assay was prepared. 0.5 μg of the native Ara h 2 protein purified from crude peanut extracts was loaded onto each member of a set of nitrocellulose membranes using a slot-blot apparatus. The membranes were then incubated with pooled patient serum (1:20) in the presence or absence of different concentrations of wild-type Ara h 2, MUT4, MUT10, and as controls rice protein or recombinant wild-type Ara h 1. Membranes were probed for bound IgE with ¹²⁵I-labeled anti-human IgE antibody. Laser densitometry of the autoradiograms was used to quantitate the relative amounts of IgE binding and the results are presented in FIG. 55. While MUT10 had a negligible effect (same as control) on IgE binding to native Ara h 2, MUT4 inhibited binding at similar levels as recombinant wild-type Ara h 2.

T-Cell Proliferation in Presence of MUT4 and MUT10 vs. Wild-Type Ara h 2

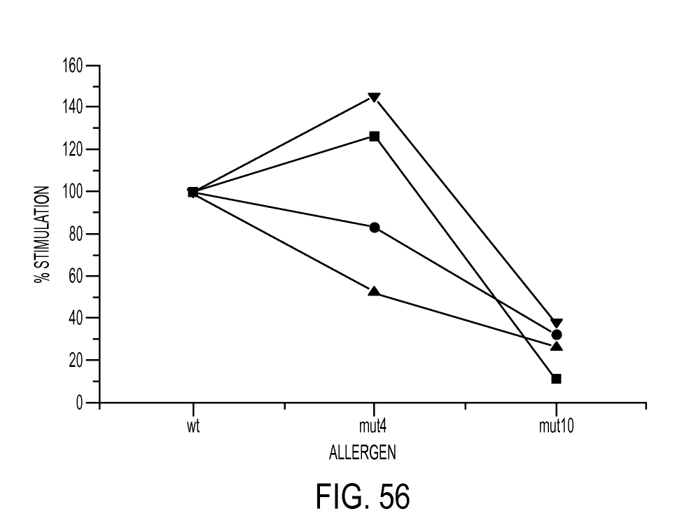

Peripheral blood mononuclear cells (PBMCs) were isolated from heparinized venous blood of peanut-sensitive patients by density gradient centrifugation on Ficoll. 2×10⁵ cells per well were incubated in triplicates for 7 days in RPMI media with 5% human AB serum in the presence of 10 μg/ml of the native Ara h 2 protein purified from the crude peanut extract or recombinant Ara h 2 proteins purified from E. coli. Cells incubated in media only were used as a control. Proliferation was measured by the incorporation of tritiated thymidine. Stimulation index (SI) is calculated as a ratio of radioactivity for the cells growing in the presence of allergen to that for the cells growing in media alone. The results are presented in FIG. 56 where each line represents the SI for PBMCs taken from an individual patient in the group. The relatively low proliferation in the presence of MUT10 suggest that T-cell epitopes may be affected by mutagenesis of overlapping IgE epitopes.

### MUT5 Binds Less IgE but Similar Amounts of IgG as Wild-Type Ara h 2

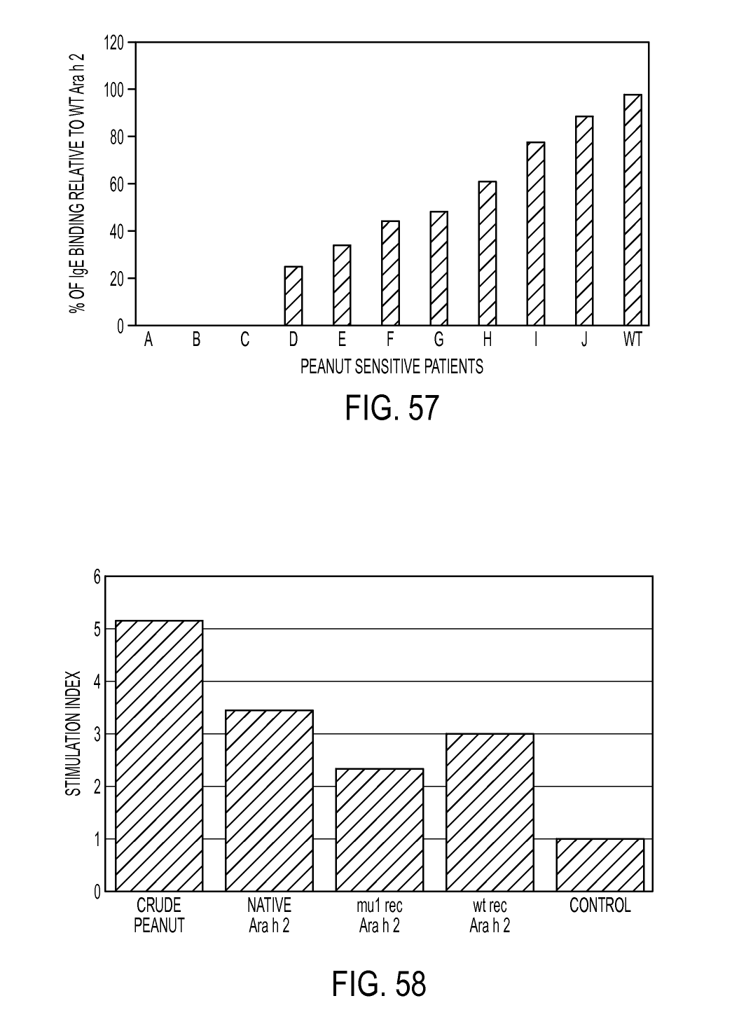

MUT5 includes mutations within IgE epitopes 1, 3, 6, and 7 that were determined to be critical to IgE binding in Example 11. MUT5 was produced and immunoblot analysis performed using serum from peanut sensitive patients. The results showed that MUT5 bound significantly less IgE than recombinant wild-type Ara h 2 (see FIG. 57) but bound similar amounts of IgG (data not shown).

### MUT5 Retains the Ability to Activate T-Cell Proliferation

MUT5 was also used in T-cell proliferation assays to determine if it retained the ability to activate T-cells from peanut sensitive individuals. Proliferation Assays were performed on T-cell lines grown in short-term culture developed from six peanut sensitive patients. T-cells lines were stimulated with either 50 μg of crude peanut extract, 10 μg of native Ara h 2, 10 μg of recombinant wild-type Ara h 2, or 10 μg of MUT5 and the amount of ³H-thyimidine incorporates was determined for each cell line. Results were expressed as the average stimulation index (SI) which reflected the fold increase in ³H-thymidine incorporation exhibited by cells challenged with allergen when compared with media treated controls (see FIG. 58).

MUT5 Elicits a Smaller Wheal and Flare in Skin Prick Tests than Wild-Type Ara h 2

MUT5 and wild-type recombinant Ara h 2 were used in a skin prick test of a peanut sensitive individual. Ten micrograms of these proteins were applied separately to the forearm of a peanut sensitive individual, the skin pricked with a sterile needle, and 10 minutes later any wheal and flare that developed was measured. The wheal and flare produced by the wild-type Ara h 2 protein (8 mm×7 mm) was approximately twice as large as that produced by MUT5 (4 mm×3 mm). A control subject (no peanut hypersensitivity) tested with the same proteins had no visible wheal and flare but, as expected, gave positive results when challenged with histamine. In addition, the test subject gave no positive results when tested with PBS alone. These results indicate that an allergen with only 50% of its IgE epitopes modified (i.e., 5/10) can give measurable reduction in reactivity in an in vivo test of a peanut sensitive patient.

### Example 14

### Effects of Enzymatic Digestion of Ara h 2

### 14.1 Introduction

Ara h 2 (17.5 kd) is a much smaller protein than Ara h 1 (63.5 kd), and does not form trimers. Instead an extensive network of intramolecular disulfide bonds stabilizes it. Upon treatment with proteases found in the digestive tract, the peptide fragments produced remain associated due to their linkage through disulfide bonds, even in the presence of denaturing detergents. These resulting peptide fragments are still relatively large and survive further proteolytic digestion for extended periods of time. Only when the disulfide linkages are reduced with dithiothreitol (DTT) do the individual fragments dissociate. These surviving peptide fragments contain the immunodominant IgE binding epitopes and numerous potential enzyme cut sites which were apparently protected from hydrolysis by the overall stable globular structure of the Ara h 2 molecule maintained by its stabilizing disulfide bonds. These results may provide a link between allergen structure and the development of immunodominant epitopes within a population of food allergic individuals.

### 14.2 Methods and Results

### Reversible Reduction of Ara h 2 Isoforms in the Presence or Absence of DTT

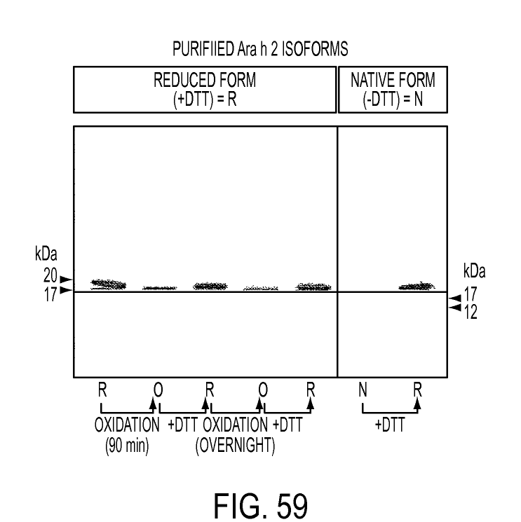

As shown in FIG. 59, molecular size shifting occurs when Ara h 2 isoforms are oxidized or reduced (by addition or removal of DTT). The two isoforms of 20 kd and 17 kd shift to 17 kd and 12 kd respectively when DTT is removed from the preparation. The shift is reversible upon addition of DTT.

Ara h 2 is More Resistant to Digestion when Oxidized than when Reduced

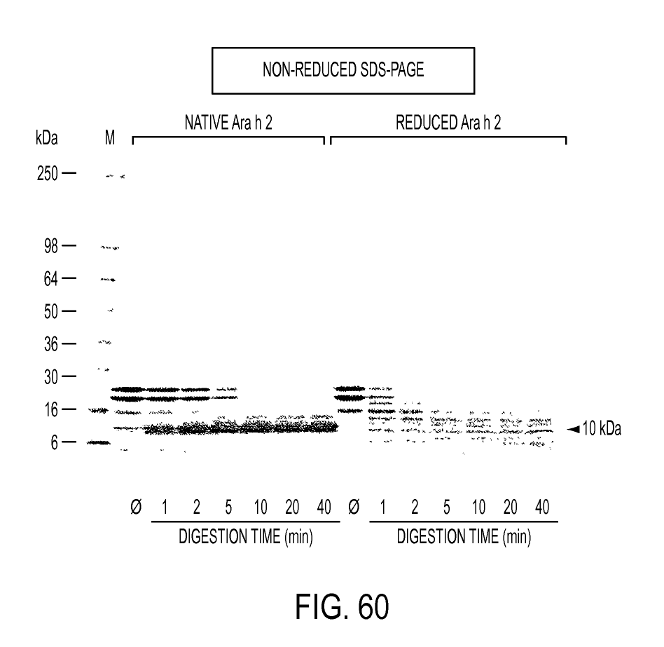

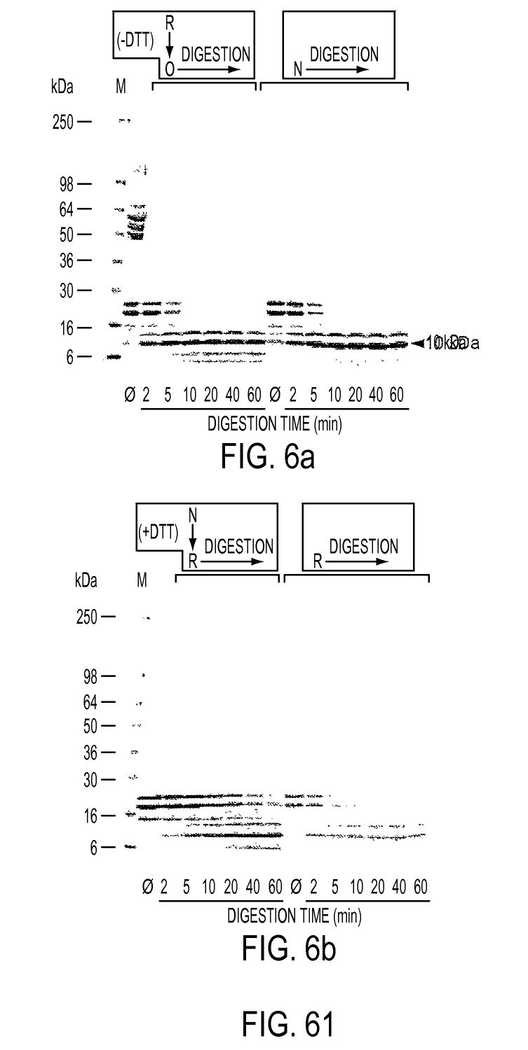

Ara h 2 was purified under two different conditions (native ‘N’ conditions, no DTT present or reduced ‘R’ conditions, DTT present) and then digested under identical conditions (i.e., time and enzyme concentration). The results are presented in FIG. 60 and indicate that native Ara h 2 is more resistant and produces a digestion resistant fragment of 10 kd when compared to the reduced form which is digested in into smaller fragments.

### Digestion of Ara h 2 in Different Oxidation States

Ara h 2 from crude peanut extracts was purified either in the presence (R=reduced) or absence (N=native) of the reducing agent DTT. When reduced protein is allowed to re-oxidize (O=oxidized) and is then digested with trypsin a resistant 10 kd peptide is formed that is identical to the digestion pattern of the native protein (FIG. 61A). When native protein is reduced and then digested with trypsin the digestive pattern is identical to that observed when reduced protein is digested (FIG. 61B). These results indicate that the disulfide bonds in the native protein aid in the stabilization of this allergen and the production of the dominant 10 kd protease resistant fragment.

### IgE Binding to Digestion Fragments of Ara h 2

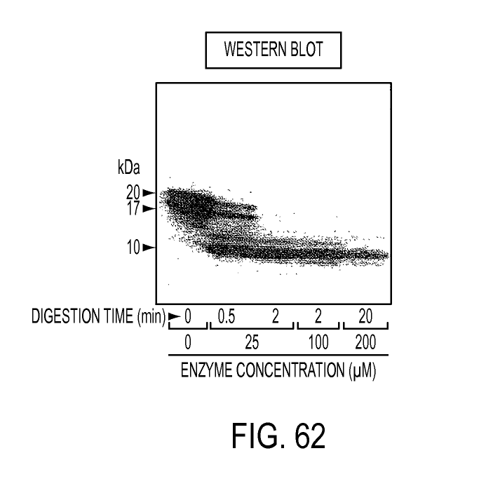

Western blots of trypsin-digested fragments of Ara h 2 are shown in FIG. 62. The 10 kd fragment is resistant to extended periods (20 minutes) of enzyme digestion at high concentrations (200 nM) of the protease. In addition, this fragment binds IgE from peanut sensitive patient sera. This fragment was purified and the amino terminus of this molecule was sequenced. The 10 kd fragment is shown as a shaded region within the Ara h 2 sequence of FIG. 49. The fragment spans a central regions of Ara h 2 (SEQ ID NO. 81, between amino acid residues 23-105 of SEQ ID NO. 63). In addition, FIG. 49 highlights the immunodominant IgE binding epitopes of Ara h 2 (epitopes 3, 6, and 7) that were identified in Example 11 and the four T-cell epitopes that were identified in Example 12. The 10 kd fragment contains all three immunodominant IgE epitopes. The epitopes are protected from digestion by the disulfide bonds although there are many potential enzyme cut sites present in the region.

### 14.3 Conclusion

Protein structure of the major peanut allergen Ara h 2 plays a significant role in its stability to protease digestion. Immunodominant IgE binding epitopes of Ara h 2 may be determined by this structure.

### Example 15

### Identification of Peanut Allergens Using Pooled IgE Sera Adsorbed to Remove Cross-Reacting Antibodies to Soybean

### 15.1 Introduction

Cross-reacting antibodies to soy were removed from the sera of two patients allergic to peanut and soy and three patients allergic to peanut by soy-affinity chromatography. Adequate removal of cross-reacting antibodies was verified by ELISA after each adsorption step. Unabsorbed sera and sera absorbed to remove cross-reacting antibodies to soy were assayed for specific IgE binding to peanut immunoblots.

Unique peanut-specific IgE antibodies (i.e., soy antibody-absorbed) were found to bind to peanut fractions at 46, 29, 25, 19, 17, 14, and 5 kd on immunoblots of whole peanut protein. The 73% reduction of IgE antibody binding to peanut by ELISA after absorption of cross-reacting antibodies indicates extensive cross-reactivity between soy and peanut antigens.

### 15.2 Methods

### Patients

Sera were obtained from five children with allergy (5 to 17 years of age: BP, BM, DH, AT, and DT) with a history of anaphylactic reactions to peanuts and with high levels of peanut-specific serum IgE antibodies (CAP-RAST FEIA; Pharmacia Diagnostics, Evansville, Ind.). Two patients (BP and BM) also were first seen with symptoms of IgE-mediated soy allergy. None of the patients were reactive to any other legume. In addition to food allergies, two patients had atopic dermatitis, asthma, and allergic rhinitis; one patient had atopic dermatitis and asthma; and one patient had asthma.

### Preparation of Soy and Peanut Extracts

Thirty grams of soy flour were incubated in 150 ml of phosphate-buffered saline (PBS) overnight on a rotating plating in a cold room at 4° C. The soy mixture was then clarified by centrifugation at 1000 g and the supernatant was removed and lyophilized. The protein content was determined by Coomassie Plus Protein Assay Reagent (Pierce Chemical Co., Rockford, Ill.) The peanut extract was obtained form three commercial lots of Florunner peanuts and processed as described in Example 1.

### Antigen-Specific Serum Antibody Absorption

Forty milligrams of soy protein was added to 10 ml of active ester agarose gel (Affi-gel 10; Bio-Rad Laboratories, Richmond, Calif.) and placed on a rocking platform for 4 hours at 4° C. One milliliter of 0.1 mol/L ethanolamine HCl (pH 8.0) was then added to the gel and mixed by rotation for 1 hour. A 1×10 cm chromatography column (Bio-Rad Laboratories) was packed with the affinity gel. Three milliliters of patient serum was added to the soy-affinity column for removal of antibodies with affinity to soy protein. After 1 to 2 hours of exposure time, the column was rinsed with 30 ml of PBS. The first 6 ml of the absorbed serum was collected and concentrated to the initial volume of 3 ml on a Centriprep 30 concentrator (Amico, Beverly, Mass.) at 1200 g for 30 minutes. The column was then rinsed with 30 ml of 0.01 mol/L sodium phosphate (pH 12.5) to elute soy-bound antibodies. The soy absorbed serum was again run over the soy-affinity column, and the rinsing and eluting were repeated until no soy-binding activity was detectable by ELISA. The complete procedure was repeated with sera from each of the five patients.

### ELISA for IgE

After each passage of sera over the soy-affinity chromatography columns, the presence of soy-specific IgE was monitored by ELISA and by CAP-RAST FEIA. Furthermore, specific IgE antibodies to Ara h 1 and Ara h 2 were determined in unabsorbed sera and sera from which soy-specific antibodies had been removed. Two rows of 96-well microtiter plates (Dynatech Laboratories, Chantilly, Va.) were filled with of a solution of soy (10 μg/ml) in coating buffer (0.1 mol/L sodium bicarbonate, pH 9.6) and incubated overnight at 4° C. Fifty microliters of sera and adsorbed sera (at 1:5 dilution) in antibody buffer (PBS+0.05% Tween [PBS-T]+2% bovine serum albumin) were incubated for 2 hours at room temperature after the plates were washed with PBS-T buffer. After incubation, plates were washed, and 50 μl of a solution of biotin-conjugated goat anti-human IgE (0.625 μg/ml; Kirkegaard & Perry Laboratories, Inc., Gaithersburg, Md.) in antibody buffer was added and; incubated for 2 hours. After washing the plates, streptavidin-peroxidase (Sigma Chemical Co., St. Louis, Mo.) in avidin buffer (PBS-T+2% bovine serum albumin+0.5% gelatin) was incubated in each well for 30 minutes, the plates were washed again and developed with Sigma FAST OPD (Sigma Chemical Co.). Optical densities were measured at 490 nm and 650 nm with an automated ELISA plate reader (Molecular Devine Corporation, Menlo Park, Calif.).

### Tricine-SDS Polyacrylamide Gels and Immunoblotting

Peanut protein extract (2 mg/ml) was mixed with an equal volume of SDS: sample buffer (50 mmol/L Tris HCl, pH 6.8), containing 4% SDS, 2% β-mercaptoethanol, 12% glycerol bromphenol blue, and pyronin Y, and boiled 10 minutes for denaturation. Separation was performed by tricine-SDS polyacrylamide gel to obtain adequate resolution of low-molecular-weight proteins, modified from the method of Schägger and von Jagow (Anal. Biochem. 166:368-379, 1987). The running gel was prepared from a stock of 49.5% wt/vol acrylamide (Sigma Chemical Co.) and 1.5% wt/vol bisacrylamide (Bio-Rad Laboratories: 49.5% T, 3% C) in 3.0 mol/L Tris (pH 8.45) with 0.3% SDS gel buffer solution and glycerol (Fisher Biotech, Fair Lawn, N.J.). A 4.5% stacking gel and 15% running gel were prepared from the stock 49.5% T. 3% C, in gel buffer solution. Both gels were polymerized with 10% ammonium persulfate and N,N,N′,N′-tetramethylenediamine. The electron tank (Hoefer Scientific Instruments, San Francisco, Calif.) was loaded with 0.1 mol/L Tris (pH 8.25), 0.1 mol/L tricine, and 0.1% SDS in the upper tank and 0.2 mol/L Tris (pH 8.9) in the lower tank. Electrophoresis was performed at 30 V through the stacking gel and at 80 V overnight through the running gel. Glycine-SDS Polyacrylamide gels (or SDS-PAGE gels) with resolution for protein fractions between 66 and 14 kd prepared according to the method of Dreyfuss et al. (Dreyfuss et al., Mol. Cell. Biol. 4:415-423, 1984) and modified as previously described (Bernhisel-Broadbent et al., 1989, supra), were performed for further immunoblotting with the unabsorbed serum and the soy-absorbed serum of one patient (BP).

The peanut proteins were electrotransferred from the polyacrylamide gel to nitrocellulose paper at 0.15 A for 6 hours in 50 mmol/L of Tris-glycine buffer (pH 9.1) containing 20% methanol. After transfer, the nitrocellulose blots were blocked overnight in PBS-T with 0.5% gelatin. Protein staining with 0.1% amido black was obtained for each gel to confirm proper electrophoresis and protein transfer on nitrocellulose paper. The blots were then incubated with non-adsorbed serum and absorbed serum (5:1 vol/vol dilution) for 2 hours at room temperature on a rocking platform. The blots were washed five times for 5 minutes with PBS-T and incubated for 2 hours with biotin-conjugated goat anti-human IgE in antibody buffer (1:1600). After five washes, the blots were incubated with streptavidin-peroxidase in avidin buffer (1:1000) for 30 minutes, washed, and developed with Sigma FAST DAB (Sigma Chemical Co.). The reaction was stopped by rinsing the blots several times in distilled water. The molecular weights of protein fractions with IgE binding were determined by scanning densitometry (Ultroscan; LKB, Broma, Sweden) and compared with molecular weight makers.

### 15.3 Results

Antigen-Specific Serum Antibody Adsorption

Soy-binding antibodies were removed form the sera of five patients allergic to peanut by soy-affinity chromatography. All five patients were initially selected because they had high levels of IgE antibodies to peanut (Table 27).

TABLE 27
Concentrations of serum IgE antibodies
to peanut and soy for each patient

Patient

| IgE antibodies | BP | BM | DH | AT | DT |
|---|---|---|---|---|---|
| Anti-peanut (IU/ml) | 2405 | 1995 | 357 | 1560 | 2225 |
| Anti-soy (IU/ml) | 75 | 110 | 8 | 15 | 88 |

Normal values are less than 0.35 IU/ml. Patients BP and BM are allergic to peanut and soy; patients DH, AT, and DT react to peanut exclusively.

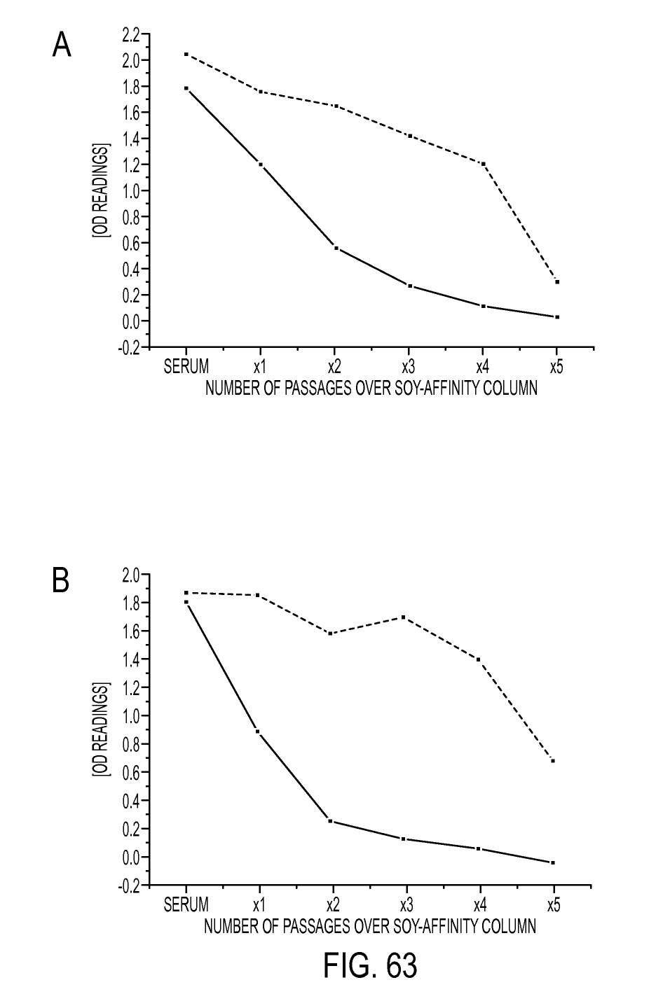

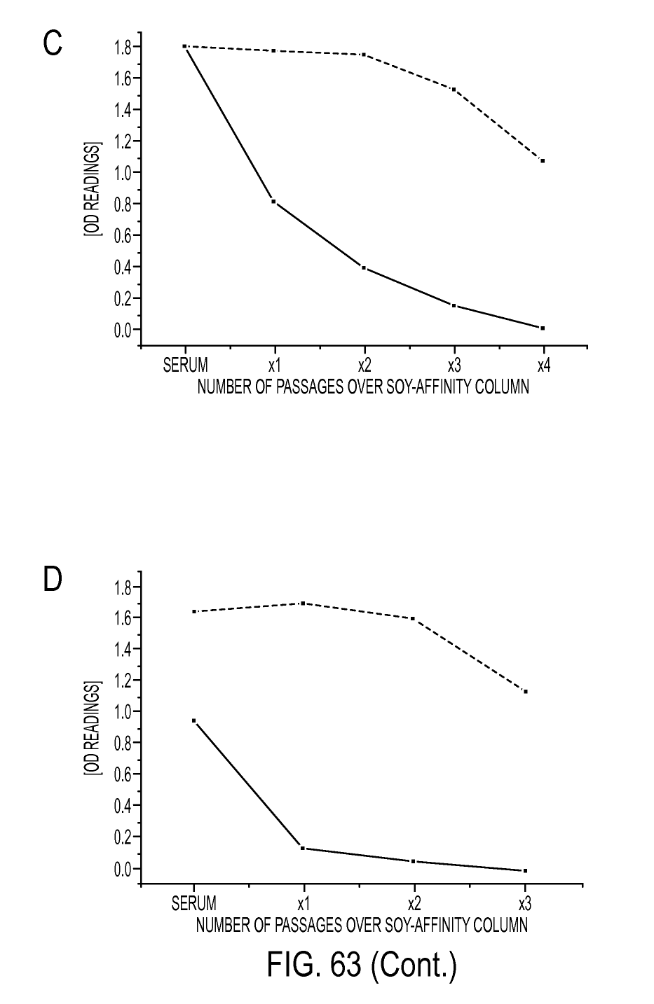

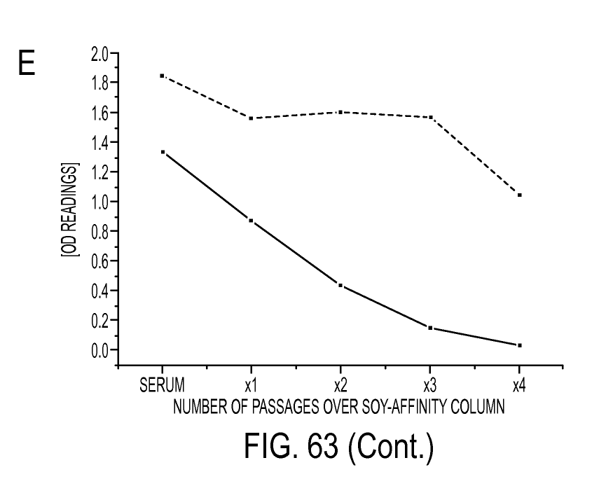

The adsorption of soy-binding IgE antibodies was monitored after each adsorption procedure of 1 hour by ELISA (FIG. 63). Repeated adsorption steps by soy-affinity chromatography progressively diminished soy-specific IgE antibody titers to background optical density readings. A total of three to five passes over the affinity column were necessary to remove all soy-specific IgE antibodies, with sera from patients allergic to soy requiring the most extensive adsorption. Progressive diminution of specific IgE binding to peanut by ELISA confirms that cross-reacting antibodies were adsorbed onto the soy-affinity column (FIG. 63). On the other hand, the serum sample run over the human serum albumin column showed no significant decrease in peanut-specific IgE antibody with about 95% recovery of the specific antibody: before adsorption, 2225 IU/ml; after adsorption, 2114 IU/ml.

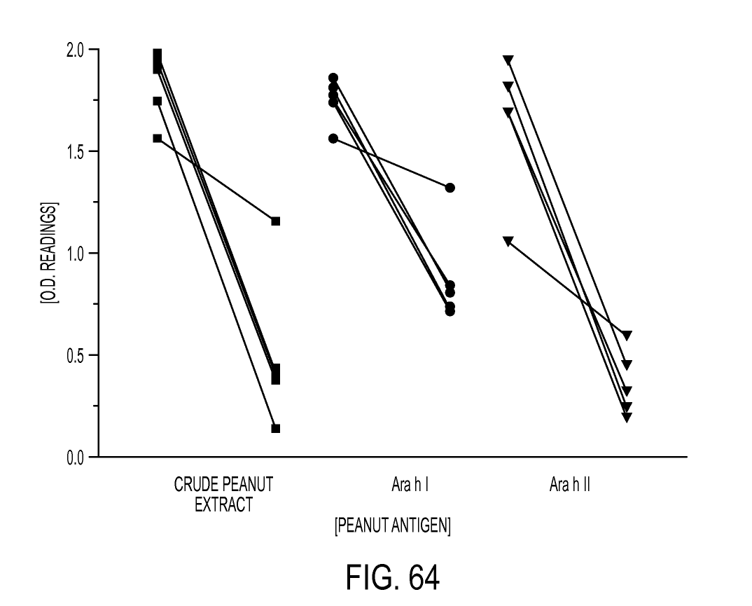

To determine the fraction of non-cross-reacting peanut-specific IgE, we compared the amount of specific IgE with crude peanut antigen, as well as with Ara h 1 and Ara h 2, before and after adsorption of cross-reacting antibodies. Because high concentrations of peanut-specific IgE in the sera might have saturated antigen-binding sites, several fivefold serial dilutions (from 1:5 to 1:625) were performed. ELISAs were then run at optimal serial dilutions to determine specific IgE binding to crude peanut antigen, Ara h 1 and Ara h 2 in non-adsorbed and soy-adsorbed sera. FIG. 64 shows that the average IgE antibody adsorption onto soy-affinity chromatography was 73% for crude peanut extract, 79% for Ara h 1, and 76% for Ara h 2. Specific IgE antibodies were less depleted in patient DH, possibly related to the low soy-specific serum IgE antibody titer (Table 28).

TABLE 28
Peanut- and soy-specific IgE antibody concentrations (IU/ml)
after successive passes over a soy-affinity column

| Patient | Non-adsorbed | Pass 1 | Pass 2 | Pass 3 | Pass 4 | Pass 5 |
|---|---|---|---|---|---|---|
| B P | | | | | | |
| Peanut | 2405 | 120 | 116 | 56 | 43 | 22 |
| Soy | 75 | 32 | 11 | 6 | 3 | 2 |
| B M | | | | | | |
| Peanut | 1995 | 244 | | 168 | 111 | 73 |
| Soy | 110 | 30 | | 6 | 4 | 2 |
| D H | | | | | | |
| Peanut | 357 | 119 | 104 | 81 | | |
| Soy | 8 | 2 | 1 | ND | | |
| A T | | | | | | |
| Peanut | 1560 | 236 | 206 | 93 | 85 | |
| Soy | 15 | 7 | 4 | 2 | 1 | |
| D T | | | | | | |
| Peanut | 2225 | 238 | 117 | 63 | 66 | |
| Soy | 88 | 11 | 6 | 5 | 1 | |

ND, Not detected

### Tricine-SDS Polyacrylamide Gels and Immunoblotting

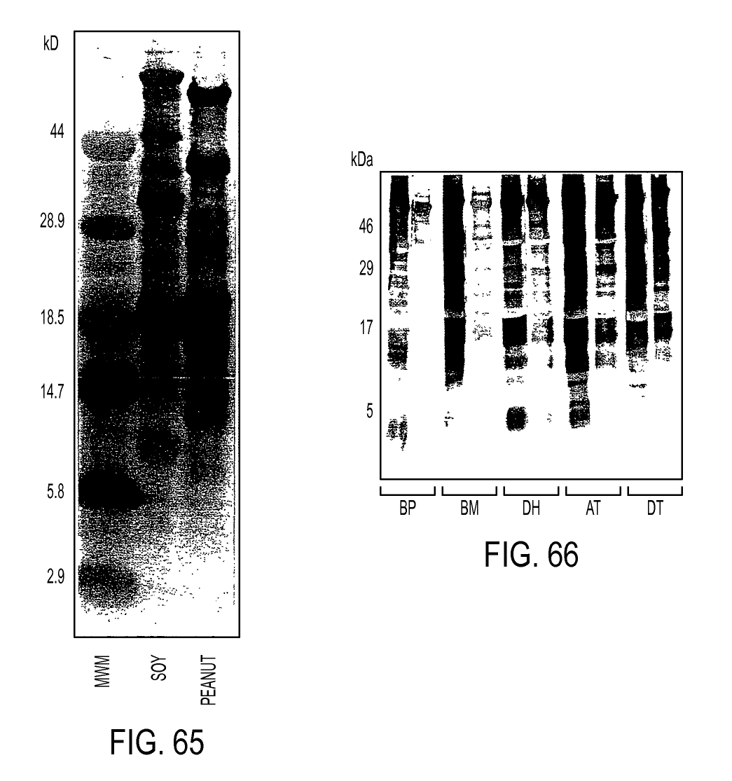

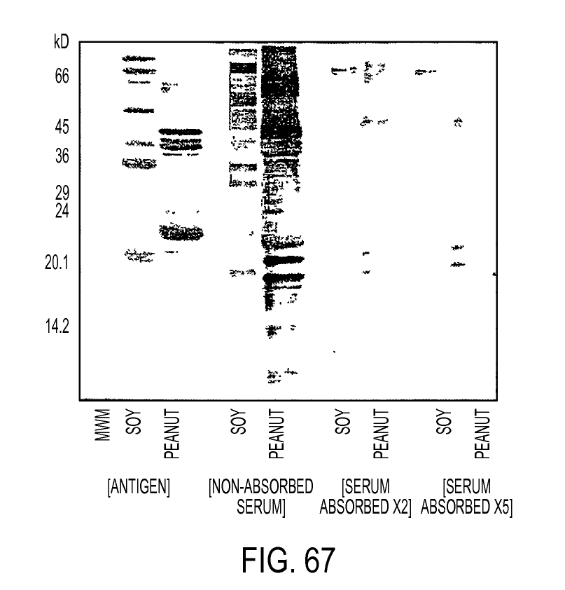

Crude peanut extract was separated by electrophoresis on a tricine-SDS polyacrylamide gel (FIG. 65). Various bands were evident after migration; and major peanut fractions were found at 39, 29, 27, 19, 17, 14, and 12 kd. Immunoblots were incubated with non-adsorbed sera and soy-adsorbed sera for IgE binding. The following peanut protein fractions were bound by specific IgE in all five non-adsorbed sera: 46, 29, 24, 19, 17 and 12 kd (FIG. 66). A double band at 4.5 and 5.5 kd was found in all but on patient. The band at 17 kd probably corresponds to Ara h 2, described as a major peanut allergen. Several minor bands disappeared after adsorption of soy-specific antibody, as well as a stronger band at 12 kd. This band may correspond to a relevant antigenic fraction of soybean protein. Serum binding at 5 kd became more prominent and is particularly evident in patients BM and AT. To study peanut-specific IgE antibody binding at higher molecular weights, peanut extracts were run over a standard glycine SDS-PAGE gel with resolution between 14 and 66 kd, and immunoblots were assayed for IgE binding with soy-adsorbed serum of one patient allergic to peanut and soy (BP). Several peanut fractions could be isolated with major bands at 63, 41, 23, and 15 kd (FIG. 50). Immunoblotting revealed serum IgE binding at 63, 41, 23, 20, 17 and 14 kd. Binding to these peanut fractions remained after adsorption of soy-specific antibodies; however, most minor bands disappeared. IgE binding to a band at 63 kd (Ara h1) could be found with both the non-adsorbed and soy-adsorbed serum, supporting the clinical relevance of Ara h1 as a major peanut allergen.

### 15.4 Conclusion

We used soy protein-specific affinity chromatography columns to remove cross-reacting antibodies from the sera of patients allergic to peanut. Five patients allergic to peanut with very high levels of specific IgE antibodies to peanut were studies. Two of these patients also experienced clinical reactions to soy and had elevated levels of specific IgE antibodies to soy; one patient who was soy-tolerant also had very high specific IgE titers to soy (Table 27). After two sequential passes over the soy-affinity column, serum IgE antibodies to soy rapidly diminished (FIG. 63). However, further passes (up to a total of 5) with increased contact time were necessary to completely remove soy-binding antibodies. The first passes most likely removed antibodies with high affinity to soy, whereas later passes with prolonged exposure time were necessary to remove antibodies with lower affinity. The concomitant decrease of IgE antibody binding to crude peanut extract confirms the presence of significant quantities of cross-reacting antibodies in the sera of patients allergic to peanut.

To determine the magnitude of cross-reactivity, IgE antibody binding to whole peanut extract, to Ara h1, and to Ara h 2 were determined before and after adsorption. Binding to whole peanut antigen was decreased by 73% after the removal of soy cross-reacting antibodies, and binding to Ara h1 was decreased by 79% and Ara h 2 by 76%. Ara h1 belongs to the family of vicilin proteins. which are also found in soybeans and other legumes. Studies indicate that vicilins of various legumes share greater than 60% sequence identity, which may explain the extensive antibody cross-reactivity to this protein.

Food proteins are comprised of several fractions of various sizes, which can be separated by various gel electrophoretic methods. Because most of the potentially antigens can be found between 15 and 50 kd, food proteins are usually separated well on SDS-PAGE. However, relevant allergenic fractions of lower molecular weight may not be identified by this method. To evaluate the low-molecular-weight fractions of peanut, we used tricine-SDS polyacrylamide gels, which efficiently separates low-molecular-weight protein fractions (Schägger and von Jagow, 1987, supra). This method is useful for separating protein fractions with molecular weights as low as 3 to 5 kd and therefore is useful for identifying low-molecular-weight allergenic fractions not seen on standard SDS-PAGE.

Peanut tricine-SDS gel strips transferred to nitrocellulose were probed with non-adsorbed and soy-adsorbed sera from the three patients allergic to peanut and the two patients allergic to both soy and peanut. Significant peanut-specific IgE antibody binding on the peanut immunoblot was found at 46, 29, 24, 19, 17 and 14 kd; and a doublet was found at 5 kd. Sera from one patient (DT) did not bind to the 5 kd fractions, but otherwise, the other four patients demonstrated essentially identical patterns of IgE antibody binding. We have previously characterized Ara h 1 (63.5 kd) and Ara h 2 (17 kd), both major allergenic fractions of peanut extract (see Examples 1 and 8). A minor fraction has been found at 31 kd, and further fractions have been identified at 17 to 25, 34, 55, and 65 kd (Hefle et al., J. Allergy Clin. Immunol. 95:837-842, 1995). Tricine-SDS polyacrylamide gels demonstrated IgE antibody binding to unique peanut fractions at 5 and 13 kd.

### Example 16

### Cloning and Sequencing of Ara h 3

### 16.1 Introduction

We have isolated a cDNA clone encoding a third peanut allergen, Ara h 3. The deduced amino acid sequence of Ara h 3 shows homology to 11S seed-storage proteins. The recombinant form of this protein was expressed in a bacterial system and was recognized by serum IgE from ˜45% of our peanut-allergic patient population.

### 16.2 Methods

### Patients

Serum from patients with documented peanut hypersensitivity was used to probe recombinant protein and identify the Ara h 3 IgE-binding epitopes. Each patient had a positive immediate skin prick test to peanut and either a positive double-blind, placebo-controlled food challenge or a convincing history of peanut anaphylaxis (laryngeal edema, severe wheezing, and/or hypotension). One individual with elevated serum IgE levels (who did not have peanut-specific IgE or exhibit peanut hypersensitivity) served as a control in these studies. In some instances a serum pool, consisting of equal aliquots of serum IgE from each of the patients, was used in immunoblot analysis experiments to determine the IgE-binding characteristics of the population. Details outlining the challenge procedure and collection of IgE serum have been discussed previously (see Example 1). All studies were approved by the Human Use Advisory Committee at the University of Arkansas for Medical Sciences.

### Isolation and Amino Acid Sequence Analysis of Peanut Allergen Ara h 3

Gel slices containing Ara h 3 were sent to the W. M. Keck Foundation (Biotechnology Resource Laboratory, Yale University, New Haven, Conn.) for amino acid sequencing. The NH₂-terminal amino acid sequence of Ara h 3 was determined by performing Edman degradation on an Applied Biosystems Inc. (Foster City, Calif.) gas-phase sequencer with an online HPLC column that was eluted with increasing concentrations of acetonitrile.

Identification of Ara h 3 cDNA Clones

A mature peanut cDNA library was screened using a γ-ATP, 5′ end-labeled, degenerate 23 bp oligonucleotide derived from the NH₂-terminal amino acid sequence (ISFRQQPEENA, SEQ ID NO. 83). Positive plaques were subjected to in vivo excision to remove phagemid from the vector using the R408Helper Phage (Stratagene, La Jolla, Calif.) according to a protocol supplied by the manufacturer. Supernatants containing the excised phagemid pBluescript packaged as filamentous phage particles were decanted into sterile tubes. For DNA preparation, rescubed phagemids were plated on LB-ampicillin plates using XL-1 Blue cells and incubated overnight at 37° C. Colonies appearing on the plate contain the pBluescript double-stranded plasmid with the cloned insert. DNA was prepared using the Plasmid Spin Miniprep kit (QIAGEN Inc., Valencia, Calif., USA) and sequenced as described later here. Several clones were identified in this manner, all of which were lacking ˜300 bp of the 5′ end.

Amplification of Glycinin cDNA Ends

The CapFinder PCR cDNA Library Construction Kit (Clontech Laboratories Inc., Palo Alto, Calif.) was used to selectively amplify the 5′ portion of the cDNA encoding Ara h 3. Poly (A)⁺ RNA was isolated as described previously (Burks et al., J. Clin. Invest. 96:1715-1721, 1995). For first-strand cDNA synthesis, 0.5 μg poly (A)⁺ RNA, 1 μl CapSwitch oligonucleotide, 5 μmol of 3FA.S. (GCACCTTCTGGTGACTATC, SEQ ID NO. 84), an antisense primer derived from a conserved nucleotide sequence present in glycinins, were incubated at 72° C. for 2 minutes, then placed on ice for 2 minutes before being added to a mixture of 5× first-strand buffer, 2 mM DTT, 1 mM dNTP, and 100 U of Moloney murine leukemia virus reverse transcriptase. This reaction proceeded at 42° C. for 1 hour and was placed on ice. To amplify double-stranded cDNA, 20 of first-strand cDNA and 5 pmol each of 3FA.S, and H1 (to serve as primers) were added to a reaction mixture containing 1× KlenTaq PCR buffer, 0.2 mM dNTP, 1× KlenTaq Polymerase Mix, and dH₂O. The PCR reaction commenced with a 1-min denaturation at 95° C., followed by 22 cycles of denaturation at 95° C. and annealing/elongation for 5 minutes at 68° C. The amplified 5′ portion of the Ara h 3 cDNA was cloned into a pGEM-T vector by standard protocols supplied by Promega Corp. (Madison, Wis.).

PCR Amplification of the Ara h 3 mRNA Sequence

Two oligonucleotides, Rab-1 (CGNCAGCAACCGGAGGAGAACGC, SEQ ID NO. 85), derived from nucleotide sequence obtained from selective amplification of the 5′ end of peanut glycinins, and T7+ (CGACTCACTATAGGGCGAATTGG, SEQ ID NO. 86), an oligonucleotide derived from the pBluescript vector sequence—served as primers for a PCR reaction to selectively amplify the nucleotide sequence encoding the Ara h 3 protein. Our mature peanut cDNA library served as template and was concentrated by standard phenol/chloroform extraction followed by ethanol precipitation. Each PCR reaction consisted of 1 μl of concentrated cDNA library, 5 μmol of each primer, 0.2 mM dNTP, and 1.25 U of Taq DNA polymerase. These reactions were carried out in a buffer containing 3 mM MgCl₂, 500 mM KCl, and 100 mM Tris-HCl (pH 9.0). After an initial denaturation cycle at 94° C. for 2 minutes, 30 cycles of PCR consisting of a 30 second denaturation step at 94° C. followed by annealing at 60° C. for 30 seconds and elongation at 72° C. for 1 minute were carried out in a thermocycler (Perking-Elmer Corp., Norwalk, Conn.). After separating by electrophoresis on a 1% agarose gel and purification, products of the appropriate size were inserted into a pGEM-T vector.

### DNA Sequence and Analysis

Sequencing was performed according to the method of Sanger et al. (Sanger et al., 1977, supra) using oligonucleotide primers directed to different regions of the clone and the femtomole DNA Cycle Sequencing System (Promega Corp.). Sequence analysis was performed on the University of Arkansas for Medical Science's Vax computer using the Wisconsin DNA analysis software package (Devereux, 1984, supra).

### Bacterial Expression and Purification of Recombinant Ara h 3

A cDNA corresponding to the Ara h 3 sequence was amplified by PCR and cloned into a pET vector. This plasmid allowed expression of a recombinant protein that included the addition of Ala¹, Ser², and Phe³ at the NH₂— terminus, three amino acids not encoded by our clone. Ser² and Phe³ coincide with amino acids in the native protein; however, Ile¹ in the native protein was altered to Ala¹ in the recombinant for ease of expression (Tobias et al., Science 254:1374-1377, 1991). Primers for PCR were designed to include an NheI site at the 5′ end of the cDNA and a SalI site at the 3′ end of the cDNA. The primers used were: 5′-TATGGCTAGCTTCCGGCAG-CAACCGGAGGAG-3′ (5′ primer, SEQ ID NO. 87) and 5′-CCGTCGACAGCCACAGCCCTC-GGAGA-3′ (3′ primer, SEQ ID NO. 88). PCR products were cloned into the NheI/SalI restriction sites of the plasmid pET 24(B)⁺ under the control of the T7 lac promoter. This expression vector contains the gene encoding kanamycin resistance and coding sequence for His₆ tag produced at the COOH-terminus of the recombinant protein. Protein expression in the E. coli strain BL21(DE3) was induced by the addition of isopropyl-B-_{D}-thio-galactopyranoside to a final concentration of 1 mM once the culture reached A₆₀₀=0.6. The cells were harvested at 1 hour intervals, resuspended in SDS sample buffer containing DTT, and boiled at 100° C. for 5 minutes. Samples were either used immediately for immunoblot analysis, or samples were pelleted, washed with 50 mM Tris-HCl, and stored for later use as a frozen pellet at −70° C.

Recombinant Ara h 3 was purified from bacterial lysates under denaturing conditions using the His-Bind Purification Kit (Novagen Inc., Madison, Wis.). Cell extracts were resuspended in 4 ml of cold Binding Buffer (5 mM imidazole, 0.5 M NaCl, 20 mM Tris-HCl, and 6 M urea; supplied with Novagen kit), sonicated to shear DNA, and incubated on ice for 1 hour. Next, the lysate was centrifuged at 12,000 g for 45 minutes to remove cellular debris. The post-centrifugation supernatant was prepared for loading onto the column by passing it through a 0.45-μm membrane using a syringe-end filter. A His-Bind Quick Column (Novagen) was packed with His-Bind metal chelation resin, washed with deionized H₂O, and charged until saturation with Charge Buffer (50 mM NiSO₄; Novagen). After equilibration of the column with Binding Buffer, 2 volumes of supernatant were loaded onto the column. The column was washed with 10 volumes of Binding Buffer and 6 volumes of Wash Buffer (20 mM imidazole, 0.5 M NaCl, 20 mM Tris-HCl, and 6 M urea). Elution was achieved with 5 volumes of Elution Buffer (1 M imidazole, 0.5 M NaCl, 20 mM Tris-HCl, and 6 M urea; Novagen). Fractions collected over the course of the experiment containing recombinant Ara h 3 were lyophilized and stored in 1×PBS.

### SDS-PAGE, Western Blots, and IgE-Binding Assay

Purified recombinant Ara h 3 was analyzed by SDS-PAGE using precast 12% Tris-glycine gels (Novex, San Diego, Calif.). Samples were electrophoresed for 90 minutes at 125 V. Proteins were visualized by either Coomassie blue staining or by using Gelcode Blue Stain Reagent (Pierce Chemical Co., Rockford, Ill.) according to the manufacturer's protocol. For immunoblot analysis, proteins were electroblotted onto nitrocellulose at 30V for 90 minutes. After transfer, blots were blocked using a solution containing Tris-NaCl and 3% BSA. Alternatively, cellulose membranes containing synthetic peptides were blocked in a solution provided by Genosys Biotechnologies, Inc. (The Woodlands, Tex.). All blots were incubated with a serum pool from patients with documented peanut hypersensitivity or individual sera diluted (1:5) in a solution containing Tris-NaCl and 1% BSA for 16 hours at 4° C. Primary antibody was detected with ¹²⁵I-labeled anti IgE antibody (Sanofi Diagnostic Pasteur Inc., Paris, France).

### 16.3 Results

Molecular Cloning and Sequence of the Ara h 3 cDNA

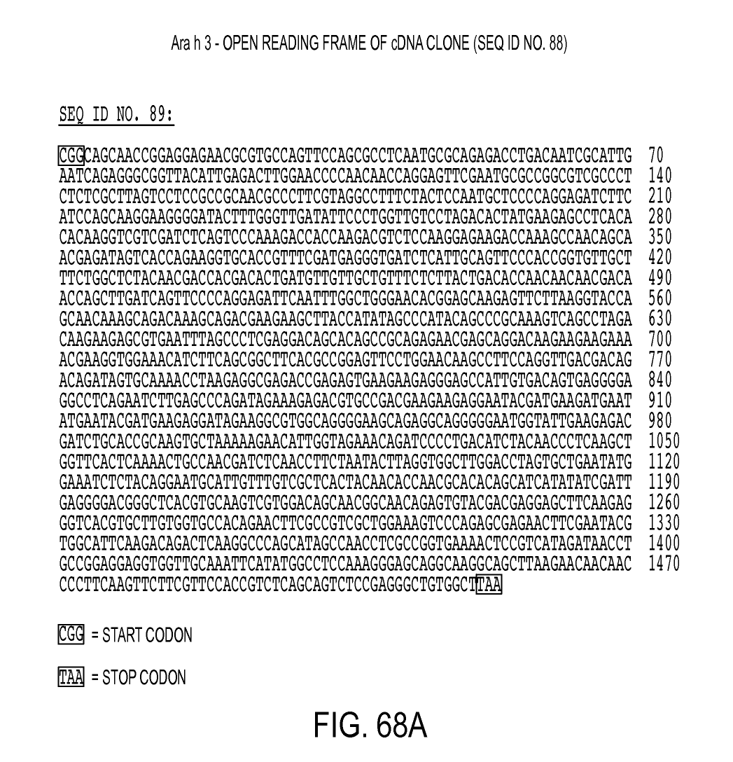

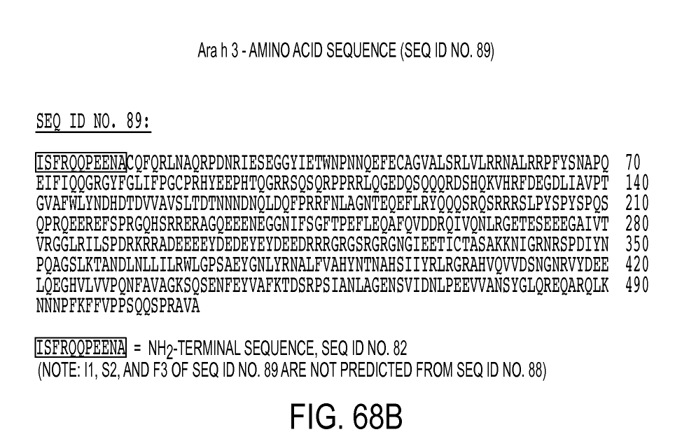

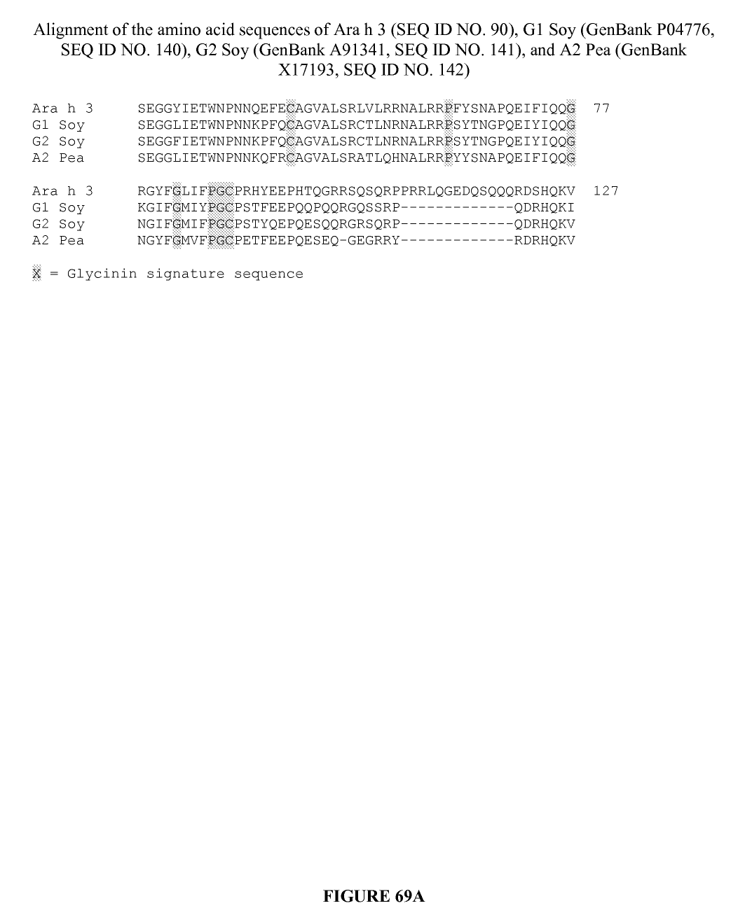

The NH₂-terminus of a purified 14 kd protein identified by soy-adsorbed IgE serum from peanut-hypersensitive patients (see Example 15) was sequenced. Degenerate oligonucleotides derived from the amino acid sequence were used to screen a mature peanut cDNA library. The sequence of the Ara h 3 cDNA (SEQ ID NO. 89) and the predicted amino acid sequence (SEQ ID NO. 90) are shown in FIGS. 68A and 68B, respectively. The Ara h 3 cDNA includes an open-reading frame (ORF) of 1,524 nucleotides (SEQ ID NO. 89), coding for 507 amino acids. This ORF starts with a CGG codon and ends with a TAA stop codon at nucleotide position 1,524 of SEQ ID NO. 89. The calculated size of the protein encoded by this open reading frame is ˜57 kd. The amino acids obtained from NH₂-terminal sequencing of the 14 kd protein (SEQ ID NO. 83) correspond to the amino acids encoded by the nucleotides located at the 5′ end of the cDNA clone. The 14 kd protein appears to be an NH₂-terminal breakdown product of a larger allergen. The cDNA clone appears to be lacking the extreme 5′ end that would encode a signal peptide and the initiator methionine. Note that amino acids 1 to 3 of SEQ ID NO. 90 are found at the sequenced NH₂-terminus of Ara h 3 (SEQ ID NO. 83), but are not encoded by the cDNA clone.

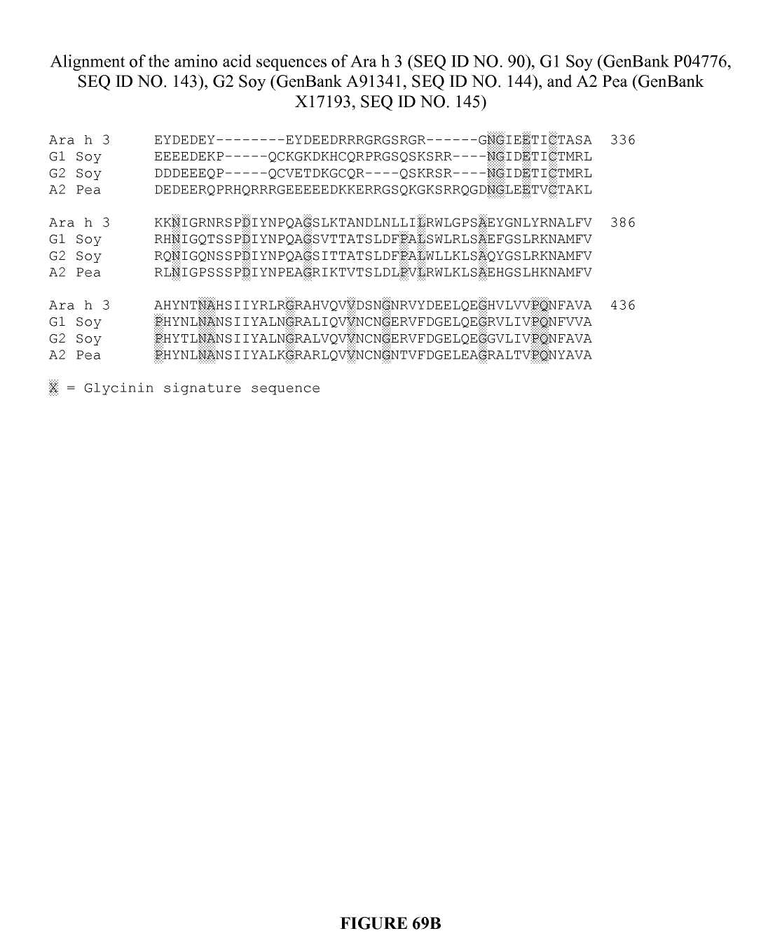

Database searches for sequence similarity revealed that the Ara h 3 cDNA encoded an 11S seed-storage protein. Ara h 3 showed 62%-72% sequence identity with other legume glycinins (FIGS. 69A and 69B). G1 Soy is the glycinin G1 precursor containing Ala-Bx chains (from Glycine max, GenBank P04776), G2 Soy is the glycinin G2 precursor containing the A2-B1a chains (from Glycine max, GenBank A91341), and A2 Pea is the legumin A2 precursor (from Pisum satvium, GenBank X17193). In particular, 24 to 26 residues thought to be important for the tertiary structure of these storage proteins (Bairoch and Bucher, Nucleic Acids Res. 22:3584-3589, 1994) are present in the Ara h 3 primary sequence, including a conserved cleavage site at Asn-325 and Gly-326 of SEQ ID NO. 90. FIG. 69A shows a conserved region near the amino terminus of the acidic chain. Shaded residues represent residues belonging to a glycinin signature sequence. FIG. 69B shows a conserved region near the amino-terminus of the basic chain. There was no homology noted between this allergen and the other major peanut allergens already identified (Ara h 1, SEQ ID NO. 7 or Ara h 2, SEQ ID NO. 63).

### Expression, Antigenicity, and Purification of Recombinant Ara h 3

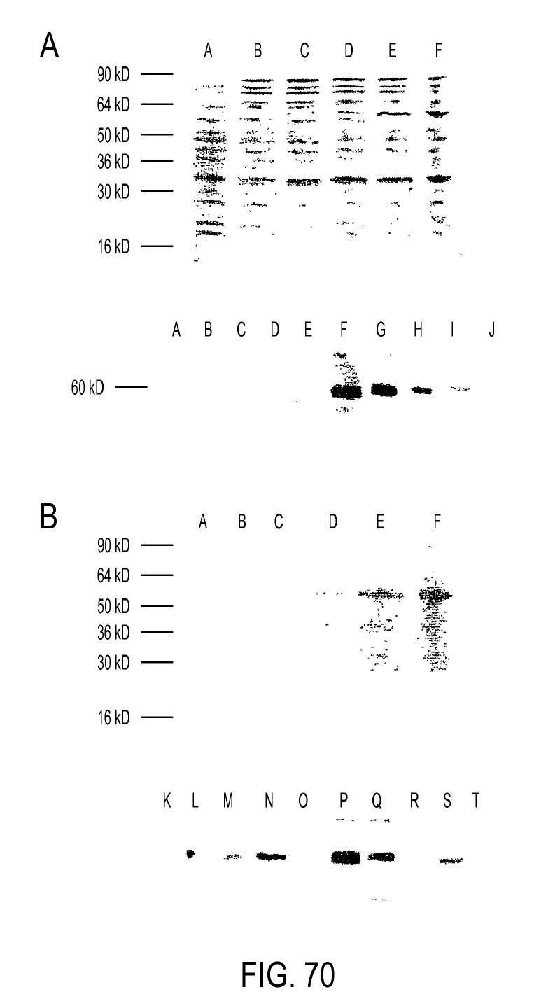

The Ara h 3 cDNA was cloned into a pET 24 plasmid and expressed in a bacterial system. Optimal expression was obtained following a four-hour induction by isoproply B-D-thiogalactopyranoside (FIG. 70A, lane F). The immunoblot in FIG. 70B was performed using serum IgE from a pool of patients with peanut hypersensitivity to determine the molecular weight and specificity of IgE binding. From the blot, the estimated size of the recombinant protein produced by bacterial cells is ˜57 kd, which corresponds to the predicted molecular mass encoded by the clone. FIG. 70C shows 20 immunoblot strips of purified recombinant Ara h 3 incubated with different patient sera. Forty-four percent (8/18) of the patients tested had IgE that recognized the recombinant protein (FIG. 70C, lanes A-R). The difference in binding intensities between Ara h 3-allergic patients could be due to the amount of peanut-specific IgE in each individual or differences in affinity of patient-specific IgE to this allergen.

### 16.4 Conclusion

We have reported the cDNA cloning, expression, and epitopes analysis of Ara h 3, an allergenic, 11S storage protein from the peanut, Arachis hypogaea. Although these are predominant proteins in legumes, this is the first time that the cDNA from an 11S storage protein has been cloned and shown to encode an allergenic protein in the peanut. 11S storage proteins are initially synthesized as 60 kd preproglobulins consisting of covalently linked acidic and basic polypeptides. The precursors are deposited in storage bodies where they aggregate into trimers, before being cleaved by an asparagine-dependent endopeptidase (Turner et al., J. Biol. Chem. 257:4016-4018, 1982 and Barton et al., J. Biol. Chem. 257:6089-6095, 1982). This results in an NH₂-terminal acidic chain of ˜35 kd and a COOH-terminal basic chain of ˜20 kd, which later become linked by a disulfide bridge (Staswick et al., J. Biol. Chem. 256:8752-8755, 1981). 11S storage proteins are then assembled into their mature form as hexameric oligomers consisting of six similar subunits (Staswick et al., J. Biol. Chem. 259:13431-13435, 1984). The Ara h 3 cDNA represents the coding region for the 60 kd preproglobulin.

We have demonstrated high-level expression of recombinant Ara h 3 in a bacterial system. Serum IgE from 44% (8/18) of our peanut-allergic patient population recognized recombinant Ara h 3, designating it as a minor allergen. This is in contrast to the other peanut allergens, Ara h 1 and Ara h 2, both of which are major allergens (see Examples 1 and 8), recognized by >90% of the patient population. All three of these allergens share similar functional properties; they are all seed-storage proteins with no enzymatic activity. However, no direct evidence exists as to why only a portion of the patient population recognizes Ara h 3. The ability of 11S storage proteins to oligomerize into hexamers and the position of the epitopes at the tertiary level of protein structure may provide insight into this issue. Another possibility is the level of sequence similarity retained between these proteins from different legumes. Ara h 3 exhibits higher sequence identity with legume storage proteins from soybean and pea (62%-72%) than Ara h 1 exhibits with vicilins (40%) or Ara h 2 exhibits with conglutinins (39%). The percentage of patients with allergen-specific IgE may depend on unique sequences not conserved between protein families of different legume species. This would account for the lower percentage of peanut-allergic patients with IgE to Ara h 3.

### Example 17

### Mapping and Mutational Analysis of the Linear IgE Epitopes of Ara h 3

### 17.1 Introduction

Serum IgE from these patients and overlapping, synthetic peptides were used to map the linear, IgE-binding epitopes of Ara h 3. Several epitopes were found within the primary sequence, with no obvious sequence motif shared by the peptides. One epitope is recognized by all Ara h 3-allergic patients. Mutational analysis of the epitopes revealed that single amino acid changes within these peptides could lead to a reduction or loss of IgE binding.

### 17.2 Methods

### Peptide Synthesis

Individual peptides were synthesized with Fluorenylmethoxycarbonyl (Fmoc) amino acids on a derivatized cellulose membrane containing free hydroxyl groups according to manufacturer's instructions (Genosys Biotechnologies). Briefly, synthesis of each peptide began by esterifying an Fmoc amino acid to the cellulose membrane. Coupling reactions are followed by acetylation with acetic anhydride in N,N-dimethylformamide to render peptides unreactive during the subsequent steps. After acetylation, Fmoc protective groups are removed by the addition of piperidine to render nascent peptides reactive. The remaining amino acids are added by this same process of coupling, blocking, and deprotection, until the desired peptide is generated. Upon addition of the last amino acid, the side chains of the peptide are deprotected with a 1:1:0.05 mixture of dichloromethane/trifluoreacetic acid/trilisobutylsilane and washed with methanol. Membranes containing synthetic peptides were either probed immediately with serum IgE or stored at −20° C. until needed.

### 17.3 Results

### Multiple IgE-Binding Regions Located Throughout the Ara h 3 Protein

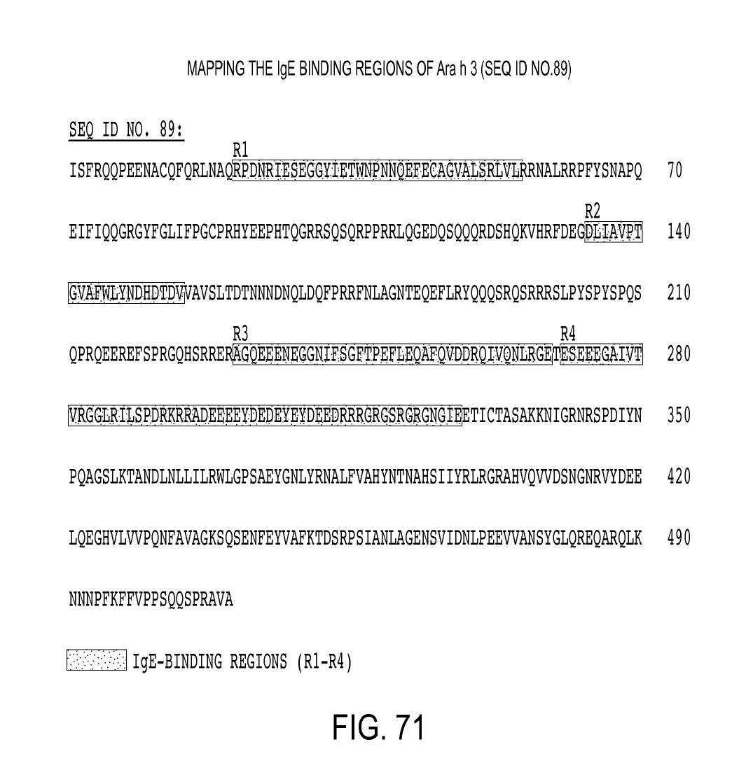

Sixty three overlapping peptides were synthesized to determine which regions of the Ara h 3 protein were recognized by serum IgE. Each peptide synthesized was 15 amino acids long and offset from the previous peptide by 8 amino acids. This approach allowed the analysis of the entire Ara h 3 primary sequence in large, overlapping fragments. These peptides were probed with a serum pool of IgE from peanut-hypersensitive patients who had previously been shown to recognize recombinant Ara h 3. FIG. 71 shows the four IgE-binding regions and their corresponding location within the Ara h 3 primary amino acid sequence. These IgE-binding regions were represented by amino acid residues 21-55, 134-154, 231-269, and 271-328 of SEQ ID NO. 90.

### Immunodominance and Characterization of the Ara h 3 Epitopes

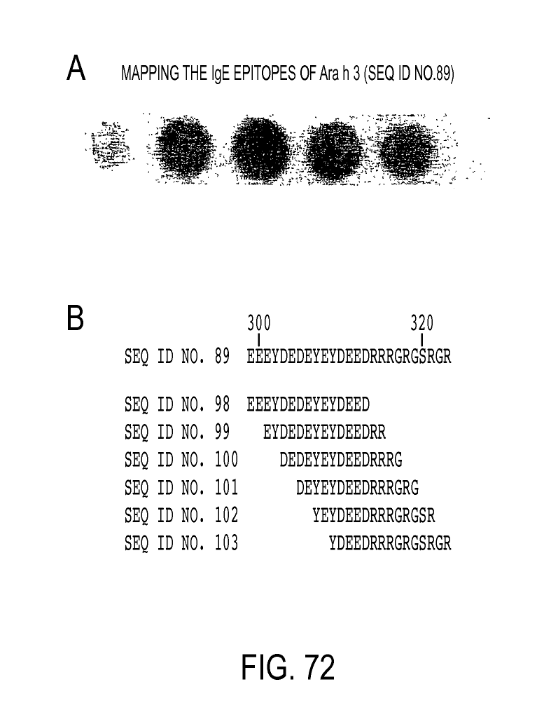

To determine the exact amino acid sequence of the IgE-binding regions, synthetic peptides (15 amino acids offset by 2 amino acids) representing the larger IgE-binding regions were generated and probed with a serum pool of IgE from patients who recognize recombinant Ara h 3. This process made it possible to distinguish individual IgE-binding epitopes within the larger IgE-binding regions of the Ara h 3 protein. FIG. 72A is an immunoblot of six synthetic peptides which span amino acid residues 299 to 323 of SEQ ID NO. 90. FIG. 72B shows the amino acid sequence representing this region and the amino acid sequences represented by each individual peptide. The shaded area in FIG. 72B represents the core epitope. The four IgE-binding epitopes identified in this manner are shown in Table 29A. To determine whether any of the four epitopes were immunodominant (within the Ara h 3-allergic population), each set of four peptides was probed individually with serum IgE form the eight patients previously shown to recognize recombinant Ara h 3 (results summarized in Table 29A as percentage recognition).

TABLE 29A
Ara h 3 IgE binding epitopes

| SEQ | | | | |
|---|---|---|---|---|
| ID | Pep- | Amino acid | Ara h 3 | |
| NO. | tide | sequence¹ | positions² | Recognition³ |
| 91 | 1 | IETWNPNNQEFECAG | 33-47 | 25% (2/8) |
| 92 | 2 | GNIFSGFTPEFLEQA | 240-254 | 38% (3/8) |
| 93 | 3 | VTVRGGLRILSPDRK | 279-293 | 100% (8/8) |
| 94 | 4 | DEDEYEYDEEDRRRG | 303-317 | 38% (3/8) |

¹The peptides are indicated as the single-letter amino acid code.
²The Ara h 3 amino acid positions are taken from SEQ ID NO. 90.
³The percent recognition is the percentage of patients previously shown to recognize recombinant Ara h 3 whose serum IgE recognized that particular synthetic epitope.

Epitope 1 was recognized by serum IgE form 25% (2/8) of the patients tested, whereas epitopes 2 and 4 were recognized by serum IgE from 38% (3/8) of the eight patients tested. Interestingly, epitopes 2 and 4 were recognized by the same three patients. Epitope 3 was recognized by serum IgE from 100% (8/8) of the Ara h 3-allergic patients, classifying it as an immunodominant epitope within the Ara h 3-allergic population. Sixty-eight percent of the amino acids constituting the epitopes were either polar uncharged or apolar residues. However, three was no obvious sequence motif with respect to position or polarity shared by the individual epitopes.

Characterization of the IgE binding regions was repeated using synthetic overlapping peptides which were 10 amino acids in length and offset by 2 amino acids. As with the 15/2 peptides, the 10/2 peptides were probed with a serum pool of IgE form patients who recognize recombinant Ara h 3. The four IgE-binding epitopes identified in this manner are shown in Table 29B. To determine whether any of the four epitopes were immunodominant (within the Ara h 3-allergic population), each set of four peptides was probed individually with serum IgE form a larger group of twenty patients previously shown to recognize recombinant Ara h 3 (results summarized in Table 29B as percentage recognition).

TABLE 29B
Ara h 3 IgE binding epitopes

| SEQ | | | | |
|---|---|---|---|---|
| ID | Pep- | Amino acid | Ara h 3 | |
| NO. | tide | sequence¹ | positions² | Recognition³ |
| 95 | 5 | EQEFLRYQQQ | 183-192 | 5% (1/20) |
| 96 | 6 | FTPEFLEQAF | 246-255 | 25% (5/20) |
| 97 | 7 | EYEYDEEDRR | 306-315 | 35% (7/20) |
| 98 | 8 | LYRNALFVAH | 379-388 | 100% (20/20) |

¹The peptides are indicated as the single-letter amino acid code.
²The Ara h 3 amino acid positions are taken from SEQ ID NO. 90.
³The percent recognition is the percentage of patients previously shown to recognize recombinant Ara h 3 whose serum IgE recognized that particular synthetic epitope.

### Mutations at Specific Residues Eliminate IgE Binding

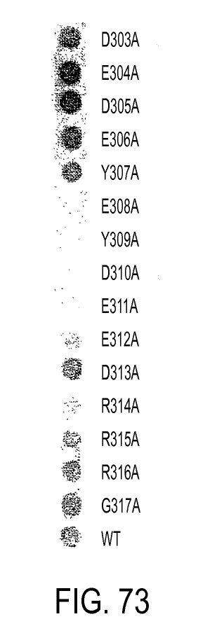

The amino acids essential for IgE binding to the Ara h 3 epitopes were determined by synthesizing multiple peptides with single amino acid changes at each position. These peptides were probed with a pool of serum IgE from patients who had previously recognized the wild-type peptide, to determine whether amino acid changes affected peanut-specific IgE binding. FIG. 73 shows an immunoblot strip containing the wild-type and mutant peptides for peptide 4 of Table 29A. The pool of serum IgE did not recognize the peptide, or a decrease in binding was observed when alanine was substituted for the wild-type amino acid at positions 308, 309, 310, 311, 312, and 314 of SEQ ID NO. 90. Interestingly, it appears as if an alanine substitution increases IgE binding at positions 304 and 305 of SEQ ID NO. 90. The remaining Ara h 3 epitopes were analyzed in the same manner. In general, each epitope could be altered to a non-IgE-binding peptide by the replacement of the wild-type amino acid residue with alanine. The critical residues for IgE binding within each peptide of Table 29A are shown in Table 30.

TABLE 30
Amino acids critical to IgE
binding in Ara h 3

| SEQ | | | |
|---|---|---|---|
| ID | | Amino acid | Ara h 3 |
| NO. | Peptide | sequence¹ | position² |
| 91 | 1 | IETWNPNNQEFECAG | 33-47 |
| 92 | 2 | GNIFSGFTPEFLEQA | 240-254 |
| 93 | 3 | VTVRGGLRILSPDRK | 279-293 |
| 94 | 4 | DEDEYEYDEEDRRRG | 303-317 |

¹The amino acids that, when altered, lead to loss of IgE binding are shown as the bold, underlined residues.
²The Ara h 3 amino acid positions are taken from SEQ ID NO. 90.

It appears that the central amino acids within each epitope are favored for mutation. All mutations that led to a significant decrease in IgE binding were located at residues found within each core epitope (as identified in FIG. 72). There was no obvious consensus in the type of amino acid that, when mutated to alanine, leads to complete loss or a decrease in IgE binding.

### 17.4 Conclusion

Given that allergen-specific IgE plays such a critical role in the etiology of allergic disease, determination of allergen-specific, IgE-binding epitopes is an important first step toward understanding the complexity of hypersensitivity reactions. By generating synthetic, overlapping peptides representing the entire primary sequence of the protein, we were able to determine that there are four distinct IgE-recognition sites distributed throughout the primary sequence of the protein. One of these sites (within peptide 3 of Table 29A) was recognized by serum IgE from every Ara h 3-allergic patient in the group, designating it as an immunodominant epitope. Interestingly, epitopes located within peptides 3 and 4 (Table 29A) are located within the hypervariable region of the acidic chain, a stretch of amino acids that is highly variable in length among 11S storage proteins. This region contains a high proportion of glutamate, aspartate, and arginine residues and will tolerate large, naturally occurring insertions or deletions. Computer predictions from other studies suggest that this region is exposed on the surface of the protein (Nielsen et al., pp. 635-640 in “NATO Advanced Study Institute on Plant Molecular Biology”, Ed. by R. Hermann and B. Larkins, Plenum Press, New York, N. Y., 1990).

### Example 18

### Ara h 3 Mutant Protein with Reduced IgE Binding

The elucidation of the major IgE-binding epitopes of Ara h 3 in Example 17, and the determination of which amino acids within these epitopes provides the information necessary to alter the Ara h 3 gene by site-directed mutagenesis to encode a protein that escapes IgE recognition.

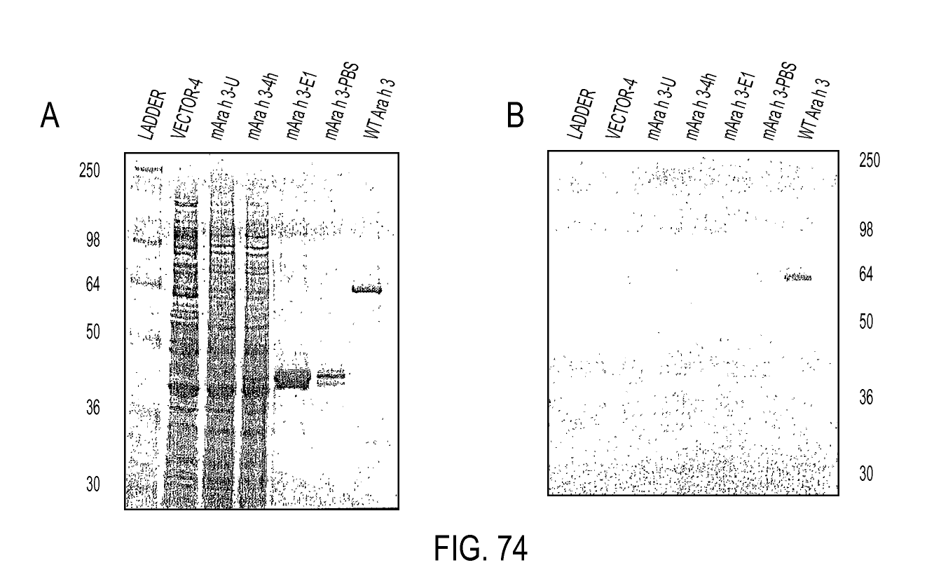

The Ara h 3 cDNA was mutated by PCR to encode alanine for one critical residue within each epitope. The cDNA encoding the 40 kd acidic chain of the 11S legumin-like storage protein was placed under the control of the T7 lac promoter and expressed in a bacterial system (see Methods in Example 5). FIG. 74A shows SDS-PAGE separation gels of the mutant recombinant Ara h 3 (mAra h 3) after expression and after various purification steps. FIG. 74A also shows a gel of the 60 kd pre-proglobulin wild-type recombinant Ara h 3 protein (WT Ara h 3) consisting of covalently attached 40 kd (acidic) and 20 kd (basic) chains. Both the mutated and wild-type recombinant proteins were purified by Ni²⁺ column chromatography.

In FIG. 74B, the proteins separated in (FIG. 74A) were blotted to nitrocellulose and probed with serum IgE from three patients previously shown to recognize recombinant Ara h 3. As seen from the blot, while the wild-type Ara h 3 protein is bound by IgE, the mutated Ara h 3 protein was not recognized by serum IgE from the Ara h 3-allergic patients.

### Example 19

### Identification of Soybean Allergens Using IgE Sera Adsorbed to Remove Cross-Reacting Antibodies to Peanuts

### 19.1 Introduction

Allergic reactions to soybeans, compared to fish and peanuts, are unique in that the clinical reaction is typically outgrown in the first 3-5 years of life. We have used amino acid homology-based data searches, peanut-specific, and soy-specific serum to screen allergens from soybeans to identify and characterize differences in peanut and soybean vicilin and glycinin seed storage proteins.

In this Example, cross-reacting antibodies to peanut were removed from the sera of a patient allergic to peanut and soy and a patients allergic to peanut by peanut-affinity chromatography. Adequate removal of cross-reacting antibodies was verified by ELISA after each adsorption step. Unabsorbed sera and sera absorbed to remove cross-reacting antibodies were assayed for specific IgE binding to soy immunoblots. Unique soy-specific IgE antibodies (i.e., peanut antibody-absorbed) were found to bind to a soy fraction at 46 kd, and to a lesser extent, to a fraction at 21 kd on immunoblots of whole soy protein.

### 19.2 Methods

### Patients

Sera were obtained from the same patients as Example 15.

### Preparation of Soy and Peanut Extracts

The soybean extract was obtained from soy flour and processed as described in Example 15. The peanut extract was obtained form three commercial lots of Florunner peanuts and processed as described in Example 1.

### Antigen-Specific Serum Antibody Absorption

An affinity column was generated with 40 mg of peanut antigen and active ester agarose gel using the same as described in Example 15. The same adsorption and elution procedures that were performed using the soy-affinity column of Example 15 were repeated using the peanut-affinity column of the present study and sera from a patient allergic to peanut and soy (BP), and a soy-tolerant patient with peanut allergy (DT).

### ELISA for IgE

After each passage of sera over the peanut-affinity chromatography columns, the presence of peanut-specific IgE was monitored by ELISA and by CAP-RAST FEIA (see Methods of Example 15).

### Tricine-SDS Polyacrylamide Gels and Immunoblotting

Soy protein extract (2 mg/ml) was mixed with an equal volume of SDS sample buffer (50 mmol/L Tris HCl, pH 6.8), containing 4% SDS, 2% β-mercaptoethanol, 12% glycerol bromphenol blue, and pyronin Y, and boiled 10 minutes for denaturation. Separation was performed by tricine-SDS polyacrylamide gel as described for peanut extract in Example 15. Electrophoresis was performed at 30 V through the stacking gel and at 80 V overnight through the running gel.

The soy proteins were electrotransferred from the polyacrylamide gel to nitrocellulose paper at 0.15 A for 6 hours in 50 mmol/L of Tris-glycine buffer (pH 9.1) containing 20% methanol. After transfer, the nitrocellulose blots were blocked overnight in PBS-T with 0.5% gelatin. Protein staining with 0.1% amido black was obtained for each gel to confirm proper electrophoresis and protein transfer on nitrocellulose paper. The blots were then incubated with non-adsorbed serum and absorbed serum (5:1 vol/vol dilution) for 2 hours at room temperature on a rocking platform. The blots were washed five times for 5 minutes with PBS-T and incubated for 2 hours with biotin-conjugated goat anti-human IgE in antibody buffer (1:1600). After five washes, the blots were incubated with streptavidin-peroxidase in avidin buffer (1:1000) for 30 minutes, washed, and developed with Sigma FAST DAB (Sigma Chemical Co.). The reaction was stopped by rinsing the blots several times in distilled water. The molecular weights of protein fractions with IgE binding were determined by scanning densitometry (Ultroscan; LKB, Broma, Sweden) and compared with molecular weight makers.

### 19.3 Results

### Peanut-Specific Serum Antibody Adsorption and Immunoblotting

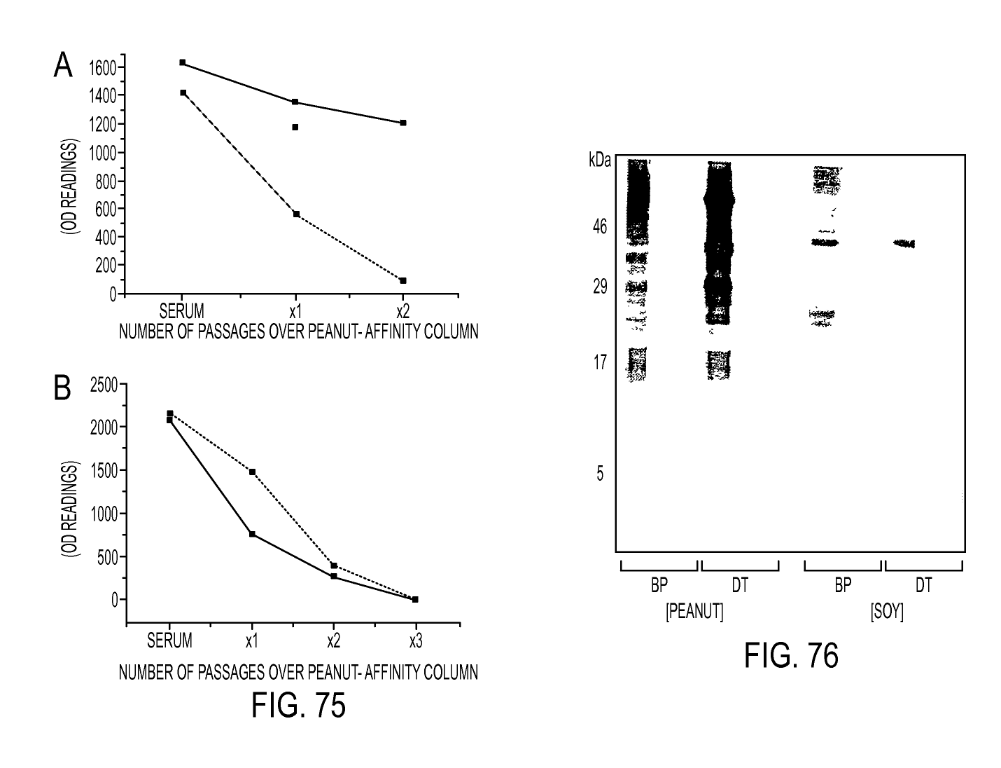

Serum from one patient allergic to soy and peanut (BP) and serum from one soy-tolerant patient with peanut allergy (DT) were depleted of peanut-specific antibody by peanut affinity chromatography. Sera were monitored by CAP-RAST FEIA (see Table 31) and by ELISA (see FIG. 75) to ensure complete removal of peanut-specific IgE antibodies.

TABLE 31
Peanut- and soy-specific IgE antibody concentrations (IU/ml)
after successive passes over a peanut-affinity column

| Patient | Non-adsorbed | Pass 1 | Pass 2 | Pass 3 | Pass 4 | Pass 5 |
|---|---|---|---|---|---|---|
| BP | | | | | | |
| Peanut | 2225 | 17 | 4 | 2 | | |
| Soy | 88 | 6 | 2 | 1 | | |
| DT | | | | | | |
| Peanut | 2405 | 61 | 8 | 6 | 4 | |
| Soy | 75 | 48 | 23 | 13 | 6 | |

All cross-reacting antibodies from the patient allergic to peanut (DT) were removed by the peanut-affinity column (FIG. 75B and Table 31), whereas unique antibodies to soy remained in the serum from the patient with peanut and soy allergy (BP, FIG. 75A and Table 31). IgE antibodies in peanut-adsorbed serum from patient BP (allergic to soy and peanut) bound to soy fractions at 45, 26, and 21 kd (FIG. 76). Antibody binding to the fraction at 45 kd, and to a lesser degree, to the fraction at 21 kd (although some non-specific binding to this fraction can be observed) persisted after peanut-antibody adsorption, suggesting that these fractions may be unique soy proteins. IgE antibodies binding to the fraction at 26 kd were removed by the peanut-affinity column and are therefore unlikely to be clinically relevant to soy allergy.

### Tricine-SDS Polyacrylamide Gels and Immunoblotting

Crude soy flour extract was separated by electrophoresis on a tricine-SDS polyacrylamide gel (FIG. 65). Various bands were evident after migration; and major soy fractions were found at 46, 40, 33, 19, and 9 kd. Immunoblots were incubated with non-adsorbed sera and adsorbed sera for IgE-binding.

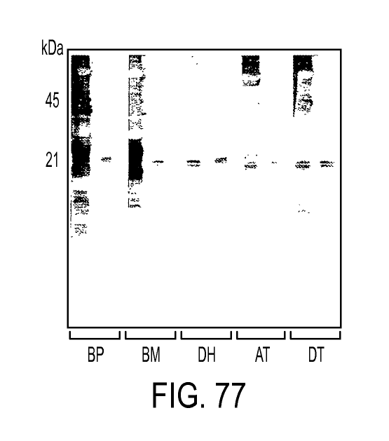

Non-adsorbed sera from the two patients allergic to soy (BP and BM) were reacted with the soy immunoblots (FIG. 77). IgE antibodies bound to protein fractions of 45 and 17 kd and to a large band at approximately 21 kd, whereas IgE antibodies from the soy-tolerant patients bound fractions at 45 kd, and to a lesser degree, at 21 kd.

### 19.4 Conclusion

We used peanut protein-specific affinity chromatography columns to remove cross-reacting antibodies from the sera of patients allergic to soy. Peanut-specific antibodies were removed from the sera of two patients (one with clinical reactivity to peanut and soy and one with reactivity to peanut but with high levels of soy-specific IgE antibodies) by peanut-affinity column chromatography. No IgE antibody binding to the peanut blots could be detected after adequate adsorption of both sera. However, with the peanut-adsorbed sera from the patient allergic to peanut and soy (BP), one band on the soy blot with strong IgE antibody binding became prominent at 46 kd, and a weaker band remained at 21 kd. To date, only partial characterization of soy antigens has been achieved, with relevant protein fractions in the 7S portion isolated by ultracentrifugation (Burks et al., 1988b, supra), as well as a minor allergen at 20 kd and several different fractions between 15 and 55 kd (Bush et al., J. Allergy Clin. Immunol. 82:251-255, 1988). Furthermore, immunoblotting showed IgE binding in sera from soy-sensitive patients with atopic dermatitis to a band at 30 kd (Ogawa et al., J. Nutr. Sci. Vitamin 37:555-565, 1991). The IgE antibody binding to a 21 kd fraction by tricine-SDS-PAGE in this study may correspond to the antigenic fraction previously described at 20 kd; the fraction at 46 kd has not been described previously.

### Example 20

### Identification and Characterization of Soybean Allergen Glycinin Subunit A2B1a, a Member of the Glycinin Family of Seed Storage Proteins

### 20.1 Introduction

Using prep cell, a two dimensional SDS-PAGE and serum from soybean-sensitive individuals, a 20-22 kd soybean allergen was identified from soybean extract using Western IgE, immunoblot analysis. N-terminal sequencing revealed this protein to be the Bla region of the soybean glycinin subunit A2B1a, a member of the glycinin family of seed storage proteins The Bla region of this subunit showed approximately 60% homology to a portion of peanut allergen Ara h 3 which was discussed in Examples 15-18.

### 20.2 Methods and Results

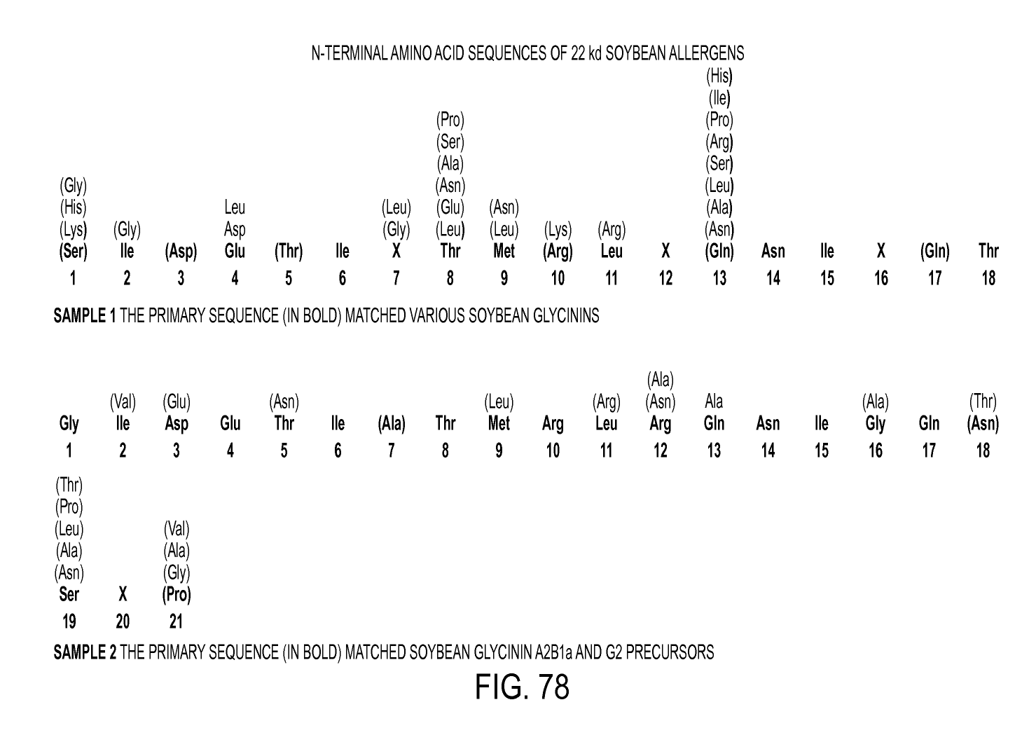

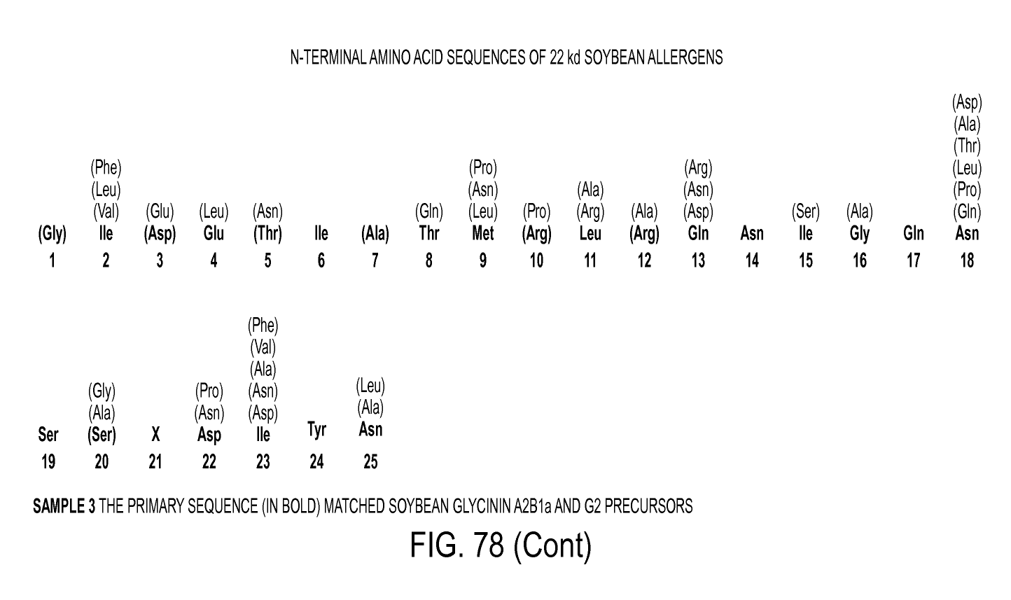

A crude soybean extract was applied to a 12.5% preparative SDS-PAGE gel and electrophoresed using a Bio-Rad prep cell. Five ml fractions were collected and aliquots were electrophoresed into a Pharmacia 24 well 10% horizontal gel, electrophoretically transferred to a nitrocellulose membrane, the remaining sites blocked using PBS/0.05% Tween 20, and analyzed for IgE-binding using serum from soybean-sensitive individuals. Fractions that bound IgE were dialyzed against 100 mM ammonium bicarbonate (×4×4 liters) for 24 hours, lyophilized, reconstituted in distilled water and analyzed by two dimensional (isoelectric focusing in the first dimension, pH 3-7, followed by a 4-20% SDS-PAGE gel molecular weight separation in the second dimension) in duplicate. The proteins in the duplicate gels were transferred to nitrocellulose membranes, one was stained with Coomassie blue for protein identification and the other was prepared for IgE immunoblot analysis. IgE-binding proteins were identified by radiolabeled anti-IgE and X-ray autoradiography. Positive IgE-binding proteins by autoradiography were compared to the Coomassie stained gel protein profile. Several samples taken from a stained blot were submitted to the Yale Biotechnology Center for amino acid sequencing. The sequencing results are illustrated in FIG. 78 and summarized in Table 32 for three of these samples.

TABLE 32
Primary N-terminal sequence of
immunoblotted 22 kd soybean
allergen samples

| | SEQ ID | |
|---|---|---|
| Sample¹ | NO. | Primary amino acid sequence |
| 1 | 105 | SIDETIXTMRLXQNIXQT |
| 2 | 106 | GIDETICTMRLRGNIGQNSXP |
| 3 | 107 | GIDETICTMRLRQNIGQNSSXDIYN |
| A2B1a² | 108 | GIDETICTMRLRQNIGQNSSPDIYN |

X = Unable to identify amino acid.
¹Samples further described in FIG. 78.
²Sequence taken from amino acids 301-325 of SEQ ID NO. 109.

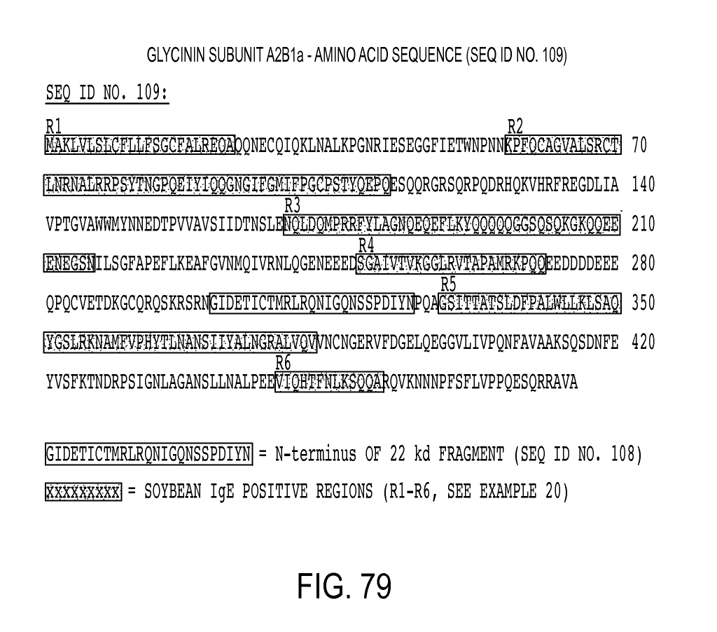

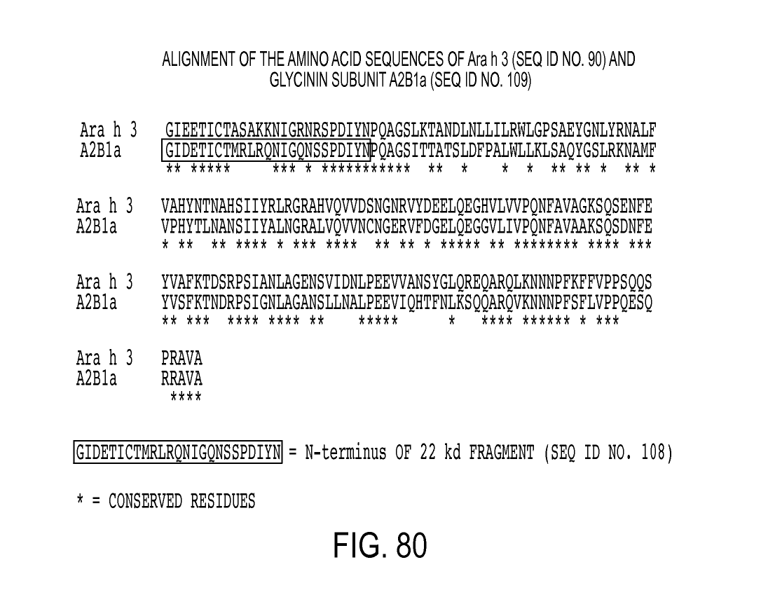

Table 32 also compares the sequenced N-termini with amino acids 301-325 of the glycinin subunit A2B1a from soybean (SEQ ID NO. 109, FIG. 79). The close homology between these sequences suggests that the 22 kd fragment is related to the B1a region of glycinin subunit A2B1a which spans amino acids 301-480 of glycinin subunit A2B1a (Shutov et al., FEBS 241:221-228, 1996). FIG. 79 shows the location of the sequenced region within the amino acid sequence of glycinin subunit A2B1a. The C-terminal half of glycinin subunit A2B1a includes the Bla region, and as shown in FIG. 80, this region is approximately 60% homologous with the C-terminal half of peanut allergen Ara h 3 (SEQ ID NO. 90).

A SPOTS membrane representing individual 15 mers offset by 8 amino acids of glycinin subunit A2Bla was incubated with pooled serum from soybean-sensitive individuals and used to identify 6 IgE binding regions at amino acid sequence positions 1-23, 57-111, 169-215, 249-271, 329-383, and 449-471 of SEQ ID NO. 109 (R1-R6 shaded in FIG. 79).

### Example 21

### Characterization of Soybean Allergen β-Conglycinin, a Member of the Vicilin Family of Seed Storage Proteins

### 21.1 Introduction

A GenBank search for amino acid homology to Ara h 1 identified a 47% amino acid sequence homology to a soybean vicilin family member, β-conglycinin. The α-chain of β-conglycinin (GenBank AAB01374, SEQ ID NO. 110) was selected for linear epitope analysis using soybean- and peanut-specific serum from sensitive individuals.

### 21.2 Methods and Results

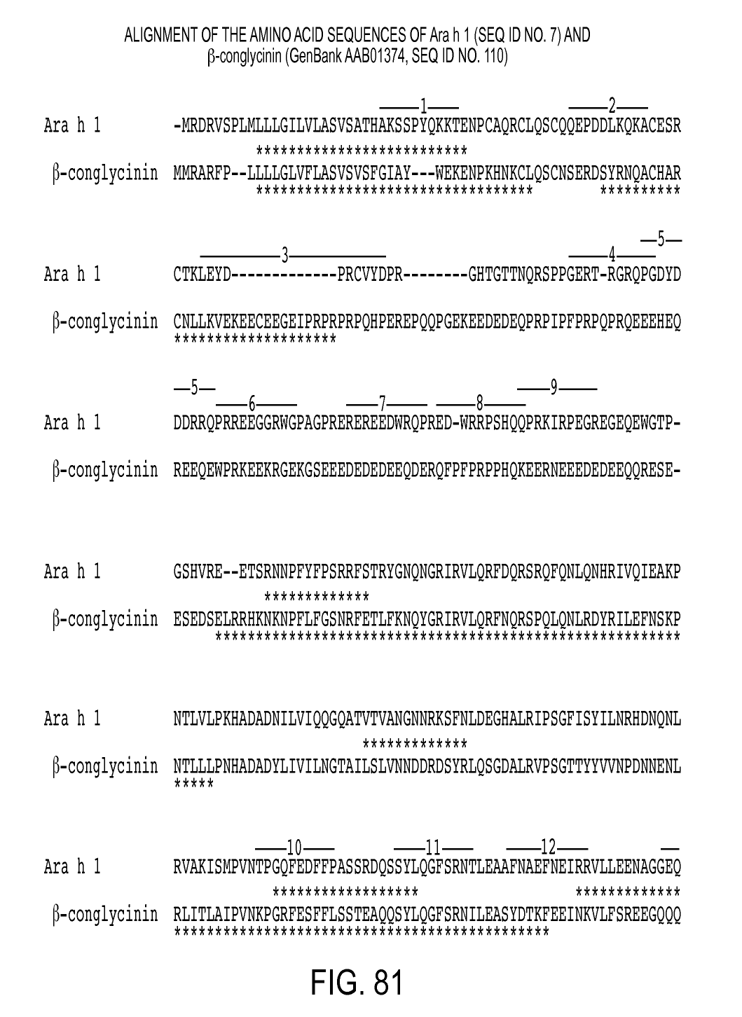

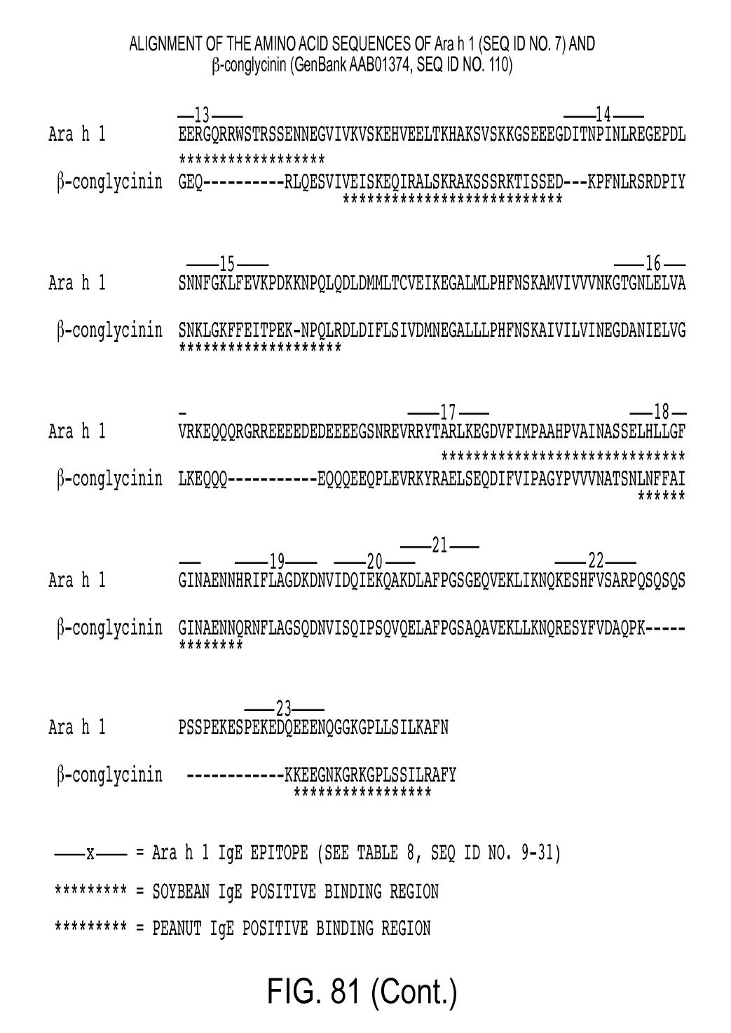

A set of 15 mers offset by 8 amino acids were prepared that together spanned the amino acid sequence of the α-chain of β-conglycinin (SEQ ID NO. 110, FIG. 81). A SPOTS membrane coated with the peptides was blocked, incubated with a serum pool taken from soybean-sensitive individuals, washed, incubated with radiolabeled anti-IgE, washed and exposed to X-ray film. Developed films were then assessed for IgE-positive binding regions. Following identification of soybean IgE-positive binding regions, the SPOTS membrane was stripped according to manufacturer's instructions and re-probed with a serum pool taken from peanut-sensitive individuals. For each region identified, 10 mers offset by 2 amino acids were synthesized and analyzed to obtain more specific IgE-binding epitope sequences.

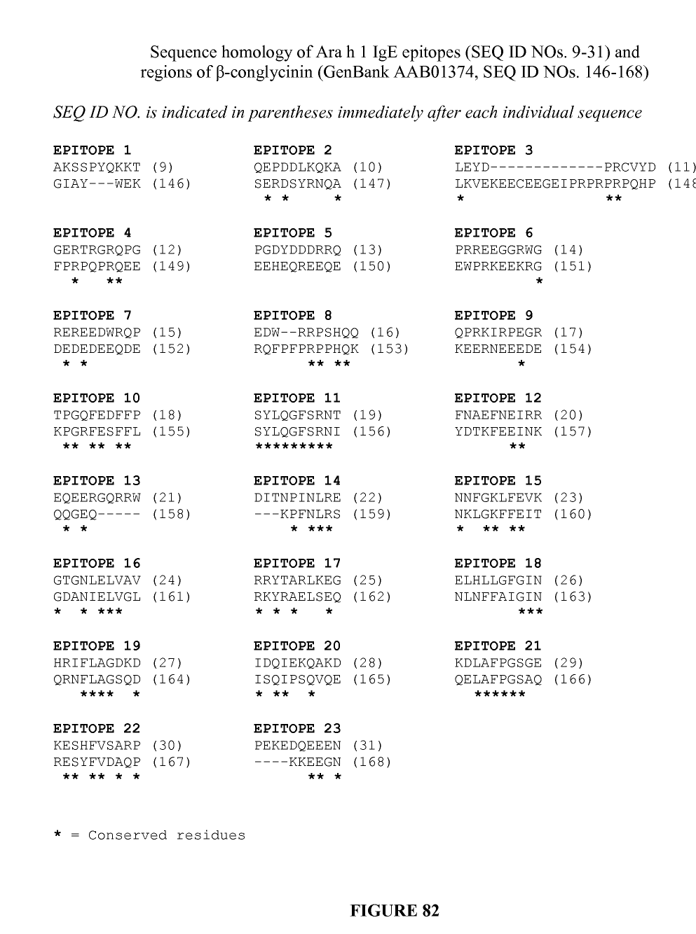

As shown in FIG. 81, our results identified 4 common IgE-binding regions (i.e., regions that are both peanut and soybean positive IgE-binding regions); however, there were 2 unique soybean positive IgE-binding regions (amino acid sequences 269-281 and 359-379 of SEQ ID NO. 110) and 5 unique peanut positive IgE binding regions (amino acid sequences 48-77, 207-250, 382-409, 422-439, and 595-612 of SEQ ID NO. 110). Finally, the homology between the peanut positive-IgE binding epitopes of Ara h 1 (SEQ ID NO. 9-31) and the corresponding regions of β-conglycinin that were identified in the alignment of FIG. 81 is highlighted in FIG. 82.

### Example 22

### Cloning of a 51 kD Allergen from the Seed Cotyledon of Soybean

### 22.1 Introduction

We have identified a seed maturation protein using serum from soybean-sensitive individuals for screening a soybean seed cotyledon cDNA expression library. Five clones representing two 1,500 and three 1,400 bp fragments were isolated using this technology. Nucleotide sequence homology of clone 3a (1,500 bp) and 4a (1,400 bp) revealed them to have shared identity to a 51 kd maturation protein functioning as a desiccant protection protein in maturing soybean seeds. Here we report the first identification of this molecule as an IgE-binding protein.

### 22.2 Methods and Results

Soybean seeds, (Glycinus max) Hutchinson variety, were obtained from a local health food store, frozen in liquid nitrogen, ground to a fine powder, and the RNA extracted using the method of Nedergaard et al (Mol. Immunol. 29:703, 1992). Briefly, 2 g frozen seed powder was added to 10 ml buffer (250 mM sucrose, 200 mM Tris-HCl, pH 8.0, 200 mM KCl, 30 mM MgCl₂, 2% polyvinylpyrrokidone-40 and 5 mM 2-mercaptoethanol) and equilibrated with 10 ml fresh phenol (4° C.). The suspension was homogenized and 10 ml of chloroform added with shaking for 5 minutes at room temperature. Phases were separated by centrifugation, 10,000 g for 20 minutes at 4° C. and the aqueous phase transferred to a clean test tube and extracted 2× with equal volumes of chloroform/phenol. Nucleic acids were precipitated with sodium acetate/ethanol at −20° C. overnight. The precipitates were collected by centrifugation at 13,000 g for 20 minutes at 4° C., washed with 70% ethanol and dried. Samples run in parallel were pooled in water and made 3 M in LiCl, and the RNA precipitated for 4 hours at −20° C. The precipitate was collected by centrifugation outlined above and resuspended in distilled water. Fifty microliters of the RNA suspension was withdrawn for OD260/280 measurements and the RNA analyzed by agarose gel electrophoresis. Three aliquots representing a total of approximately 3 mg total RNA was sent to STRATAGENE for purification of mRNA and the preparation of a Uni-Zap XR custom library.

The expression custom library was screened with serum from soybean-sensitive individuals and positive clones were subcloned to homogeneity with respect to IgE-binding. Five clones were isolated from an initial screen and the plasmids purified from LB/amphcilin broth cultures using an Ameresco kit. The plasmid DNA from each clone was PCR amplified and analyzed in agarose gels. Two plasmid preparations had relative by of approximately 1,300 (clone 3a) and the remaining three 1,400 (clone 4a).

While clone 3a showed 88.2% identity over a 76 bp overlap (between nucleotides 187-262 of clone 3a, data not shown) with the nucleotide sequence of the Shi-Shi 51 kd seed maturation from Glycinus max, clone 4a showed 96.5% identity over a 114 bp overlap (between nucleotides 423-536 of clone 4a, data not shown).

### Example 23

### Characterization of Soybean Allergen Gly m Bd 30K

As was described in Examples 15 and 19, soybean proteins share a large number of cross-reacting proteins with other members of the legume family; however, studies have demonstrated that soy-allergic patients rarely react clinically to other members of the legume family. An IgE-binding protein Gly m Bd 30K (Glycine max band) with a molecular weight of 30 kD has been identified in soybean extracts by SDS-PAGE/IgE-immunoblot analysis (Ogawa et al., 1991, supra and Ogawa et al., Biosci. Biotech. Biochem. 57:1030-1033, 1993). This monomeric allergen was shown to have N-terminal amino acid sequence identical to that of a seed vacuole, 34 kd protein (P34) (Kalinski et al., J. Biol. Chem. 265:13843-13848, 1990 and Kalinski et al., J. Biol. Chem. 267:12068-12076, 1992). We used pooled serum from clinically soybean-sensitive patients to identify IgE-binding sites in Electron Microscopy (EM) sections of soybean seeds and to determine IgE-specific epitopes in the protein. IgE-binding to EM sections of soybean seeds showed intense staining throughout the vacuolar bodies localizing the allergen in seed cotyledons. IgE epitope mapping revealed 10 regions of IgE-binding activity using an overlapping peptide strategy of 15 mers offset by 8 throughout the P34 sequence. Peptide synthesis of 10 mers offset by 2 amino acids revealed 16 distinct linear epitopes, 9 of which were mapped to the mature protein. Individual patient serum and amino acid substitutions of immunodominant epitopes will be used to identify the core amino acids necessary for IgE-binding.

### Example 24

### Identification and Characterization of a 50 kD Wheat Allergen

Wheat is a major cause of food hypersensitivity, but information concerning specific wheat allergens is limited. The focus of this study was to isolate and characterize the clinically relevant allergens in wheat protein. Whole wheat extracts were prepared (1:10 w/v in PBS). The extracts (1 mg/ml) were separated with 10% SDS-PAGE and 10 Coomassie-stained protein bands (range: 16-65 kd) were obtained. The crude wheat extract was separated with a stepwise salt gradient (0-1.5 M NaCl) on a Mono-Q/FPLC anion exchange column resulting in two major protein peaks (Peak I and II). SDS-PAGE (10%) analysis of Peak I revealed protein bands ranging from 16-10 kd while Peak II contained wheat proteins greater than 45 kd. A 50 kd protein band was isolated from Peak II using 8% preparative cell-SDS-PAGE. An ELISA was designed to screen for serum-specific IgE antibodies to the isolated 50 kd wheat protein band. Seven wheat-allergic patients (range: 1-17 years, median: 2 years) confirmed by prick skin tests blinded challenges and/or convincing histories of anaphylaxis after wheat ingestion were studied. Sera from 3 patients without food allergy served as controls. Four of the 7 patient sera had significant IgE binding to the 50 kd wheat protein in the ELISA when compared to a negative control (range: 160-1200%, median: 365%). IgE immunoblotting studies revealed that serum-specific IgE antibodies from all the wheat-allergic patients bound to this 50 kd protein. No binding was demonstrated with normal control sera. These studies demonstrate that serum IgE antibodies from wheat-allergic pediatric patients binds a 50 kd protein from crude wheat extracts.

### Example 25

### Identification and Characterization of Walnut Allergens

Walnut allergies affect about 0.6% of the population. Clinical symptoms can be severe. Both English (Juglans regia) and Black (Juglans nigra) walnuts are used in food. Two allergens from English walnuts named Jug r 1 and Jug r 2, have been identified. Jug r 1, described by Teuber et al., JACI 101:807-814, 1998, is a 2S albumin seed storage protein recognized by 68% of walnut-sensitive patient sera. Jug r 2, described by Teuber et al., JACI 104:1311-1320, 1998, is a vicilin-like seed storage protein, and is recognized by 60% of walnut-sensitive patient sera.

In this study two allergens from Black walnuts named Jug n 1 and Jug n 2 were cloned and their IgE epitopes were determined by following the principles and methods that were used in Examples 4, 11, and 17 to characterize the peanut allergens Ara h 1, Ara h 2, and Ara h 3. Jug n 1 is 96% identical to Jug r 1 and Jug n 2 is 98% identical to Jug r 2. The IgE epitopes that were identified are listed in Table 33 (Jug n 1) and Table 34 (Jug n 2).

TABLE 33
Jug n 1 IgE-binding epitopes

| SEQ | | | |
|---|---|---|---|
| ID | | Amino acid | Jug n 1 |
| NO. | Epitope | sequence | positions |
| 111 | 1 | CIFHTFSLT | 7-15 |
| 112 | 2 | VALLFVAN | 27-34 |
| 113 | 3 | RRRGEGCQ | 56-63 |
| 114 | 4 | NLNHCQYY | 71-78 |
| 115 | 5 | QHFRQCCQ | 95-102 |
| 116 | 6 | QCEGLRQA | 112-119 |
| 117 | 7 | RGEEMEEM | 134-142 |
| 118 | 8 | KECGISSQR | 151-159 |

TABLE 34
Jug n 2 IgE-binding epitopes

| Epitope | Jug n 2 positions |
|---|---|
| 1 | 11-19 |
| 2 | 23-31 |
| 3 | 35-43 |
| 4 | 73-81 |
| 5 | 89-97 |
| 6 | 122-130 |
| 7 | 140-148 |
| 8 | 178-186 |
| 9 | 240-248 |
| 10 | 262-270 |
| 11 | 292-300 |
| 12 | 370-378 |
| 13 | 401-409 |
| 14 | 447-454 |
| 15 | 479-487 |
| 16 | 511-519 |
| 17 | 531-539 |

### Example 26

### IgE Fab cDNA Library to Peanut Allergens

### 26.1 Introduction

In order to quantitatively characterize the interaction of human IgE antibodies with the corresponding epitopes that they recognize a combinatorial IgE library was constructed from a patient with documented peanut hypersensitivity. cDNAs encoding the heavy and light chains of IgE were obtained by RT-PCR using mRNA isolated from the patient's peripheral blood lymphocytes. A series of ten primers were used to amplify the seven light chain genes and ten primers were used to amplify the 8 heavy chain genes of IgE and each reaction was used to develop a separate library in the expression vector pCOMb3H. The inserts from these libraries were then randomly combined to produce a phage display library of 1.1 and 10⁸ primary phage. Phage which recognized the major peanut allergen Ara h 2 were selected by attaching the purified allergen to microtiter wells and then adding the phage library to this mix under conditions which promote antibody/epitope interactions. After extensively washing the plates, bound phage were eluted and the process was repeated in order to ensure specificity of binding. After each selection the titer of Ara h 2 specific phage increased indicating that the phage were specific for Ara h 2. Forty clones were selected at random and characterized. Individually, each clone contained a heavy and light chain insert that verified that binding of the allergen was most likely through a Fab fragment. Sequence analysis and epitope specificity of each phage is currently underway to determine which of the 10 Ara h 2 epitopes are recognized by these Fabs.

### 26.2 Methods and Results

Construction of a Recombinant IgE Fab Library

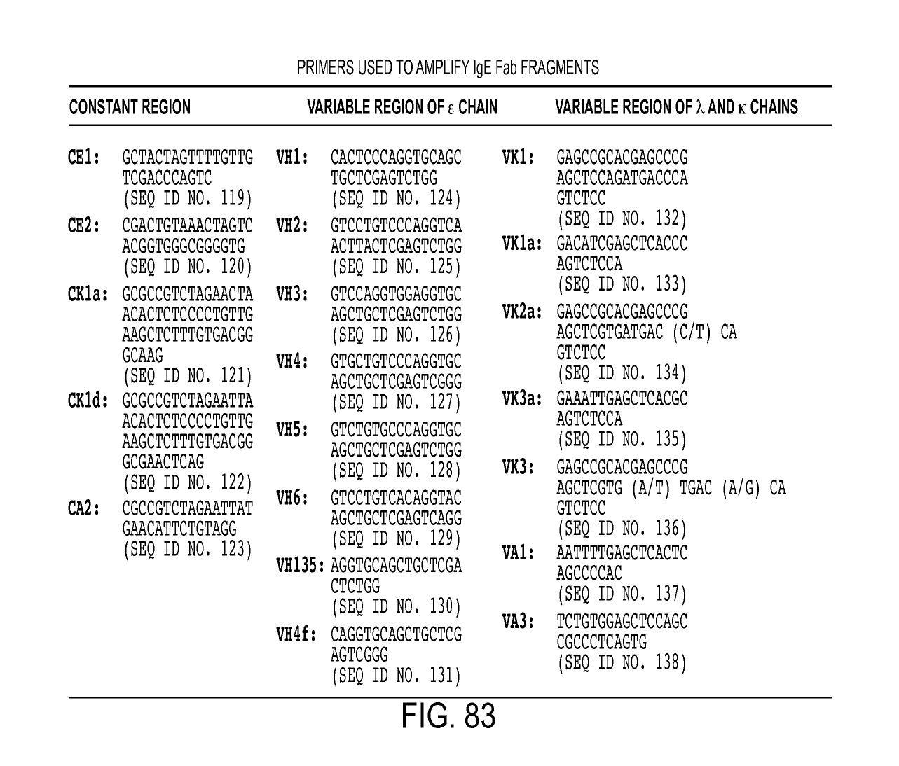

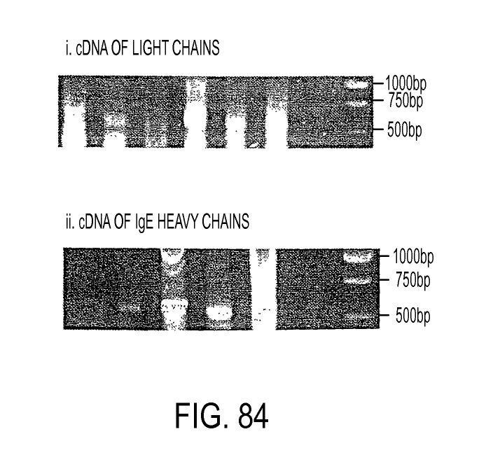

Total RNA was isolated from PBMCs of a peanut allergic patient and the primers in FIG. 83 were utilized to amplify the IgE heavy and light chains. Portions of each reaction were electrophoresed on agarose gels and analyzed for the presence of a primer specific amplification product (FIG. 84).

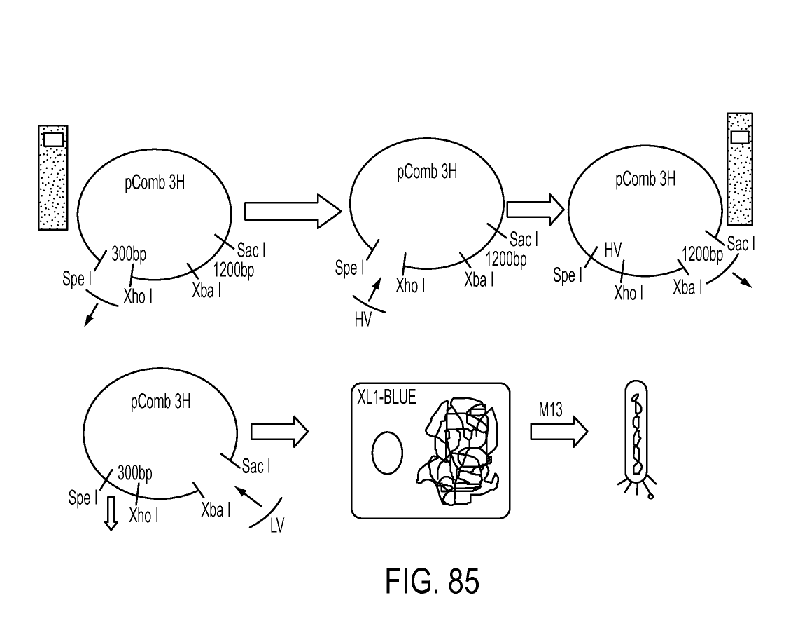

Expression constructs were then prepared as illustrated in FIG. 85. The expression vector pComb3H was first digested with SpeI and XhoI to release an approximately 300 bp vector fragment. Heavy chain fragments (HV) were then ligated into this site. pComb3H vectors containing the heavy chain fragments were digested with SacI and XbaI and the light chain fragments were then ligated into this site. The recombinant vectors containing both heavy and light chain fragments were used to transform E. coli XL1-blue cells. A phage display library of ˜1.1×10⁸ clones was obtained.

Analysis of Phage from the Recombinant IgE Fab Library

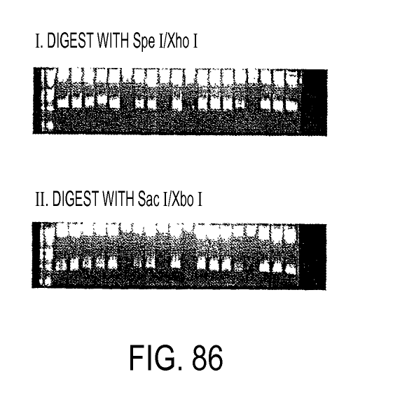

Nineteen clones were randomly picked from the recombinant IgE Fab library and analyzed by restriction enzyme digestion and agarose gel electrophoresis. Heavy chain inserts were released by digestion with SpeI and XhoI and light chain inserts were released by digestion with SacI and XboI. Fifteen out of the nineteen clones (i.e., 79%) contained both heavy and light chain inserts (FIG. 86).

### Selection of Clones Producing Peanut Allergen-Specific IgE Fab Fragments

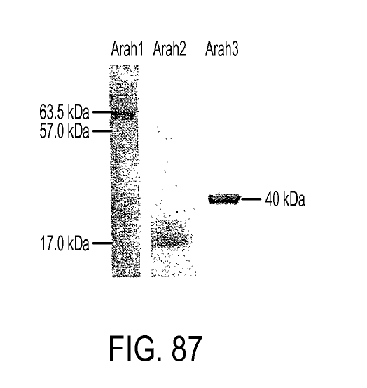

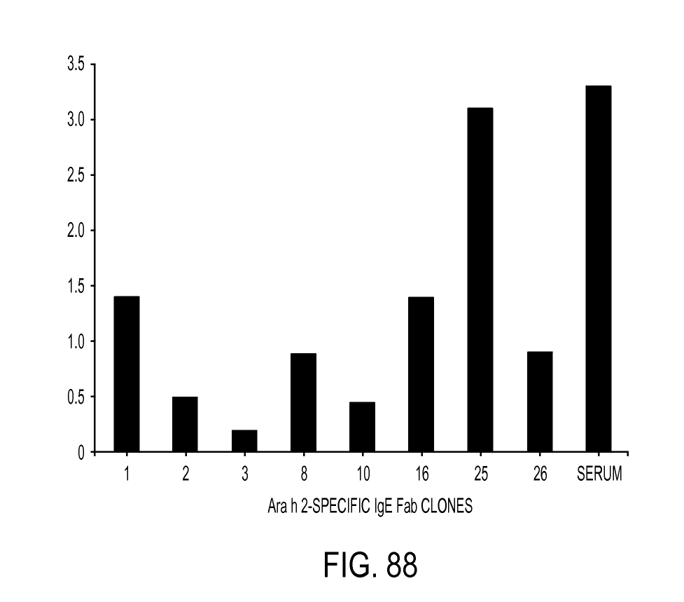

Peanut allergens Ara h 1 and Ara h 2 were purified from defatted peanut powder while Ara h 3 was expressed recombinantly and purified using affinity chromatography (see FIG. 87). Using purified peanut allergens Ara h 1, Ara h 2, and Ara h 3 three pools of phage were selected from the recombinant IgE Fab library. Specifically of the phage selected with Ara h 2 was determined by running an ELISA assay using IgE Fab fragments produced by the selected clones (clones 1, 2, 3, 8, 10, 16, 25, and 26) and then detecting the amount of IgE Fab bound with an anti-human IgE reporter antibody. IgE bound to Ara h 2 from the serum of a peanut sensitive patient is included for comparison. Results are shown in FIG. 88 expressed as a fold increase over binding when no primary antibody is used.

### 26.3 Conclusion

A display phage cDNA library of human IgE was constructed from a peanut sensitive patient mRNA by using RT-PCR and the pComb3H vector. The titer of the original library was ˜1.1×10⁸ pfu. 79% of clones contained in the library have both heavy chain and light chain cDNA inserts. Three pools of recombinant clones were selected from the IgE Fab library using purified peanut allergens Ara h 1, Ara h 2, and Ara h 3. Selected Ara h 2-specific IgE Fab clones were tested in an ELISA assay and shown to bind Ara h 2a similar level as IgE found in the serum of a peanut sensitive patient. These Fab fragments are important tools for studying the affinity of antibody/epitope interactions and for the development of novel immunotherapeutics for the treatment of peanut allergic patients.

### Example 27

### Evaluation of Heat Killed E. Coli Expressing Modified Ara h 1, 2, and 3 for the Desensitization of Peanut-Allergic Mice

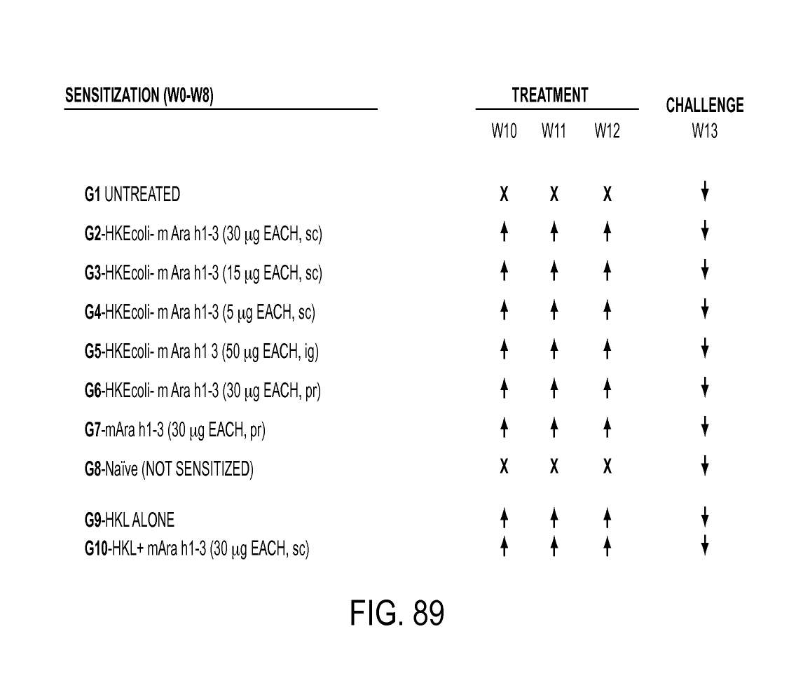

Ten groups of mice (G1-G10, FIG. 89) were used for in vivo desensitization experiments. The 5 week old female C3H/HeJ mice (approx. 10 per group) were first sensitized with crude peanut extract and cholera toxin over a period of 8 weeks (W0-W8). The mice were then treated according to ten different desensitization protocols at weeks 10, 11, and 12 (W10-W12). Finally the mice were challenged with crude peanut extract at week 13 (W13). G1 mice were sham desensitized at weeks 10-12, i.e., treated with a placebo. G2, G3, and G4 mice were desensitized via the subcutaneous (sc) route with Heat Killed E. coli (HKEc) expressing modified Ara h 1, 2, and 3 (30, 15, and 5 μg of each, respectively). G5 mice were desensitized via the intragastric (ig) route with Heat Killed E. coli (HKEc) expressing modified Ara h 1, 2, and 3 (50 μg of each). G6 mice were desensitized via the rectal (pr) route with Heat Killed E. coli (HKEc) expressing modified Ara h 1, 2, and 3 (30 μg of each). G7 mice were desensitized via the rectal (pr) route with modified Ara h 1, 2, and 3 (30 μg of each) alone. G8 mice were naïve, i.e., were not sensitized with crude peanut extract and cholera toxin during weeks 0-8. G9 mice were desensitized via the subcutaneous (sc) route with Heat Killed Listeria (HKL) alone. G10 mice were desensitized via the subcutaneous (sc) route with Heat Killed Listeria (HKL) expressing modified Ara h 1, 2, and 3 (30 μg of each).

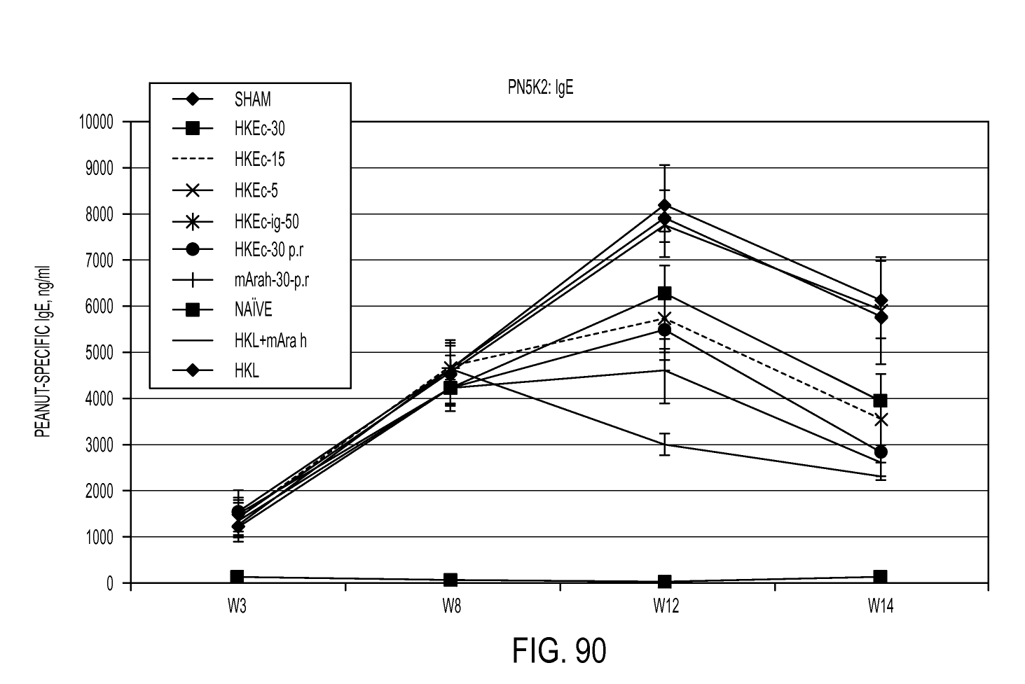

The average IgE levels (ng/ml) at weeks 3, 8, 12, and 14 for the ten groups of mice (G1-G10) are shown in FIG. 90. As compared to the sham desensitized mice, the increase in IgE levels during the desensitization period (W10-W12) was dramatically reduced in all the desensitized groups except for the mice that were treated via the intragastric (ig) route with Heat Killed Listeria alone (G9) or Heat Killed E. coli expressing modified allergens.

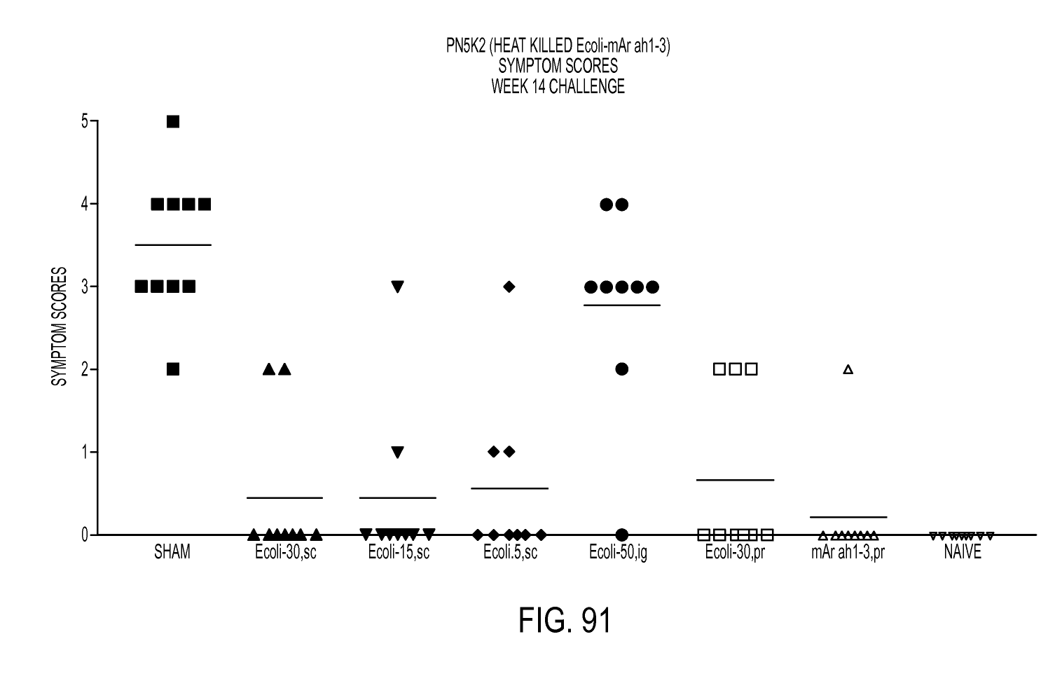

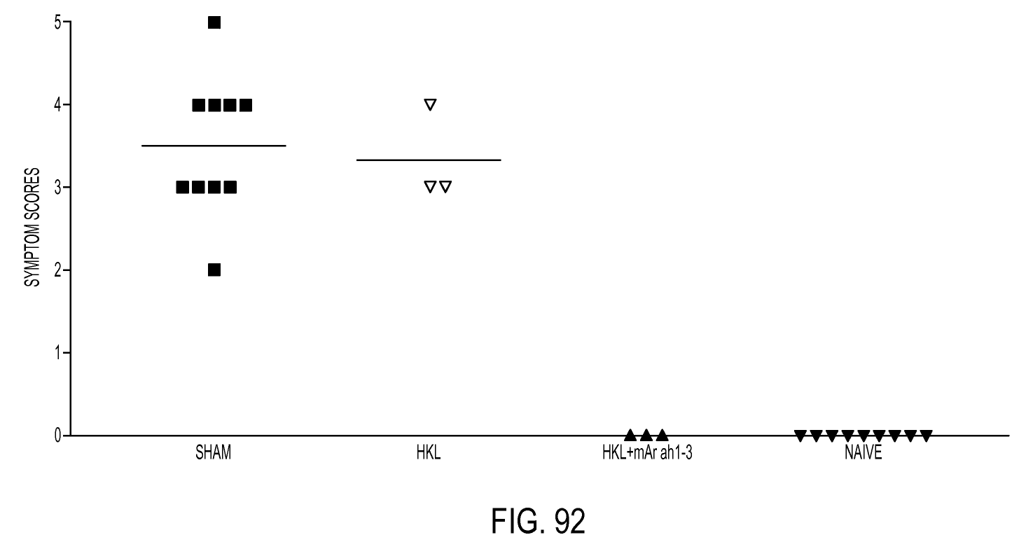

The individual (symbols) and average (solid line) symptom scores (0-5) at week 14 for the ten groups of mice are compared in FIGS. 91 and 92. The improvement in symptom scores parallel the IgE data with dramatic improvements (from an average score of 3.5 to average score scores of 0.4 or less) except for the group of mice that were treated with Heat Killed Listeria only (average score of 3.4) or via the intragastric (ig) route with Heat Killed E. coli expressing modified allergens (average score of 3.0).

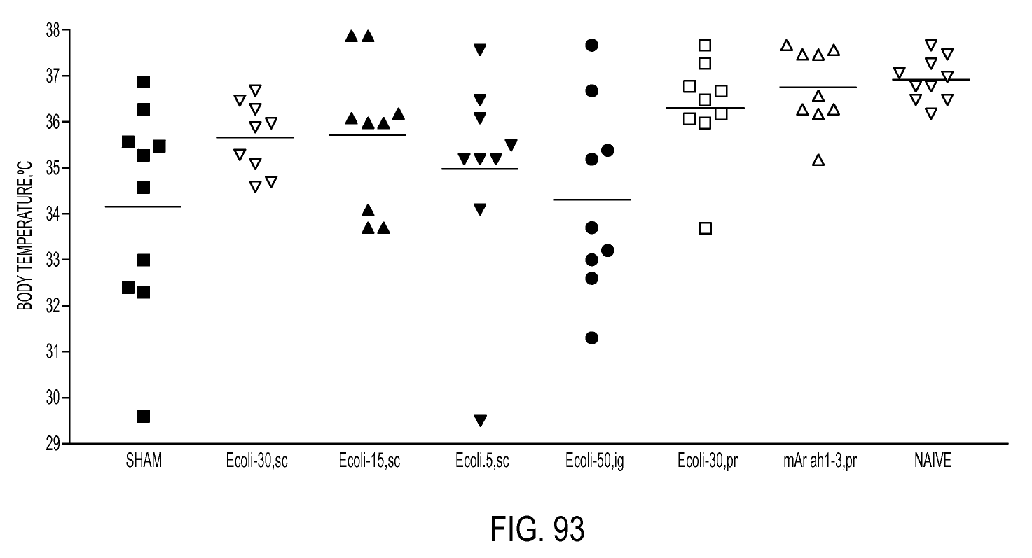

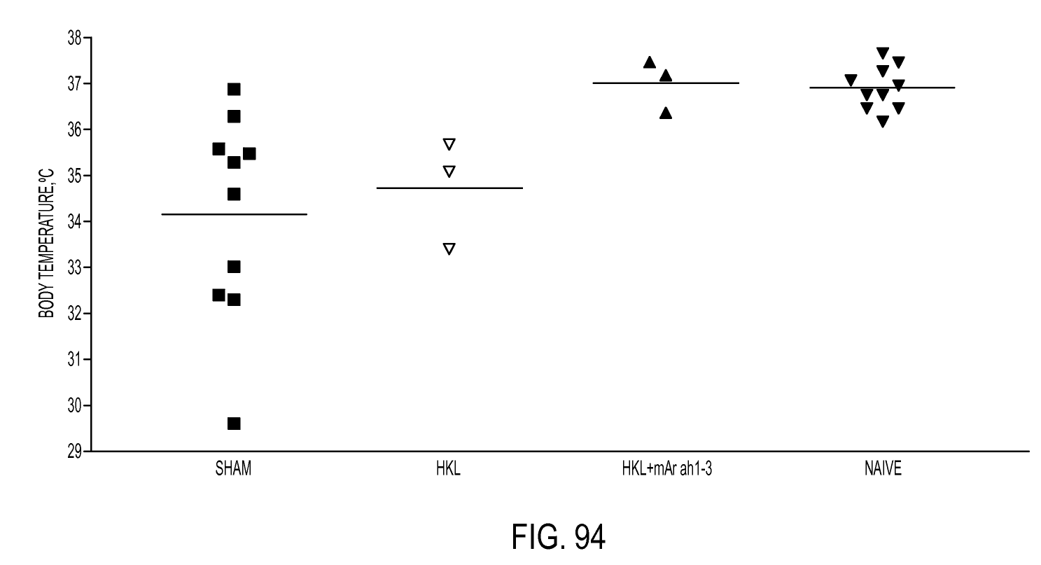

The individual (symbols) and average (solid line) body temperatures (° C.) at week 14 for the ten groups of mice are compared in FIGS. 93 and 94. The trend in average body temperature correlates well with the results in FIGS. 90-92. In all treated groups the average body temperatures at week 14 is higher than in the sham sensitized group. However, the increase is smallest for the group of mice that were treated with Heat Killed Listeria only or via the intragastric (ig) route with Heat Killed E. coli expressing modified allergens.

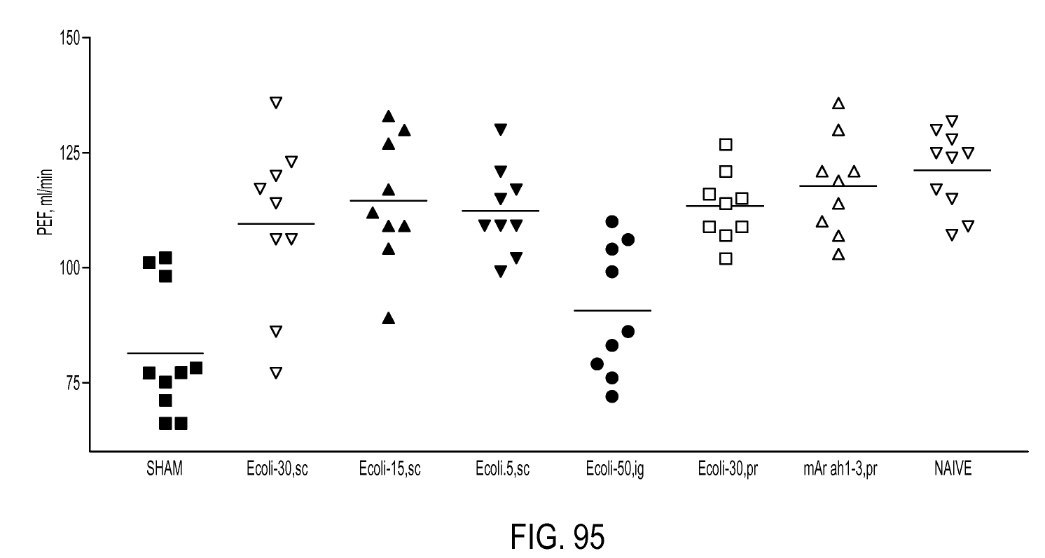

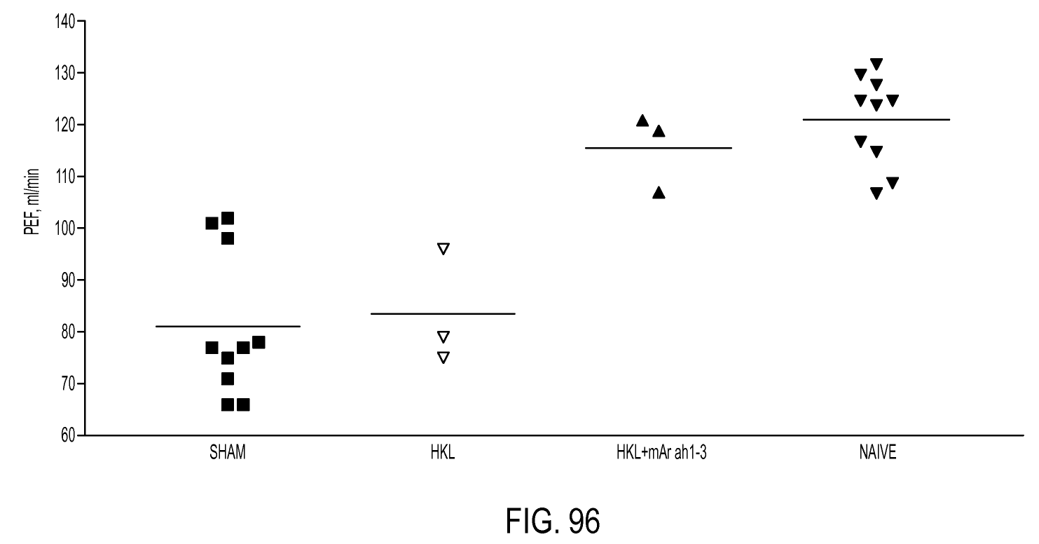

The individual (symbols) and average (solid line) airway responses (peak respiratory flow in ml/min) at week 14 for the ten groups of mice are compared in FIGS. 95 and 96. Peak flow values are dramatically improved in most groups except for the group of mice that were treated with Heat Killed Listeria only or via the intragastric (ig) route with Heat Killed E. coli expressing modified allergens.

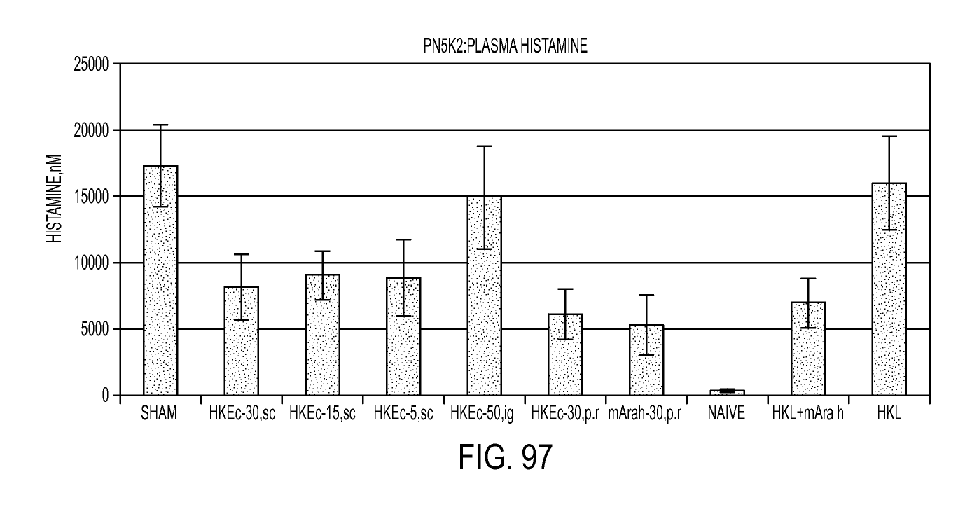

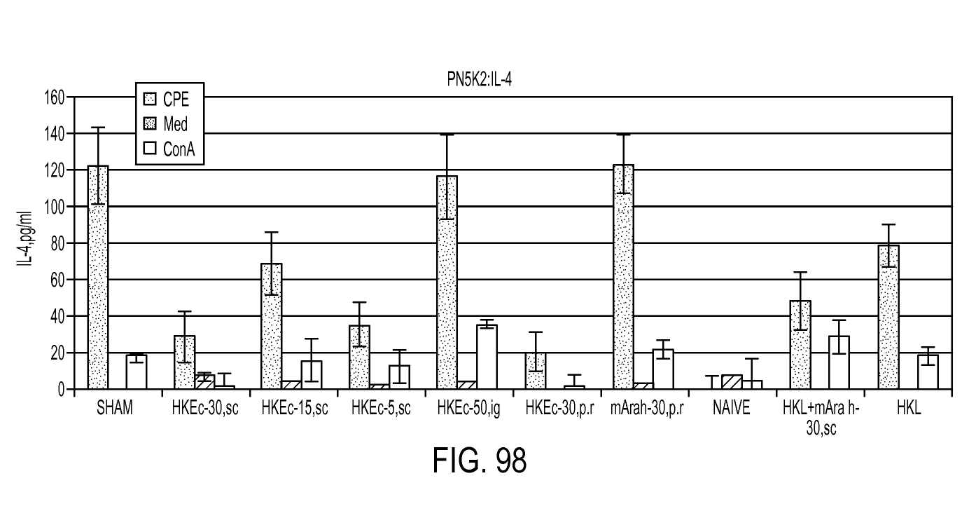

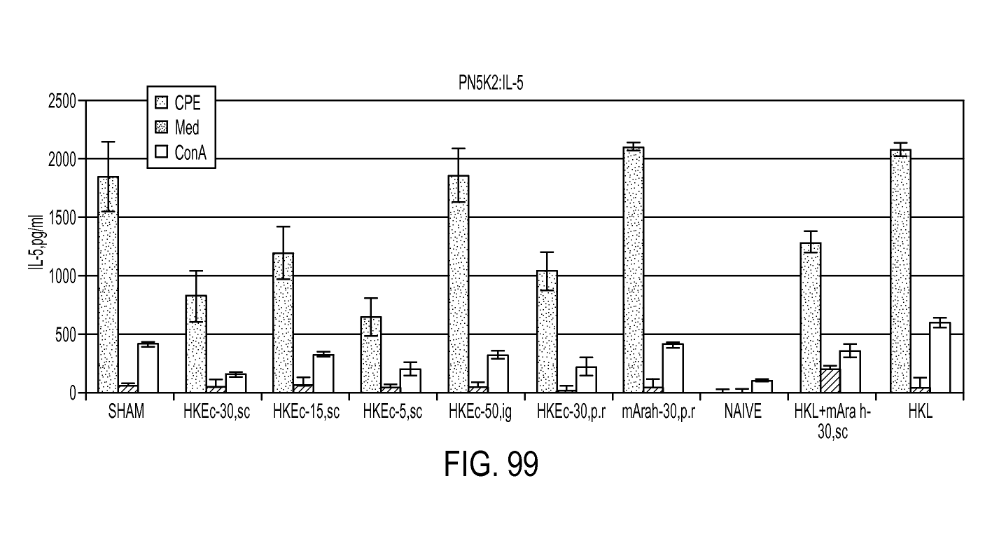

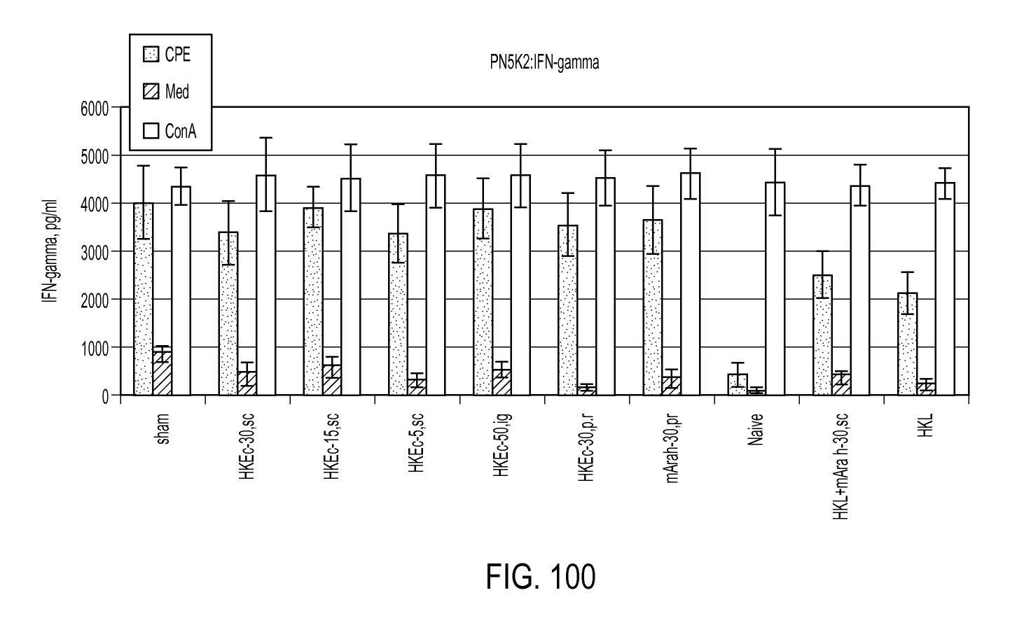

FIGS. 97, 98, 99, and 100 compare the plasma histamine (nM), IL-4 (pg/ml), IL-5 (pg/ml), and IFNγ (pg,/ml) concentrations at week 14 for the ten groups of mice (G1-G10).

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the claims that follow the appendices.

APPENDIX 1
WEED POLLENS

| | | | | |
|---|---|---|---|---|
| | SYSTEMATIC AND | | SEQUENCE | ACCESSION NO. |
| ALLERGEN SOURCE | ORIGINAL NAMES | MW (KD) | DATA | OR REFERENCES |

Asterales

| | | | | |
|---|---|---|---|---|
| Ambrosia | Amb a 1; antigen E | 38 | C | 8, 20 |
| artemisiifolia | Amb a 2; antigen K | 38 | C | 8, 21 |
| (short ragweed) | Amb a 3; Ra3 | 11 | C | 22 |
| | Amb a 5; Ra5 | 5 | C | 11, 23 |
| | Amb a 6; Ra6 | 10 | C | 24, 25 |
| | Amb a 7; Ra7 | 12 | P | 26 |
| | Amb a ? | 11 | C | 27 |
| Ambrosia trifida | Amb t 5; Ra5G | 4.4 | C | 9, 10, 28 |
| (giant ragweed) | | | | |
| Artemisia vulgaris | Art v 1 | 27-29 | C | 28A |
| (mugwort) | Art v 2 | 35 | P | 29 |
| Helianthus annuus | Hel a 1 | 34 | — | 29a |
| (sunflower) | Hel a 2; profilin | 15.7 | C | Y15210 |
| Mercurialis annua | Mer a 1; profilin | 14-15 | C | Y13271 |

APPENDIX 2
GRASS POLLENS

| | | | | |
|---|---|---|---|---|
| | SYSTEMATIC AND | | SEQUENCE | ACCESSION NO. |
| ALLERGEN SOURCE | ORIGINAL NAMES | MW (KD) | DATA | OR REFERENCES |

Poales

| | | | | |
|---|---|---|---|---|
| Cynodon | Cyn d 1 | 32 | C | 30, S83343 |
| dactylon | Cyn d 7 | | C | 31, X91256 |
| (Bermuda grass) | Cyn d 12; profilin | 14 | C | 31a, Y08390 |
| Dactylis | Dac g 1; AgDg1 | 32 | P | 32 |
| glomerata | Dac g 2 | 11 | C | 33, S45354 |
| (orchard grass) | Dac g 3 | | C | 33a, U25343 |
| | Dac g 5 | 31 | P | 34 |
| Holcus lanatus | Hol l 1 | | C | Z27084, Z68893 |
| (velvet grass) | | | | |
| Lolium perenne | Lol p 1; group I | 27 | C | 35, 36 |
| (rye grass) | Lol p 2; group II | 11 | C | 37, 37a, X73363 |
| | Lol p 3; group III | 11 | C | 38 |
| | Lol p 5; Lol p IX, | 31/35 | | 34, 39 |
| | Lol p Ib | | | |
| | Lol p 11; trypsin | 16 | | 39a |
| | inh. Related | | | |
| Phalaris aquatica | Pha a 1 | | C | 40, S80654 |
| (canary grass) | | | | |
| Phleum pratense | Phl p 1 | 27 | C | X78813 |
| (timothy) | Phl p 2 | | C | 41, X75925 |
| | Phl p 4 | | P | 41A |
| | Phl p 5; Ag25 | 32 | C | 42 |
| | Phl p 6 | | C | 43, Z27082 |
| | Phl p 12; profilin | | C | 44, X77583 |
| | Phl p 13; | 55-60 | C | AJ238848 |
| | polygalacturonase | | | |
| Poa pratensis | Poa p 1; group I | 33 | P | 46 |
| (Kentucky blue | Poa p 5 | 31/34 | C | 34, 47 |
| grass) | | | | |
| Sorghum | Sor h 1 | | C | 48 |
| halepense | | | | |
| (Johnson grass) | | | | |

APPENDIX 3
TREE POLLENS

| | | | | |
|---|---|---|---|---|
| | SYSTEMATIC AND | | SEQUENCE | ACCESSION NO. |
| ALLERGEN SOURCE | ORIGINAL NAMES | MW (KD) | DATA | OR REFERENCES |

Fagales

| | | | | |
|---|---|---|---|---|
| Alnus glutinosa | Aln g 1 | 17 | C | S50892 |
| (alder) | | | | |
| Betula verrucosa | Bet v 1 | 17 | C | see list of |
| (birch) | | | | isoallergens |
| | Bet v 2; profilin | 15 | C | M65179 |
| | Bet v 3 | 8 | C | X79267 |
| | Bet v 4 | | C | X87153/S54819 |
| | Bet v 5; isoflavone | 33.5 | C | AF135127 |
| | reductase homologue | | | |
| Carpinus betulus | Car b 1 | 17 | C | 51 |
| (hornbeam) | | | | |
| Castanea sativa | Cas s 1; Bet v 1 homologue | 22 | P | 52 |
| (chestnut) | | | | |
| Corylus avelana | Cor a 1 | 17 | C | 53 |
| (hazel) | | | | |
| Quercus alba | Que a 1 | 17 | P | 54 |
| (white oak) | | | | |
| Cryptomeria | Cry j 1 | 41-45 | C | 55, 56 |
| japonica (sugi) | Cry j 2 | | C | 57, D29772 |
| Juniperus ashei | Jun a 1 | 43 | P | P81294 |
| (mountain cedar) | Jun a 3 | 30 | P | P81295 |
| Juniperus | Jun o 2; calmodulin-like | 29 | C | AF031471 |
| oxycedrus | | | | |
| (prickly juniper) | | | | |
| Juniperus | Jun s 1 | 50 | P | 58 |
| sabinoides | | | | |
| (mountain cedar) | | | | |
| Juniperus | Jun v 1 | 43 | P | P81825 |
| virginiana | | | | |
| (eastern red cedar) | | | | |

Oleales

| | | | | |
|---|---|---|---|---|
| Fraxinus excelsior | Fra e 1 | 20 | P | 58A |
| (ash) | | | | |
| Ligustrum vulgare | Lig v 1 | 20 | P | 58A |
| (privet) | | | | |
| Olea europea | Ole e 1 | 16 | C | 59, 60 |
| (olive) | Ole e 2; profilin | 15-18 | C | 60A |
| | Ole e 3 | 9.2 | | 60B |
| | Ole e 4 | 32 | P | P80741 |
| | Ole e 5; superoxide | 16 | P | P80740 |
| | dismutase | | | |
| | Ole e 6 | 10 | C | U86342 |
| Syringa vulgaris | Syr v 1 | 20 | P | 58A |
| (lilac) | | | | |

APPENDIX 4
MITE ALLERGENS

| | SYSTEMATIC AND | | SEQUENCE | ACCESSION NO. |
|---|---|---|---|---|
| ALLERGEN SOURCE | ORIGINAL NAMES | MW (KD) | DATA | OR REFERENCES |
| Acarus siro | Aca s 13; fatty | 14* | C | AJ006774 |
| (mite) | acid-bind. prot. | | | |
| Blomia tropicalis | Blo t 5 | | C | U59102 |
| (mite) | Blo t 12; Bt11a | | C | U27479 |
| | Blo t 13; Bt6 fatty | | C | U58106 |
| | acid-binding prot | | | |
| Dermatophagoides | Der p 1; antigen P1 | 25 | C | 61 |
| pteronyssinus | Der p 2 | 14 | C | 62 |
| (mite) | Der p 3; trypsin | 28/30 | C | 63 |
| | Der p 4; amylase | 60 | C | 64 |
| | Der p 5 | 14 | P | 65 |
| | Der p 6; chymotrypsin | 25 | C | 66 |
| | Der p 7 | 22-28 | C | 67 |
| | Der p 8; glutathione | | P | 67A |
| | transferase | | | |
| | Der p 9; collagenolytic | | C | 67B |
| | serine prot. | | | |
| | Der p 10; tropomyosin | 36 | | Y14906 |
| Dermatophagoides | Der m 1 | 25 | P | 68 |
| microceras (mite) | | | | |
| Dermatophagoides | Der f 1 | 25 | C | 69 |
| farinae (mite) | Der f 2 | 14 | C | 70, 71 |
| | Der f 3 | 30 | C | 63 |
| | Der f 10; tropomyosin | | C | 72 |
| Lepidoglyphus | Lep d 2.0101 | 15 | C | 73, 74, 75 |
| destructor | Lep d 2.0102 | 15 | C | 75 |
| (storage mite) | | | | |

APPENDIX 5
ANIMAL ALLERGENS

| | | | | |
|---|---|---|---|---|
| | SYSTEMATIC AND | | SEQUENCE | ACCESSION NO. |
| ALLERGEN SOURCE | ORIGINAL NAMES | MW (KD) | DATA | OR REFERENCES |

ANIMALS

| | | | | |
|---|---|---|---|---|
| Bos domesticus | Bos d 2; Ag3, lipocalin | 20 | C | 76, L42867 |
| (domestic cattle) | Bos d 4; | 14.2 | C | M18780 |
| (see also foods) | alpha-lactalbumin | | | |
| | Bos d 5 | 18.3 | C | X14712 |
| | beta-lactoglobulin | | | |
| | Bos d 6; serum albumin | 67 | C | M73993 |
| | Bos d 7; | 160 | | 77 |
| | immunoglobulin | | | |
| | Bos d 8; caseins | 20-30 | | 77 Bos d 8; |
| | | | | caseins |
| Canis familiaris | Can f 1 | 25 | C | 78, 79 |
| (dog) | Can f 2 | 27 | C | 78, 79 |
| | Can f ?; albumin | | C | S72946 |
| Equus caballus | Equ c 1; lipocalin | 25 | C | U70823 |
| (domestic horse) | Equ c 2; lipocali | 18.5 | P | 79A, 79B |
| Felis domesticus | Fel d 1; cat-1 | 38 | C | 15 |
| (cat saliva) | | | | |
| Mus musculus | Mus m 1; MUP | 19 | C | 80, 81 |
| (mouse urine) | | | | |
| Rattus norvegius | Rat n 1 | 17 | C | 82, 83 |
| (rat urine) | | | | |

APPENDIX 6
FUNGI ALLERGENS

| | | | | |
|---|---|---|---|---|
| | SYSTEMATIC AND | | SEQUENCE | ACCESSION NO. |
| ALLERGEN SOURCE | ORIGINAL NAMES | MW (KD) | DATA | OR REFERENCES |

Ascomycota
Dothidiales

| | | | | |
|---|---|---|---|---|
| Alternaria | Alt a 1; | 28 | C | U82633 |
| alternata | Alt a 2; | 25 | C | |
| | Alt a 3; heat shock | 70 | C | U87807, U87808 |
| | prot | | | |
| | Alt a 6; ribosomal | 11 | C | X78222, U87806 |
| | protein | | | |
| | Alt a 7; YCP4 protein | 22 | C | X78225 |
| | Alt a 10; aldehyde | 53 | C | X78227, P42041 |
| | dehydrogenase | | | |
| Cladosporium | Cla h 1; | 13 | | 83a, 83b |
| herbarum | Cla h 2; | 23 | | 83a, 83b |
| | Cla h 3; aldehyde | 53 | C | X78228 |
| | dehydrogenase | | | |
| | Cla h 4; ribosomal | 11 | C | X78223 |
| | protein | | | |
| | Cla h 5; YCP4 protein | 22 | C | X78224 |
| | Cla h 6; enolase | 46 | C | X78226 |

Eurotiales

| | | | | |
|---|---|---|---|---|
| | Asp fl 13; alkaline | 34 | | 84 |
| | serine proteinase | | | |
| Aspergillus | Asp f 1 | 18 | C | M83781, S39330 |
| Fumigatus | Asp f 2 | 37 | C | U56938 |
| | Asp f 3; peroxisomal | 19 | C | U20722 |
| | protein | | | |
| | Asp f 4 | 30 | C | AJ001732 |
| | Asp f 5 | 42 | C | Z30424 |
| | metalloprotease | | | |
| | Asp f 6; Mn | 26.5 | C | U53561 |
| | superoxide dismutase | | | |
| | Asp f 7 | 12 | C | AJ223315 |
| | Asp f 8; ribosomal | 11 | C | AJ224333 |
| | protein P2 | | | |
| | Asp f 9 | 34 | C | AJ223327 |
| | Asp f 10; aspartic | 34 | | X85092 |
| | protease | | | |
| | Asp f 11; | 24 | | 84a |
| | peptidyl-prolyl isom | | | |
| | Asp f 12; heat shock | 65 | C | U92465 |
| | prot. P70 | | | |
| | Asp f 13; alkaline | 34 | | 84b |
| | serine proteinase | | | |
| | Asp f 15 | 16 | C | AJ002026 |
| | Asp f 16 | 43 | C | g3643813 |
| | Asp f 17 | | | AJ224865 |
| | Asp f 18; vacuolar | 34 | C | 84c |
| | serine | | | |
| | Asp f ? | 90 | P | 85 |
| | Asp f ? | 55 | P | 86 |
| Aspergillus niger | Asp n 14 | 105 | C | AF108944 |
| | beta-xylosidase | | | |
| | Asp n 18; | 34 | C | 84b |
| | vacuolar serine | | | |
| | proteinase | | | |
| | Asp n ? | 85 | C | Z84377 |
| Aspergillus | Asp o 2 | 53 | C | D00434, M33218 |
| oryzae | TAKA-amylase A | | | |
| | Asp o 13; alkaline | 34 | C | X17561 |
| | serine proteinase | | | |
| Penicillium | Pen b 13; alkaline | 33 | | 86a |
| brevicompactum | serine Proteinase | | | |
| Penicillium | Pen c 1; heat shock | 70 | C | U64207 |
| citrinum | prot. P70 | | | |
| | Pen c 3; peroxisomal | | | 86b |
| | membrane | | | |
| | protein | | | |
| | Pen c 13; alkaline | | | |
| | serine proteinase | 33 | | 86a |
| Penicillium | Pen n 1; N-acetyl | 68 | | 87 |
| notatum | glucosaminidase | | | |
| | Pen n 13; alkaline | 34 | | 89 |
| | serine proteinase | | | |
| | Pen n 18; vacuolar | 32 | | 89 |
| | serine proteinase | | | |
| Penicillium | Pen o 18; vacuolar | 34 | | 89 |
| oxalicum | serine proteinase | | | |

Onygenales

| | | | | |
|---|---|---|---|---|
| Trichophyton | Tri r 2 | | C | 90 |
| rubrum | Tri r 4; serine protease | | C | 90 |
| Trichophyton | Tri t 1 | 30 | P | 91 |
| tonsurans | Tri t 4; serine protease | 83 | C | 90 |

Saccharomycetales

| | | | | |
|---|---|---|---|---|
| Candida albicans | Cand a 1 | 40 | C | 88 |
| Candida boidinii | Cand b 2 | 20 | C | J04984, J04985 |

Basidiomycota
Basidiolelastomyce-tes

| | | | | |
|---|---|---|---|---|
| Malassezia furfur | Mal f 1 | | | 91a |
| | Mal f 2; MF1 | 21 | C | AB011804 |
| | peroxisomal | | | |
| | membrane protein | | | |
| | Mal f 3; MF2 | 20 | C | AB011805 |
| | peroxisomal | | | |
| | membrane protein | | | |
| | Mal f 4 | 35 | C | Takesako, p.c. |
| | Mal f 5 | 18* | C | AJ011955 |
| | Mal f 6; cyclophilin | 17* | C | AJ011956 |
| | homologue | | | |

Basidiomycetes

| | | | | |
|---|---|---|---|---|
| Psilocybe | Psi c 1; | 16 | | 91b |
| cubensis | Psi c 2; cyclophilin | | | |
| Coprinus comatus | Cop c 1; | 11 | C | AJ132235 |
| (shaggy cap) | Cop c 2; | | | |

APPENDIX 7
INSECT ALLERGENS

| | SYSTEMATIC AND | | SEQUENCE | ACCESSION NO. |
|---|---|---|---|---|
| ALLERGEN SOURCE | ORIGINAL NAMES | MW (KD) | DATA | OR REFERENCES |
| Apis mellifera | Api m 1; phospholipase A2 | 16 | C | 92 |
| (honey bee) | Api m 2; hyaluronidase | 44 | C | 93 |
| | Api m 4; melittin | 3 | C | 94 |
| Bombus | Bom p 1; phospholipase | 16 | P | 95 |
| pennsylvanicus | Bom p 4; protease | | P | 95 |
| (bumble bee) | | | | |
| Blattella | Bla g 1; Bd90k | 36 | C | 96 |
| germanica | Bla g 2; aspartic protease | 21 | C | 97 |
| (German | Bla g 4; calycin | 22 | C | 98 |
| cockroach) | Bla g 5; glutathione transf. | 27 | C | 98 |
| | Bla g 6; troponin C | | C | |
| Periplaneta | Per a 1; Cr-PII | 72-78 | C | 98A |
| americana | Per a 3; Cr-PI | | C | |
| (American | Per a 7; tropomyosin | 37 | C | Y14854 |
| cockroach) | | | | |
| Chironomus | Chi t 1-9; hemoglobin | 16 | C | 99 |
| thummi thummi | Chi t 1.01; component III | 16 | C | P02229 |
| (midges) | Chi t 1.02; component IV | 16 | C | P02230 |
| | Chi t 2.0101; component I | 16 | C | P02221 |
| | Chi t 2.0102; component IA | 16 | C | P02221 |
| | Chi t 3; component II-beta | 16 | C | P02222 |
| | Chi t 4; component IIIA | 16 | C | P02231 |
| | Chi t 5; component VI | 16 | C | P02224 |
| | Chi t 6.01; component VIIA | 16 | C | P02226 |
| | Chi t 6.02; component IX | 16 | C | P02223 |
| | Chi t 7; component VIIB | 16 | C | P02225 |
| | Chi t 8; component VIII | 16 | C | P02227 |
| | Chi t 9; component X | 16 | C | P02228 |
| Dolichovespula | Dol m 1; phospholipase A1 | 35 | C | 100 |
| maculata | Dol m 2; hyaluronidase | 44 | C | 101 |
| (white face | Dol m 5; antigen 5 | 23 | C | 102, 103 |
| hornet) | | | | |
| Dolichovespula | Dol a 5; antigen 5 | 23 | C | 104 |
| arenaria | | | | |
| (yellow hornet) | | | | |
| Polistes | Pol a 1; phospholipase A1 | 35 | P | 105 |
| annularies | Pol a 2; hyaluronidase | 44 | P | 105 |
| (wasp) | Pol a 5; antigen 5 | 23 | C | 104 |
| Polistes | Pol d 1; | 32-34 | C | DR Hoffman |
| dominulus | Pol d 4; serine protease | | | DR Hoffman |
| (Mediterranean | Pol d 5; | | | P81656 |
| paper wasp) | | | | |
| Polistes | Pol e 1; phospholipase A1 | 34 | P | 107 |
| exclamans | Pol e 5; antigen 5 | | | |
| (wasp) | | 23 | C | 104 |
| Polistes fuscatus | Pol f 5; antigen 5 | 23 | C | 106 |
| (wasp) | | | | |
| Polistes metricus | Pol m 5; antigen 5 | 23 | P | 106 |
| (wasp) | | | | |
| Vespa crabo | Vesp c 1; phospholipase | 34 | P | 107 |
| (European hornet) | Vesp c 5.0101; antigen 5 | 23 | C | 106 |
| | Vesp c 5.0102; antigen 5 | 23 | C | 106 |
| Vespa mandarina | Vesp m 1.01; | | | DR Hoffman |
| (giant asian | Vesp m 1.02; | | | DR Hoffman |
| hornet) | Vesp m 5; | | | P81657 |
| Vespula | Ves f 5; antigen 5 | 23 | C | 106 |
| flavopilosa | | | | |
| (yellowjacket) | | | | |
| Vespula | Ves g 5; antigen 5 | 23 | C | 106 |
| germanica | | | | |
| (yellowjacket) | | | | |
| Vespula | Ves m 1; phospholipase A1 | 33.5 | C | 108 |
| maculifrons | Ves m 2; hyaluronidase | 44 | P | 109 |
| (yellowjacket) | Ves m 5; antigen 5 | 23 | 23 | 104 |
| Vespula | Ves p 5; antigen 5 | 23 | C | 106 |
| pennsylvanica | | | | |
| (yellowjacket) | | | | |
| Vespula | Ves s 5; antigen 5 | 23 | C | 106 |
| squamosa | | | | |
| (yellowjacket) | | | | |
| Vespula vidua | Ves vi 5; | 23 | C | 106 |
| (wasp) | | | | |
| Vespula vulgaris | Ves v 1; phopholipase A1 | 35 | C | 105A |
| (yellowjacket) | Ves v 2; hyaluronidase | 44 | P | 105A |
| | Ves v 5; antigen 5 | 23 | C | 104 |
| Myrmecia | Myr p 1; | | C | X70256 |
| pilosula | Myr p 2; | | C | S81785 |
| (Australian | | | | |
| jumper ant) | | | | |
| Solenopsis | Sol g 2; | | | DR Hoffman |
| geminata | Sol g 4 | | | DR Hoffman |
| (tropical fire ant) | | | | |
| Solenopsis invicta | Sol i 2; | 13 | C | 110, 111 |
| (fire ant) | Sol i 3; | 24 | C | 110 |
| | Soli 4; | 13 | C | 110 |
| Solenopsis | Sols 2; | | | DR Hoffman |
| saevissima | | | | |
| (brazilian fire ant) | | | | |

APPENDIX 8
FOOD ALLERGENS

| | SYSTEMATIC AND | | SEQUENCE | ACCESSION NO. |
|---|---|---|---|---|
| ALLERGEN SOURCE | ORIGINAL NAMES | MW (KD) | DATA | OR REFERENCES |
| Gadus callarias | Gad c 1; allergen M | 12 | C | 112, 113 |
| (cod) | | | | |
| Salmo salar | Sals 1; parvalbumin | 12 | C | X97824 |
| (Atlantic salmon) | | | | X97825 |
| Bos domesticus | Bos d 4; alpha- | 14.2 | C | M18780 |
| (domestic cattle) | lactalbumin | 18.3 | C | X14712 |
| | Bos d 5; beta- | | | |
| | lactoglobulin | 67 | C | M73993 |
| | Bos d 6; serum albumin | 160 | | 77 |
| | Bos d 7; immunoglobulin | 20-30 | | 77 |
| | Bos d 8; caseins | | | |
| Gallus | Gal d 1; ovomucoid | 28 | C | 114, 115 |
| domesticus | Gald 2; ovalbumin | 44 | C | 114, 115 |
| (chicken) | Gald 3; conalbumin | 78 | C | 114, 115 |
| | (Ag22) | | | |
| | Gald 4; lysozyme | 14 | C | 114, 115 |
| Metapenaeus | Met e 1; tropomyosin | | C | U08008 |
| ensis | | | | |
| (shrimp) | | | | |
| Penaeus aztecus | Pen a 1; tropomyosin | 36 | P | 116 |
| (shrimp) | | | | |
| Penaeus indicus | Pen i 1; tropomyosin | 34 | C | 117 |
| (shrimp) | | | | |
| Todarodes | Tod p 1; tropomyosin | 38 | P | 117A |
| pacificus (squid) | | | | |
| Haliotis Midae | Hal m 1 | 49 | — | 117B |
| (abalone) | | | | |
| Apium | Api g 1; Bet v 1 | 16* | C | Z48967 |
| graveolens | homologue | | | |
| (celery) | | | | |
| Brassica juncea | Bra j 1; 2S albumin | 14 | C | 118 |
| (oriental mustard) | | | | |
| Brassica rapa | Bra r 2; prohevein-like | 25 | ? | P81729 |
| (turnip) | protein | | | |
| Hordeum vulgare | Hor v 1; BMAI-1 | 15 | C | 119 |
| (barley) | | | | |
| Malus domestica | Mal d 1; Bet v 1 | | C | X83672 |
| (apple) | homologue | | | |
| | Mal d 3; lipid transfer | 9 | C | Pastorello |
| | protein | | | |
| Oryza sativa | Ory s 1; | | C | U31771 |
| (rice) | | | | |
| Persea americana | Pers a 1; endochitmase | 32 | C | Z78202 |
| (avocado) | | | | |
| Prunus armeniaca | Pru ar 1; Bet v 1 | | C | U93165 |
| (apricot) | homologue | | | |
| | Pru ar 3; lipid transfer | 9 | P | |
| | protein | | | |
| Prunus avium | Pru av 1; Bet v 1 | | C | U66076 |
| (sweet cherry) | homologue | | | |
| | Pru av 2; thaumatin | | C | U32440 |
| | homologue | | | |
| Prunus persica | Pru p 3; lipid transfer | 10 | P | P81402 |
| (peach) | protein | | | |
| Sinapis alba | Sin a 1; 2S albumin | 14 | C | 120 |
| (yellow mustard) | | | | |
| Glycine max | Gly m 1.0101; HPS | 7.5 | P | 121 |
| (soybean) | Gly m 1.0102; HPS | 7 | P | 121 |
| | Gly m 2 | 8 | P | A57106 |
| | Gly m 3; profilin | 14 | C | AJ223982 |
| Arachis hypogaea | Ara h 1; vicilin | 63.5 | C | L34402 |
| (peanut) | Ara h 2; conglutin | 17 | C | L77197 |
| | Ara h 3; glycinin | 14 | C | AF093541 |
| | Ara h 4; glycinin | 37 | C | AF086821 |
| | Ara h 5; profilin | 15 | C | AF059616 |
| | Ara h 6; conglutin | 15 | C | AF092846 |
| | homolog | | | |
| | Ara h 7; conglutin | 15 | C | AF091737 |
| | homolog | | | |
| Actinidia | Act c 1; cysteine protease | 30 | P | P00785 |
| chinensis | | | | |
| (kiwi) | | | | |
| Solanum | Sol t 1; patatin | 43 | P | P15476 |
| tuberosum | | | | |
| (potato) | | | | |
| Bertholletia | Ber e 1; 2S albumin | 9 | C | P04403, M17146 |
| excelsa | | | | |
| (Brazil nut) | | | | |
| Juglans regia | Jug r 1; 2S albumin | 44 | C | U66866 |
| (English walnut) | Jug r 2; vicilin | | C | AF066055 |
| Ricinus | Rice 1; 2S albumin | | C | P01089 |
| communis | | | | |
| (Castor bean) | | | | |

APPENDIX 9
OTHER ALLERGENS

| | SYSTEMATIC AND | | SEQUENCE | ACCESSION NO. |
|---|---|---|---|---|
| ALLERGEN SOURCE | ORIGINAL NAMES | MW (KD) | DATA | OR REFERENCES |
| Ascaris suum | Asc s 1 | 10 | P | 122 |
| (worm) | | | | |
| Aedes aegyptii | Aed a 1; apyrase | 68 | C | L12389 |
| (mosquito) | Aed a 2 | 37 | C | M33157 |
| Hevea | Hev b 1; elongation | 58 | P | 123, 124 |
| brasiliensis | factor | | | |
| (rubber) | Hev b 2; 1,3-glucanase | 58 | P | 123, 124 |
| | Hev b 2; 1,3-glucanase | 34/36 | C | 125 |
| | Hev b 3 | 24 | P | 126, 127 |
| | Hev b 4; component of | 100/110/115 | P | 128 |
| | microhelix protein | | | |
| | complex | | | |
| | Hev b 5 | 16 | C | U42640 |
| | Hev b 6.01; hevein | 20 | C | M36986/p02877 |
| | precursor | | | |
| | Hev b 6.02; hevein | 5 | C | M36986/p02877 |
| | Hev b 6.03; C-terminal | 14 | C | M36986/p02877 |
| | fragment | | | |
| | Hev b 7; patatin | 46 | C | U80598 |
| | homologue | | | |
| | Hev b 8; profilin | 14 | C | Y15042 |
| | Hev b 9; enolase | 51 | C | AJ132580/AJ132581 |

### APPENDIX 10

### References

1. Marsh, D. G., and L. R. Freidhoff. 1992. ALBE, an allergen database. IUIS, Baltimore, Md., Edition 1.0.
2. Marsh, D. G., L. Goodfriend, T. P. King, H. Lowenstein, and T. A. E. Platts-Mills. 1986. Allergen nomenclature. Bull WHO 64:767-770.
3. King, T. P., P. S. Norman, and J. T. Cornell. 1964. Isolation and characterization of allergen from ragweed pollen. II. Biochemistry 3:458-468.
4. Lowenstein, H. 1980. Timothy pollen allergens. Allergy 35:188-191.
5. Aukrust, L. 1980. Purification of allergens in Cladosporium herbarum. Allergy 35:206-207.
6. Demerec, M., E. A. Adelberg, A. J. Clark, and P. E. Hartman. 1966. A proposal for a uniform nomenclature in bacterial genetics. Genetics 54:61-75.
7. Bodmer, J. G., E. D. Albert, W. F. Bodmer, B. Dupont, H. A. Erlich, B. Mach, S. G. E. Marsh, W. R. Mayr, P. Parham, T. Sasuki, G. M. Th. Schreuder, J. L. Strominger, A. Svejgaard, and P. I. Terasaki. 1991. Nomenclature for factors of the HLA system, 1990. Immunogenetics 33:301-309.
8. Griffith, I. J., J. Pollock, D. G. Mapper, B. L. Rogers, and A. K. Nault. 1991. Sequence polymorphism of Amb a I and Amb a II, the major allergens in Ambrosia artemisiifolia (short ragweed). Int. Arch. Allergy Appl. Immunol. 96:296-304.
9. Roebber, M., D. G. Mapper, L. Goodfriend, W. B. Bias, S. H. Hsu, and D. G. Marsh. 1985. Immunochemical and genetic studies of Amb t V (Ra5G), an Ra5 homologue from giant ragweed pollen. J. Immunol. 134:3062-3069.
10. Metzler, W. J., K. Valentine, M. Roebber, M. Friedrichs, D. G. Marsh, and L. Mueller. 1992. Solution structures of ragweed allergen Amb t V. Biochemistry 31:5117-5127.
11. Metzler, W. J., K. Valentine, M. Roebber, D. G. Marsh, and L. Mueller. 1992. Proton resonance assignments and three-dimensional solution structure of the ragweed allergen Amb a V by nuclear magnetic resonance spectroscopy. Biochemistry 31:8697-8705.
12. Goodfriend, L., A. M. Choudhury, J. Del Carpio, and T. P. King. 1979. Cytochromes C: New ragweed pollen allergens. Fed. Proc. 38:1415.
13. Ekramoddoullah, A. K. M., F. T. Kisil, and A. H. Sehon. 1982. Allergenic cross reactivity of cytochrome c from Kentucky bluegrass and perennial ryegrass pollens. Mol. Immunol. 19:1527-1534.
14. Ansari, A. A., E. A. Killoran, and D. G. Marsh. 1987. An investigation of human response to perennial ryegrass (Lolium perenne) pollen cytochrome c (Lol p X). J. Allergy Clin. Immunol. 80:229-235.
15. Morgenstern, J. P., I. J. Griffith, A. W. Brauer, B. L. Rogers, J. F. Bond, M. D. Chapman, and M. Kuo. 1991. Amino acid sequence of Fel d I, the major allergen of the domestic cat: protein sequence analysis and cDNA cloning. Proc. Natl. Acad. Sci. USA 88:9690-9694.
16. Griffith, I. J., S. Craig, J. Pollock, X. Yu, J. P. Morgenstern, and B. L. Rogers. 1992. Expression and genomic structure of the genes encoding FdI, the major allergen from the domestic cat. Gene 113:263-268.
17. Weber, A., L. Marz, and F. Altmann. 1986. Characteristics of the asparagine-linked oligosaccharide from honey-bee venom phospholipase A2. Comp. Biochem. Physiol. 83B:321-324.
18. Weber, A., H. Schroder, K. Thalberg, and L. Marz. 1987. Specific interaction of IgE antibodies with a carbohydrate epitope of honey bee venom phospholipase A2. Allergy 42:464-470.
19. Stanworth, D. R., K. J. Dorrington, T. E. Hugh, K. Reid, and M. W. Turner. 1990. Nomenclature for synthetic peptides representative of immunoglobulin chain sequences. Bulletin WHO 68:109-111.
20. Rafnar, T., I. J. Griffith, M. C. Kuo, J. F. Bond, B. L. Rogers, and D. G. Klapper. 1991. Cloning of Amb a I (Antigen E), the major allergen family of short ragweed pollen. J. Biol. Chem. 266: 1229-1236.
21. Rogers, B. L., J. P. Morgenstern, I. J. Griffith, X. B. Yu, C. M. Counsell, A. W. Brauer, T. P. King, R. D. Garman, and M. C. Kuo. 1991. Complete sequence of the allergen Amb a II: recombinant expression and reactivity with T-cells from ragweed allergic patients. J. Immunol. 147:2547-2552.
22. Klapper, D. G., L. Goodfriend, and J. D. Capra. 1980. Amino acid sequence of ragweed allergen Ra3. Biochemistry 19:5729-5734.
23. Ghosh, B., M. P. Perry, T. Rafnar, and D. G. Marsh. 1993. Cloning and expression of immunologically active recombinant Amb a V allergen of short ragweed (Ambrosia artemisiifolia) pollen. J. Immunol. 150:5391-5399.
24. Roebber, M., R. Hussain, D. G. Klapper, and D. G. Marsh. 1983. Isolation and properties of a new short ragweed pollen allergen, Ra6. J. Immunol. 131:706-711.
25. Lubahn, B., and D. G. Klapper. 1993. Cloning and characterization of ragweed allergen Amb a VI (abst). J. Allergy Clin. Immunol. 91:338.
26. Roebber, M., and D. G. Marsh. 1991. Isolation and characterization of allergen Amb a VII from short ragweed pollen. J. Allergy Clin. Immunol. 87:324.
27. Rogers, B. L., J. Pollock, D. G. Klapper, and I. J. Griffith. 1993. Cloning, complete sequence, and recombinant expression of a novel allergen from short ragweed pollen (abst). J. Allergy Clin. Immunol. 91:339.
28. Goodfriend, L., A. M. Choudhury, D. G. Klapper, K. M. Coulter, G. Dorval, J. DelCarpio, and C. K. Osterland. 1985. Ra5G, a homologue of Ra5 in giant ragweed pollen: isolation, HLA-DR-associated activity and amino acid sequence. Mol. Immunol. 22:899-906.
28A. Breitenbach M, pers. comm.
29. Nilsen, B. M., K. Sletten, M. O'Neill, B. Smestead Paulsen, and H. van Halbeek. 1991. Structural analysis of the glycoprotein allergen Art v II from pollen of mugwort (Artemesia vulgaris). J. Biol. Chem. 266:2660-2668.
29A. Jimenez A, Moreno C, Martinez J, Martinez A, Bartolome B, Guerra F, Palacios R 1994. Sensitization to sunflower pollen: only an occupational allergy? Int Arch Allergy Immunol 105:297-307.
30. Smith, P. M., Suphioglu, C., Theriault, K., Knox, R. B. and Singh, M. B. 1996. Cloning and expression in yeast Pichia pastoris of a biologically active form of Cyn d 1, the major allergen of Bermuda grass pollen. J. Allergy Clin. Immunol. 98:331-343.
31. Suphioglu, C., Ferreira, F. and Knox, R. B. 1997. Molecular cloning and immunological characterisation of Cyn d 7, a novel calcium-binding allergen from Bermuda grass pollen. FEBS Lett. 402:167-172.
31A. Asturias J A, Arilla M C, Gomez-Bayon N, Martinez J, Martinez A, and Palacios R. 1997. Cloning and high level expression of Cynodon dactylon (Bermuda grass) pollen profilin (Cyn d 12) in Escherichia coli: purification and characterization of the allergen. Clin Exp Allergy 27:1307-1313.
32. Mecheri, S., G. Peltre, and B. David. 1985. Purification and characterization of a major allergen from Dactylis glomerata pollen: The Ag Dg 1. Int. Arch. Allergy Appl. Immunol. 78:283-289.
33. Roberts, A. M., L. J. Bevan, P. S. Flora, I. Jepson, and M. R. Walker. 1993. Nucleotide sequence of cDNA encoding the Group II allergen of Cocksfoot/Orchard grass (Dactylis glomerata), Dac g II. Allergy 48:615-623.
33A. Guerin-Marchand, C., Senechal, H., Bouin, A. P., Leduc-Brodard, V., Taudou, G., Weyer, A., Peltre, G. and David, B. 1996. Cloning, sequencing and immunological characterization of Dac g 3, a major allergen from Dactylis glomerata pollen. Mol. Immunol. 33:797-806.
34. Klysner, S., K. Welinder, H. Lowenstein, and F. Matthiesen. 1992. Group V allergens in grass pollen IV. Similarities in amino acid compositions and amino terminal sequences of the group V allergens from Lolium perenne, Poa pratensis and Dactylis glomerata. Clin. Exp. Allergy 22: 491-497.
35. Perez, M., G. Y. Ishioka, L. E. Walker, and R. W. Chesnut. 1990. cDNA cloning and immunological characterization of the rye grass allergen Lol p I. J. Biol. Chem. 265:16210-16215.
36. Griffith, I. J., P. M. Smith, J. Pollock, P. Theerakulpisut, A. Avjioglu, S. Davies, T. Hough, M. B. Singh, R. J. Simpson, L. D. Ward, and R. B. Knox. 1991. Cloning and sequencing of Lol p I, the major allergenic protein of rye-grass pollen. FEBS Letters 279:210-215.
37. Ansari, A. A., P. Shenbagamurthi, and D. G. Marsh. 1989. Complete amino acid sequence of a Lolium perenne (perennial rye grass) pollen allergen, Lol p II. J. Biol. Chem. 264:11181-11185.
37A. Sidoli, A., Tamborini, E., Giuntini, I., Levi, S., Volonte, G., Paini, C., De Lalla, C., Siccardi, A. G., Baralle, F. E., Galliani, S, and Arosio, P. 1993. Cloning, expression, and immunological characterization of recombinant Lolium perenne allergen Lol p II. J. Biol. Chem. 268:21819-21825.
38. Ansari, A. A., P. Shenbagamurthi, and D. G. Marsh. 1989. Complete primary structure of a Lolium perenne (perennial rye grass) pollen allergen, Lol p III: Comparison with known Lol p I and II sequences. Biochemistry 28:8665-8670.
39. Singh, M. B., T. Hough, P. Theerakulpisut, A. Avjioglu, S. Davies, P. M. Smith, P. Taylor, R. J. Simpson, L. D. Ward, J. McCluskey, R. Puy, and R. B. Knox.
1991. Isolation of cDNA encoding a newly identified major allergenic protein of rye-grass pollen: Intracellular targeting to the amyloplost. Proc. Natl. Acad. Sci. 88:1384-1388.
39A. van Ree R, Hoffman D R, van Dijk W, Brodard V, Mahieu K, Koeleman C A, Grande M, van Leeuwen W A, Aalberse R C. 1995. Lol p XI, a new major grass pollen allergen, is a member of a family of soybean trypsin inhibitor-related proteins. J. Allergy Clin Immunol 95:970-978.
40. Suphioglu, C. and Singh, M. B. 1995. Cloning, sequencing and expression in Escherichia coli of Pha a 1 and four isoforms of Pha a 5, the major allergens of canary grass pollen. Clin. Exp. Allergy 25:853-865.
41. Dolecek, C., Vrtala, S., Laffer, S., Steinberger, P., Kraft, D., Scheiner, O. and Valenta, R. 1993. Molecular characterization of Phl p II, a major timothy grass (Phleum pratense) pollen allergen. FEBS Lett. 335:299-304.
41A. Fischer S, Grote M, Fahlbusch B, Muller W D, Kraft D, Valenta R. 1996. Characterization of Phl p 4, a major timothy grass (Phleum pratense) pollen allergen. J. Allergy Clin Immunol 98:189-198.
42. Matthiesen, F., and H. Lowenstein. 1991. Group V allergens in grass pollens. I. Purification and characterization of the group V allergen from Phleum pratense pollen, Phl p V. Clin. Exp. Allergy 21:297-307.
43. Petersen, A., Bufe, A., Schramm, G., Schlaak, M. and Becker, W. M. 1995. Characterization of the allergen group VI in timothy grass pollen (Phl p 6). II. cDNA cloning of Phl p 6 and structural comparison to grass group V. Int. Arch. Allergy Immunol. 108:55-59.
44. Valenta, R., Ball, T., Vrtala, S., Duchene, M., Kraft, D. and Scheiner, O. 1994. cDNA cloning and expression of timothy grass (Phleum pratense) pollen profilin in Escherichia coli: comparison with birch pollen profilin. Biochem. Biophys. Res. Commun. 199:106-118.
46. Esch, R. E., and D. G. Klapper. 1989. Isolation and characterization of a major cross-reactive grass group I allergenic determinant. Mol. Immunol. 26:557-561.
47. Olsen, E., L. Zhang, R. D. Hill, F. T. Kisil, A. H. Sehon, and S. Mohapatra. 1991. Identification and characterization of the Poa p IX group of basic allergens of Kentucky bluegrass pollen. J. Immunol. 147:205-211.
48. Avjioglu, A., M. Singh, and R. B. Knox. 1993. Sequence analysis of Sor h I, the group I allergen of Johnson grass pollen and it comparison to rye-grass Lol p I (abst). J. Allergy Clin. Immunol. 91:340.
51. Larsen, J. N., P. Str^{o}man, and H. Ipsen. 1992. PCR based cloning and sequencing of isogenes encoding the tree pollen major allergen Car b I from Carpinus betulus, hornbeam. Mol. Immunol. 29:703-711.
52. Kos T, Hoffmann-Sommergruber K, Ferreira F, Hirschwehr R, Ahorn H, Horak F, Jager S, Sperr W, Kraft D, Scheiner O. 1993. Purification, characterization and N-terminal amino acid sequence of a new major allergen from European chestnut pollen Cas s 1. Biochem Biophys Res Commun 196:1086-92.
53. Breiteneder, H., F. Ferreira, K. Hoffman-Sommergruber, C. Ebner, M. Breitenbach, H. Rumpold, D. Kraft, and O, Scheiner. 1993. Four recombinant isoforms of Cor a I, the major allergen of hazel pollen. Europ. J. Biochem. 212:355-362.
54. Ipsen, H., and B. C. Hansen. 1991. The NH2-terminal amino acid sequence of the immunochemically partial identical major allergens of alder (Alnus glutinosa) Aln g I, birch (Betula verrucosa) Bet v I, hornbeam (Carpinus betulus) Car b I and oak (Quercus alba) Que a I pollens. Mol. Immunol. 28:1279-1288.
55. Taniai, M., S. Ando, M. Usui, M. Kurimoto, M. Sakaguchi, S. Inouye, and T. Matuhasi. 1988. N-terminal amino acid sequence of a major allergen of Japanese cedar pollen (Cry j I). FEBS Lett. 239:329-332.
56. Griffith, I. J., A. Lussier, R. Garman, R. Koury, H. Yeung, and J. Pollock. 1993. The cDNA cloning of Cry j I, the major allergen of Cryptomeria japonica (Japanese cedar) (abst). J. Allergy Clin. Immunol. 91:339.
57. Sakaguchi, M., S. Inouye, M. Taniai, S. Ando, M. Usui, and T. Matuhasi. 1990. Identification of the second major allergen of Japanese cedar pollen. Allergy 45:309-312.
58. Gross G N, Zimburean J M, Capra J D 1978. Isolation and partial characterization of the allergen in mountain cedar pollen. Scand J. Immunol 8:437-41
58A. Obispo T M, Melero J A, Carpizo J A, Carreira J, Lombardero M 1993. The main allergen of Olea europaea (Ole e I) is also present in other species of the oleaceae family. Clin Exp Allergy 23:311-316.
59. Cardaba, B., D. Hernandez, E. Martin, B. de Andres, V. del Pozo, S. Gallardo, J. C. Fernandez, R. Rodriguez, M. Villalba, P. Palomino, A. Basomba, and C. Lahoz. 1993. Antibody response to olive pollen antigens: association between HLA class II genes and IgE response to Ole e I (abst). J. Allergy Clin. Immunol. 91:338.
60. Villalba, M., E. Batanero, C. Lopez-Otin, L. M. Sanchez, R. I. Monsalve, M. A. Gonzalez de la Pena, C. Lahoz, and R. Rodriguez. 1993. Amino acid sequence of Ole e I, the major allergen from olive tree pollen (Olea europaea). Europ. J. Biochem. 216:863-869.
60A. Asturias J A, Arilla M C, Gomez-Bayon N, Martinez J, Martinez A, Palacios R 1997. Cloning and expression of the panallergen profilin and the major allergen (Ole e 1) from olive tree pollen. J. Allergy Clin Immunol 100:365-372.
60B. Batanero E, Villalba M, Ledesma A Puente X S, Rodriguez R. 1996. Ole e 3, an olive-tree allergen, belongs to a widespread family of pollen proteins. Eur J Biochem 241: 772-778.
61. Chua, K. Y., G. A. Stewart, and W. R. Thomas. 1988. Sequence analysis of cDNA encoding for a major house dust mite allergen, Der p I. J. Exp. Med. 167:175-182.
62. Chua, K. Y., C. R. Doyle, R. J. Simpson, K. J. Turner, G. A. Stewart, and W. R. Thomas. 1990. Isolation of cDNA coding for the major mite allergen Der p II by IgE plaque immunoassay. Int. Arch. Allergy Appl. Immunol. 91:118-123.
63. Smith W A, Thomas W R. 1996. Comparative analysis of the genes encoding group 3 allergens from Dermatophagoides pteronyssinus and Dermatophagoides farinae. Int Arch Allergy Immunol 109: 133-40.
64. Lake, F. R., L. D. Ward, R. J. Simpson, P. J. Thompson, and G. A. Stewart. 1991. House dust mite-derived amylase: Allergenicity and physicochemical characterisation. J. Allergy Clin. Immunol. 87:1035-1042.
65. Tovey, E. R., M. C. Johnson, A. L. Roche, G. S. Cobon, and B. A. Baldo. 1989. Cloning and sequencing of a cDNA expressing a recombinant house dust mite protein that binds human IgE and corresponds to an important low molecular weight allergen. J. Exp. Med. 170:1457-1462.
66. Yasueda, H., T. Shida, T. Ando, S. Sugiyama, and H. Yamakawa. 1991. Allergenic and proteolytic properties of fourth allergens from Dermatophagoides mites. In: “Dust Mite Allergens and Asthma. Report of the 2nd international workshop” A. Todt, Ed., UCB Institute of Allergy, Brussels, Belgium, pp. 63-64.
67. Shen, H.-D., K.-Y. Chua, K.-L. Lin, K.-H. Hsieh, and W. R. Thomas. 1993. Molecular cloning of a house dust mite allergen with common antibody binding specificities with multiple components in mite extracts. Clin. Exp. Allergy 23:934-40.
67A. O'Neil G M, Donovan G R, Baldo B A. 1994. Cloning and characterisation of a major allergen of the house dust mite Dermatophagoides pteronyssinus, homologous with glutathione S-transferase. Biochim Biophys Acta, 1219:521-528.
67B. King C, Simpson R J, Moritz R L, Reed G E, Thompson P J, Stewart G A. 1996. The isolation and characterization of a novel collagenolytic serine protease allergen (Der p 9) from the dust mite Dermatophagoides pteronyssinus. J. Allergy Clin Immunol 98:739-47.
68. Lind P, Hansen O C, Horn N. 1988. The binding of mouse hybridoma and human IgE antibodies to the major fecal allergen, Der p I of D. pteronyssinus. J. Immunol. 140:4256-4262.
69. Dilworth, R. J., K. Y. Chua, and W. R. Thomas. 1991. Sequence analysis of cDNA coding for a mojor house dust allergn Der f I. Clin. Exp. Allergy 21:25-32.
70. Nishiyama, C., T. Yunki, T. Takai, Y. Okumura, and H. Okudaira. 1993. Determination of three disulfide bonds in a major house dust mite allergen, Der f II. Int. Arch. Allergy Immunol. 101:159-166.
71. Trudinger, M., K. Y. Chua, and W. R. Thomas. 1991. cDNA encoding the major dust mite allergen Der f II. Clin. Exp. Allergy 21:33-38.
72. Aki T, Kodama T, Fujikawa A, Miura K, Shigeta S, Wada T, Jyo T, Murooka Y, Oka S, Ono K. 1995. Immunochemical characterization of recombinant and native tropomyosins as a new allergen from the house dust mite Dermatophagoides farinae. J. Allergy Clin Immunol 96:74-83.
73. van Hage-Hamsten, M., T. Bergman, E. Johansson, B. Persson, H. Jornvall, B. Harfast, and S. G. O. Johansson. 1993. N-terminal amino acid sequence of major allergen of the mite lepidoglyphus destructor (abst). J. Allergy Clin. Immunol. 91:353.
74. Varela J, Ventas P, Carreira J, Barbas J A, Gimenez-Gallego G, Polo F. Primary structure of Lep d I, the main Lepidoglyphus destructor allergen. Eur J. Biochem 225:93-98, 1994.
75. Schmidt M, van der Ploeg I, Olsson S, van Hage Hamsten M. The complete cDNA encoding the Lepidoglyphus destructor major allergen Lep d 1. FEBS Lett 370:11-14, 1995.
76. Rautiainen J, Rytkonen M, Pelkonen J, Pentikainen J, Perola O, Virtanen T, Zeiler T, Mantyjarvi R. BDA20, a major bovine dander allergen characterized at the sequence level is Bos d 2. Submitted.
77. Gjesing B, Lowenstein H. Immunochemistry of food antigens. Ann Allergy 53:602, 1984.
78. de Groot, H., K. G. H. Goei, P. van Swieten, and R. C. Aalberse. 1991. Affinity purification of a major and a minor allergen from dog extract: Serologic activity of affinity-purified Can f I and Can f I-depleted extract. J. Allergy Clin. Immunol. 87:1056-1065.
79. Konieczny, A. Personal communication; Immunologic Pharmaceutical Corp.
79A. Bulone, V. 1998. Separation of horse dander allergen proteins by two-dimensional electrophoresis. Molecular characterisation and identification of Equ c 2.0101 and Equ c 2.0102 as lipocalin proteins. Eur J. Biochem 253:202-211.
79B. Swiss-Prot acc. P81216, P81217.
80. McDonald, B., M. C. Kuo, J. L. Oilman, and L. J. Rosenwasser. 1988. A 29 amino acid peptide derived from rat alpha 2 euglobulin triggers murine allergen specific human T-cells (abst). J. Allergy Clin. Immunol. 83:251.
81. Clarke, A. J., P. M. Cissold, R. A. Shawi, P. Beattie, and J. Bishop. 1984. Structure of mouse urinary protein genes: differential splicing configurations in the 3′-non-coding region. EMBO J. 3:1045-1052.
82. Longbottom, J. L. 1983. Characterization of allergens from the urines of experimental animals. McMillan Press, London, pp. 525-529.
83. Laperche, Y., K. R. Lynch, K. P. Dolans, and P. Feigelsen. 1983. Tissue-specific control of alpha 2u globulin gene expression: constitutive synthesis in submaxillary gland. Cell 32:453-460.
83A. Aukrust L, Borch S M. 1979. Partial purification and characterization of two Cladosporium herbarum allergens. Int Arch Allergy Appl Immunol 60:68-79.
83B. Sward-Nordmo M, Paulsen B S, Wold J K. 1988. The glycoprotein allergen Ag-54 (Cla h II) from Cladosporium herbarum. Structural studies of the carbohydrate moiety. Int Arch Allergy Appl Immunol 85:288-294.
84. Shen, et al. J. Allergy Clin. Immunol. 103:S157, 1999.
84A. Crameri R. Epidemiology and molecular basis of the involvement of Aspergillus fumigatus in allergic diseases. Contrib. Microbiol. Vol. 2, Karger, Basel (in press).
84B. Shen, et al. (manuscript submitted), 1999
84C. Shen H D, Ling W L, Tan M F, Wang S R, Chou H, Han S I H. Vacuolar serine proteinase: A major allergen of Aspergillus fumigatus. 10th International Congress of Immunology, Abstract, 1998.
85. Kumar, A., L. V. Reddy, A. Sochanik, and V. P. Kurup. 1993. Isolation and characterization of a recombinant heat shock protein of Aspergillus fumigatus. J. Allergy Clin. Immunol. 91:1024-1030.
86. Teshima, R., H. Ikebuchi, J. Sawada, S. Miyachi, S. Kitani, M. Iwama, M. Irie, M. Ichinoe, and T. Terao. 1993. Isolation and characterization of a major allergenic component (gp55) of Aspergillus fumigatus. J. Allergy Clin. Immunol. 92:698-706.
86A. Shen H D, Lin W L, Tsai J J, Liaw S F, Han S H. 1996. Allergenic components in three different species of Penicillium: crossreactivity among major allergens. Clin Exp Allergy 26:444-451.
86B. Shen, et al. Abstract; The XVIII Congress of the European Academy of Allergology and Clinical Immunology, Brussels, Belgium, 3-7 Jul. 1999.
87. Shen H D, Liaw S F, Lin W L, Ro L H, Yang H L, Han S H. 1995. Molecular cloning of cDNA coding for the 68 kd allergen of Penicillium notatum using MoAbs. Clin Exp Allergy 25:350-356.
88. Shen, H. D., K. B. Choo, H. H. Lee, J. C. Hsieh, and S. H. Han. 1991. The 40 kd allergen of Candida albicans is an alcohol dehydrogenease: molecular cloning and immunological analysis using monoclonal antibodies. Clin. Exp. Allergy 21:675-681.
89. Shen, et al. Clin. Exp. Allergy (in press), 1999.
90. Woodfolk J A, Wheatley L M, Piyasena R V, Benjamin D C, Platts-Mills T A. 1998. Trichophyton antigens associated with IgE antibodies and delayed type hypersensitivity. Sequence homology to two families of serine proteinases. J. Biol Chem 273:29489-96.
91. Deuell, B., L. K. Arruda, M. L. Hayden, M. D. Chapman and T. A. E. Platts-Mills. 1991. Trichophyton tonsurans Allergen I. J. Immunol. 147:96-101.
91A. Schmidt M, Zargari A, Holt P, Lindbom L, Hellman U, Whitley P, van der Ploeg I, Harfast B, Scheynius A. 1997. The complete cDNA sequence and expression of the first major allergenic protein of Malassezia furfur, Mal f 1. Eur J. Biochem 246:181-185.
91B. Horner W E, Reese G, Lehrer S B. 1995. Identification of the allergen Psi c 2 from the basidiomycete Psilocybe cubensis as a fungal cyclophilin. Ent Arch Allergy Immunol 107:298-300.
92. Kuchler, K., M. Gmachl, M. J. Sippl, and G. Kreil. 1989. Analysis of the cDNA for phospholipase A2 from honey bee venom glands: The deduced amino acid sequence reveals homology to the corresponding vertebrate enzymes. Eur. J. Biochem. 184:249-254.
93. Gmachl, M., and G. Kreil. 1993. Bee venom hyaluronidase is homologous to a membrane protein of mammalian sperm. Proc. Natl. Acad. Sci. USA 90:3569-3573.
94. Habermann, E. 1972. Bee and wasp venoms. Science 177:314-322.
95. Jacobson, R. S., and D. R. Hoffman. 1993. Characterization of bumblebee venom allergens (abst). J. Allergy Clin. Immunol. 91:187.
96. Arruda L K, Vailes L D, Mann B J, Shannon J, Fox J W, Vedvick T S, Hayden M L, Chapman M D. Molecular cloning of a major cockroach (Blattella germanica) allergen, Bla g 2. Sequence homology to the aspartic proteases. J. Biol Chem 270:19563-19568, 1995.
97. Arruda L K, Vailes L D, Hayden M L, Benjamin D C, Chapman M D. Cloning of cockroach allergen, Bla g 4, identifies ligand binding proteins (or calycins) as a cause of IgE antibody responses. J. Biol Chem 270:31196-31201, 1995.
98. Arruda L K, Vailes L D, Benjamin D C, Chapman M D. Molecular cloning of German Cockroach (Blattella germanica) allergens. Int Arch Allergy Immunol 107:295-297, 1995.
98A. Wu C H, Lee M F, Liao S C. 1995. Isolation and preliminary characterization of cDNA encoding American cockroach allergens. J. Allergy Clin Immunol 96: 352-9.
99. Mazur, G., X. Baur, and V. Liebers. 1990. Hypersensitivity to hemoglobins of the Diptera family Chironomidae: Structural and functional studies of their immunogenic/allergenic sites. Monog. Allergy 28:121-137.
100. Soldatova, L., L. Kochoumian, and T. P. King. 1993. Sequence similarity of a hornet (D. maculata) venom allergen phospholipase A1 with mammalian lipases. FEBS Letters 320:145-149.
101. Lu, G., L. Kochoumian and T. P. King. Whiteface hornet venom allergen hyaluronidase: cloning and its sequence similarity with other proteins (abst.). 1994. J. Allergy Clin. Immunol. 93:224.
102. Fang, K. S. F., M. Vitale, P. Fehhier, and T. P. King. 1988. cDNA cloning and primary structure of a white-faced hornet venom allergen, antigen 5. Proc. Natl. Acad. Sci., USA 85:895-899.
103. King, T. P., D. C. Moran, D. F. Wang, L. Kochoumian, and B. T. Chait. 1990. Structural studies of a hornet venom allergen antigen 5, Dol m V and its sequence similarity with other proteins. Prot. Seq. Data Anal. 3:263-266.
104. Lu, G., M. Villalba, M. R. Coscia, D. R. Hoffman, and T. P. King. 1993. Sequence analysis and antigen cross reactivity of a venom allergen antigen 5 from hornets, wasps and yellowjackets. J. Immunol. 150: 2823-2830.
105. King, T. P. and Lu, G. 1997. Unpublished data.
105A. King T P, Lu G, Gonzalez M, Qian N and Soldatova L. 1996. Yellow jacket venom allergens, hyaluronidase and phospholipase: sequence similarity and antigenic cross-reactivity with their hornet and wasp homologs and possible implications for clinical allergy. J. Allergy Clin. Immunol. 98:588-600.
106. Hoffman, D. R. 1993. Allergens in hymenoptera venom XXV: The amino acid sequences of antigen 5 molecules and the structural basis of antigenic cross-reactivity. J. Allergy Clin. Immunol. 92:707-716.
107. Hoffman, D. R. 1992. Unpublished data.
108. Hoffman, D. R. 1993. The complete amino acid sequence of a yellowjacket venom phospholipase (abst). J. Allergy Clin. Immunol. 91:187.
109. Jacobson, R. S., D. R. Hoffman, and D. M. Kemeny. 1992. The cross-reactivity between bee and vespid hyaluronidases has a structural basis (abst). J. Allergy Clin. Immunol. 89:292.
110. Hoffman, D. R. 1993. Allergens in Hymenoptera venom XXIV: The amino acid sequences of imported fire ant venom allergens Sol i II, Sol i III, and Sol i IV. J. Allergy Clin. Immunol. 91:71-78.
111. Schmidt, M., R. B. Walker, D. R. Hoffman, and T. J. McConnell. 1993. Nucleotide sequence of cDNA encoding the fire ant venom protein Sol i II. FEBS Letters 319:138-140.
112. Elsayed S, Bennich H. The primary structure of Allergen M from cod. Scand J Immunol 3:683-686, 1974.
113. Elsayed S, Aas K, Sletten K, Johansson S G O. Tryptic cleavage of a homogeneous cod fish allergen and isolation of two active polypeptide fragments. Immunochemistry 9:647-661, 1972.
114. Hoffman, D. R. 1983. Immunochemical identification of the allergens in egg white. J. Allergy Clin. Immunol. 71:481-486.
115. Langeland, T. 1983. A clinical and immunological study of allergy to hen's egg white. IV. specific IgE antibodies to individual allergens in hen's egg white related to clinical and immunological parameters in egg-allergic patients. Allergy 38:493-500.
116. Daul, C. B., M. Slattery, J. E. Morgan, and S. B. Lehrer. 1993. Common crustacea allergens: identification of B cell epitopes with the shrimp specific monoclonal antibodies. In: “Molecular Biology and Immunology of Allergens” (D. Kraft and A. Sehon, eds.). CRC Press, Boca Raton. pp. 291-293.
117. K. N. Shanti, B. M. Martin, S, Nagpal, D. D. Metcalfe, P. V. Subba Rao. 1993. Identification of tropomyosin as the major shrimp allergen and characterization of its IgE-binding epitopes. J. Immunol. 151:5354-5363.
117A. M. Miyazawa, H. Fukamachi, Y. Inagaki, G. Reese, C. B. Daul, S. B. Lehrer, S. Inouye, M. Sakaguchi. 1996. Identification of the first major allergen of a squid (Todarodes pacificus). J. Allergy Clin. Immunol. 98:948-953.
117B A. Lopata et al. 1997. Characteristics of hypersensitivity reactions and identification of a uniques 49 kd IgE binding protein (Hal-m-1) in Abalone (Haliotis midae). J. Allergy Clin. Immunol. Submitted
118. Monsalve, R. I., M. A. Gonzalez de la Pena, L. Menendez-Arias, C. Lopez-Otin, M. Villalba, and R. Rodriguez. 1993. Characterization of a new mustard allergen, Bra j IE. Detection of an allergenic epitope. Biochem. J. 293:625-632.
119. Mena, M., R. Sanchez-Monge, L. Gomez, G. Salcedo, and P. Carbonero. 1992. A major barley allergen associated with baker's asthma disease is a glycosylated monomeric inhibitor of insect alpha-amylase: cDNA cloning and chromosomal location of the gene. Plant Molec. Biol. 20:451-458.
120. Menendez-Arias, L., I. Moneo, J. Dominguez, and R. Rodriguez: 1988. Primary structure of the major allergen of yellow mustard (Sinapis alba L.) seed, Sin a I. Eur. J. Biochem. 177:159-166.
121. Gonzalez R, Varela J, Carreira J, Polo F. Soybean hydrophobic protein and soybean hull allergy. Lancet 346:48-49, 1995.
122. Christie, J. F., B. Dunbar, I. Davidson, and M. W. Kennedy. 1990. N-terminal amino acid sequence identity between a major allergen of Ascaris lumbricoides and Ascaris suum and MHC-restricted IgE responses to it. Immunology 69:596-602.
123. Czuppon A. B., Chen Z., Rennert S., Engelke T., Meyer H. E., Heber M, Baur X. The rubber elongation factor of rubber trees (Hevea brasiliensis) is the major allergen in latex. J. Allergy Clin Immunol 92:690-697, 1993.
124. Attanayaka D. P. S. T. G., Kekwick R. G. O., Franklin F. C. H.1991. Molecular cloning and nucleotide sequencing of the rubber elongation factor gene from hevea brasiliensis. Plant Mol Biol 16:1079-1081.
125. Chye M. L., Cheung K. Y. 1995. (1,3-glucanase is highly expressed in Laticifers of Hevea brasiliensis. Plant Mol Biol 26:397-402.
126. Alenius H., Palosuo T., Kelly K., Kurup V., Reunala T., Makinen-Kiljunen S., Turjanmaa K., Fink J. 1993. IgE reactivity to 14-kD and 27-kD natural rubber proteins in Latex-allergic children with Spina bifida and other congenital anomalies. Int Arch Allergy Immunol 102:61-66.
127. Yeang H. Y., Cheong K. F., Sunderasan E., Hamzah S., Chew N. P., Hamid S., Hamilton R. G., Cardosa M. J. 1996. The 14.6 kD (REF, Hey b 1) and 24 kD (Hey b 3) rubber particle proteins are recognized by IgE from Spina Bifida patients with Latex allergy. J. Allerg Clin Immunol in press.
128. Sunderasan E., Hamzah S., Hamid S., Ward M. A., Yeang H. Y., Cardosa M. J. 1995. Latex B-serum (-1,3-glucanase (Hey b 2) and a component of the microhelix (Hey b 4) are major Latex allergens. J nat Rubb Res 10:82-99.

1. A modified allergic allergen whose amino acid sequence is substantially identical to that of a natural allergen, which natural allergen includes at least one cysteine residue that participates in a disulfide bond when the natural allergen is in its native conformation, except that the at least one cysteine residue has been modified so that it cannot participate in the disulfide bond.

2. The modified allergic allergen of claim 1, being characterized in that, when contacted with serum IgE taken from an individual who is allergic to the natural allergic allergen, the modified allergic allergen shows reduced ability to bind IgE as compared with the natural allergic allergen.

3. The modified allergic allergen of claim 1, being characterized in that, when contacted with a pool of sera IgE taken from a group of at least two individuals that are allergic to the natural allergic allergen, the modified allergic allergen shows reduced ability to bind IgE as compared with the natural allergic allergen.

4. The modified allergic allergen of claim 1, being characterized in that, when contacted with a pool of sera IgE taken from a group of at least fifteen individuals that are allergic to the natural allergic allergen, the modified allergic allergen shows reduced ability to bind IgE as compared with the natural allergic allergen.

5. (canceled)

6. The modified allergic allergen of claim 1, wherein the at least one cysteine residue in the amino acid sequence of the natural allergic allergen has been modified by deletion at one or more cysteine residues.

7. The modified allergic allergen of claim 1, wherein the at least one cysteine residue in the amino acid sequence of the natural allergic allergen has been modified by substitution of one or more cysteine residues.

8. The modified allergic allergen of claim 7, wherein the at least one cysteine residue in the amino acid sequence of the natural allergic allergen has been substituted by a natural amino acid selected from the group consisting of serine, threonine, alanine, valine, glycine, leucine, isoleucine, histidine, tyrosine, phenylalanine, tryptophan, and methionine.

9-10. (canceled)

11. The modified allergic allergen of claim 1 identified or made by a process that includes steps of:
reducing at least one disulfide bond of a natural allergic allergen and subsequently capping at least one cysteine residue;
screening for IgE binding to the modified allergic allergen; and
selecting a modified allergic allergen with decreased binding to IgE as compared to the natural allergic allergen.

12. The modified allergic allergen of claim 1, wherein at least one cysteine residue in the amino acid sequence of the natural allergic allergen has been modified by a chemical means to an amino acid with a side chain having the chemical formula —CH₂—X wherein X is selected from the group consisting of SO₃⁻ and S—SO₃⁻.

13. The modified allergic allergen of claim 1 made by a process that includes steps of:
irreversibly oxidizing at least one disulfide bond of a natural allergic allergen;
screening for IgE binding to the modified allergic allergen; and
selecting a modified allergic allergen with decreased binding to IgE as compared to the natural allergic allergen.

14-40. (canceled)

41. The modified allergic allergen of claim 12 made by a process that includes steps of:
irreversibly oxidizing at least one disulfide bond of a natural allergic allergen;
screening for IgE binding to the modified allergic allergen; and
selecting a modified allergic allergen with decreased binding to IgE as compared to the natural allergic allergen.
